SEQUENCE LISTING (145 sequences)

SEQ ID NO: 1 - DNA, 1233 bp, synthetic construct
  source 1..1233: mol_type = other DNA
gccaccatgaagtggaaagtgtctgttctcgcctgcatcctccacgtgcggttcccaggagcagaggcacagagctttggtctgctggaccccaaactctgctacttgctagatggaatcctcttcatctacggagtcatcatcacagccctgtacctgagagcaaaattcagcaggagtgcagagactgctgccaacctgcaggaccccaaccagctcttcaatgagctcaatctagggcgaagagaggaatttgacgtcttggagaagaagcgggctcgggacccagagatgggaggcaaacagcagaggaggaggaacccccaggaaggcgtattcaatgcactgcagaaagacaagatggcagaagccttcagtgagatcggcacaaaaggcgagaggcggagaggcaaggggcacgatggccttttccagggtctcagcactgccaccaaggacacctttgatgccctgcatatgcagaccctggcccctcgcccacccgtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtataagtga

SEQ ID NO: 2 - protein, 32 aa, synthetic construct
MRVKEKYQHLWRWGWKWGTMLLGILMICSATE

SEQ ID NO: 3 - protein, 4 aa, synthetic construct
CAAL

SEQ ID NO: 4 - protein, 6 aa, synthetic construct
KKKKKK

SEQ ID NO: 5 - protein, 5 aa, synthetic construct
RRRRR

SEQ ID NO: 6 - protein, 21 aa, synthetic construct
MRLLLALLGVLLSVPGPPVLS

SEQ ID NO: 7 - protein, 12 aa, synthetic construct
CSIMNLMCGSTC

SEQ ID NO: 8 - protein, 45 aa, synthetic construct
GHKSEEKREKMKRTLLKDWKTRLSYFLQNSSTPGKPKTGKKSKQQ

SEQ ID NO: 9 - protein, 18 aa, synthetic construct
RSTLKLTTLQCQYSTVMD

SEQ ID NO: 10 - protein, 18 aa, synthetic construct
RQGLHNMEDVYELIENSH

SEQ ID NO: 11 - protein, 30 aa, synthetic construct
MDCRKMARFSYSVIWIMAISKVFELGLVAG

SEQ ID NO: 12 - protein, 16 aa, synthetic construct
MPAWGALFLLWATAEA

SEQ ID NO: 13 - protein, 4 aa, synthetic construct
RDYR

SEQ ID NO: 14 - protein, 55 aa, synthetic construct
KMALRVALNNKQSGQITVKTSSSDHLSLAIAGLVPIALSIYQKFKPGVSPSYSIY

SEQ ID NO: 15 - protein, 21 aa, synthetic construct
MGSKIVQVFLMLALFATSALA

SEQ ID NO: 16 - protein, 21 aa, synthetic construct
MNSKAMQALIFLGFLATSCLA

SEQ ID NO: 17 - protein, 12 aa, synthetic construct
MGAAASIQTTVN

SEQ ID NO: 18 - protein, 11 aa, synthetic construct
FALLGTHGASG

SEQ ID NO: 19 - protein, 7 aa, synthetic construct
RRRTFLK

SEQ ID NO: 20 - protein, 10 aa, synthetic construct
MGGKWSKSSV

SEQ ID NO: 21 - protein, 6 aa, synthetic construct
DDPERE

SEQ ID NO: 22 - protein, 16 aa, synthetic construct
EEANTGENNSLLHPMS

SEQ ID NO: 23 - protein, 6 aa, synthetic construct
SRRGLV

SEQ ID NO: 24 - protein, 6 aa, synthetic construct
SRRRFL

SEQ ID NO: 25 - protein, 6 aa, synthetic construct
SRRQFI

SEQ ID NO: 26 - protein, 6 aa, synthetic construct
QRRDFL

SEQ ID NO: 27 - protein, 52 aa, synthetic construct
MNKIYSIKYSAATGGLIAVSELAKKVICKTNRKISAALLSLAVISYTNIIYA

SEQ ID NO: 28 - protein, 40 aa, synthetic construct
MNPNQKIITIGSICMVIGIVSLMLQIGNIISIWVSHSIQT

SEQ ID NO: 29 - protein, 16 aa, synthetic construct
LRCLACSCFRTPVWPR

SEQ ID NO: 30 - protein, 10 aa, synthetic construct
MGCGCSSHPE

SEQ ID NO: 31 - protein, 9 aa, synthetic construct
MPFVNKQFN

SEQ ID NO: 32 - protein, 28 aa, synthetic construct
DEQNAKNAAQDRNSNKSSKGFFSKLGCC

SEQ ID NO: 33 - protein, 10 aa, synthetic construct
MLCCMRRTKQ

SEQ ID NO: 34 - protein, 14 aa, synthetic construct
VTNGSTYILVPLSH

SEQ ID NO: 35 - protein, 7 aa, synthetic construct
AETENFV

SEQ ID NO: 36 - protein, 18 aa, synthetic construct
RARHRRNVDRVSIGSYRT

SEQ ID NO: 37 - protein, 4 aa, synthetic construct
YEDQ

SEQ ID NO: 38 - protein, 19 aa, synthetic construct
LLVTSLLLCELPHPAFLIP

SEQ ID NO: 39 - protein, 164 aa, unidentified
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 40-41 - skipped (2 entries)

SEQ ID NO: 42 - DNA, 2217 bp, synthetic construct
  source 1..2217: mol_type = other DNA
atggccctgcccgtgaccgccctcctcctgccactcgccctgctgctgcatgcagctcggcctgacatccagatgacccagactacatcttccctgagcgcaagcctgggggatagggtgacaatatcctgccgggccagccaggatatcagcaaatacctgaactggtatcagcagaagcccgatggaaccgtgaagctgctcatctatcacacgagccggctgcactcaggcgtgcccagcaggttctctggctctggcagcggtactgattattcactgacaatcagcaatctggaacaggaggacatcgccacctacttctgccagcagggtaacaccctgccctacacttttggcgggggaaccaagctggagatcaccggcggcggcggcagcggcgggggaggctccggtgggggcgggtccgaggtgaagctgcaagagagcggccccggcctcgtggccccttctcagagcctgtccgtgacctgcactgtgtcaggagtgagcctgccagattatggggtgagctggatcagacagcccccccggaagggcctggagtggctgggcgtgatctggggtagcgagaccacatactataactctgccctgaaaagcagactgaccatcattaaggataacagcaagtctcaggtgttcctgaaaatgaactcccttcagacagacgacacagccatctattactgcgcaaaacactactactatgggggctcctacgctatggattattggggccaaggcaccagcgtgactgtgagcagtgactataaggatgacgacgataagaccggtacccctgctcctagacctcctacaccagctcctacaatcgccagccagcctctgtctctgaggccagaagcttgtagacctgctgctggcggagccgtgcatacaagaggactggatttcgcctgcgacttctgggtgctcgtggttgttggcggagtgctggcctgttacagcctgctggttaccgtggccttcatcatcttttgggtccgaagcaagcggagccggctgctgcacagcgactacatgaacatgacccctagacggcccggacctaccagaaagcactaccagccttacgctcctcctagagacttcgccgcctacagatctagagtgaagtttagccggagcgccgatgcccctgcctatcagcagggacagaaccagctgtataacgagctgaacctgggcaggagagaggagtacgatgtgctggataaacgccgcggaagagaccccgaaatgggcggcaagccacgccgcaaaaacccacaggagggcctttacaacgagctgcagaaggacaagatggccgaggcctactccgaaattggcatgaaaggggaacggcgcaggggaaagggccacgacggcctgtaccagggcctgtcaaccgccaccaaggatacctacgatgctctgcatatgcaggccctgccacctcgcccccctgtggctacaatggtgagtaagggcgaggagctgatcaaggagaatatgagaatgaaggtcgtgatggagggctccgtgaacggacaccagtttaagtgtacaggagagggggagggcagaccgtacgagggggtgcagaccatgcgcattaaggtgatcgagggcgggcctctgccctttgctttcgacatcctggcaaccagcttcatgtatggctccaggacatttatcaagtaccctgccgacatccccgattttttcaagcagagcttccccgagggcttcacctgggagagagtcacccgctacgaggacggaggggtggtgacagtgacacaggacaccagcctggaggatggagagctggtgtacaacgtcaaggtgcggggagtgaatttcccatcaaatggacccgtcatgcagaagaagaccaagggatgggagcctaatactgagatgatgtaccctgccgacggaggcctgaggggatacacagatatcgccctgaaggtggacgggggaggccatctgcattgcaatttcgtgaccacataccggagcaagaagaccgtcggcaacatcaagatgcccggcgtgcatgcagtggaccacaggctggagcggatcgaagagtctgataacgagacctacgtggtgcagagggaggtggccgtcgccaaatacagcaatctcggcgggggcatggacgagctgtacaagtaa

SEQ ID NO: 43-100 - skipped (58 entries)

SEQ ID NO: 101 - DNA, 2187 bp, synthetic construct
  source 1..2187: mol_type = other DNA
gccaccatgggagtgcctacacagctgctgggactgctgctgctgtggatcacagacgccatctgcgacatccagatgacacagagccctgccagcctgagcacatctctgggagagacagtgaccatccagtgccaggccagcgaggacatctactctggactggcctggtatcagcagaagcccggcaaatctccccagctgctgatctacggtgccagcgatctgcaggatggcgtgccatctagattcagcggctctggctctggcacccagtacagcctgaagatcaccagcatgcagaccgaggacgagggcgtgtacttctgtcagcaaggcctgacataccccagaaccttcggcggaggcaccaagctggaacttaaaggcggcggaggatctggcggaggtggaagtggcggaggcggatctgaagttcagctgcagcaatctggcgccgaactcgtcagacctggcacaagcgtgaagctgagctgcaaggtgtccggcgacaccatcaccttctactacatgcacttcgtgaagcagaggcccggacagggactcgagtggatcggaagaatcgaccctgaggacgagagcaccaagtactccgagaagttcaagaacaaggccacactgaccgccgacaccagcagcaacacagcctacctgaagctgtccagcctgacctccgaggacaccgccacctatttctgtatctacggcggctactacttcgactactggggccagggcgtgatggtcaccgtgtctagcactagtaccaccaccaagcctgtgctgagaaccccttctcctgtgcaccctaccggcacaagccagcctcaaagacctgaggactgcagacctagaggcagcgtgaaaggcacaggcctggacttcgcctgtgatttctgggctcttgtggtggtggctggcgtgctgttttgttacggcctgctggtcacagtggccctgtgtgtgatctggaccaacagcagaagaaacagactgctgcagagcgactacatgaacatgacccctagaaggcctggcctgaccagaaagccttaccagccttacgctcccgccagagatttcgccgcctacagaccaagagccaagttctccagaagcgccgagacagccgctaacctgcaggatcctaaccagctgtacaacgagctgaacctggggagaagagaagagtacgacgtgctggaaaagaagagagccagggatcctgagatgggcggcaagcagcagagaagaagaaaccctcaagaaggcgtgtacaacgccctgcagaaagacaagatggccgaggcctacagcgagatcggaactaagggcgaacgcagaagaggcaagggccacgatggactgtaccagggcctgtctacagccaccaaggacacatacgatgccctgcacatgcagacactggcccctagacctccagtgatgcatatggtgtccaagggcgaagaactgatcaaagaaaacatgcggatgaaggtggtcatggaaggctccgtgaacggccaccagttcaagtgtactggcgaaggcgagggcagaccctatgaaggcgtgcagaccatgagaatcaaagtgatcgaaggcggccctctgcctttcgcctttgacatcctggccaccagctttatgtacggcagcaggaccttcatcaagtaccccgctgacatccccgatttcttcaagcagagcttccccgagggcttcacatgggagagagtgacaagatacgaggacggcggcgtcgtgacagtgacacaggacacaagtctggaagatggcgagctggtgtacaatgtgaaagtgcggggcgtgaacttccccagcaacggacctgtgatgcagaaaaagaccaaaggctgggagcccaacaccgagatgatgtaccctgctgatggcggcctgagaggctacacagatatcgccctgaaagtggatggcggcggacatctgcactgcaacttcgtgaccacctacaggtccaagaaaaccgtgggcaacatcaagatgcccggggtgcacgccgtggaccacagactggaaagaatcgaggaaagcgacaacgagacatacgtggtgcagcgcgaagtggccgtggccaagtattctaaccttggaggcggcatggacgaactgtacaaataa

SEQ ID NO: 102 - DNA, 1578 bp, synthetic construct
  source 1..1578: mol_type = other DNA
gccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtataagggaccaggatcggcgtctggggaaggactgccaggatcagccggaccaggaatgcccgatcccgcggcgcatctgccattcttctatggcagcatctcgcgggctgaggccgaggagcacctgaagctggcaggcatggccgacgggctgttcctcctgcgccagtgtttgcgctccctgggcggctacgtgctgtcgttggtgcacgacgtgcgcttccaccatttccccatcgagcgccaactcaacggcacgtacgccatcgcgggcgggaaggcgcactgcggcccggccgagctctgccagttctactctcaggaccccgatgggctgccctgcaacctgcgtaagccgtgtaaccggccgcccggactggagccacagcccggggtcttcgactgcctgcgtgatgctatggtgtgcgactacgtgcgccagacctggaagctggagggcgatgcactagagcaggccatcatcagccaggccccacaggtggagaagctcattgctaccacagcccacgagcgaatgccctggtatcacagcagcctgactcgtgaggaggccgagcgcaaactctattccggccagcagaccgacggcaagttcctgctgaggccccggaaggagcagggcacatatgcactgtccctggtctatgggaaaactgtataccactatctcatcagccaggacaaggctggcaagtactgcatccccgaaggcaccaagtttgacacgctctggcagctggtggagtacctgaagctgaaggcggatggactaatttaccgcttgaaggaggtctgtcccaacagcagcgccccagggcaccatcatcatcatcattaa

SEQ ID NO: 103 - protein, 8 aa, synthetic construct
SIINFEKL

SEQ ID NO: 104 - protein, 11 aa, Human immunodeficiency virus 1
YGRKKRRQRRR

SEQ ID NO: 105 - protein, 16 aa, unidentified
RQIKIWFQNRRMKWKK

SEQ ID NO: 106 - protein, 9 aa, synthetic construct
RRRRRRRRR

SEQ ID NO: 107 - protein, 36 aa, synthetic construct
GPSQPTYPGDDAPVRDLIRFYRDLQRYLNVVTRHRY

SEQ ID NO: 108 - protein, 27 aa, unidentified
GWTLNSAGYLLGKINLKALAALAKKIL

SEQ ID NO: 109 - protein, 24 aa, Human alphaherpesvirus 1
DAATATRGRSAASRPTERPRAPAR

SEQ ID NO: 110 - protein, 21 aa, Simian virus 40
KETWWETWWTEWSQPKKKRKV

SEQ ID NO: 111 - protein, 18 aa, unidentified
VRLPPPVRLPPPVRLPPP

SEQ ID NO: 112 - protein, 22 aa, Homo sapiens
LGTYTQDFNKTFPQTAIGVGAP

SEQ ID NO: 113 - protein, 18 aa, Mus sp.
LLIILRRRIRKQAHAHSK

SEQ ID NO: 114 - protein, 236 aa, synthetic construct
NASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYA

SEQ ID NO: 115 - protein, 701 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCNHRNRRRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGGDYKDDDDKGSNASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYA

SEQ ID NO: 116 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 117 - protein, 408 aa, synthetic construct
MKWKVSVLACILHVRFPGAEAQSFGLLDPKLCYLLDGILFIYGVIITALYLRAKFSRSAETAANLQDPNQLFNELNLGRREEFDVLEKKRARDPEMGGKQQRRRNPQEGVFNALQKDKMAEAFSEIGTKGERRRGKGHDGLFQGLSTATKDTFDALHMQTLAPRPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 118 - protein, 10 aa, synthetic construct
MGCVCSSNPE

SEQ ID NO: 119 - protein, 254 aa, synthetic construct
MGCVCSSNPELPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 120 - protein, 407 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 121 - protein, 515 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGPGSASGEGLPGSAGPGMPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSA

SEQ ID NO: 122 - protein, 514 aa, synthetic construct
MGNASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAGPGSASGEGLPGSAGPGMPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSA

SEQ ID NO: 123 - protein, 413 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGGDYKDDDDKGSNASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYA

SEQ ID NO: 124 - protein, 607 aa, synthetic construct
MSNTVLADSAWGITLLSWVTVFLLGTSSADSGVVQSPRHIIKEKGGRSVLTCIPISGHSNVVWYQQTLGKELKFLIQHYEKVERDKGFLPSRFSVQQFDDYHSEMNMSALELEDSAMYFCASSRANYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSATNFSLLKQAGDVEENPGPMDKILTASFLLLGLHLAGVNGQQQEKRDQQQVRQSPQSLTVWEGETAILNCSYEDSTFNYFPWYQQFPGEGPALLISIRSVSDKKEDGRFTIFFNKREKKLSLHITDSQPGDSATYFCAASDNYQLIWGSGTKLIIKPDIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 125 - protein, 408 aa, synthetic construct
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 126 - protein, 515 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGPGSASGEGLPGSAGPGMPDPAAHLPFFYGSISRAEAEEHLKLAGMADGLFLLRQCLRSLGGYVLSLVHDVRFHHFPIERQLNGTYAIAGGKAHCGPAELCQFYSQDPDGLPCNLRKPCNRPPGLEPQPGVFDCLRDAMVCDYVRQTWKLEGDALEQAIISQAPQVEKLIATTAHERMPWYHSSLTREEAERKLYSGQQTDGKFLLRPRKEQGTYALSLVYGKTVYHYLISQDKAGKYCIPEGTKFDTLWQLVEYLKLKADGLIYRLKEVCPNSSA

SEQ ID NO: 127 - protein, 618 aa, synthetic construct
MANLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGDGPVGSENLYFQSGGGSMPDPAAHLPFFYGSISRAEAEEHLKLAGMADGLFLLRQCLRSLGGYVLSLVHDVRFHHFPIERQLNGTYAIAGGKAHCGPAELCQFYSQDPDGLPCNLRKPCNRPPGLEPQPGVFDCLRDAMVCDYVRQTWKLEGDALEQAIISQAPQVEKLIATTAHERMPWYHSSLTREEAERKLYSGQQTDGKFLLRPRKEQGTYALSLVYGKTVYHYLISQDKAGKYCIPEGTKFDTLWQLVEYLKLKADGLIYRLKEVCPNSSA

SEQ ID NO: 128 - protein, 514 aa, synthetic construct
MGNASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAGPGSASGEGLPGSAGPGMPDPAAHLPFFYGSISRAEAEEHLKLAGMADGLFLLRQCLRSLGGYVLSLVHDVRFHHFPIERQLNGTYAIAGGKAHCGPAELCQFYSQDPDGLPCNLRKPCNRPPGLEPQPGVFDCLRDAMVCDYVRQTWKLEGDALEQAIISQAPQVEKLIATTAHERMPWYHSSLTREEAERKLYSGQQTDGKFLLRPRKEQGTYALSLVYGKTVYHYLISQDKAGKYCIPEGTKFDTLWQLVEYLKLKADGLIYRLKEVCPNSSA

SEQ ID NO: 129 - protein, 247 aa, synthetic construct
MGCVCSSNPELPVATMVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 130 - protein, 692 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCNHRNRRRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 131 - protein, 571 aa, synthetic construct
METDTLLLWVLLLWVPGSTGGAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSTTTPAPRPPTPAPTIASQPLSLRPEACRPAAGGAVHTRGLDFACDIYIWAPLAGTCGVLLLSLVITLYCNHRNRRRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 132 - DNA, 1575 bp, synthetic construct
  source 1..1575: mol_type = other DNA
gccaccatgggcaacgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgccatggccagcaacttcacccagttcgtgctggtggacaacggcggcaccggcgacgtgaccgtggcccccagcaacttcgccaacggcatcgccgagtggatcagcagcaacagcagaagccaggcctacaaggtgacctgcagcgtgagacagagcagcgcccagaacagaaagtacaccatcaaggtggaggtgcccaagggcgcctggagaagctacctgaacatggagctgaccatccccatcttcgccaccaacagcgactgcgagctgatcgtgaaggccatgcagggcctgctgaaggacggcaaccccatccccagcgccatcgccgccaacagcggcatctacgccggaccaggatcggcgtctggggaaggactgccaggatcagccggaccaggaatgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccccagggcaccatcatcatcatcattaa

SEQ ID NO: 133 - DNA, 1248 bp, synthetic construct
  source 1..1248: mol_type = other DNA
gccaccatgaagtggaaggcgcttttcaccgcggccatcctgcaggcacagttgccgattacagaggcacagagctttggcctgctggaccccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtataatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtagcggtggtgattacaaggacgacgatgacaagggtagcaacgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgccatggccagcaacttcacccagttcgtgctggtggacaacggcggcaccggcgacgtgaccgtggcccccagcaacttcgccaacggcatcgccgagtggatcagcagcaacagcagaagccaggcctacaaggtgacctgcagcgtgagacagagcagcgcccagaacagaaagtacaccatcaaggtggaggtgcccaagggcgcctggagaagctacctgaacatggagctgaccatccccatcttcgccaccaacagcgactgcgagctgatcgtgaaggccatgcagggcctgctgaaggacggcaaccccatccccagcgccatcgccgccaacagcggcatctacgcctag

SEQ ID NO: 134 - DNA, 841 bp, synthetic construct
  source 1..841: mol_type = other DNA
gccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtataagtaaagcgccgcgtgactacggtacttattgccaagaaagcacgagcatcagccgtgcctccaggtcgaatcttcaaacgacgacgatcacgcgtcgctccagtattccagggttcatc

SEQ ID NO: 135 - DNA, 771 bp, synthetic construct
  source 1..771: mol_type = other DNA
gccaccatgggctgcgtgtgcagcagcaaccccgagctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 136 - DNA, 1230 bp, synthetic construct
  source 1..1230: mol_type = other DNA
gccaccatgaagtggaaggcgcttttcaccgcggccatcctgcaggcacagttgccgattacagaggcacagagctttggcctgctggaccccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtataatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcccacccgtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtataagtga

SEQ ID NO: 137 - DNA, 1824 bp, synthetic construct
  source 1..1824: mol_type = other DNA
atgtctaacactgtcctcgctgattctgcctggggcatcaccctgctatcttgggttactgtctttctcttgggaacaagttcagcagattctggggttgtccagtctccaagacacataatcaaagaaaagggaggaaggtccgttctgacgtgtattcccatctctggacatagcaatgtggtctggtaccagcagactctggggaaggaattaaagttccttattcagcattatgaaaaggtggagagagacaaaggattcctacccagcagattctcagtccaacagtttgatgactatcactctgaaatgaacatgagtgccttggaactggaggactctgctatgtacttctgtgccagctctcgggccaattatgaacagtacttcggtcccggcaccaggctcacggttttagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattccgccacgaacttctctctgttaaagcaagcaggagacgtggaagaaaaccccggtcctatggacaagatcctgacagcatcgtttttactcctaggccttcacctagctggggtgaatggccagcagcaggagaaacgtgaccagcagcaggtgagacaaagtccccaatctctgacagtctgggaaggagagaccgcaattctgaactgcagttatgaggacagcacttttaactacttcccatggtaccagcagttccctggggaaggccctgcactcctgatatccatacgttcagtgtccgataaaaaggaagatggacgattcacaatcttcttcaataaaagggagaaaaagctctccttgcacatcacagactctcagcctggagactcagctacctacttctgtgcagcaagtgacaactatcagttgatctggggctctgggaccaagctaattataaagccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtcctcctaa

SEQ ID NO: 138 - DNA, 1233 bp, synthetic construct
  source 1..1233: mol_type = other DNA
gccaccatgaagtggaaagtgtctgttctcgcctgcatcctccacgtgcggttcccaggagcagaggcacagagctttggtctgctggaccccaaactctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggacccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgcccacccgtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtataagtga

SEQ ID NO: 139 - DNA, 762 bp, synthetic construct
  source 1..762: mol_type = other DNA
tccggagccaccatgggctgcgtgtgcagcagcaaccccgagctacccgtcgccaccatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtataagtaaggatcc

SEQ ID NO: 140 - DNA, 2097 bp, synthetic construct
  source 1..2097: mol_type = other DNA
tccggagccaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagggatcc

SEQ ID NO: 141 - DNA, 1734 bp, synthetic construct
  source 1..1734: mol_type = other DNA
tccggagccaccatggagacagacacactcctgctatgggtgctgctgctctgggttccaggttccacaggtggcgcagaggcgggtatcaccgggacatggtacaaccaacacggaagcacatttacagtcaccgctggagcagacgggaatctgaccggacagtacgagaacagggctcaggggacaggttgtcagaacagtccgtatactctgactgggaggtacaatggcacgaagctggagtggcgagtcgagtggaataattccacggaaaactgtcacagtagaacagagtggaggggacagtaccaggggggagcagaggcccggatcaacacccaatggaacttgacatatgaaggcgggtcaggccccgcgacagagcaaggacaggatacattcacgaaggtcaagccaagcgcagcctctggctctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagggatcc

SEQ ID NO: 142 - DNA, 711 bp, synthetic construct
  source 1..711: mol_type = other DNA
aacgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgccatggccagcaacttcacccagttcgtgctggtggacaacggcggcaccggcgacgtgaccgtggcccccagcaacttcgccaacggcatcgccgagtggatcagcagcaacagcagaagccaggcctacaaggtgacctgcagcgtgagacagagcagcgcccagaacagaaagtacaccatcaaggtggaggtgcccaagggcgcctggagaagctacctgaacatggagctgaccatccccatcttcgccaccaacagcgactgcgagctgatcgtgaaggccatgcagggcctgctgaaggacggcaaccccatccccagcgccatcgccgccaacagcggcatctacgcctag

SEQ ID NO: 143 - DNA, 835 bp, synthetic construct
  source 1..835: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtataagtaaagcgccgcgtgactacggtacttattgccaagaaagcacgagcatcagccgtgcctccaggtcgaatcttcaaacgacgacgatcacgcgtcgctccagtattccagggttcatc

SEQ ID NO: 144 - DNA, 1887 bp, synthetic construct
  source 1..1887: mol_type = other DNA
gccaccatggctaatttactgaccgtacaccaaaatttgcctgcattacccgtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcgaatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagacatctcacgtactgacggtgggagaatgttaatccatattggcagaacgaaaacgctggttagcaccgcaggtgtggagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgatggacccgtcggtagcgaaaacctgtactttcagtccggtggtggtagcatgcccgatcccgcggcgcacctgccattcttctatggcagcatctcgcgggctgaggccgaggagcacctgaagctggcaggcatggccgacgggctgttcctcctgcgccagtgtttgcgctccctgggcggctacgtgctgtcgttggtgcacgacgtgcgcttccaccatttccccatcgagcgccaactcaacggcacgtacgccatcgcgggcgggaaggcgcactgcggcccggccgagctctgccagttctactctcaggaccccgatgggctgccctgcaacctgcgtaagccgtgtaaccggccgcccggactggagccacagcccggggtcttcgactgcctgcgtgatgctatggtgtgcgactacgtgcgccagacctggaagctggagggcgatgcactagagcaggccatcatcagccaggccccacaggtggagaagctcattgctaccacagcccacgagcgaatgccctggtatcacagcagcctgactcgtgaggaggccgagcgcaaactctattccggccagcagaccgacggcaagttcctgctgaggccccggaaggagcagggcacatatgcactgtccctggtctatgggaaaactgtataccactatctcatcagccaggacaaggctggcaagtactgcatccccgaaggcaccaagtttgacacgctctggcagctggtggagtacctgaagctgaaggcggatggactaatttaccgcttgaaggaggtctgtcccaacagcagcgccccagggcaccatcatcatcatcattaa

SEQ ID NO: 145 - DNA, 1575 bp, synthetic construct
  source 1..1575: mol_type = other DNA
gccaccatgggcaacgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgccatggccagcaacttcacccagttcgtgctggtggacaacggcggcaccggcgacgtgaccgtggcccccagcaacttcgccaacggcatcgccgagtggatcagcagcaacagcagaagccaggcctacaaggtgacctgcagcgtgagacagagcagcgcccagaacagaaagtacaccatcaaggtggaggtgcccaagggcgcctggagaagctacctgaacatggagctgaccatccccatcttcgccaccaacagcgactgcgagctgatcgtgaaggccatgcagggcctgctgaaggacggcaaccccatccccagcgccatcgccgccaacagcggcatctacgccggaccaggatcggcgtctggggaaggactgccaggatcagccggaccaggaatgcccgatcccgcggcgcacctgccattcttctatggcagcatctcgcgggctgaggccgaggagcacctgaagctggcaggcatggccgacgggctgttcctcctgcgccagtgtttgcgctccctgggcggctacgtgctgtcgttggtgcacgacgtgcgcttccaccatttccccatcgagcgccaactcaacggcacgtacgccatcgcgggcgggaaggcgcactgcggcccggccgagctctgccagttctactctcaggaccccgatgggctgccctgcaacctgcgtaagccgtgtaaccggccgcccggactggagccacagcccggggtcttcgactgcctgcgtgatgctatggtgtgcgactacgtgcgccagacctggaagctggagggcgatgcactagagcaggccatcatcagccaggccccacaggtggagaagctcattgctaccacagcccacgagcgaatgccctggtatcacagcagcctgactcgtgaggaggccgagcgcaaactctattccggccagcagaccgacggcaagttcctgctgaggccccggaaggagcagggcacatatgcactgtccctggtctatgggaaaactgtataccactatctcatcagccaggacaaggctggcaagtactgcatccccgaaggcaccaagtttgacacgctctggcagctggtggagtacctgaagctgaaggcggatggactaatttaccgcttgaaggaggtctgtcccaacagcagcgccccagggcaccatcatcatcatcattaa

(19) United States
(12) Patent Application Publication
(10) Pub. No.: US 2025/0059569 A1
(43) Pub. Date: Feb. 20, 2025

(54) COMPOSITIONS AND METHODS FOR DELIVERY OF THERAPEUTIC AGENTS TO ACCEPTOR CELLS

(71) Applicant: FLAGSHIP PIONEERING INNOVATIONS VI, LLC, Cambridge, MA (US)

(72) Inventors: Bradley Joseph Kubick, Watertown, MA (US); Douglas Ewen Cameron, Auburndale, MA (US); Christa Elyse Osuna, Newton, MA (US); Nicolas Cordero, Charlestown, MA (US); Edwin Xavier Arauz Diaz, Cambridge, MA (US); Syandan Chakraborty, Medford, MA (US); Lee John Flaherty, Somerville, MA (US); Alok Das Mohapatra, Farmington, CT (US); Pratibha Singh, Natick, MA (US)

(21) Appl. No.: 18/723,205

(22) PCT Filed: Dec. 21, 2022
(86) PCT No.: PCT/US2022/082165

§ 371 Date: Jun. 21, 2024

Related U.S. Application Data
(60) Provisional application No. 63/327,879, filed on Apr. 6, 2022.
(60) Provisional application No. 63/292,892, filed on Dec. 22, 2021.

(51) Int. Cl. C12N 15/90; A61K 39/00; C07K 14/725; C12N 5/0783; C12N 9/22; C12N 15/11

(52) U.S. Cl. CPC C12N 15/907; C07K 14/7051; C12N 5/0636; C12N 9/22; C12N 15/11; A61K 39/46; C12N 2310/20

(57) ABSTRACT

Compositions comprising donor cells, acceptor cells, and methods involving the same are described herein. In some embodiments, the donor cell is deficient in at least one endogenous function, for instance cytotoxic activity. In some embodiments, the donor cell comprises a T cell receptor (TCR) and a cargo, and the acceptor cell comprises an MHC. In some embodiments, the TCR facilitates transfer of the cargo to the acceptor cell. In some embodiments, the donor cell comprises a chimeric antigen receptor (CAR) and a cargo, and the acceptor cell comprises an antigen bound by the CAR. In some embodiments, the CAR facilitates transfer of the cargo to the acceptor cell.

### CROSS-REFERENCE TO RELATED APPLICATIONS

This application claims the benefit of U.S. Provisional Application Nos. 63/292,892, filed Dec. 22, 2021, and 63/327,879, filed Apr. 6, 2022. The contents of the aforementioned applications are hereby incorporated by reference in their entirety.

### BACKGROUND

A number of therapeutic strategies comprise modifying a subject's cells, either ex vivo (followed by returning said cells to the subject) or in vivo. However, delivery of modifications to only specific cells remains difficult, especially in vivo. There is a need for alternative methods for delivering therapeutics to cells.

### SUMMARY

The present disclosure relates to donor cells engineered to transfer a membrane-associated agent (e.g., a T cell receptor (TCR), a chimeric antigen receptor (CAR), or a functional fragment or variant thereof) and/or a cargo molecule, e.g., from a first membrane of the donor cell to a second membrane (e.g., of a target cell, e.g., an acceptor cell), as well as systems and compositions comprising the same, and methods of making and using the same. Such donor cells can be referred to herein, in some instances, as “cytografts.” Without wishing to be bound by theory, it is thought that natural processes in which a portion of the cell membrane of a cell is transferred to another cell, can be adapted as a method of modifying a cell (e.g., to express an exogenous protein, to alter expression of one or more genes, or alter a biological property of the acceptor cell).

In some aspects, the disclosure provides a donor cell (e.g., a cytograft as described herein) comprising (a) a membrane-associated agent comprising: (i) a membrane-associated moiety, (ii) a first docking moiety, and (iii) an intracellular moiety; and (b) a cargo molecule (e.g., an exogenous cargo molecule); wherein the donor cell is deficient in at least one endogenous function, or is T cell receptor (TCR)-deficient, or is MHC-deficient. In some aspects, the disclosure provides a donor cell (e.g., a cytograft as described herein) comprising: (a) a membrane-associated agent comprising: (i) a membrane-associated moiety, (ii) a first docking moiety, and (iii) an intracellular moiety; and (b) a cargo molecule (e.g., an exogenous cargo molecule); wherein the membrane-associated agent comprises a CAR or an exogenous TCR; and wherein the donor cell substantially lacks cytotoxic activity, e.g., wherein the donor cell is a regulatory T cell (Treg).

In some aspects, the disclosure provides a donor cell (e.g., a cytograft as described herein) comprising: (a) a membrane-associated agent comprising: (i) a membrane-associated moiety, (ii) a first docking moiety, and (iii) an intracellular moiety; and (b) a cargo molecule (e.g., an exogenous cargo molecule); wherein the donor cell has one or more (e.g., 1, 2, 3, 4, 5, 6, 7, 8, 9, or 10) of the following characteristics:
(i) the cargo molecule is attached to (e.g., covalently or noncovalently bound to) a cell-penetrating peptide;
(ii) the membrane-associated agent is attached to (e.g., covalently or noncovalently bound to) a cell-penetrating peptide;
(iii) the donor cell comprises a cell-penetrating peptide;
(iv) the donor cell further comprises a fusogen (e.g., a vsv-g protein, e.g., as described in Example 25);
(v) the donor cell expresses a secreted exogenous effector;
(vi) the donor cell comprises a virus-like particle (VLP), optionally wherein the VLP infects the acceptor cell;
(vii) the donor cell inserts a tunneling nanotube to an acceptor cell upon binding of the first docking moiety of the membrane-associated agent to a second docking moiety of the acceptor cell;
(vii) the donor cell comprises: (1) a nucleic acid molecule comprising a promoter (e.g., a tissue-specific promoter) and a gene encoding the membrane-associated agent and/or a gene encoding the cargo molecule; or (2) a nucleic acid molecule (e.g., an RNA, e.g., an mRNA) encoding the membrane-associated agent and/or a nucleic acid molecule (e.g., an RNA, e.g., an mRNA) encoding the cargo molecule;
(viii) the donor cell comprises an inducible cell death agent (e.g., an inducible cell death protein, e.g., an inducible caspase, e.g., inducible Caspase 9); and/or
(ix) the donor cell is or is derived from (e.g., differentiated from) an induced pluripotent stem cell (iPSC).

In some aspects, the disclosure provides a method of modifying an acceptor cell, the method comprising: contacting the acceptor cell with a donor cell (e.g., a cytograft as described herein) under conditions suitable for transfer of a membrane-associated agent and/or a cargo molecule to the acceptor cell; wherein the donor cell comprises:
(a) a membrane-associated agent comprising: (i) a membrane-associated moiety, (ii) a first docking moiety, and (iii) an intracellular moiety; and
(b) a cargo molecule (e.g., an exogenous cargo molecule);
wherein the donor cell is deficient in at least one endogenous function;
wherein the acceptor cell comprises a second docking moiety that binds specifically to the first docking moiety; and
wherein after the transfer the acceptor cell comprises an increased amount of the membrane-associated agent and/or cargo molecule; thereby modifying the acceptor cell.

In some aspects, the disclosure provides a method producing a donor cell (e.g., a donor cell according to any of the preceding claims, e.g., a cytograft as described herein), the method comprising: (i) inactivating at least one endogenous function of a cell; and (ii) introducing into or expressing in the cell:
(a) a membrane-associated agent comprising: (i) a membrane-associated moiety, (ii) a first docking moiety, and (iii) an intracellular moiety; and
(b) a cargo molecule (e.g., an exogenous cargo molecule); thereby producing a donor cell.

In some aspects, the disclosure provides a donor cell (e.g., a cytograft as described herein) comprising an membrane-associated agent (e.g., comprising a TCR, CAR, or a functional fragment or variant thereof), the agent comprising a membrane-associated moiety, and one or both of an extracellular moiety or an intracellular moiety, wherein the membrane-associated agent is configured to be transferred to an acceptor cell. In some embodiments, the donor cell comprises a cargo molecule configured to be transferred to an acceptor cell. In some embodiments, at least one of the membrane-associated moiety, extracellular moiety, intracellular moiety, or the cargo molecule is exogenous to the donor cell.

In another aspect, the disclosure provides a donor cell (e.g., a cytograft as described herein) comprising: a membrane-associated agent comprising a membrane-associated moiety, and an extracellular moiety, an intracellular moiety, a cargo molecule, or a combination thereof. In some embodiments, at least one of the membrane-associated moiety, extracellular moiety, intracellular moiety, or cargo molecule is present at a different level in the donor cell than a source cell (e.g., from which the donor cell was derived), e.g., is differentially expressed. In some embodiments, the membrane-associated agent is transferred to an acceptor cell.

In another aspect, the disclosure provides an acceptor cell comprising: a membrane-associated agent (e.g., comprising a TCR, CAR, or a functional fragment or variant thereof), e.g., after delivery of the membrane-associated agent to the acceptor cell by a donor cell, e.g., as described herein (e.g., a cytograft as described herein). In some embodiments, the acceptor cell does not comprise a nucleic acid encoding the membrane-associated agent. In some embodiments, the acceptor cell comprises a cargo molecule, e.g., received from a donor cell, e.g., as described herein. In some embodiments, at least one of the membrane-associated moiety, extracellular moiety, intracellular moiety, or cargo molecule is exogenous to the acceptor cell. In some embodiments, the acceptor cell comprises, e.g., has received, the membrane-associated agent and/or the cargo molecule from a donor cell.

In another aspect, the disclosure provides an acceptor cell comprising: a membrane-associated agent comprising: a membrane-associated moiety, and one or both of an extracellular moiety or an intracellular moiety. In some embodiments, the acceptor cell does not substantially express, e.g., does not express, a nucleic acid encoding the membrane-associated agent. In some embodiments, the acceptor cell comprises a cargo molecule, e.g., received from a donor cell. In some embodiments, the acceptor cell received the membrane-associated agent from the donor cell.

In another aspect, the disclosure provides a composition, e.g., a preparation, comprising a plurality of donor cells (e.g., cytografts) or acceptor cells described herein.

In another aspect, the disclosure provides a system, e.g., a reaction mixture, comprising: a donor cell described herein (e.g., a cytograft as described herein), and an acceptor cell, wherein the donor cell and acceptor cell are provided under conditions suitable for transfer of the membrane-associated agent and/or cargo molecule from the donor cell to the acceptor cell. In some embodiments, the acceptor cell does not comprise a nucleic acid encoding the membrane-associated agent and/or the cargo molecule, or differentially expresses the membrane-associated agent and/or cargo molecule, e.g., relative to an endogenously-expressed membrane-associated agent and/or cargo molecule, if any, or relative to the acceptor cell prior to transfer.

In another aspect, the disclosure provides a pharmaceutical composition comprising a donor cell (e.g., plurality of donor cells) described herein (e.g., a cytograft as described herein), an acceptor cell (e.g., plurality of acceptor cells), or a combination thereof. In some embodiments, the pharmaceutical composition comprises one or more pharmaceutically acceptable excipients or carriers.

In another aspect, the disclosure provides a method of modifying an acceptor cell, comprising: contacting the acceptor cell with a donor cell or system described herein (e.g., a cytograft as described herein), under conditions suitable for transfer of the membrane-associated agent and/or cargo molecule to the acceptor cell, thereby modifying the acceptor cell. In some embodiments, the acceptor cell does not comprise a nucleic acid encoding the membrane-associated agent and/or cargo molecule. In some embodiments, after the transfer the acceptor cell comprises an increased amount of the membrane-associated agent and/or cargo molecule.

In another aspect, the disclosure provides a method of making a modified cell, comprising: providing an unmodified cell, and contacting the unmodified cell with a donor cell or system described herein (e.g., a cytograft as described herein), under conditions suitable for transfer of the membrane-associated agent and/or cargo molecule to the unmodified cell, thereby making a modified cell. In some embodiments, neither the unmodified cell or modified cell comprise a nucleic acid encoding the membrane-associated agent. In some embodiments after the transfer the modified cell comprises an increased amount of the membrane-associated agent and/or cargo molecule than the unmodified cell. In some embodiments, neither the unmodified cell or modified cell comprise a nucleic acid encoding the membrane-associated agent and after the transfer the modified cell comprises an increased amount of the membrane-associated agent and/or cargo molecule than the unmodified cell.

In another aspect, the disclosure provides a method of delivering a cargo molecule to a cell, comprising: providing a donor cell or a system described herein (e.g., a cytograft as described herein), wherein the donor cell comprises comprising the cargo molecule; providing an acceptor cell that does not comprise a nucleic acid encoding the membrane-associated agent and/or cargo molecule or does not substantially express (e.g., does not express) a nucleic acid encoding the membrane-associated agent and/or cargo molecule; and contacting the acceptor cell with the donor cell or system under conditions suitable for transfer of the membrane-associated agent to the acceptor cell, thereby delivering the cargo molecule to the cell.

In another aspect, the disclosure provides a method of modulating, e.g., enhancing or decreasing, a biological function in a subject, a target tissue, or a cell, comprising administering to the subject, or contacting the target tissue or the cell with, a donor cell, acceptor cell, system, or a pharmaceutical composition described herein, thereby modulating the biological function in the subject.

Any of the aspects herein, e.g., the donor cells (e.g., cytografts), acceptor cells, compositions or preparations thereof, and methods above, can be combined with one or more of the embodiments herein, e.g., one or of the embodiments described herein.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Dec. 22, 2021. When one gene or protein references a plurality of sequence accession numbers, all of the sequence variants are encompassed. In addition, the materials, methods, and examples are illustrative only and not intended to be limiting.

### BRIEF DESCRIPTION OF THE DRAWINGS

The following detailed description of the invention will be better understood when read in conjunction with the appended drawings. For the purpose of illustrating the invention, there are shown in the drawings described herein certain embodiments, which are presently exemplified. It should be understood, however, that the invention is not limited to the precise arrangement and instrumentalities of the embodiments shown in the drawings.

FIGS. 1A and 1B show flow cytometry measurement of surface protein and DMF5 TCR. Mart-1 tetramer staining and biotinylated surface protein staining with streptavidin-AF750 are shown for T2 acceptors. A) shows T2 acceptors that were not pulsed with Mart-1 and B) shows T2 acceptors that were pulsed with Mart-1. Histograms show relative fluorescent intensity of each acceptor group (solid fill) versus the donor input (dotted outline).

FIG. 2 shows transfer quantification for a variety of co-culture inputs. Co-culture input combinations are shown in the far left column and resulting transfer phenotypes are summarized in the far right column. The middle columns show general surface protein transfer (streptavidin-AF750) and specific TCR transfer (MART-1 tetramer).

FIGS. 3A-3C show the results of a TCR transfer time course. T2 acceptors were pulsed with different peptides before co-culture with J76-DMF5 donors. (A) Acquisition of DMF5 TCR by acceptors are shown as a function of time in co-culture with J76-DMF5 cells. Percent of TCR positive cells are indicated in each tetramer-positive gate. (B) shows quantification of TCR acquisition and (C) shows amount of TCR transferred by mean fluorescent intensity (MFI).

FIG. 4 shows the results of a time course in which T2 acceptors were pulsed with different peptides before co-culture with J76-DMF5 donors. Activation (GFP) and DMF5 TCR (MART-1 tetramer) is shown for J76-DMF5 donor cells over time in co-culture.

FIG. 5 is a diagram showing exemplary approaches for transfer of RNA cargo via direct or indirect TCR fusions to an RNA-binding motif Left panel: Fusion of MCP RNA-binding domain to ZAP70 SH2 domains, which in turn bind to CD3 zeta. Right panel: Fusion of MCP RNA-binding domain to CD3 zeta.

FIG. 6 is a graph showing increase in GFP signal in peptide-pulsed conditions compared to no-peptide conditions for the Lck, CD3z, and ZAP70 versions of the MCP/EGFP mRNA construct.

FIG. 7A-7C is a series of diagrams showing cargo co-transfer with TCR via four different exemplary approaches. The transfer of EGFP fusion proteins was tested in the model established in examples 1 and 2. (A) J76-DMF5 donors were transfected with 4 different EGFP-expressing constructs versus mock transfection. (B) Flow cytometry measuring acquisition of DMF5 TCR and EGFP by acceptor T2 cells are shown with and without pre-pulsing the T2 cells with MART-1 peptide. (C) Quantification of the data in (B).

FIG. 8A-8B are a series of graphs showing that cargo transfer receptors were able to deliver cargo to target cells in vivo. (A) Detection of injected T cells in pancreatic islets by flow cytometry. (B) Transfer of OT-1 TCR to pancreatic β cells.

FIG. 9 is a diagram showing an exemplary construct expressing a CAR that specifically binds to CD19. The CAR comprises an extracellular domain comprising an scFv that binds to CD19 and an intracellular domain comprising a CD3 zeta signaling domain and a C-terminal mCherry fluorescent protein.

FIG. 10 is a graph showing transfer of CD19 CAR-mCherry to acceptor cells as the percentage of acceptor cells that exhibited mCherry signal, indicating acquisition of the CAR. Shown are the results for no-coculture negative control, coculture with J76-CAR donors, and coculture with both J76-CAR donors and blocking antibody.

FIG. 11 is a diagram showing an exemplary construct expressing a retargetable CAR comprising an intracellular cargo domain, a transmembrane domain, and an extracellular acceptor cell binding domain comprising a retargetable monovalent streptavidin mutant (mSA2). Shown is targeting of HLA-G using a biotinylated anti-HLA-G antibody.

FIG. 12 is a series of diagrams showing an exemplary strategy for co-transfer of RNA cargo using CARs. The left panel shows an exemplary CAR engineered to include an extracellular CD19-binding domain, a transmembrane domain, and an intracellular CD3 zeta domain attached to an MCP capable of binding to an MS2 RNA hairpin. The cargo is an EGFP-MS2 mRNA encoding EGFP and comprising an MS2 stem loop, as shown. The right panel shows the results of transfer of GFP cargo to Ramos cells in a mock setting, a CAR-MCP-only setting, a GFP-MS2 mRNA-only setting, and when GFP-MS2 mRNA was co-expressed with CAR-MCP.

FIG. 13 is a diagram showing exemplary approaches for cargo transfer to an acceptor cell expressing CD19 on its surface. Shown are a mock donor cell as well as specific transfer strategies for transfer of a cargo molecule. One transfer strategy utilizes a CD19 CAR fused to a reporter (mCherry). A second transfer strategy utilizes a truncated human ZAP70 containing only its SH2 domains, which is fused to a reporter (GFP). A third transfer strategy involves co-transfection with both the CD19 CAR and the ZAP70 constructs to transfer two reporter cargoes.

FIG. 14 is a graph showing transfer of CAR-mCherry or ZAP70-GFP constructs to CD19-expressing Ramos acceptor cells.

FIG. 15 is a series of diagrams showing an exemplary strategy for co-transfer of RNA cargo using CARs. The left panel shows a K562 donor cell expressing a CD19 CAR fused to mCherry and a ZAP70 SH2 domain fused to GFP, and a Ramos acceptor cell expressing CD19. The right panel shows the results of transfer of GFP and mCherry cargo to the Ramos cells in a mock setting, a CAR-mCherry-only setting, a ZAP70-GFP-only setting, and when ZAP70-GFP was co-epxressed with CAR-mCherry.

FIGS. 16A-16C are a series of diagrams showing specific transfer from donor cells to target acceptor cells present in a mixed population of cells. (A) Schematic showing four donor conditions, including mock electroporated J76 cells, J76 cells comprising a CD19 CAR fused to mCherry, J76 cells comprising a ZAP70-GFP fusion protein, and J76 cells comprising both the CD19 CAR-mCherry fusion and the ZAP70-GFP fusion. (B) Transfer of mCherry from each of the four donor cell conditions to various cell types in PBMCs, as indicated. (C) Transfer of GFP from each of the four donor cell conditions to various cell types in PBMCs, as indicated.

FIG. 17 is a series of graphs showing expression of a CAR shuttle in Tregs (right panel) compared to wild-type Tregs (left right), as determined by flow cytometry.

FIG. 18 is a diagram showing an exemplary chassis for a cytograft comprising an scFv-based membrane-associated agent (shuttle) comprising an extracellular anti-CD19 scFv, a transmembrane domain, an intracellular CD3-zeta domain, and an intracellular mRuby fluorescent protein. The cytograft also comprises a cargo molecule (GFP) attached to ZAP70 SH2, which binds to the CD3-zeta domain of the membrane-associated agent.

FIG. 19 is a series of graphs showing transfer of a CD19CAR-mRuby shuttle and EGFP effector from a cytograft to a target cell. ZAP70-GFP cargo was only transferred to target cells when the CD19 CAR shuttle was also present in the donor cell.

FIG. 20 is a series of diagrams showing transfer of GFP cargo from cytografts expressing CD19-CAR shuttles and a vsv-g fusogen to target HEK293T cells.

FIG. 21 is a series of graphs showing that transduced Tregs expressed CD19CAR-mRuby, as measured by mRuby expression.

FIG. 22 is a series of graphs showing acquisition of CD19CAR by Ramos cells from cytografts after co-culture.

FIG. 23 is a series of graphs showing cell viability, live cell number, and knockout efficiency in primary human CD3+ T cells for which the endogenous TCRα and TCRβ genes were knocked out using CRISPR.

FIG. 24 is a series of diagrams showing successful transfer of a GFP cargo to acceptor cells using, as a shuttle, a CD19 CAR-mRuby lacking a CD3-zeta domain.

FIG. 25 is a series of diagrams showing successful transfer of cargo to acceptor cells using, as a shuttle, a CAR containing a mono-streptavidin moiety (StrepCAR). This shuttle permits retargeting of the shuttle using biotinylated antibodies, so that a separate CAR is not needed for each target antigen.

FIG. 26 is a series of diagrams showing successful transfer of cargo to acceptor cells using, as a shuttle, a CAR targeting an MHC-presented antigen (i.e., a SIINFEKL peptide).

FIG. 27 is a series of diagrams showing successful transfer of cargo between a homotypic donor-acceptor pair. In particular, cargo was transferred to HEK 293T acceptor cells using HEK 293T donor cells comprising a vsv-g fusogen. A Cre recombinase was used to recombine a Cre reporter in the nucleus of acceptor cells in a fusogen-dependent manner.

FIG. 28 is a series of graphs showing successful transfer of cargo to acceptor cells, using primary human regulatory T cells (Tregs) as donor cells. The Treg donor cells have been transduced with lentiviruses to introduce a CD19 CAR-mRuby shuttle.

FIG. 29 is a diagram showing an exemplary structure for a CD19 CAR-mRuby complex comprising an extracellular region comprising an anti-human CD19 scFv and a Flag tag, a transmembrane region, and an intracellular region comprising mRuby3.

FIG. 30 is a diagram showing transfer of CAR to B cells in various tissues from recipient animals. Significant CAR transfer to B cells was observed in blood and lung from animals injected with hTreg-CAR donor cells. Transfer of CAR to B cells was also observed in blood and lung from animals that received J76-CAR (Clone 4) donor cells.

FIG. 31 is a graph showing detection of mRuby in tissues obtained from receipient animals, with the highest mRuby signal observed in animals that had received hTreg-CAR donor cells, and a lower level of mRuby observed in animals receiving J76-CAR (Clone 4) donor cells. Little or no mRuby signal was detected in animals that received J76 donor cells.

FIG. 32 is a series of graphs showing transfer incidence of CD19 CAR-mRuby into target cells after co-culture with wild-type adipocyte stem cells (ASCs) (left panel), or ASCs engineered to express CD19 CAR-mRuby (right panel).

FIGS. 33A-33D are a series of diagrams showing insertion of an exogenous CAR into a TRAC locus using CRISPR, thereby removing the endogenous TCR and inserting the exogenous CAR into a reliable genomic locus in a single.step.

FIGS. 34A-34B are a series of diagrams showing delivery of Flag-tagged CD19 CAR-mRuby mRNAs to primary regulatory T cells (Tregs), as measured by flow cytometry detection of Flag and mRuby signal.

### DETAILED DESCRIPTION

The invention describes donor cells, acceptor cells, membrane-associated agents, and related methods including methods of modifying acceptor cells, methods of delivering an membrane-associated agent and optionally one or more cargo molecules to an acceptor cell, methods of treating a subject, methods of transferring an membrane-associated agent from a donor cell to an acceptor cell, and methods of making said cells. Without wishing to be bound by theory, it is thought that a number of natural and artificially induced processes facilitate the transfer of membrane-associated agents from a first cell (e.g., a donor cell) to a second cell (e.g., an acceptor cell). Employing such processes to modify a feature of the second cell has a number of advantages over other cellular modification techniques. For example, such a method may not, in some instances, require genetic modification of the second cell (e.g., acceptor cell). A donor cell or acceptor cell comprising an membrane-associated agent or cargo molecule (e.g., a polypeptide, nucleic acid molecule, or other cargo molecule of interest) administered to a subject may be able to travel to a target cell, e.g., target tissue, e.g., target organ, in a subject more effectively than the agent or cargo molecule alone (e.g., delivering a higher amount of, a more specifically targeted amount of, or delivering with improved pharmacokinetics (e.g., a higher or lower half-life) the membrane-associated agent or cargo molecule).

### Definitions

As used herein, the term “acceptor cell” refers to a cell (e.g., a purified cell) capable of receiving a membrane-associated agent from a donor cell, membrane-associated body or membrane-enclosed body. In some embodiments, an acceptor cell is a cell within a tissue, e.g., within an organ, e.g., within a subject (e.g., a human subject). In some embodiments, an acceptor cell is a purified cell. In some embodiments, an acceptor cell does not comprise the membrane-associated agent (e.g., the acceptor cell has not yet received the membrane-associated agent). In some embodiments, an acceptor cell comprises the membrane-associated agent (e.g., having received the membrane-associated agent from a donor cell). An acceptor cell may comprise one or more modifications (e.g., in addition to the membrane-associated agent) relative to a source cell (e.g., from which the acceptor cell was derived), e.g., that enhance the acceptor cell's capability to receive a membrane-associated agent (e.g., from a donor cell). In some embodiments, an acceptor cell is capable of receiving a membrane-associated agent and/or a cargo molecule (e.g., as described herein) from a donor cell. In some embodiments, an acceptor cell is a cell within a tissue, e.g., within an organ, e.g., within a subject (e.g., a human subject). In some embodiments, an acceptor cell is a purified cell. In some embodiments, an acceptor cell does not comprise the membrane-associated agent (e.g., the acceptor cell has not yet received the membrane-associated agent). In some embodiments, an acceptor cell comprises the membrane-associated agent (e.g., having received the membrane-associated agent from a donor cell). An acceptor cell may, in some embodiments, comprise a modification (e.g., in addition to the membrane-associated agent) relative to a source cell (e.g., from which the acceptor cell was derived), e.g., that enhances the acceptor cell's capability to receive a membrane-associated agent (e.g., from a donor cell). In some embodiments, an acceptor cell comprises a docking moiety (e.g., an MHC (e.g., an HLA), e.g., bound to a peptide) specifically bound by a membrane-associated agent of a donor cell.

In general, the term “agent”, as used herein, may be used to refer to a compound or entity including, for example, a peptide, a polypeptide, a nucleic acid (e.g., DNA, a chromosome (e.g. a human artificial chromosome), RNA, mRNA, siRNA, miRNA), a saccharide or a polysaccharide, a lipid, a small molecule, or a combination or complex thereof. The term may refer to an entity that is or comprises an organelle, or a fraction, extract, or component thereof.

As used herein, the term “antibody molecule” refers to a protein, e.g., an immunoglobulin chain or a fragment thereof, comprising at least one immunoglobulin variable domain sequence. The term “antibody molecule” includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibody molecules can be monoclonal or polyclonal. The antibody molecule can also be a human, humanized, CDR-grafted, or in vitro generated antibody. The antibody molecule can have a heavy chain constant region chosen from, e.g., IgG1, IgG2, IgG3, or IgG4. The antibody molecule can also have a light chain chosen from, e.g., kappa or lambda. The term “immunoglobulin” (Ig) is used interchangeably with the term “antibody” herein.

As used herein, “cargo molecule” comprises an agent which may be delivered to a target cell (e.g., acceptor cell), or by an acceptor cell to another target cell (e.g., acceptor cell). The cargo molecule is: non-covalently associated with a component of the membrane-associated agent (e.g., an extracellular moiety, intracellular moiety, or membrane-associated moiety); or covalently associated with said a component of the membrane-associated agent (e.g., an extracellular moiety, intracellular moiety, or membrane-associated moiety) via a non-peptide bond; or not associated with the membrane-associated agent, e.g., the cargo molecule is operably associated or linked (e.g., tethered) to the membrane of a donor cell or membrane-enclosed body (e.g., separately from the membrane-associated agent). In some embodiments the cargo is neither membrane-associated nor associated with the membrane-associated agent. In some embodiments a cargo molecule comprises one or more a protein, e.g., an enzyme, a transmembrane protein, a receptor, or an antibody; a nucleic acid, e.g., a circular or linear nucleic acid, e.g., DNA, a chromosome (e.g. a human artificial chromosome), or RNA, e.g., mRNA, siRNA, miRNA, piRNA, or lncRNA; a lipid; or a small molecule (e.g., a signaling molecule (e.g., a second messenger) or a drug molecule). In some embodiments, a cargo is or comprises an organelle. In some embodiments, an agent may be delivered (e.g., from a donor cell) to a target cell (e.g., acceptor cell), e.g., in association with a membrane-associated agent. In some embodiments, the cargo molecule is non-covalently associated with a component of the membrane-associated agent (e.g., an extracellular moiety, intracellular moiety, or membrane-associated moiety). In some embodiments, the cargo molecule is covalently associated with a component of the membrane-associated agent (e.g., an extracellular moiety, intracellular moiety, or membrane-associated moiety) via a non-peptide bond. In some embodiments, the cargo molecule is not associated with the membrane-associated agent, e.g., the cargo molecule is operably associated or linked (e.g., tethered) to the membrane of a donor cell (e.g., separately from the membrane-associated agent).

As used herein, “configured to be transferred” refers to a status of an agent that is associated with a membrane of a first cell (e.g., donor cell), first membrane-containing substrate, or first membrane-enclosed body and capable of being transferred to a membrane of a second cell (e.g., acceptor cell), second membrane-containing substrate, or second membrane-enclosed body by a membrane transfer process upon contacting the first cell (e.g., donor cell), first membrane-containing substrate, or first membrane-enclosed body with the second cell (e.g., acceptor cell), second membrane-containing substrate, or second membrane-enclosed body.

As used herein the term “cognate peptide” refers to a peptide bound to (e.g., presented by) a major histocompatibility complex (MHC) molecule (e.g., an HLA molecule), which when bound to the MHC molecule, is capable of binding specifically to a TCR of interest (or a functional fragment or variant thereof), e.g., wherein the TCR is a membrane-associated agent, e.g., on the surface of a donor cell. In some embodiments, the MHC molecule does not substantially bind to the TCR of interest when the TCR not bound to the cognate peptide.

As used herein, a cell that is “deficient in at least one endogenous function” refers to a cell wherein an endogenous function was reduced or eliminated by artificial intervention, e.g., relative to an otherwise identical cell not subjected to the artificial intervention. The artificial intervention may be performed on the cell itself or on a parent cell from which the cell descends (e.g., wherein the cell is a daughter cell, granddaughter cell, or further generations relative to the parent cell). A cell may be made deficient in at least endogenous function by, for example, knockout, gene editing, or knockdown of a gene contributing to the endogenous function, expression or overexpression of a gene that inhibits the endogenous function, or introduction of an inhibitor of that the endogenous function into the cell, or introduction of an activator of a pathway that inhibits the endogenous function into the cell. The endogenous function may be, for example, an immune effector cell activity such as cytotoxic activity.

As used herein, a cell that is “deficient” for an endogenous protein or complex (e.g., TCR or MHC) refers to a cell that: a) does not substantially comprise (e.g., does not comprise detectable quantities of, e.g., does not comprise) the protein or a component of the complex (e.g., the cell comprises a gene encoding the protein, but does not express the gene); b) does not substantially comprise (e.g., does not comprise detectable quantities of, e.g., does not comprise e.g., does not comprise) a gene encoding the protein or a component of the complex; c) does not substantially comprise (e.g., does not comprise detectable quantities of, e.g., does not comprise e.g., does not comprise) a functional copy of the protein or a component of the complex (e.g., the cell comprises non-functional copies of the protein), or d) does not substantially comprise (e.g., does not comprise detectable quantities of, e.g., does not comprise e.g., does not comprise) a gene encoding a functional copy of the protein or a component of the complex. The protein may be non-functional because, for example, it comprises a mutation (e.g., an inactivating substitution, deletion, addition, truncation, insertion, and/or frameshift) or because it is not localized to the appropriate part of the cell. The protein may be non-functional because, for example, it lacks one or more of its normal biological functions, e.g., lacks all of its normal biological functions. As an example of a cell deficient for an endogenous complex, a TCR-deficient cell may, in some instances, be a cell that does not substantially comprise one or more components (e.g., TCRα or TCRβ) of the endogenous TCR complex, and/or comprises nonfunctional mutants of one or more components (e.g., TCRα or TCRβ) of the endogenous TCR complex. As another example, an MHC-deficient cell does not substantially comprise one or more components of the endogenous MHC complex. A cell can, in some embodiments, be deficient in an endogenous protein by naturally not expressing (e.g., silencing) a gene encoding the endogenous protein, naturally inhibiting the endogenous protein, or through artificial intervention. As an example, a regulatory T cell (Treg) can be TCR-deficient without artificial intervention.

As used herein, the term “donor cell” refers to a cell (e.g., a purified cell) comprising a membrane-associated agent configured to be transferred to a target cell, e.g., an acceptor cell. In some embodiments, a donor cell comprises one or more nucleic acids encoding the membrane-associated agent. In some embodiments, the donor cell comprises one or more modifications (e.g., in addition to the membrane-associated agent) relative to a source cell (e.g., from which the donor cell was derived), e.g., that enhance the donor cell's capability to transfer a membrane-associated agent to a target cell (e.g., an acceptor cell). In some embodiments, the donor cell comprises a cargo molecule to be transferred to the target cell, e.g., the acceptor cell.

As used herein, the term “exogenous” refers to an agent (e.g., a protein or lipid) that is not naturally found in a relevant system (e.g., a cell, a tissue, an organism, a source cell or a target cell, etc.). In embodiments, the agent is engineered and/or introduced into the relevant system. For example, in some embodiments, a donor cell, acceptor cell, or a membrane-enclosed preparation may be said to contain one or more “exogenous” lipids and/or proteins when the relevant lipids and/or proteins are not naturally found in a source cell from which the donor cell, acceptor cell, or membrane-enclosed preparation is obtained or derived (e.g., the source cell of the donor cell, acceptor cell, or membrane-enclosed. In some embodiments, an exogenous membrane-associated agent is or comprises a variant of an endogenous agent, such as, for example, a protein variant that differs in one or more structural aspects such as amino acid sequence, post-translational modification, etc from a reference endogenous protein, etc). In some embodiments, an exogenous protein is one that is expressed (e.g., overexpressed) from a transgene in the cell, wherein the cell may naturally express an endogenous protein having the same sequence as the exogenous protein.

As used herein, “membrane-associated agent” refers to an agent configured for transfer from a first cell (e.g., a donor cell) or membrane-enclosed body to a second cell (e.g., a target cell, e.g., an acceptor cell). A membrane-associated agent comprises a membrane-associated moiety and both of an extracellular moiety and an intracellular moiety. In some embodiments, a membrane-associated agent comprises more than one membrane-associated moiety, extracellular moiety or intracellular moiety. In some embodiments, at least one moiety (e.g., membrane-associated moiety, extracellular moiety, or intracellular moiety) of a membrane-associated agent is exogenous to 1) a donor comprising the membrane-associated agent, 2) the target cell, e.g., acceptor cell, to which the membrane-associated agent was or will be transferred, or 3) both. In some embodiments, an exogenous membrane-associated agent is a fusion protein. In some embodiments, a membrane-associated agent comprises a T cell receptor (TCR), chimeric antigen receptor (CAR), or a functional fragment or variant thereof. In some embodiments, a membrane-associated agent comprises a single molecule (e.g., a single polypeptide). In some embodiments, a membrane-associated agent comprises a complex (e.g., a complex comprising a plurality of polypeptide and/or nucleic acid subunits). In embodiments, a membrane-associated agent comprises a TCR complex, e.g., comprising one or more of: a TCR alpha molecule, a TCR beta molecule, a TCR gamma molecule, a TCR delta molecule, a CD3 zeta molecule, or any functional fragments or variants thereof. In embodiments, the membrane-associated agent is associated with (e.g., attached covalently or noncovalently to) a cargo molecule, e.g., as described herein.

As used herein, “membrane-associated moiety” refers to an agent that associates with (e.g., is localized in and/or on) or is capable of associating with a membrane (e.g., a cell membrane). In some embodiments, a membrane-associated moiety comprises a domain that at least partially (e.g., completely) spans a membrane, e.g., cell membrane. In some embodiments, a membrane-associated moiety is a transmembrane moiety that completely spans a membrane, e.g., cell membrane. In some embodiments a membrane-associated moiety is or comprises a transmembrane protein or the transmembrane domain of a transmembrane protein. In some embodiments, a membrane-associated moiety comprises a lipidation modification sequence, e.g., a N-myristoylation, N-palmitoylation, or S-palmitoylation sequence, or a hydrophobic signal sequence suitable for addition of Glycosylphosphatidylinositol (GPI), e.g., comprises a myristoyl, palmitoyl, or GPI modification. In some embodiments, a membrane-associated moiety is associated with an interior (e.g., cytosolic) portion of a membrane lipid bilayer. In some embodiments a membrane-associated moiety is associated with an exterior portion of a membrane lipid bilayer (e.g., with a cell surface or with a surface of a donor cell, acceptor cell, or a membrane-enclosed preparation as described herein). In some embodiments, a membrane-associated moiety is associated with an exterior portion of a membrane lipid bilayer and is or comprises a cell surface protein. In some embodiments a membrane-associated moiety is a naturally occurring protein. In some embodiments a membrane-associated moiety is an engineered and/or synthetic protein (e.g., a chimeric antigen receptor). In some embodiments a membrane-associated moiety is a therapeutic agent. In some embodiments, a membrane-associated moiety is operably associated or linked (e.g., tethered) to one or more of an intracellular moiety, an extracellular moiety, or a cargo molecule. In some embodiments, a membrane-associated moiety comprises a domain that is tethered to a membrane, e.g., to one leaflet of the membrane (e.g., the inner leaflet of a cell membrane).

As used herein, a “membrane transfer process” is any process capable of moving a portion of a membrane (e.g., one or more components of said membrane, e.g., a membrane-associated agent) from a first cell (e.g., donor cell) or membrane-enclosed body to a second cell (e.g., target cell, e.g., acceptor cell) or membrane-enclosed body when the first cell or membrane-enclosed body is in contact or close proximity with the second cell or membrane-enclosed body. Exemplary membrane transfer processes include, but are not limited to, a membrane fusion event, a receptor-ligand interaction, a cell bridging event (e.g., an antibody molecule (e.g., a bispecific antibody), or cell to cell contact event. Membrane transfer processes may adapt or use in part components or mechanisms of naturally occurring membrane transfer processes, e.g., trogocytosis or endocytosis.

As used herein, “operably associated” or “linked” refers to the state of two entities being connected in a functional way. For example, a promoter and a gene may both be situated on a nucleic acid: if they are operably linked, the promoter can promote expression of the gene (e.g., when the nucleic acid is appropriately situated in a cell, etc.); if they are not operably linked, the promoter cannot promote expression of the gene. In said example, operably linked encompasses the functional alignment of several details a skilled person would understand to be relevant to the functional connection of a promoter and a gene, e.g., the distance between the promoter and the gene, the reading frame of the gene, the position of the transcription start site, etc. As a further example, a membrane-associated moiety may be operably associated or linked with an extracellular moiety; such a status may imply that the moieties are part of a fusion protein wherein both moieties assume their native structures, provide any functions they are capable of, and/or the membrane-associated agent comprising said moieties is functional (e.g., configured for transfer or able to provide the functions of its component parts). As yet a further example, an intracellular moiety may be operably associated or linked with a cargo molecule, wherein the intracellular moiety is non-covalently associated with the cargo molecule and the intracellular moiety and cargo molecule are, e.g., able to assume their native structures, provide any functions they are capable of, and/or the membrane-associated agent comprising said moieties is functional (e.g., configured for transfer or able to provide the functions of its component parts). In some embodiments, operably associated or linked comprises a non-covalent interaction. In some embodiments, operably associated or linked comprises a covalent interaction, e.g., a peptide bond or a linker.

As used herein, the term “target cell moiety” is used to refer to a feature of a target cell (e.g., an acceptor cell) which may be used to specifically (relative to at least one other cell in the relevant system) target a donor cell or membrane-enclosed body to the cell. In some embodiment, a target cell moiety may be used to promote transfer of a membrane-associated agent and optionally one or more cargo molecules from a donor cell or membrane-enclosed body to a target cell, e.g., acceptor cell. In some embodiments, a target cell moiety is a feature of a target cell (e.g., an acceptor cell) which may be used to specifically (relative to at least one other cell in the relevant system) target a donor cell to the cell. In some embodiments, a target cell moiety comprises an MHC molecule (e.g., an HLA molecule). In some embodiments, a target cell moiety comprises a peptide (e.g., a cognate peptide relative to a TCR comprised in a membrane-associated agent of a donor cell), e.g., presented by the MHC. In some embodiments, a target cell moiety comprises a moiety specifically bound by a CAR comprised by the donor cell.

As used herein, the term “targeting domain” is an agent (e.g., a polypeptide) which associates or interacts with (e.g., binds) a target cell moiety. In some embodiments, a target cell moiety comprises an MHC/cognate peptide complex on the surface of a target cell, e.g., an acceptor cell. In some embodiments, a targeting domain is a docking moiety of a membrane-associated agent, e.g., as described herein. In some embodiments, a targeting domain (e.g., a docking moiety of a membrane-associated agent) comprises an antibody molecule (e.g., an antibody or a functional fragment or variant thereof, e.g., an scFv).

A “TCR molecule,” as used herein, refers to a polypeptide capable of complexing with other TCR molecules to form a TCR. In some embodiments, the TCR molecule comprises the sequence of a TCR alpha, a TCR beta, a TCR gamma, a TCR delta, or a CD3 zeta, or a functional fragment or variant thereof. In some embodiments, a TCR molecule is a wild-type protein, e.g., a wild-type protein from a mammal (e.g., a human). In some embodiments, a TCR molecule is engineered (e.g., a non-natural and/or modified TCR molecule, e.g., a mutated TCR molecule or a fusion protein). In some embodiments, a TCR molecule is a functional fragment or variant of a corresponding wild-type TCR protein.

As used herein, the term “therapeutic agent” refers to an agent capable of exerting a therapeutic effect on a subject when contacted with an acceptor cell (e.g., when transferred from a donor cell to an acceptor cell) in the subject. In some embodiments, the therapeutic agent comprises a polypeptide (e.g., a peptide or a protein).

### Cells

The present disclosure is directed, in part, to cells (e.g., donor cells or acceptor cells) comprising a membrane-associated agent and optionally one or more cargo molecules. In some embodiments, a donor cell described herein can be used to deliver a membrane-associated agent and optionally one or more cargo molecules to target cell, e.g., an acceptor cell. In some embodiments, a cell (e.g., a source cell, donor cell, or acceptor cell) is a purified cell, e.g., isolated from a culture, sample (e.g., apheresis sample), tissue, organ, or subject. In some embodiments, a cell (e.g., an acceptor cell) is part of a sample (e.g., apheresis sample), tissue, organ, or subject.

In some embodiments, the cell is a donor cell comprising a membrane-associated agent configured to be transferred to a target cell, e.g., an acceptor cell, and optionally comprising one or more cargo molecules also configured to be transferred to said target cell, e.g., an acceptor cell. In some embodiments, the donor cell is an immune cell (e.g., a T cell or an NK cell). In some embodiments, the donor cell is an autologous T cell or NK cell obtained from a patient, e.g., modified to comprise a membrane-associated agent (e.g., a TCR, CAR, or a functional fragment or variant thereof) and/or a cargo molecule, e.g., as described herein. In some embodiments, the donor cell is a T cell or NK cell from a cell line, e.g., a Jurkat cell. In some embodiments, the donor cell is a mesenchymal stromal cell. In some embodiments, the donor cell is a mesenchymal stem cell.

In some embodiments, the cell is an acceptor cell comprising a membrane-associated agent and optionally comprising one or more cargo molecules, wherein the acceptor cell does not comprise a nucleic acid encoding the membrane-associated agent or cargo molecule(s). In some embodiments, the acceptor cell received said membrane-associated agent and optionally one or more cargo molecules from a donor cell. In some embodiments, the acceptor cell is an antigen-presenting cell (APC), e.g., a professional APC (e.g., a dendritic cell, macrophage, or B cell) or a non-professional APC. In some embodiments, the acceptor cell comprises an MHC molecule bound to a peptide (e.g., a cognate peptide, e.g., wherein the MHC/cognate peptide complex is specifically bound by a membrane-associated agent, e.g., on a donor cell).

In some embodiments, the cell is an acceptor cell capable of receiving a membrane-associated agent and optionally one or more cargo molecules from a donor cell. In some embodiments, said acceptor cell does not comprise a membrane-associated agent or cargo molecule (e.g., yet). In some embodiments, the acceptor cell comprises one or more modifications increasing the efficacy and/or likelihood of receiving a membrane-associated agent or cargo molecule from a donor cell.

A cell (e.g., donor cell or acceptor cell) may be a naturally occurring cell, e.g., a mammalian cell, e.g., a human cell. In some embodiments, a donor cell is a naturally occurring cell modified to comprise a membrane-associated agent and optionally one or more cargo molecules. In some embodiments, an acceptor cell is a naturally occurring cell modified to comprise one or more modifications increasing the efficacy and/or likelihood of receiving a membrane-associated agent or cargo molecule from a donor cell.

In some embodiments, a donor cell or acceptor cell is derived from a source cell. In some embodiments, deriving said cell from a source cell comprising modifying the source cell, e.g., to contain or express a membrane-associated agent and optionally one or more cargo molecules, or to comprise one or more modifications increasing the efficacy and/or likelihood of receiving a membrane-associated agent or cargo molecule from a donor cell.

In some embodiments, DNA in the donor cell or acceptor cell or DNA in the source cell from which the aforementioned is derived is edited using a gene editing technology, e.g. a guide RNA and CRISPR-Cas9/Cpf1, or using a different targeted endonuclease (e.g., Zinc-finger nucleases, transcription-activator-like nucleases (TALENs)). Non-limiting examples of edits to DNA include small insertions/deletions, large deletions, gene corrections with template DNA, or large insertions of DNA. In some embodiments, gene editing is accomplished with non-homologous end joining (NHEJ) or homology directed repair (HDR). In some embodiments, the edit is a knockout. In some embodiments, the edit is a knock-in. In some embodiments, both alleles of DNA are edited. In some embodiments, a single allele is edited. In some embodiments, multiple edits are made. In some embodiments, the donor cell (or source cell from which the donor cell is derived) is derived from a subject, or is genetically matched to the subject, or is immunologically compatible with the subject (e.g. having similar MHC).

In some embodiments, the cell (e.g., source cell, donor cell, or acceptor cell) is a stem cell, red blood cell, white blood cell, neutrophil, eosinophil, basophil, lymphocyte, platelet, nerve cell, neuroglial cell, muscle cell (e.g., skeletal, cardiac, or smooth muscle cell), cartilage cell, bone cell (e.g., osteoclast, osteoblast, or osteocyte), lining cell, skin cell, endothelial cell, epithelial cell, fat cell, or sex cell (e.g., spermatozoa or ova).

In some embodiments, the cell (e.g., source cell, donor cell, or acceptor cell) is a primary cell or an immortalized cell, e.g., a cell line (e.g., a human cell line). In some embodiments, the cell (e.g., source cell, donor cell, or acceptor cell) is chosen from an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeLa, HEK293, HFF-1, MRC-5, WI-38, IMR 90, IMR 91, PER.C6, HT-1080, or BJ cell). In some embodiments, the cell (e.g., source cell, donor cell, or acceptor cell) is other than a 293 cell, HEK cell, human endothelial cell, or a human epithelial cell, monocyte, macrophage, dendritic cell, or stem cell. In some embodiments, the cell (e.g., source cell, donor cell, or acceptor cell) is a white blood cell or a stem cell. In some embodiments, the cell (e.g., source cell, donor cell, or acceptor cell) is selected from a neutrophil, a lymphocyte (e.g., a T cell, a B cell, or a natural killer (NK) cell), a macrophage, a granulocyte, a mesenchymal stem cell, a bone marrow stem cell, an induced pluripotent stem cell, an embryonic stem cell, or a myeloblast.

In some embodiments, the cell (e.g., donor cell or acceptor cell) is a synthetic cell. In some embodiments, the cell (e.g., donor cell or acceptor cell) is a recombinant cell, e.g., a cell comprising a non-naturally occurring nucleic acid sequence.

In some embodiments, the cell (e.g., source cell, donor cell, or acceptor cell) is an immune effector cell or is derived from an immune effector cell. In some embodiments, an immune effector cell refers to a cell that is involved in an immune response, e.g., in the promotion of an immune effector response. Examples of immune effector cells include, but are not limited to, T cells, e.g., CD4+ and CD8+ T cells, alpha/beta T cells and gamma/delta T cells, B cells, natural killer (NK) cells, natural killer T (NKT) cells, and mast cells.

In some embodiments, the membrane-associated agent or cargo molecule is present, per cell (e.g., donor cell or acceptor cell), at a copy number of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies, e.g., as measured by quantitative flow cytometry. In some embodiments, the membrane-associated agent or cargo molecule is present at a copy number of at least 1,000 copies, e.g., as measured by quantitative flow cytometry. In some embodiments, at least 10%, 20%, 30%, 40%, 50%, 60%, 70%, 80%, 90%, 95%, 96%, 97%, 98%, or 99% of the membrane-associated agent or cargo molecule comprised by the cell (e.g., donor cell or acceptor cell) is disposed in the cell membrane. In embodiments, the cell (e.g., donor cell or acceptor cell) also comprises cargo molecule internally, e.g., in the cytoplasm or an organelle.

In some embodiments, the cell (e.g., donor cell or acceptor cell) comprises a therapeutic agent at a copy number per cell of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies, e.g., as measured by quantitative flow cytometry. In some embodiments, the cell (e.g., donor cell or acceptor cell) comprises a protein therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies, e.g., as measured by quantitative flow cytometry. In some embodiments, the cell (e.g., donor cell or acceptor cell) comprises a nucleic acid therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cell (e.g., donor cell or acceptor cell) comprises a DNA therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cell (e.g., donor cell or acceptor cell) comprises an RNA therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cell (e.g., donor cell or acceptor cell) comprises a therapeutic agent that is exogenous relative to the source cell at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cell (e.g., donor cell or acceptor cell) comprises a protein therapeutic agent that is exogenous relative to the source cell at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cell (e.g., donor cell or acceptor cell) comprises a nucleic acid (e.g., DNA or RNA) therapeutic agent that is exogenous relative to the source cell at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the ratio of the copy number of the membrane-associated agent to the copy number of the therapeutic agent is between 1,000,000:1 and 100,000:1, 100,000:1 and 10,000:1, 10,000:1 and 1,000:1, 1,000:1 and 100:1, 100:1 and 50:1, 50:1 and 20:1, 20:1 and 10:1, 10:1 and 5:1, 5:1 and 2:1, 2:1 and 1:1, 1:1 and 1:2, 1:2 and 1:5, 1:5 and 1:10, 1:10 and 1:20, 1:20 and 1:50, 1:50 and 1:100, 1:100 and 1:1,000, 1:1,000 and 1:10,000, 1:10,000 and 1:100,000, or 1:100,000 and 1:1,000,000. In some embodiments, the ratio of the copy number of the membrane-associated agent to the copy number of the cargo molecule is between 1,000,000:1 and 100,000:1, 100,000:1 and 10,000:1, 10,000:1 and 1,000:1, 1,000:1 and 100:1, 100:1 and 50:1, 50:1 and 20:1, 20:1 and 10:1, 10:1 and 5:1, 5:1 and 2:1, 2:1 and 1:1, 1:1 and 1:2, 1:2 and 1:5, 1:5 and 1:10, 1:10 and 1:20, 1:20 and 1:50, 1:50 and 1:100, 1:100 and 1:1,000, 1:1,000 and 1:10,000, 1:10,000 and 1:100,000, or 1:100,000 and 1:1,000,000.

In some embodiments, the cell (e.g., donor cell) delivers to a target cell (e.g., acceptor cell) at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a therapeutic agent. In some embodiments, the cell (e.g., donor cell) delivers to a target cell (e.g., acceptor cell) at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a protein therapeutic agent. In some embodiments, the cell (e.g., donor cell) delivers to a target cell (e.g., acceptor cell) at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a nucleic acid therapeutic agent. In some embodiments, the cell (e.g., donor cell) delivers to a target cell (e.g., acceptor cell) at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of an RNA therapeutic agent. In some embodiments, the cell (e.g., donor cell) delivers to a target cell (e.g., acceptor cell) at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies of a DNA therapeutic agent.

In some embodiments, the cell (e.g., donor cell) delivers to a target cell (e.g., acceptor cell) at least 10%, 20%, 30%, 40%, 50%, 60%, 70%, 80%, 90%, 95%, 96%, 97%, 98%, or 99% of a membrane-associated agent or cargo molecule (e.g., a therapeutic agent, e.g., a therapeutic agent that is endogenous or exogenous relative to the source cell) comprised by the cell (e.g., donor cell) delivers. In some embodiments, the cells (e.g., donor cell) that interact with the target cell(s) (e.g., acceptor cell) deliver to the target cell an average of at least 10%, 20%, 30%, 40%, 50%, 60%, 70%, 80%, 90%, 95%, 96%, 97%, 98%, or 99% of the membrane-associated agent or cargo molecule comprised by the cells (e.g., donor cell) that interact with the target cell(s). In some embodiments, the donor cell composition delivers to a target tissue (e.g., comprising a plurality of target cells, e.g., acceptor cells) at least 10%, 20%, 30%, 40%, 50%, 60%, 70%, 80%, 90%, 95%, 96%, 97%, 98%, or 99% of the membrane-associated agents or cargo molecules comprised by the donor cell composition.

In some embodiments, a provided cell (e.g., donor cell or acceptor cell), and/or compositions or preparations thereof, comprise 0.00000001 mg exogenous membrane-associated agent or cargo molecule to 1 mg exogenous membrane-associated agent or cargo molecule per mg of total protein in the cell, e.g., 0.00000001-0.0000001, 0.0000001-0.000001, 0.000001-0.00001, 0.00001-0.0001, 0.0001-0.001, 0.001-0.01, 0.01-0.1, or 0.1-1 mg exogenous membrane-associated agent or cargo molecule per mg of total protein in the cell. In some embodiments, a provided cell (e.g., donor cell or acceptor cell), and/or compositions or preparations thereof, comprises 0.00000001 mg exogenous membrane-associated agent or cargo molecule to 5 mg exogenous membrane-associated agent or cargo molecule per mg of lipid in the cell, e.g., 0.00000001-0.0000001, 0.0000001-0.000001, 0.000001-0.00001, 0.00001-0.0001, 0.0001-0.001, 0.001-0.01, 0.01-0.1, 0.1-1, or 1-5 mg exogenous membrane-associated agent or cargo molecule per mg of lipid in the cell.

In some embodiments, provided cells (e.g., donor cells or acceptor cells), and/or compositions or preparations thereof, meet a pharmaceutical or good manufacturing practices (GMP) standard. In some embodiments, provided cells (e.g., donor cells or acceptor cells), and/or compositions or preparations thereof, were made according to good manufacturing practices (GMP). In some embodiments, provided cells (e.g., donor cells or acceptor cells), and/or compositions or preparations thereof, are characterized by a pathogen level below a predetermined reference value, e.g., are substantially free of pathogens. In some embodiments, provided cells (e.g., donor cells or acceptor cells), and/or compositions or preparations thereof, have a contaminant (e.g., nuclear component such as nuclear DNA) level below a predetermined reference value, e.g., are substantially free of one or more specified contaminants. In some embodiments, provided cells (e.g., donor cells or acceptor cells), and/or compositions or preparations thereof, are characterized by low immunogenicity, e.g., as described herein.

In some embodiments, a donor cell comprises a membrane-associated agent and optionally a cargo molecule configured for transfer to an acceptor cell. In some embodiments, the donor cell comprises a T cell and the acceptor cell comprises an NK cell. In some embodiments, the donor cell comprises a dendritic cell and the acceptor cell comprises a T cell. In some embodiments, the donor cell comprises a dendritic cell and the acceptor cell comprises a NK cell. In some embodiments, the donor cell comprises a T cell and the acceptor cell comprises a T cell. In some embodiments, the membrane-associated agent and/or cargo molecule comprises a T cell Receptor (TCR), e.g., as described herein. In some embodiments, the membrane-associated agent and/or cargo molecule comprises a CAR, e.g., as described herein.

In some embodiments, a T cell (e.g., a T cell to be modified to produce a donor cell as described herein) has endogenous cytotoxic activity. Cytotoxic activity of a T cell generally includes the ability to selectively destroy a target cell recognized by the T cell. In naturally occurring systems, the target cell is often a cell virus-infected cell with a pathogen (e.g., a virus) or a cancer cell. Cytotoxic activity by the T cell can include induction of apoptosis in the target cell and use of cytotoxic granules (e.g., comprising perforin and/or granzymes). Cytotoxicity can be assessed, for example, by a ⁵¹Cr-release assay, e.g., as described in paragraph 701 of International Application WO2015/142675. In some embodiments, a method described herein reduces the cytotoxic activity of a cell (e.g., T cell) to less than 90%, 80%, 70%, 60%, 50%, 40%, 30%, 20%, or 10% of that of an otherwise similar, unmodified cell, e.g., as measured using an assay described herein.

In some embodiments, a donor cell comprises a membrane-associated agent and optionally a cargo molecule configured for transfer to an acceptor cell. In some embodiments, the donor cell is a T cell and the acceptor cell is a cancer cell.

In some embodiments, a donor cell comprises a membrane-associated agent and optionally a cargo molecule configured for transfer to an acceptor cell. In some embodiments, the donor cell is a dendritic cell and the acceptor cell is a cell that has been transplanted into a subject (e.g., as part of a tissue or organ transplant).

In some embodiments, a donor cell comprises a membrane-associated agent and optionally a cargo molecule configured for transfer to an acceptor cell. In some embodiments, the donor cell is a progenitor cell and the acceptor cell is a differentiated cell. In some embodiments, the membrane-associated agent and/or cargo molecule comprises a reprogramming factor.

In some embodiments, a donor cell comprises a receptor (e.g., a TCR, CAR, or a functional fragment or variant thereof) and an acceptor cell comprises a ligand of said receptor (e.g., an MHC presenting a cognate peptide, wherein the MHC/cognate peptide complex is specifically bound by the TCR). In some embodiments, a donor cell comprises a receptor (e.g., a CAR) and an acceptor cell comprises a ligand of said receptor (e.g., a molecule comprising an epitope recognized by the first docking moiety of the CAR). In some embodiments, a membrane-associated agent comprises the receptor, or a functional portion thereof, e.g., as part of an extracellular moiety (e.g., a targeting domain) or membrane-associated moiety. In some embodiments, a targeting domain (e.g., not operably associated with a membrane-associated agent or cargo molecule) comprises the receptor, the ligand, or a functional portion thereof.

### Donor Cells

The present disclosure provides, in an aspect, a donor cell comprising: (a) a membrane-associated agent comprising: (i) a membrane-associated moiety, (ii) a first docking moiety, and (iii) an intracellular moiety; and (b) a cargo molecule (e.g., an exogenous cargo molecule). In some embodiments, the donor cell is deficient in at least one endogenous function, or is T cell receptor (TCR)-deficient, or is MHC-deficient. In some embodiments, the membrane-associated agent comprises a CAR or an exogenous TCR and the donor cell substantially lacks cytotoxic activity, e.g., the donor cell is a regulatory T cell (Treg). In some embodiments, the donor cell has one or more (e.g., 1, 2, 3, 4, 5, 6, 7, 8, 9, or 10) of the following characteristics:
(i) the cargo molecule is attached to (e.g., covalently or noncovalently bound to) a cell-penetrating peptide;
(ii) the membrane-associated agent is attached to (e.g., covalently or noncovalently bound to) a cell-penetrating peptide;
(iii) the donor cell comprises a cell-penetrating peptide;
(iv) the donor cell further comprises a fusogen (e.g., a vsv-g protein, e.g., as described in Example 25);
(v) the donor cell expresses a secreted exogenous effector;
(vi) the donor cell comprises a virus-like particle (VLP), optionally wherein the VLP infects the acceptor cell;
(vii) the donor cell inserts a tunneling nanotube to an acceptor cell upon binding of the first docking moiety of the membrane-associated agent to a second docking moiety of the acceptor cell;
(vii) the donor cell comprises: (1) a nucleic acid molecule comprising a promoter (e.g., a tissue-specific promoter) and a gene encoding the membrane-associated agent and/or a gene encoding the cargo molecule; or (2) a nucleic acid molecule (e.g., an RNA, e.g., an mRNA) encoding the membrane-associated agent and/or a nucleic acid molecule (e.g., an RNA, e.g., an mRNA) encoding the cargo molecule;
(viii) the donor cell comprises an inducible cell death agent (e.g., an inducible cell death protein, e.g., an inducible caspase, e.g., inducible Caspase 9); and/or
(ix) the donor cell is or is derived from (e.g., differentiated from) an induced pluripotent stem cell (iPSC) or hematopoietic stem cell (HSC).

In some embodiments, a donor cell is deficient in at least one endogenous function. In certain embodiments, the donor cell is deficient in cytotoxic activity (e.g., exhibits decreased cytotoxic activity or does not exhibit substantial cytotoxic activity), e.g., compared to an otherwise similar cell of the same type (e.g., a T cell).

In some embodiments, a donor cell is T cell receptor (TCR)-deficient. In certain embodiments, the donor cell comprises a knockout of one or more TCR genes (e.g., as described herein, e.g., TCRα or TCRβ). In embodiments, the one or more TCR genes are knocked out using CRISPR. In embodiments, the one or more TCR genes are knocked out using a transposase (e.g., a Sleeping Beauty transposase). In certain embodiments, the donor cell comprises a knockdown of one or more TCR genes (e.g., as described herein, e.g., TCRα or TCRβ). In certain embodiments, the donor cell comprises a mutant (e.g., an inactivating mutant, e.g., a substitution, deletion, truncation, insertion, or frameshift) of one or more TCR genes (e.g., as described herein, e.g., TCRα or TCRβ). In some embodiments, an exogenous nucleic acid sequence (e.g., an exogenous nucleic acid sequence encoding a membrane-associated agent or a cargo molecule, e.g., as described herein) is knocked-in to a TCR gene (e.g., as described herein, e.g., TCRα or TCRβ) of the donor cell.

In some embodiments, a donor cell is MHC-deficient. In certain embodiments, the donor cell comprises a knockout of one or more MHC genes (e.g., as described herein). In embodiments, the one or more MHC genes are knocked out using CRISPR. In embodiments, the one or more MHC genes are knocked out using a transposase (e.g., a Sleeping Beauty transposase). In certain embodiments, the donor cell comprises a knockdown of one or more MHC genes (e.g., as described herein). In certain embodiments, the donor cell comprises a mutant (e.g., an inactivating mutant, e.g., a substitution, deletion, truncation, insertion, or frameshift) of one or more MHC genes (e.g., as described herein). In some embodiments, an exogenous nucleic acid sequence (e.g., an exogenous nucleic acid sequence encoding a membrane-associated agent or a cargo molecule, e.g., as described herein) is knocked-in to a MHC gene (e.g., as described herein) of the donor cell.

In some embodiments, a donor cell is immortalized. In certain embodiments, one or more immortalization genes are introduced into the donor cell (e.g., via a lentivirus). In embodiments, the one or more immortalization genes include, without limitation, hTERT, SV40 Large T antigen, CDK4, and/or HPV16-E6/E7.

In some embodiments, a donor cell comprises a membrane-associated agent configured to be transferred to a target cell, e.g., an acceptor cell, and optionally one or more cargo molecules also configured to be transferred to the target cell (e.g., the acceptor cell).

In some embodiments, a donor cell or the source cell used to make a donor cell is a T cell, a B cell, an NK cell, or a cell derived from any thereof. In some embodiments, the donor cell is a T cell. In some embodiments, the donor cell is an NK cell.

In some embodiments, a donor cell or source cell used to make a donor cell is a cell from a cell line (e.g., an immortalized cell line), e.g., Jurkat, K562, Ramos, HUVEC, 293T, RAW264.7, BT-474, SK-BR-3, MDA-MB-231, or BT-20 cell (e.g., as available from ATCC).

In some embodiments, a donor cell is a T cell or is derived from a T cell. In some embodiments, a donor cell is an effector T cell or is derived from an effector T cell. In some embodiments, the donor cell does not substantially exhibit T cell effector activity. In some embodiments, the intracellular moiety of the membrane-associated agent does not stimulate (e.g., promote, activate, or increase) T cell effector activity. In some embodiments, T cell effector activity includes targeted killing of another cell, e.g., upon recognition of an antigen on the other cell. In some embodiments, T cell effector activity includes release of effector molecules (e.g., cytokines or tumor necrosis factors).

In some embodiments, a donor cell is derived from a Jurkat cell. In some embodiments, a donor cell is derived from J76 cell. In some embodiments, a donor cell is derived from a K562 cell. In some embodiments, a donor cell is derived from a PBMC. In some embodiments, a donor cell is derived from a JEG-3 cell. In some embodiments, a donor cell is derived from a THP1 cell. In some embodiments, a donor cell is derived from a hepatocyte (e.g., a primary hepatocyte). In some embodiments, a donor cell is derived from a primary cell (e.g., a primary T cell, e.g., as described in the following section).

In some embodiments, a donor cell is capable of transferring a membrane-associated agent (e.g., a TCR, a CAR, or a functional fragment or variant thereof) and optionally one or more cargo molecules to at least two, at least three, at least four, at least five, or more (e.g., any or all) types of acceptor cells. In some embodiments, a donor cell is capable of transferring the membrane-associated agent and optionally one or more cargo molecules to a specific acceptor cell type (e.g., and not to other acceptor cell types). In some embodiments, a donor cell is capable of transferring the membrane-associated agent and optionally one or more cargo molecules to a specific acceptor cell type and not to at least one, two, three, four, five, or more (e.g., all other) types of cells.

In some embodiments, donor cells, and/or compositions or preparations thereof, are characterized by a half-life in a subject, e.g., in a mouse, that is within 1%, 2%, 3%, 4%, 5%, 10%, 20%, 30%, 40%, 50%, 60%, 70%, 80%, 90%, 100% of the half life of a reference cell, e.g., the source cell. In some embodiments, donor cells, and/or compositions or preparations thereof, are characterized by a half-life in a subject, e.g., in a mouse, that is at least 1 hour, 2 hours, 3 hours, 4 hours, 5 hours, 6 hours, 12 hours, or 24 hours, e.g., in a human subject or in a mouse. In some embodiments, donor cells, and/or compositions or preparations thereof, are capable of delivering (e.g., deliver) a exogenous membrane-associated agent or cargo molecule (e.g., a therapeutic agent) that is characterized by a half-life in a subject that is longer than the half-life of the donor cell, e.g., by at least 10%, 20%, 50%, 2-fold, 5-fold, or 10-fold. For instance, the donor cell may deliver the therapeutic agent to the target cell (e.g., acceptor cell), and the therapeutic agent may be present after the donor cell is no longer present or detectable.

In some embodiments, a characteristic of a provided cell (e.g., donor cell or acceptor cell), and/or of a composition or preparations thereof, is described by comparison to a reference cell. In embodiments, the reference cell is the source cell from which the donor cell, or acceptor cell was derived. In embodiments, the reference cell is a Huvec, K562, THP-1, Jurkat, KHYG-1, Ramos, PBMC, and isolated PBMC subset cell. In some embodiments, a characteristic of a population of donor cells, acceptor cells, and/or of a composition or preparation thereof, is described by comparison to a population of reference cells, e.g., a population of source cells, or a population of Huvec, K562, THP-1, Jurkat, KHYG-1, Ramos, PBMC, and isolated PBMC subset cell.

In some embodiments, a donor cell comprises a T cell receptor (TCR), e.g., as described herein.

In some embodiments, a donor cell (e.g., a donor cell comprising a CAR, e.g., as described herein) does not comprise a TCR. In some embodiments, a donor cell does not substantially express a TCR. In some embodiments, the level of TCRs in a donor cell is reduced (e.g., by at least 50%, 60%, 70%, 80%, 90%, 95%, 96%, 97%, 98%, 99%, or 100%) relative to a source cell from which the donor cell is derived. In some embodiments, a native TCR gene of the donor cell has been knocked out.

Donor Cells Generated from Primary Cells

Compositions of donor cells may be generated from primary source cells, for example primary mammalian cells, e.g., primary human cells or primary murine cells. The cells may be progenitor cells or non-progenitor (e.g., differentiated) cells. In some embodiments, the primary cells are T cells. In embodiments, the primary cells are CD3+ T cells, CD4+ T cells, or CD8+ T cells. In an embodiment, the primary cells are CD4+/CD25+ T cells. In embodiments, the primary cells are regulatory T cells (Tregs). In embodiments, the primary cells are natural killer (NK) cells. In embodiments, the primary donor T cells are used to target B cells (e.g., Ramos cells) as acceptor cells.

In some embodiments, a membrane-associated agent (e.g., as described herein) is introduced into the primary cells. In some embodiments, a nucleic acid molecule encoding a membrane-associated agent is introduced into the primary cells (e.g., by transfection, electroporation, or viral infection, e.g., using a lentivirus).

In some embodiments, a cargo molecule (e.g., as described herein) is introduced into the primary cells. In some embodiments, a nucleic acid molecule encoding a cargo molecule is introduced into the primary cells (e.g., by transfection, electroporation, or viral infection, e.g., using a lentivirus).

Donor Cells Generated from Culture Cells

Compositions of donor cells may be generated from source cells in culture, for example cultured mammalian cells, e.g., cultured human cells. The cells may be progenitor cells or non-progenitor (e.g., differentiated) cells. The cells may be primary cells or cell lines (e.g., a mammalian, e.g., human, cell line described herein).

In some embodiments, the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), an immortalized cell (e.g., HeLa, HEK293), or a Huvec, K562, THP-1, Jurkat, KHYG-1, Ramos, PBMC, or isolated PBMC subset cell. In some embodiments, the source cell is a Jurkat cell or a variant thereof.

The cultured cells may be from epithelial, connective, muscular, or nervous tissue or cells, and combinations thereof. Donor cells can be generated from cultured cells from any eukaryotic (e.g., mammalian) organ system, for example, from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof. In embodiments, the cells are from a highly mitotic tissue (e.g., a highly mitotic healthy tissue, such as epithelium, embryonic tissue, bone marrow, intestinal crypts). In embodiments, the tissue sample is a highly metabolic tissue (e.g., skeletal tissue, neural tissue, cardiomyocytes).

In some embodiments a donor cell (or a source cell used to derive the same) is a suspension cell. In some embodiments a donor cell (or a source cell used to derive the same) is an adherent cell.

In some embodiments, the a donor cell (or a source cell used to derive the same) are from a young donor, e.g., a donor 25 years, 20 years, 18 years, 16 years, 12 years, 10 years, 8 years of age, 5 years of age, 1 year of age, or less. In some embodiments, the donor cell (or a source cell used to derive the same) are from fetal tissue.

In some embodiments, the donor cells are derived from cells from a subject and administered to the same subject or a subject with a similar genetic signature (e.g., MHC-matched).

Donor cells may be generated from cells generally cultured according to methods known in the art. In some embodiments, the cells may be cultured in 2 or more “phases”, e.g., a growth phase, wherein the cells are cultured under conditions to multiply and increase biomass of the culture, and a “production” phase, wherein the cells are cultured under conditions to alter cell phenotype (e.g., to maximize mitochondrial phenotype, to increase number or diameter of mitochondria, to increase oxidative phosphorylation status). There may also be an “expression” phase, wherein the cells are cultured under conditions to maximize expression of exogenous membrane-associated agent, cargo molecules, or other agents exogenous relative to the source cell, on the cell membrane and to restrict transfer in other phases.

In some embodiments, donor cells are generated from cells synchronized, e.g., during a growth phase or the production phase. For example, cells may be synchronized at G1 phase by elimination of serum from the culture medium (e.g., for about 12-24 hours) or by the use in the culture media of DNA synthesis inhibitors such as thymidine, aminopterin, hydroxyurea and cytosine arabinoside. Additional methods for mammalian cell cycle synchronization are known and disclosed, e.g., in Rosner et al. 2013. Nature Protocols 8:602-626 (specifically Table 1 in Rosner).

In some embodiments, the cells can be evaluated and optionally enriched for a desirable phenotype or genotype for use as a source for donor cell composition as described herein. For example, cells can be evaluated and optionally enriched, e.g., before culturing, during culturing (e.g., during a growth phase or a production phase) or after culturing but before donor cell production, for example, for one or more of: membrane potential (e.g., a membrane potential of −5 to −200 mV; cardiolipin content (e.g., between 1-20% oftotal lipid); cholesterol, phosphatidylethanolamine (PE), diglyceride (DAG), phosphatidic acid (PA), or fatty acid (FA) content; genetic quality>80%, >85%, >90%; exogenous membrane-associated agent expression or content; or cargo molecule expression or content.

In some embodiments, donor cells are generated from a cell clone identified, chosen, or selected based on a desirable phenotype or genotype for use as a source for a donor cell composition described herein. For example, a cell clone is identified, chosen, or selected based on low mitochondrial mutation load, long telomere length, differentiation state, or a particular genetic signature (e.g., a genetic signature to match a recipient).

A donor cell composition described herein may be comprised of donor cells from one cellular or tissue source, or from a combination of sources. For example, a donor cell composition may comprise donor cells from xenogeneic sources (e.g., animals, tissue culture of the aforementioned species' cells), allogeneic, autologous, from specific tissues resulting in different protein concentrations and distributions (liver, skeletal, neural, adipose, etc.), from cells of different metabolic states (e.g., glycolytic, respiring). A composition may also comprise donor cells in different metabolic states, e.g. coupled or uncoupled, as described elsewhere herein.

In some embodiments, donor cells are generated from source cells expressing a membrane-associated agent (and optionally one or more cargo molecules), e.g., a membrane-associated agent described herein. In some embodiments, the membrane-associated agent is disposed in a membrane of the source cell, e.g., a lipid bilayer membrane, e.g., a cell surface membrane, or a subcellular membrane (e.g., lysosomal membrane). In some embodiments, donor cells are generated from source cells with a membrane-associated agent disposed in a cell surface membrane.

In some embodiments, donor cells are generated by inducing budding of an exosome, microvesicle, membrane vesicle, extracellular membrane vesicle, plasma membrane vesicle, giant plasma membrane vesicle, apoptotic body, mitoparticle, pyrenocyte, lysosome, or other membrane enclosed vesicle.

For avoidance of doubt, it is understood that in many cases the source cell actually used to make the donor cell will not be available for testing after the donor cell is made. Thus, a comparison between a source cell and a donor cell does not need to assay the source cell that was actually modified (e.g., enucleated) to make the donor cell. Rather, cells otherwise similar to the source cell, e.g., from the same culture, the same genotype same tissue type, or any combination thereof, can be assayed instead.

### Modifications to Cells Prior to Donor Cell Generation

In some aspects, a modification is made to a cell, such as modification of a subject, tissue or cell, prior to donor cell generation. Such modifications can be effective to, e.g., improve transfer of the membrane-associated agent (and optionally one or more cargo molecules), targeting of a target cell (e.g., acceptor cell), exogenous membrane-associated agent expression or activity, structure or function of the cargo molecule, or structure or function of the target cell.

In some embodiments, a modification is made to a source cell or donor cell derived therefrom to modulate which membrane-associated agents (e.g., membrane proteins (e.g., endogenous membrane proteins), receptors, ligands, or cell surface markers) are configured for transfer to a target cell (e.g., acceptor cell). Such a modification can decrease the level of one or more (e.g., all) membrane-associated agents that are configured to transfer. Without wishing to be bound by theory, such modification may be desirable to control which membrane-associated agents a donor cell is capable of transferring to a target cell (e.g., acceptor cell). Such modifications include, but are not limited to: decreasing (e.g., eliminating) expression of a membrane-associated agent (e.g., transiently or stably); altering localization of a membrane-associated agent (e.g., away from the cell membrane); and tethering a membrane-associated agent to an agent not configured to be transferred (e.g., linking a membrane-associated agent to a component of the cytoskeleton or an organelle).

### Physical Modifications

In some embodiments, a cell is physically modified prior to generating the donor cell. For example, as described elsewhere herein, a membrane-associated agent, one or more cargo molecules, and/or one or more targeting domains may be linked to the surface of the cell.

In embodiments, a donor cell, acceptor cell, comprises increased or decreased levels of an endogenous molecule. For instance, the donor cell, acceptor cell, may comprise an endogenous molecule that also naturally occurs in the naturally occurring source cell but at a higher or lower level than in the donor cell or acceptor cell. In some embodiments, the polypeptide is expressed from an exogenous nucleic acid in the source cell, donor cell, or acceptor cell. In some embodiments, the polypeptide is isolated from a source and loaded into or conjugated to a source cell, donor cell, or acceptor cell.

In some embodiments, a cell is treated with a chemical agent, e.g., small molecule, prior to generating the donor cell, acceptor cell, to increase the expression or activity of an endogenous agent, e.g., targeting domain, in the cell (e.g., in some embodiments, endogenous relative to the source cell, and in some embodiments, endogenous relative to the target cell). In some embodiments, a small molecule may increase expression or activity of a transcriptional activator of the endogenous agent (e.g., targeting domain). In some embodiments, a small molecule may decrease expression or activity of a transcriptional repressor of the endogenous agent (e.g., targeting domain). In some embodiments, a small molecule is an epigenetic modifier that increases expression of the endogenous agent (e.g., targeting domain).

In some embodiments, a source cell is physically modified with, e.g., CRISPR activators, prior to generating a donor cell to add or increase the concentration of exogenous membrane-associated agent, cargo molecule, or targeting domain.

In some embodiments, the cell is physically modified to increase or decrease the presence of an endogenous agent on the cell membrane. In some embodiments, the physical modification increases the level of an endogenous agent, e.g., targeting domain, on the cell membrane. In some embodiments, the physical modification decreases the level of other cell membrane components (e.g., cell membrane components that are not the membrane-associated agent, targeting domain, or cargo molecule) in the cell membrane. Without wishing to be bound by theory, it is thought that transfer of a membrane-associated agent and optionally one or more cargo molecules can be improved by decreasing the level of unnecessary or interfering endogenous agents on the cell membrane and/or increasing the level of endogenous agents that promote transfer (e.g., targeting domains) on the cell membrane.

### Genetic Modifications

In some embodiments, a cell is genetically modified prior to generating the donor cell to increase the expression of an endogenous agent (e.g., targeting domain) in the cell (e.g., endogenous relative to the source cell or endogenous relative to the target cell). In some embodiments, a genetic modification may increase expression or activity of a transcriptional activator of the endogenous agent (e.g., targeting domain). In some embodiments, a genetic modification may decrease expression or activity of a transcriptional repressor of the endogenous agent (e.g., targeting domain). In some embodiments the activator or repressor is a nuclease-inactive Cas9 (dCas9) linked to a transcriptional activator or repressor that is targeted to the endogenous agent or nucleic acid encoding the same by a guide RNA. In some embodiments, a genetic modification epigenetically modifies an endogenous agent-encoding gene to increase its expression. In some embodiments the epigenetic activator a nuclease-inactive Cas9 (dCas9) linked to an epigenetic modifier that is targeted to the endogenous agent by a guide RNA.

In some embodiments, a cell is genetically modified prior to generating the donor cell to increase the expression of a membrane-associated agent or cargo molecule in the cell, e.g., via delivery of a transgene. In some embodiments, a nucleic acid, e.g., DNA, mRNA or siRNA, is transferred to the cell prior to generating the donor cell, e.g., to increase or decrease the expression of a cell surface molecule (protein, glycan, lipid or low molecular weight molecule) used for organ, tissue, or cell targeting, e.g., a targeting domain. In some embodiments, the nucleic acid targets a repressor of a membrane-associated agent, targeting domain, or cargo molecule, e.g., an shRNA, siRNA construct. In some embodiments, the nucleic acid encodes an inhibitor of a membrane-associated agent, targeting domain, or cargo molecule repressor.

In some embodiments, the method comprises introducing a nucleic acid that is exogenous relative to the source cell into the source cell, wherein the nucleic acid encodes one or more of a membrane-associated agent, targeting domain, or cargo molecule. The exogenous nucleic acid may be, e.g., DNA or RNA. In some embodiments the exogenous nucleic acid may be e.g., a DNA, a gDNA, a cDNA, an RNA, a pre-mRNA, an mRNA, an miRNA, an siRNA, etc. In some embodiments, the exogenous DNA may be linear DNA, circular DNA, or an artificial chromosome. In some embodiments the DNA is maintained episomally. In some embodiments the DNA is integrated into the genome. The exogenous RNA may be chemically modified RNA, e.g., may comprise one or more backbone modification, sugar modifications, noncanonical bases, or caps. Backbone modifications include, e.g., phosphorothioate, N3′ phosphoramidite, boranophosphate, phosphonoacetate, thio-PACE, morpholino phosphoramidites, or PNA. Sugar modifications include, e.g., 2′-O-Me, 2′F, 2′F-ANA, LNA, UNA, and 2′-O-MOE. Noncanonical bases include, e.g., 5-bromo-U, and 5-iodo-U, 2,6-diaminopurine, C-5 propynyl pyrimidine, difluorotoluene, difluorobenzene, dichlorobenzene, 2-thiouridine, pseudouridine, and dihydrouridine. Caps include, e.g., ARCA. Additional modifications are discussed, e.g., in Deleavey et al., “Designing Chemically Modified Oligonucleotides for Targeted Gene Silencing” Chemistry & Biology Volume 19, Issue 8, 24 Aug. 2012, Pages 937-954, which is herein incorporated by reference in its entirety.

In some embodiments, a cell is treated with a chemical agent, e.g. a small molecule, prior to generating a donor cell to increase the expression, stability, or activity of a membrane-associated agent, cargo molecule, or targeting domain that is exogenous relative to the source cell. In some embodiments, a small molecule may increase expression or activity of a transcriptional activator of the membrane-associated agent, cargo molecule, or targeting domain. In some embodiments, a small molecule may decrease expression or activity of a transcriptional repressor of the membrane-associated agent, cargo molecule, or targeting domain. In some embodiments, a small molecule is an epigenetic modifier that increases expression of the membrane-associated agent, cargo molecule, or targeting domain.

In some embodiments, the nucleic acid encodes a modified exogenous membrane-associated agent, cargo molecule, or targeting domain. For example, a membrane-associated agent that has regulatable transfer activity, e.g., specific cell-type, tissue-type or local microenvironment activity. Such regulatable transfer activity may include, activation and/or initiation of transfer activity by low pH, high pH, heat, infrared light, extracellular enzyme activity (eukaryotic or prokaryotic), or exposure of a small molecule, a protein, or a lipid. For example, a modified exogenous membrane-associated agent with regulatable transfer activity may only be configured for transfer from a donor cell to an acceptor cell in the presence of a particular small molecule or class of small molecules and not be configured for transfer in the absence of the small molecule. In some embodiments, the small molecule, protein, or lipid is displayed on a target cell.

In some embodiments, a cell (e.g., a source cell) is genetically modified prior to generating the donor cell to alter (i.e., upregulate or downregulate) the expression of signaling pathways (e.g., membrane metabolism, e.g., TOR pathway, e.g., ALG-2 signaling). In some embodiments, a cell (e.g., source cell) is genetically modified prior to generating the donor cell to alter (e.g., upregulate or downregulate) the expression of a gene or genes of interest. In some embodiments, a cell (e.g., a source cell) is genetically modified prior to generating the donor cell to alter (e.g., upregulate or downregulate) the expression of a nucleic acid (e.g. a miRNA or mRNA) or nucleic acids of interest. In some embodiments, nucleic acids, e.g., DNA, mRNA or siRNA, are transferred to the cell (e.g., source cell) prior to generating the donor cell, e.g., to increase or decrease the expression of signaling pathways, genes, or nucleic acids. In some embodiments, the nucleic acid targets a repressor of a signaling pathway, gene, or nucleic acid, or represses a signaling pathway, gene, or nucleic acid. In some embodiments, the nucleic acid encodes a transcription factor that upregulates or downregulates a signaling pathway, gene, or nucleic acid. In some embodiments the activator or repressor is a nuclease-inactive cas9 (dCas9) linked to a transcriptional activator or repressor that is targeted to the signaling pathway, gene, or nucleic acid by a guide RNA. In some embodiments, a genetic modification epigenetically modifies an endogenous signaling pathway, gene, or nucleic acid to its expression. In some embodiments the epigenetic activator a nuclease-inactive cas9 (dCas9) linked to a epigenetic modifier that is targeted to the signaling pathway, gene, or nucleic acid by a guide RNA. In some embodiments, a cell's DNA is edited prior to generating the donor cell to alter (e.g., upregulate or downregulate) the expression of signaling pathways (e.g. membrane metabolism, e.g., TOR pathway, e.g., ALG-2 signaling), gene, or nucleic acid. In some embodiments, the DNA is edited using a guide RNA and CRISPR-Cas9/Cpf1 or other gene editing technology.

A cell (e.g., source cell) may be genetically modified using recombinant methods. A nucleic acid sequence coding for a desired gene can be obtained using recombinant methods, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter, and incorporating the construct into an expression vector. The vectors can be suitable for replication and integration in eukaryotes. Typical cloning vectors contain transcription and translation terminators, initiation sequences, and promoters useful for expression of the desired nucleic acid sequence.

In some embodiments, a cell may be genetically modified with one or more expression regions, e.g., a gene. In some embodiments, the cell may be genetically modified with an exogenous gene (e.g., capable of expressing an exogenous gene product such as an RNA or a polypeptide product) and/or an exogenous regulatory nucleic acid. In some embodiments, the cell may be genetically modified with an exogenous sequence encoding a gene product that is endogenous to a source cell or target cell and/or an exogenous regulatory nucleic acid capable of modulating expression of an endogenous gene. In some embodiments, the cell may be genetically modified with an exogenous gene and/or a regulatory nucleic acid that modulates expression of an exogenous gene. In some embodiments, the cell may be genetically modified with an exogenous gene and/or a regulatory nucleic acid that modulates expression of an endogenous gene. It will be understood by one of skill in the art that the cell described herein may be genetically modified to express a variety of exogenous genes that encode proteins or regulatory molecules, which may, e.g., act on a gene product of the endogenous or exogenous genome of a source cell or target cell. In some embodiments, such genes confer characteristics to the donor cell, e.g., modulate transfer with a target cell (e.g., acceptor cell). In some embodiments, the cell may be genetically modified to express an endogenous gene and/or regulatory nucleic acid. In some embodiments, the endogenous gene or regulatory nucleic acid modulates the expression of other endogenous genes. In some embodiments, the cell may be genetically modified to express an endogenous gene and/or regulatory nucleic acid which is expressed differently (e.g., inducibly, tissue-specifically, constitutively, or at a higher or lower level) than a version of the endogenous gene and/or regulatory nucleic acid on other chromosomes.

The promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a tissue-specific promoter, metallothionine promoter, a glucocorticoid promoter, a progesterone promoter, and a tetracycline promoter. In some embodiments, expression of an endogenous agent, exogenous membrane-associated agent, targeting domain, or cargo molecule is upregulated before donor cells are generated, e.g., 3, 6, 9, 12, 24, 26, 48, 60, or 72 hours before donor cells are generated.

The expression vector to be introduced into the source cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transfected cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, beta-lactamase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, a cell may be genetically modified to alter expression of one or more proteins. Expression of the one or more proteins may be modified for a specific time, e.g., development or differentiation state of the source. In some embodiments, donor cells are generated from a source of cells genetically modified to alter expression of one or more proteins, e.g., exogenous membrane-associated agents, cargo molecules, or other proteins that affect targeting of an acceptor cell or transfer of the membrane-associated agent or cargo molecule. Expression of the one or more proteins may be restricted to a specific location(s) or widespread throughout the source.

In some embodiments, the expression of an endogenous agent, e.g., targeting domain, is modified. In some embodiments, donor cells are generated from source cells with modified expression of an endogenous agent (e.g., targeting domain), e.g., an increase or a decrease in expression of an endogenous agent by at least 10%, 15%, 20%, 30%, 40%, 50%, 60%, 75%, 80%, 90% or more.

In some embodiments, cells may be engineered to express a cytosolic enzyme (e.g., proteases, phosphatases, kinases, demethylases, methyltransferases, acetylases) that targets a membrane-associated agent, cargo molecule, or targeting domain. In some embodiments, the cytosolic enzyme affects one or more exogenous membrane-associated agents, cargo molecules, or targeting domains by altering post-translational modifications. Post-translational protein modifications of proteins may affect responsiveness to nutrient availability and redox conditions, and protein-protein interactions. In some embodiments, a donor cell comprises exogenous membrane-associated agents, cargo molecules, or targeting domains with altered post-translational modifications, e.g., an increase or a decrease in post-translational modifications by at least 10%, 15%, 20%, 30%, 40%, 50%, 60%, 75%, 80%, 90% or more.

Methods of introducing a modification into a cell include physical, biological and chemical methods. See, for example, Geng. & Lu, Microfluidic electroporation for cellular analysis and delivery. Lab on a Chip. 13(19):3803-21. 2013; Sharei, A. et al. A vector-free microfluidic platform for intracellular delivery. PNAS vol. 110 no. 6. 2013; Yin, H. et al., Non-viral vectors for gene-based therapy. Nature Reviews Genetics. 15: 541-555. 2014. Suitable methods for modifying a cell for use in generating the donor cells or acceptor cells described herein include, for example, diffusion, osmosis, osmotic pulsing, osmotic shock, hypotonic lysis, hypotonic dialysis, ionophoresis, electroporation, sonication, microinjection, calcium precipitation, membrane intercalation, lipid mediated transfection, detergent treatment, viral infection, receptor mediated endocytosis, use of protein transduction domains, particle firing, membrane fusion, freeze-thawing, mechanical disruption, and filtration.

Confirming the presence of a genetic modification includes a variety of assays. Such assays include, for example, molecular biological assays, such as Southern and Northern blotting, RT-PCR and PCR; biochemical assays, such as detecting the presence or absence of a particular peptide, e.g., by immunological means (ELISAs and Western blots) or by assays described herein.

### Modifications to Donor Cells

In some aspects, a modification is made to the donor cell. Such modifications can be effective to, e.g., improve targeting, function, or structure.

In some embodiments, the donor cell is treated with a membrane-associated agent, targeting domain, or cargo molecule that may non-covalently or covalently link to the surface of the membrane. In some embodiments, the donor cell is treated with a membrane-associated agent, targeting domain, or cargo molecule, e.g., a protein or a lipid, that may non-covalently or covalently link or embed itself in the membrane

As described herein, additives that are not exogenous membrane-associated agents may be added to the donor cell to modify their structure and/or properties. For example, either cholesterol or sphingomyelin may be added to the membrane to help stabilize the structure and to prevent the leakage of, e.g., cargo molecules. Further, membranes can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi: 10.1155/2011/469679 for review).

In some embodiments, the donor cell comprises one or more targeting domains on the exterior surface to target a specific cell or tissue type (e.g., cardiomyocytes). These targeting domains include without limitation receptors, ligands, antibodies, and the like. In some embodiments, the targeting domain is comprised in a TCR, a TCR, or a functional fragment or variant thereof. These targeting domains bind their partner on the target cells' (e.g., acceptor cells') surface (e.g., an MHC presenting a cognate peptide, wherein the TCR binds specifically to the MHC and/or the cognate peptide; or a ligand or binding partner specifically bound by the CAR). In embodiments, the targeting domain is specific for a target cell moiety, e.g., a cell surface marker on a target cell (e.g., acceptor cell) described herein, e.g., an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeLa, HEK293, HFF-1, MRC-5, WI-38, IMR 90, IMR 91, PER.C6, HT-1080, or BJ cell).

In some embodiments, the targeting domain binds a cell surface marker on a target cell (e.g., acceptor cell). In embodiments, the cell surface marker comprises a protein, glycoprotein, receptor, cell surface ligand, class I transmembrane protein, class II transmembrane protein, or class III transmembrane protein, or a fragment or peptide therefrom.

In some embodiments, the donor cell or acceptor cell described herein is functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium.

In some embodiments, a donor cell as described herein comprises a cell-penetrating peptide (CPP), e.g., as described herein. In certain embodiments, the cell-penetrating peptide is attached to (e.g., covalently or noncovalently bound to) a cargo molecule, e.g., as described herein. In embodiments, the cell-penetrating peptide is attached to (e.g., covalently or noncovalently bound to) a molecule associated with a membrane-associated agent, e.g., as described herein (e.g., a polypeptide comprising a ZAP70 domain). In certain embodiments, the cell-penetrating peptide is attached to (e.g., covalently or noncovalently bound to) a membrane-associated agent, e.g., as described herein. In certain embodiments, the cell-penetrating peptide is attached to (e.g., covalently or noncovalently bound to) a molecule comprised in, embedded in, or tethered to the cell membrane of the donor cell. In certain embodiments, the cell-penetrating peptide is exposed on the surface of the donor cell.

In certain embodiments, the cell-penetrating peptide comprises a peptide as listed in Table CP1, or a peptide having at least 75%, 80%, 85%, 90%, 95%, 96%, 97%, 98%, or 99% amino acid sequence identity thereto, or a peptide having no more than 1, 2, 3, 4, 5, 6, 7, 8, 9, or 10 amino acid differences relative thereto.

TABLE CP1
Exemplary cell-penetrating peptides

| Name | Description | Amino Acid Sequence |
|---|---|---|
| Tat (transcription- | HIV Tat protein | YGRKKRRQRRR |
| transactivating) | | |
| Penetratin (PEN) | Homeodomain | RQIKIWFQNRRMKWKK |
| Oligoarginine (R9) | Chimeric | RRRRRRRRR |
| 5.3R (and also a variant | | GPSQPTYPGDDAPVRDLIRFYRDLQRYLNV |
| named ZF 5.3R) | | VTRHRY |
| Transportan | Galanin-mastoparan | GWTLNSAGYLLGKINLKALAALAKKIL |
| VP-22 | HSV-1 structural | DAATATRGRSAASRPTERPRAPAR |
| | protein | |
| Pep-1 | Trp-rich motif-SV40 | KETWWETWWTEWSQPKKKRKV |
| | NLS | |
| SAP | Proline-rich motif | VRLPPPVRLPPPVRLPPP |
| hCT (9-32) | Human calcitonin | LGTYTQDENKTFPQTAIGVGAP |
| Pvec | Murine VE-cadherin | LLIILRRRIRKQAHAHSK |

In some embodiments, the cell penetrating peptide is associated with (e.g., attached to, e.g., covalently or noncovalently bound to) a secretion signal, e.g., as described herein. In certain embodiments, the cell-penetrating peptide is secreted by the donor cell (e.g., upon binding of a membrane-associated agent of the donor cell to a cognate docking moiety on an acceptor cell).

In some embodiments, a donor cell as described herein comprises a fusogen. In some embodiments, the fusogen promotes binding of the donor cell or the acceptor cell. In some embodiments, the fusogen is transferred from the donor cell to the acceptor cell. In some embodiments, the fusogen is transferred alongside the membrane-associated agent and/or the cargo molecule. In some embodiments, the fusogen is a polypeptide (e.g., a protein). In some embodiments, the fusogen comprises a polypeptide complex. In some embodiments, the fusogen is a viral fusogen. In some embodiments, the fusogen is pH-selective (e.g., active at pH below physiological pH, e.g., active at pH of less than pH 7.4). In an embodiment, the fusogen is vsv-g.

In some embodiments, a donor cell as described herein comprises a plurality of viral capsid proteins capable of forming a virus-like particle (VLP). The VLPs, in certain embodiments, can encapsidate a cargo molecule (e.g., a polypeptide cargo and/or a nucleic acid cargo, e.g., an RNA cargo). In certain embodiments, VLPs formed inside the donor cell are released by the donor cell. In certain embodiments, VLPs released from the donor cell can infect nearby cells (e.g., acceptor cells). In certain embodiments, VLPs that infect acceptor cells can deliver the encapsidated cargo to the acceptor cell. In certain embodiments, a membrane-associated agent as described herein comprises a PEG10 binding sequence. In certain embodiments, a cargo molecule as described herein comprises a PEG10 binding sequence.

In some embodiments, a donor cell as described herein generates a tunneling nanotube to an acceptor cell, e.g., upon binding of a membrane-associated agent on the donor cell to a cognate docking moiety on the acceptor cell. In certain embodiments, the donor cell introduces a cargo molecule (e.g., a polypeptide cargo molecule or a nucleic acid cargo molecule) to the acceptor cell via the tunneling nanotube. In embodiments, the donor cell is a Treg or is derived from a Treg. In embodiments, the donor cell comprises a nucleic acid molecule encoding US3 (e.g., under the control of an NFAT promoter).

In some embodiments, a donor cell as described herein comprises a kill switch molecule, e.g., an inducible agent that promotes cell death of the donor cell. In certain embodiments, the kill switch molecule comprises an inducible cell death protein. In embodiments, the kill switch molecule comprises an inducible caspase (e.g., an inducible caspase 9), or a functional fragment or variant thereof. In embodiments, cell death is induced by contacting the kill switch molecule and/or the donor cell with an activator of the kill switch molecule (e.g., a small molecule dimerizer).

In some embodiments, a donor cell as described herein comprises a selectable marker. In certain embodiments, the selectable marker comprises a fluorophore (e.g., a fluorescent protein). In certain embodiments, the selectable marker comprises an epitope tag (e.g., a FLAG tag). In embodiments, the selectable marker allows for selection and/or isolation of the donor cell using a binding moiety (e.g., an antibody or an antigen-binding fragment thereof) specific to the selectable marker, e.g., a magnetic antibody or antigen-binding fragment thereof).

In some embodiments, a donor cell is an induced pluripotent stem cell (iPSC). In some embodiments, a donor cell is derived from and/or descended from an iPSC. In certain embodiments, an iPSC is modified (e.g., using CRISPR, transposons, or viral transduction, e.g., using a lentivirus) to comprise and/or express a membrane-associated agent and/or a cargo molecule, e.g., as described herein. In certain embodiments, an iPSC is differentiated into a cell type of interest, e.g., a T cell, e.g., a CD3+, CD4+, or CD8+ T cell, a Treg), or an NK cell, e.g., as described herein.

### Immunogenicity

In some embodiments of any of the aspects described herein, the donor cell composition is substantially non-immunogenic. Immunogenicity can be quantified, e.g., as described herein.

In some embodiments, the donor cell composition comprises elevated levels of an immunosuppressive agent as compared to a reference cell, e.g., an unmodified cell otherwise similar to the source cell. In some embodiments, the elevated level is at least 5%, 10%, 20%, 30%, 40%, 50%, 60%, 70%, 80%, 90%, 2-fold, 3-fold, 5-fold, 10-fold, 20-fold, 50-fold, or 100-fold. In some embodiments, the donor cell composition comprises an immunosuppressive agent that is absent from the reference cell. In some embodiments, the donor cell composition comprises reduced levels of an immune activating agent as compared to a reference cell, e.g., an unmodified cell otherwise similar to the source cell. In some embodiments, the reduced level is at least 5%, 10%, 20%, 30%, 40%, 50%, 60%, 70%, 80%, 90%, 95%, 98%, or 99% compared to the reference cell. In some embodiments, the immune activating agent is substantially absent from the donor cell or acceptor cell.

In some embodiments, the donor cell composition, or the source cell from which the donor cell or acceptor cell composition is derived from, has one, two, three, four, five, six, seven, eight, nine, ten, eleven, twelve, or more of the following characteristics:
a) less than 50%, 40%, 30%, 20%, 15%, 10%, or 5% or lesser expression of MHC class I or MHC class II, compared to a reference cell, e.g., an unmodified cell otherwise similar to the source cell, or a HeLa cell;
b) less than 50%, 40%, 30%, 20%, 15%, 10%, or 5% or lesser expression of one or more co-stimulatory proteins including but not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4, compared to a reference cell, e.g., an unmodified cell otherwise similar to the source cell, or a reference cell described herein;
c) expression of surface proteins which suppress macrophage engulfment e.g., CD47, e.g., detectable expression by a method described herein, e.g., more than 1.5-fold, 2-fold, 3-fold, 4-fold, 5-fold, 10-fold, or more expression of the surface protein which suppresses macrophage engulfment, e.g., CD47, compared to a reference cell, e.g., an unmodified cell otherwise similar to the source cell;
d) expression of soluble immunosuppressive cytokines, e.g., IL-10, e.g., detectable expression by a method described herein, e.g., more than 1.5-fold, 2-fold, 3-fold, 4-fold, 5-fold, 10-fold, or more expression of soluble immunosuppressive cytokines, e.g., IL-10, compared to a reference cell, e.g., an unmodified cell otherwise similar to the source cell;
e) expression of soluble immunosuppressive proteins, e.g., PD-L1, e.g., detectable expression by a method described herein, e.g., more than 1.5-fold, 2-fold, 3-fold, 4-fold, 5-fold, 10-fold, or more expression of soluble immunosuppressive proteins, e.g., PD-L1, compared to a reference cell e.g., an unmodified cell otherwise similar to the source cell;
f) less than 50%, 40%, 30%, 20%, 15%, 10%, or 5% or lesser expression of soluble immune stimulating cytokines, e.g., IFN-gamma or TNF-α, compared to a reference cell, e.g., an unmodified cell otherwise similar to the source cell;
g) expression of, e.g., detectable expression by a method described herein, HLA-E or HLA-G, compared to a reference cell, e.g., an unmodified cell otherwise similar to the source cell;
h) surface glycosylation profile, e.g., containing sialic acid, which acts to, e.g., suppress NK cell activation;
i) less than 50%, 40%, 30%, 20%, 15%, 10%, or 5% or lesser expression of TCRα/β, compared to a reference cell, e.g., an unmodified cell otherwise similar to the source cell;
j) less than 50%, 40%, 30%, 20%, 15%, 10%, or 5% or lesser expression of Minor Histocompatibility Antigen (MHA), compared to a reference cell, e.g., an unmodified cell otherwise similar to the source cell; or
k) has less than 10%, 9%, 8%, 7%, 6%, 5%, 4%, 3%, 2%, 1%, or less, of mitochondrial MHAs, compared to a reference cell e.g., an unmodified cell otherwise similar to the source cell, or has no detectable mitochondrial MHAs.

In some embodiments, the donor cell composition does not substantially elicit an immunogenic response by the immune system, e.g., innate immune system. In some embodiments, the an immunogenic response by the innate immune system comprises a response by innate immune cells including, but not limited to NK cells, macrophages, neutrophils, basophils, eosinophils, dendritic cells, mast cells, or gamma/delta T cells. In some embodiments, an immunogenic response by the innate immune system comprises a response by the complement system which includes soluble blood components and membrane bound components.

In some embodiments, the donor cell composition does not substantially elicit an immunogenic response by the immune system, e.g., adaptive immune system. In some embodiments, an immunogenic response by the adaptive immune system comprises an immunogenic response by an adaptive immune cell including, but not limited to a change, e.g., increase, in number or activity of T lymphocytes (e.g., CD4 T cells, CD8 T cells, and or gamma-delta T cells), or B lymphocytes. In some embodiments, an immunogenic response by the adaptive immune system includes increased levels of soluble blood components including, but not limited to a change, e.g., increase, in number or activity of cytokines or antibodies (e.g., IgG, IgM, IgE, IgA, or IgD).

In some embodiments, the donor cell composition is modified to have reduced immunogenicity. Immunogenicity can be quantified, e.g., as described herein. In some embodiments, the donor cell composition has an immunogenicity less than 5%, 10%, 20%, 30%, 40%, or 50% lesser than the immunogenicity of a reference cell, e.g., an unmodified cell otherwise similar to the source cell.

In some embodiments of any of the aspects described herein, the donor cell composition is derived from a source cell, e.g., a mammalian cell, having a modified genome, e.g., modified using a method described herein, to reduce, e.g., lessen, immunogenicity. Immunogenicity can be quantified, e.g., as described herein.

In some embodiments, the donor cell composition is derived from a source cell, e.g., a mammalian cell, wherein the mammalian cell comprises a therapeutic agent.

In some embodiments, the donor cell composition is derived from a source cell, e.g., a mammalian cell, wherein the mammalian cell is a recombinant cell.

In some embodiments, the surface of the donor cell, or the surface of the mammalian cell the donor cell is derived from, is covalently or non-covalently modified with a polymer, e.g., a biocompatible polymer that reduces immunogenicity and immune-mediated clearance, e.g., PEG.

In some embodiments, the surface of the donor cell, or the surface of the mammalian cell the donor cell is derived from is covalently or non-covalently modified with a sialic acid, e.g., a sialic acid comprising glycopolymers, which contain NK-suppressive glycan epitopes.

In some embodiments, the surface of the donor cell, or the surface of the mammalian cell the donor cell is derived from is enzymatically treated, e.g., with glycosidase enzymes, e.g., α-N-acetylgalactosaminidases, to remove ABO blood groups.

In some embodiments, the surface of the donor cell, or the surface of the mammalian cell the donor cell is derived from is enzymatically treated, to give rise to, e.g., induce expression of, ABO blood groups which match the recipient's blood type.

### Acceptor Cells

The present disclosure is directed, in part, to acceptor cells capable of receiving a membrane-associated agent and optionally one or more cargo molecules. In addition, the present disclosure is directed, in part, to acceptor cells comprising said exogenous membrane-associated agent and optionally one or more cargo molecules (e.g., having received the membrane-associated agent and optionally one or more cargo molecules from a donor cell) but not comprising a nucleic acid encoding the membrane-associated agent and optionally one or more cargo molecules. The present disclosure is further directed, in part, to compositions comprising such acceptor cells (e.g., acceptor cells comprising cargo molecules and/or membrane-associated agents after transfer from a donor cell as described herein). In some embodiments, the acceptor cell is not a cancer cell. In some embodiments, the acceptor cell is in a subject that does not have cancer. In some embodiments, the acceptor cell is ex vivo and is from subject that does not have cancer.

In some embodiments, an acceptor cell is modified in vivo, e.g., by a donor cell administered to a tissue, organ, or subject. In embodiments, the modification comprises transferring a cargo molecule and/or a membrane-associated agent from the donor cell to the acceptor cell.

In some embodiments, the target cell, e.g., acceptor cell, is an immune cell (e.g., an immune effector cell), e.g., a T cell, B cell, NK cell, a PMN (e.g., a granulocyte), a monocyte, a dendritic cell, or a macrophage, or is derived from the same. In some embodiments, the target cell, e.g., acceptor cell, is an antigen-presenting cell (APC), e.g., comprising on its surface an MHC molecule, e.g., presenting a cognate peptide to a TCR, or a functional fragment or variant thereof, on the surface of a donor cell as described herein. In embodiments, the APC is selected from the group consisting of: a professional APC (e.g., a dendritic cell, macrophage, or B cell) and a non-professional APC (e.g., any other cell type as described herein).

In some embodiments, the target cell comprises on its surface a second docking moiety (e.g., a ligand of the first docking moiety of a membrane-associated agent as described herein). In some embodiments, the target cell, e.g., acceptor cell, is an antigen-presenting cell (APC), e.g., comprising on its surface an MHC molecule, e.g., presenting a peptide, or a functional fragment or variant thereof. In embodiments, the APC is selected from the group consisting of: a professional APC (e.g., a dendritic cell, macrophage, or B cell) and a non-professional APC (e.g., any other cell type as described herein).

In some embodiments, a target cell, e.g., acceptor cell, is a cell from a cell line (e.g., an immortalized cell line), e.g., NK92, THP1, Jurkat, RAW264.7, BT-474, SK-BR-3, MDA-MB-231, BT-20, or KHYG-1 (e.g., as available from ATCC or AcceGen).

In some embodiments, the target cell, e.g., acceptor cell, is in an organism. In some embodiments, the target cell, e.g., acceptor cell, is a primary cell isolated from an organism. In some embodiments, the targeting domain interacts with a target cell moiety on the target cell (e.g., acceptor cell), e.g., a cell surface feature. In some embodiments, the donor cell does not comprise said target cell moiety. In some embodiments, the donor cell comprises a membrane-associated agent or targeting domain which interacts with a binding partner on the target cell (e.g., acceptor cell), thereby allowing the donor cell to bind to the target cell (e.g., acceptor cell) and/or transfer a membrane-associated agent and optionally one or more cargo molecules to the target cell (e.g., acceptor cell). In some embodiments, the donor cell does not comprise said binding partner. In some embodiments, the targeting domain is not part of the membrane-associated agent or cargo molecule. In some embodiments, the membrane-associated agent comprises the targeting domain. In some embodiments, the binding partner is or is a portion of a different entity from the target cell moiety. In some embodiments, the binding partner is or is a portion of the target cell moiety.

In some embodiments, the target cell (e.g., acceptor cell) or tissue comprising the same is modified (e.g., by inducing stress or cell division) to increase the rate of transfer prior to, at the same time, or after the delivery of donor cell. Some nonlimiting examples include, inducing ischemia, treatment with chemotherapy, antibiotic, irradiation, toxin, inflammation, inflammatory molecules, anti-inflammatory molecules, acid injury, basic injury, burn, polyethylene glycol, neurotransmitters, myelotoxic drugs, growth factors, or hormones, tissue resection, starvation, and/or exercise.

In some embodiments, the target cell (e.g., acceptor cell) or tissue comprising the same is treated with a vasodilator (e.g. nitric oxide (NO), carbon monoxide, prostacyclin (PGI2), nitroglycerine, phentolamine) or vasoconstrictors (e.g. angiotensin (AGT), endothelin (EDN), norepinephrine)) to increase the rate of donor cell transport to the tissue.

In some embodiments, the target cell (e.g., acceptor cell) or tissue comprising the same is treated with a chemical agent, e.g., a chemotherapeutic. In such embodiments, the chemotherapeutic induces damage to the target cell (e.g., acceptor cell) or tissue that enhances transfer of a membrane-associated agent or cargo molecule to the target cells (e.g., acceptor cells) or tissue.

In some embodiments, the target cell (e.g., acceptor cell) or tissue comprising the same is treated with an activating agent that stimulates or promotes the receipt of a membrane-associated agent and/or cargo molecule from a donor cell. In some embodiments, the activating agent is DMSO or PMA, or a combination thereof.

### Target Cell Moiety

A target cell is generally a cell capable of receiving a membrane-associated agent and optionally one or more cargo molecules. In some embodiments, a target cell is an acceptor cell, e.g., as described herein. In some embodiments, a target cell receives a membrane-associated agent or optionally one or more cargo molecules from a donor cell. In some embodiments, a target cell, e.g., acceptor cell, is present in vivo.

In some embodiments, a target cell (e.g., acceptor cell) comprises one or more target cell moieties. In some embodiments, a target cell moiety may be used to promote transfer of a membrane-associated agent and optionally one or more cargo molecules from a donor cell to a target cell, e.g., acceptor cell. In some embodiments, a target cell moiety is a surface feature of a target cell. In some embodiments, a target cell moiety is or is a portion of a protein associated with the cell membrane of a target cell. In some embodiments, a target cell moiety is, or is a portion of, a peptide or protein associated with the membrane of a target cell. In some embodiments, a target cell moiety is or is a portion of a lipid associated with the membrane of a target cell. In some embodiments, a target cell moiety is or is a portion of a saccharide associated with the membrane of a target cell. In some embodiments, the target cell moiety is endogenous to the target cell (e.g., acceptor cell). In some embodiments, the target cell moiety comprises an MHC. In some embodiments, the target cell moiety comprises an MHC presenting a peptide, wherein the peptide is a cognate peptide to a membrane-associated agent on a donor cell (e.g., a TCR, or a functional fragment or variant thereof, on the surface of the donor cell), e.g., wherein the TCR, or the functional fragment or variant thereof, binds specifically to the MHC-cognate peptide complex. In some embodiments, the target cell moiety comprises a ligand, e.g., comprising an epitope specifically bound by a membrane-associated agent (e.g., a CAR).

A target cell moiety may be used to target a donor cell to a target cell (e.g., acceptor cell). A target cell moiety may be used to promote transfer of a membrane-associated agent and/or one or more cargo molecules from a donor cell to a target cell (e.g., acceptor cell). In some embodiments, a donor cell binds (e.g., via a membrane-associated agent and/or targeting domain) to a target cell moiety on a target cell, e.g., acceptor cell.

In some embodiments, a target cell moiety is, e.g., a protein, disposed in a membrane (e.g., a lipid bilayer), of a target cell (e.g., acceptor cell) disclosed herein (e.g., an MHC-cognate peptide complex, e.g., as described herein). In some embodiments, a target cell moiety can be endogenously expressed, overexpressed, or exogenously expressed (e.g., by a method described herein). In some embodiments, the target cell moiety can cluster with other target cell moieties at the membrane.

In some embodiments, the presence of a target cell moiety, or a plurality of target cell moieties, in a membrane of a target cell (e.g., acceptor cell), creates an interface that can facilitate the interaction, e.g., binding, between a target cell moiety on a target cell (e.g., an acceptor cell), and a membrane-associated agent or targeting domain on a donor cell. In some embodiments, the membrane-associated agent or targeting domain on a donor cell interacts with, e.g., binds to, a target cell moiety on a target cell (e.g., acceptor cell), e.g., on the membrane (e.g., lipid bilayer), of a target cell, to induce transfer of the membrane-associated agent and/or one or more cargo molecules from the donor cell to the target cell (e.g., acceptor cell) membrane.

### Membrane-Associated Agents

The donor cells and acceptor cells described herein may comprise a membrane-associated agent. A membrane-associated agent comprises, minimally, a membrane-associated moiety and one or more of an extracellular moiety, an intracellular moiety, or a cargo molecule. Using the methods described herein, a donor cell comprising a membrane-associated agent can transfer said agent and/or one or more cargo molecules to a target cell, e.g., an acceptor cell.

In some embodiments, a membrane-associated agent comprises more than one membrane-associated moiety, extracellular moiety, intracellular moiety, or cargo molecule. In some embodiments, at least one moiety (e.g., membrane-associated moiety, extracellular moiety, intracellular moiety, or cargo molecule) of a membrane-associated agent is exogenous to 1) a donor comprising the membrane-associated agent, 2) the target cell, e.g., acceptor cell, to which the membrane-associated agent was or will be transferred, or 3) both. In some embodiments, an exogenous membrane-associated agent is a fusion protein. In some embodiments, a membrane-associated agent comprises a TCR, or a functional fragment or variant thereof, e.g., as described herein, e.g., comprising a TCR alpha molecule and/or a TCR beta molecule, or a TCR gamma molecule and/or a TCR delta molecule.

In some embodiments, for a given exogenous membrane-associated agent, at least one of the membrane-associated moiety, extracellular moiety, intracellular moiety, or cargo molecule is exogenous to the donor cell, acceptor cell, or a source cell from which the aforementioned were derived. In some embodiments, at least one of (e.g., one, two, three, or all of) the membrane-associated moiety, extracellular moiety, intracellular moiety, or cargo molecule is exogenous to the donor cell or a source cell from which the donor cell was derived. In some embodiments, at least one of (e.g., one, two, three, or all of) the membrane-associated moiety, extracellular moiety, intracellular moiety, or cargo molecule is exogenous to the acceptor cell or a source cell from which the acceptor cell was derived. In some embodiments, at least one of (e.g., one, two, three, or all of) the membrane-associated moiety, extracellular moiety, intracellular moiety, or cargo molecule is exogenous to the donor cell, acceptor cell, or a source cell from which the donor or acceptor cell was derived.

In some embodiments, for a given exogenous membrane-associated agent, none of the membrane-associated moiety, extracellular moiety, intracellular moiety, or cargo molecule are exogenous to the donor cell, acceptor cell, or a source cell from which the aforementioned were derived but the combination of said one or more moieties and/or molecule(s) in a single agent is exogenous to the donor cell, acceptor cell, or a source cell from which the aforementioned were derived. In some embodiments, each of the membrane-associated moiety, extracellular moiety, intracellular moiety, and/or cargo molecule are endogenous to the donor cell, acceptor cell, or a source cell from which the aforementioned were derived, but the combination of said one or more moieties and/or molecule(s) in a single agent is exogenous to the donor cell, acceptor cell, or a source cell from which the aforementioned were derived.

A membrane-associated agent may target a donor cell to a target cell, e.g., acceptor cell. In some embodiments, a membrane-associated agent may specifically target a donor cell to a target cell (e.g., acceptor cell) type, and not target the donor cell to one or more (e.g., two, three, four, five, six, or more) non-target cell types. In some embodiments, targeting comprises a membrane-associated agent binding to a target cell moiety, e.g., on the surface of the target cell, e.g., acceptor cell. In some embodiments, the extracellular moiety binds specifically to the target cell moiety (e.g., and not to other moieties or receptors on the surface of non-target cells). In some embodiments, the extracellular moiety binds specifically to a plurality of target cell moieties. Without wishing to be bound by theory, it is thought that increasing the number of target cell moieties the membrane-associated agent, targeting domain, or extracellular moiety bind may increase specificity in a combinatorial manner.

A membrane-associated agent may promote transfer of the membrane-associated agent and/or one or more cargo molecules from a donor cell to a target cell, e.g., acceptor cell. In some embodiments, a membrane-associated agent may specifically promote transfer from a donor cell to a target cell (e.g., acceptor cell) type, and not promote transfer from the donor cell to one or more (e.g., two, three, four, five, six, or more) non-target cell types. In some embodiments, promoting transfer comprises a membrane-associated agent binding to a target cell moiety, e.g., on the surface of the target cell, e.g., acceptor cell. In some embodiments, promoting transfer comprises the extracellular moiety binding specifically to the target cell moiety as described above.

In some embodiments, a membrane-associated agent comprises a membrane-associated moiety (e.g., a transmembrane domain from a TCR protein, or a transmembrane domain from a CD28, CD8, or CD3 zeta protein) and an extracellular moiety (e.g., operably associated or linked to (e.g., tethered to) the membrane-associated moiety), e.g., an extracellular domain from a TCR protein, or, e.g., comprising an antibody molecule (e.g., an antibody or a functional fragment or variant thereof, e.g., an scFv). In some embodiments, a membrane-associated agent comprises a membrane-associated moiety and an intracellular moiety (e.g., operably associated or linked to (e.g., tethered to) the membrane-associated moiety), e.g., an intracellular domain from a TCR protein (e.g., CD3 zeta). In some embodiments, a membrane-associated agent comprises a membrane-associated moiety and a cargo molecule (e.g., operably associated or linked to (e.g., tethered to) the membrane-associated moiety). In some embodiments, operably associated or linked comprises a non-covalent interaction. In some embodiments, operably associated or linked comprises a covalent interaction, e.g., a peptide bond or a linker.

In some embodiments, a membrane-associated agent comprises a membrane-associated moiety, an extracellular moiety, and an intracellular moiety, e.g., from a TCR. In some embodiments, the extracellular moiety and intracellular moiety are operably associated or linked to (e.g., tethered to) the membrane-associated moiety.

In some embodiments, a membrane-associated agent comprises a membrane-associated moiety, an extracellular moiety, and one or more cargo molecules. In some embodiments, the extracellular moiety is operably associated or linked to (e.g., tethered to) the membrane-associated moiety, and the one or more cargo molecules are operably associated or linked to (e.g., tethered to) the extracellular moiety. In some embodiments, the extracellular moiety and one or more cargo molecules are operably associated or linked to (e.g., tethered to) the membrane-associated moiety. In some embodiments, the extracellular moiety and one or more cargo molecules are operably associated or linked to (e.g., tethered to) the membrane-associated moiety, and one or more different cargo molecules are operably associated or linked to (e.g., tethered to) the extracellular moiety.

In some embodiments, a membrane-associated agent comprises a membrane-associated moiety, an intracellular moiety, and one or more cargo molecules. In some embodiments, the intracellular moiety is operably associated or linked to (e.g., tethered to) the membrane-associated moiety, and the one or more cargo molecules are operably associated or linked to (e.g., tethered to) the intracellular moiety. In some embodiments, the intracellular moiety and one or more cargo molecules are operably associated or linked to (e.g., tethered to) the membrane-associated moiety. In some embodiments, the intracellular moiety and one or more cargo molecules are operably associated or linked to (e.g., tethered to) the membrane-associated moiety, and one or more different cargo molecules are operably associated or linked to (e.g., tethered to) the intracellular moiety.

In some embodiments, a membrane-associated agent comprises a membrane-associated moiety, an extracellular moiety, an intracellular moiety, and one or more cargo molecules. In some embodiments, the extracellular moiety and intracellular moiety are operably associated or linked to (e.g., tethered to) the membrane-associated moiety, and the one or more cargo molecules are operably associated or linked to (e.g., tethered to) the extracellular moiety. In some embodiments, the extracellular moiety and intracellular moiety are operably associated or linked to (e.g., tethered to) the membrane-associated moiety, and the one or more cargo molecules are operably associated or linked to (e.g., tethered to) the intracellular moiety. In some embodiments, one or more cargo molecules are operably associated or linked (e.g., tethered) with multiple different moieties of the membrane-associated agent. For example, a membrane-associated agent may comprise a membrane-associated moiety, an extracellular moiety, and an intracellular moiety, wherein one or more cargo molecules are operably associated or linked to (e.g., tethered to) a first and a second moiety, or a first, second, and third moiety chosen from the membrane-associated moiety, an extracellular moiety, and an intracellular moiety.

In some embodiments, a membrane-associated agent comprises a peptide, a polypeptide, a nucleic acid (e.g., DNA, RNA, mRNA, siRNA, miRNA), a saccharide or a polysaccharide, a lipid, a small molecule, or a combination or complex thereof. In some embodiments, a membrane-associated agent is or comprises a fusion protein. In some embodiments, the fusion protein comprises the membrane-associated moiety and one or both of an extracellular moiety and intracellular moiety. In some embodiments, the fusion protein comprises a cargo molecule. In some embodiments, a membrane-associated agent comprises a fusion protein (e.g., comprising the membrane-associated moiety and optionally one or both of an extracellular moiety and intracellular moiety) and a cargo molecule, wherein the cargo molecule is operably associated or linked (e.g., tethered) to the fusion protein. In some embodiments, said cargo molecule is not connected to the fusion protein by a peptide bond.

In some embodiments, the membrane-associated agent comprises one or more linkers. In some embodiments, the membrane-associated moiety is connected to another moiety (e.g., an extracellular moiety or intracellular moiety) or cargo molecule by a linker. In some embodiments, the extracellular moiety is connected to a cargo molecule by a linker. In some embodiments, the intracellular moiety is connected to a cargo molecule by a linker. In some embodiments, the portions of an extracellular moiety (e.g., specificity portion and accessory portion) or intracellular moiety (e.g., functional portion and accessory portion) are connected by a linker. A linker comprises a covalent connection or series of connections between two agents, e.g., between two moieties. In some embodiments, a linker comprises a peptide, e.g., and the two agents are connected via peptide bonds. In some embodiments, a linker comprises non-amino acid components. In some embodiments, a linker for use in a membrane-associated agent is flexible linker.

In some embodiments, a membrane-associated agent is expressed from a nucleic acid molecule comprised in a donor cell. In some embodiments, the donor cell stably expresses the membrane-associated agent (e.g., comprises a nucleic acid sequence encoding the membrane-associated agent in its genome). In some embodiments, the donor cell transiently expresses the membrane-associated agent. In some embodiments, a nucleic acid molecule encoding a membrane-associated agent is introduced into a donor cell. In certain embodiments, the nucleic acid molecule encoding the membrane-associated agent is inserted into the genome of the donor cell, e.g., using a transposase system (e.g., a Sleeping Beauty transposase system). In certain embodiments, the nucleic acid molecule encoding the membrane-associated agent is introduced into the donor cell by electroporation.

In some embodiments, a membrane-associated agent is expressed from an exogenous nucleic acid molecule introduced into a donor cell or an exogenous nucleic acid sequence inserted into the genome of a donor cell. In some embodiments, the membrane-associated agent is encoded in the exogenous nucleic acid molecule or the exogenous insert sequence under the control of a promoter (e.g., a lineage-restricted, tissue-specific, or cell type-specific promoter). In an embodiment, the membrane-associated agent is under the control of an NFAT promoter. In an embodiment, the membrane-associated agent is under the control of a FoxP3 promoter.

### T Cell Receptors (TCRs)

A donor or acceptor cell, as described herein, may comprise a T cell receptor (TCR) molecule (e.g., a TCR). Generally, a TCR is a protein complex, or functional fragments or variants thereof, typically found on the surface of T cell, which is capable of binding to a major histocompatibility complex (MHC) molecule (e.g., an HLA molecule) presenting a cognate peptide. In some embodiments, a TCR as described herein is a wild-type TCR, e.g., a wild-type TCR from a mammal (e.g., a human). In some embodiments, a TCR as described herein is an engineered TCR (e.g., a non-natural and/or modified TCR, e.g., a mutated TCR or comprising a fusion protein). In some embodiments, a TCR as described herein comprises one or more TCR molecule that is a functional fragment or variant of a corresponding wild-type TCR protein.

In some embodiments, a donor cell or acceptor cell comprises a TCR molecule, e.g., as described herein.

In some embodiments, a donor cell or acceptor cell comprises a polypeptide comprising a T cell receptor (TCR), e.g., a T-cell receptor fusion protein (TFP). In some embodiments, a membrane-associated agent or a cargo molecule comprises a T cell receptor, e.g., a TFP. In some embodiments, the TFP comprises a recombinant polypeptide derived from the various polypeptides comprising the TCR that is generally capable of i) binding to a surface antigen on target cells and ii) interacting with other polypeptide components of the intact TCR complex, typically when co-located in or on the surface of a T-cell. In some embodiments, the TFP incorporates into a TCR when expressed in a T-cell. In some embodiments, the membrane-associated agent or cargo molecule comprises (i) an antigen binding domain operatively linked to (ii) a TCR domain.

In some embodiments, the TFP includes an extracellular domain of a TCR subunit that comprises an extracellular domain or portion thereof of a protein selected from the group consisting of the alpha or beta chain of the T cell receptor, CD3 delta, CD3 epsilon, or CD3 gamma, or a functional fragment or variant thereof. In some embodiments, the TCR domain includes a transmembrane domain, e.g., at least a transmembrane region of a transmembrane domain of a TCR alpha chain, a TCR beta chain, a CD3 epsilon TCR subunit, a CD3 gamma TCR subunit, a CD3 delta TCR subunit, or a CD3 zeta TCR subunit, or a functional fragment or variant thereof.

In further embodiments, the TCR domain comprises a TCR intracellular domain comprising a stimulatory domain selected from an intracellular signaling domain of CD3 epsilon, CD3 gamma, or CD3 delta, or a variant thereof.

In further embodiments, the TCR domain comprises (i) a TCR extracellular domain, (ii) a TCR transmembrane domain, and (iii) a TCR intracellular domain, wherein at least two or all three of (i), (ii), and (iii) are from the same TCR subunit.

In some embodiments, the TCR domain comprises CD3ε or a functional fragment or variant thereof. In some embodiments, the TCR domain (e.g., CD3ε-based TCR domain) binds endogenous CD3ζ. In some embodiments, the TCR domain (e.g., CD3ε-based TCR domain) binds endogenous CD3γ and/or endogenous CD3δ. In some embodiments, the TCR domain comprises CD3α or a functional fragment or variant thereof. In some embodiments, the TCR domain comprises CD3β or a functional fragment or variant thereof. In some embodiments, the TCR domain (e.g., CD3α-based or CD3β-based TCR domain) binds endogenous CD3ζ. In some embodiments, the TCR domain (e.g., CD3α-based TCR domain) binds endogenous CD3β. In some embodiments, the TCR domain (e.g., CD3β-based TCR domain) binds endogenous CD3α. In some embodiments, the TCR domain (e.g., CD3α-based or CD3β-based TCR domain) binds endogenous CD3δ.

### T Cell Receptors (TCRs) as Membrane-Associated Agents

In some embodiments, a membrane-associated agent comprises a TCR protein (e.g., a TCR alpha molecule, TCR beta molecule, TCR gamma molecule, and/or TCR delta molecule), or a functional fragment or variant thereof. In some embodiments, a membrane-associated agent comprises a TCR alpha and/or a TCR beta. In some embodiments, a membrane-associated agent comprises a TCR gamma and/or a TCR delta. In some embodiments, a membrane-associated agent comprises a CD3 zeta molecule, or a functional fragment or variant thereof. Exemplary human TCR proteins are known in the art. In some embodiments, the TCR protein comprises a DMF5 TCR, or a functional fragment or variant thereof. In some embodiments, the TCR protein comprises an OT-1 TCR, or a functional fragment or variant thereof. In some embodiments, a TCR, CAR, or functional fragment thereof specifically binds to an MHC presenting a cognate peptide (e.g., on the surface of an acceptor cell). In some embodiments, a TCR or functional fragment thereof comprises a TCR alpha molecule and/or a TCR beta molecule bound to a CD3 zeta molecule. In some embodiments, the TCR molecule is endogenous to the donor cell. In some embodiments, the TCR molecule is exogenous to the donor cell.

In some embodiments, a membrane-associated agent comprises a docking moiety (e.g., a first docking moiety as described herein). In embodiments, the docking moiety comprises an extracellular portion (e.g., the extracellular domain) of a TCR molecule, or a functional fragment or variant thereof. In embodiments, the docking moiety comprises an extracellular portion (e.g., the extracellular domain) of a TCR alpha molecule, or a functional fragment or variant thereof. In embodiments, the docking moiety comprises an extracellular portion (e.g., the extracellular domain) of a TCR beta molecule, or a functional fragment or variant thereof. In embodiments, the docking moiety comprises an extracellular portion (e.g., the extracellular domain) of a TCR gamma molecule, or a functional fragment or variant thereof. In embodiments, the docking moiety comprises an extracellular portion (e.g., the extracellular domain) of a TCR delta molecule, or a functional fragment or variant thereof.

In some embodiments, a membrane-associated agent comprises an intracellular moiety. In embodiments, the intracellular moiety comprises an intracellular portion of a TCR molecule, or a functional fragment or variant thereof. In embodiments, the intracellular moiety comprises an intracellular portion (e.g., the intracellular domain) of a TCR alpha molecule, or a functional fragment or variant thereof. In embodiments, the intracellular moiety comprises an intracellular portion (e.g., the intracellular domain) of a TCR beta molecule, or a functional fragment or variant thereof. In embodiments, the intracellular moiety comprises an intracellular portion (e.g., the intracellular domain) of a TCR gamma molecule, or a functional fragment or variant thereof. In embodiments, the intracellular moiety comprises an intracellular portion (e.g., the intracellular domain) of a TCR delta molecule, or a functional fragment or variant thereof. In embodiments, the intracellular moiety comprises an intracellular portion (e.g., the intracellular domain) of a CD3 zeta molecule, or a functional fragment or variant thereof (e.g., a CD3 zeta mutant, e.g., comprising one or more (e.g., 1, 2, 3, 4, 5, or 6) mutations of tyrosine residues (e.g., to phenylalanine). In an embodiment, the intracellular moiety comprises a CD3 zeta YFx6 domain, e.g., as described herein.

In some embodiments, a membrane-associated agent comprises a membrane-associated moiety (e.g., a transmembrane domain). In embodiments, the membrane-associated moiety comprises the transmembrane domain of a TCR molecule, or a functional fragment or variant thereof. In embodiments, the membrane-associated moiety comprises a membrane-associated portion (e.g., the transmembrane domain) of a TCR alpha molecule, or a functional fragment or variant thereof. In embodiments, the membrane-associated moiety comprises a membrane-associated portion (e.g., the transmembrane domain) of a TCR beta molecule, or a functional fragment or variant thereof. In embodiments, the membrane-associated moiety comprises a membrane-associated portion (e.g., the transmembrane domain) of a TCR gamma molecule, or a functional fragment or variant thereof. In embodiments, the membrane-associated moiety comprises a membrane-associated portion (e.g., the transmembrane domain) of a TCR delta molecule, or a functional fragment or variant thereof. In embodiments, the membrane-associated moiety comprises a membrane-associated portion (e.g., the transmembrane domain) of a CD3 zeta molecule, or a functional fragment or variant thereof.

In some embodiments, a membrane-associated agent is associated with (e.g., is covalently bound to, non-covalently bound to, is present in a membrane with, or is colocalized in a membrane with) a cargo molecule, e.g., as described herein. In embodiments, the cargo molecule is not bound to the membrane-associated agent, but is attached to the cell membrane (e.g., comprises a transmembrane domain and/or is tethered to the membrane, e.g., via an inner leaflet tether, e.g., a palmitoylation/myristoylatoin domain of Lck, e.g., as described herein). In embodiments, the cargo molecule and the membrane-associated agent are present in the same lipid raft in the membrane. In some embodiments, a membrane-associated agent is present in the cytoplasm of the cell.

In embodiments, the cargo molecule is covalently attached to the membrane-associated agent (e.g., the cargo molecule and at least a portion of the membrane-associated agent are a fusion protein). In an embodiment, the cargo molecule is covalently bound to a TCR alpha molecule, a TCR beta molecule, a TCR gamma molecule, or a TCR delta molecule. In an embodiment, the cargo molecule is covalently bound to a CD3 zeta molecule, e.g., at the C-terminus of the CD3 zeta molecule.

In embodiments, the cargo molecule is non-covalently bound to the membrane-associated agent (e.g., the TCR, CAR, or functional fragment or variant thereof). In an embodiment, the cargo molecule is non-covalently bound to a TCR alpha molecule. In an embodiment, the cargo molecule is non-covalently bound to a TCR beta molecule. In an embodiment, the cargo molecule is non-covalently bound to a TCR gamma molecule. In an embodiment, the cargo molecule is non-covalently bound to a TCR delta molecule. In an embodiment, the cargo molecule is non-covalently bound to a CD3 zeta molecule. In an embodiment, the cargo molecule comprises a ZAP70 molecule, or a functional fragment or variant thereof (e.g., the SH2 domains of a ZAP70 molecule). In an embodiment, the CAP70 molecule is a human ZAP70 molecule.

In some embodiments, the membrane-associated agent comprises a nucleic acid binding domain (e.g., in the intracellular moiety), e.g., wherein the cargo molecule is a nucleic acid molecule (e.g., a DNA or RNA), e.g., as described herein. In embodiments, the membrane-associated agent comprises a DNA binding domain, e.g., in the intracellular moiety. In embodiments, the membrane-associated agent comprises an RNA binding domain, e.g., in the intracellular moiety. In embodiments, the RNA binding domain comprises an RNA binding domain from a viral protein (e.g., a viral coat protein, e.g., a bacteriophage coat protein, e.g., an MS2 coat protein). In embodiments, the nucleic acid binding domain is associated with (e.g., covalently or non-covalently bound to) a domain or subunit of the membrane-associated agent. In embodiments, the nucleic acid binding domain is covalently attached to a CD3 zeta molecule, or a functional fragment or variant thereof, in the membrane-associated agent. In embodiments, the nucleic acid binding domain is bound to a ZAP70 molecule, or a functional fragment or variant thereof (e.g., the SH2 domains of a ZAP70 molecule), which in turn binds non-covalently to a CD3 zeta molecule, or a functional fragment or variant thereof, in the membrane-associated agent.

In some embodiments, a membrane-associated agent (e.g., comprising a TCR, CAR, or a functional fragment or variant thereof) induces or promotes activation of the donor cell (e.g., a T cell or NK cell). In embodiments, activation of the donor cell comprises upregulation of the membrane-associated agent (e.g., TCR, CAR, or functional fragment or variant thereof) by the donor cell.

### Chimeric Antigen Receptors (CARs)

The present disclosure provides, in some embodiments, donor cells and/or acceptor cells comprising a chimeric antigen receptor (CAR), or a functional fragment thereof. Generally, a CAR is a recombinant polypeptide construct comprising an antigen binding domain (which is extracellular), a transmembrane domain, and an intracellular moiety (e.g., comprising a functional signaling domain, e.g., derived from a stimulatory molecule). In some embodiments, the intracellular moiety comprises a functional signaling domain. In some embodiments, the intracellular moiety does not comprise a signaling domain. In some embodiments, the intracellular moiety comprises a nonfunctional signaling domain. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains. In some embodiments, the antigen binding domain comprises an antibody molecule (e.g., an antibody or a functional fragment or variant thereof, e.g., an scFv). In some embodiments, the signaling domain comprises a primary signaling domain (e.g., a primary signaling domain of CD3-zeta). In some embodiments, the signaling domain comprises one or more functional signaling domains derived from at least one costimulatory molecule, e.g., 4-1BB, CD27, and/or CD28. In some embodiments, the CAR comprises a chimeric fusion protein comprising an antigen binding domain, a transmembrane domain and a signaling domain comprising a functional signaling domain derived from a stimulatory molecule. In some embodiments, the CAR comprises a chimeric fusion protein comprising an antigen binding domain, a transmembrane domain and a signaling domain comprising a functional signaling domain derived from a costimulatory molecule and a functional signaling domain derived from a stimulatory molecule. In some embodiments the CAR comprises an optional leader sequence at the amino-terminus of the CAR fusion protein, wherein the leader sequence is optionally cleaved from the antigen binding domain (e.g., a scFv) during cellular processing and localization of the CAR to the cellular membrane. In some embodiments, the signaling domain induces or promotes signaling in the donor cell, e.g., when the CAR is bound to a target moiety (e.g., a second docking moiety) on a target cell (e.g., an acceptor cell). In embodiments, the signaling domain induces or promotes activation of the donor cell (e.g., activation of a donor cell that is a T cell or NK cell).

In some instances, a CAR comprises 1, 2, or all 3 of: (i) an extracellular moiety, e.g., comprising a first docking moiety (e.g., comprising a binding domain that binds specifically to a target moiety (e.g., a second docking moiety) on a target cell, e.g., an acceptor cell), (ii) a membrane-associated moiety (e.g., comprising a transmembrane domain, e.g., as described herein), and/or (iii) an intracellular moiety (e.g., as described herein). In some embodiments, a first docking moiety of a CAR comprises an antigen-binding domain (e.g., an antibody molecule, e.g., an antibody or a functional fragment or variant thereof, e.g., an scFv). In some embodiments, a membrane-associated moiety of a CAR comprises a transmembrane domain. In some embodiments, an intracellular moiety of a CAR comprises a signaling domain.

In some embodiments, the CAR is comprised in a membrane-associated agent of a donor cell. In some embodiments, the CAR is comprised in a membrane-associated agent in an acceptor cell, e.g., after transfer of the CAR from a donor cell. In some embodiments, a cargo molecule is bound to a CAR (e.g., by its extracellular moiety or intracellular moiety). In some embodiments, a cargo molecule is operably associated or linked (e.g., tethered) to a CAR (e.g., by its extracellular moiety or intracellular moiety).

In some embodiments, a donor cell or acceptor cell comprises a CAR (e.g., a first generation CAR) or a nucleic acid encoding a first generation CAR. In some embodiments, a membrane-associated agent or a cargo molecule comprises a CAR (e.g., a first generation CAR) or a nucleic acid encoding a first generation CAR. In some embodiments, a first generation CAR comprises an antigen binding domain, a transmembrane domain, and signaling domain. In some embodiments a signaling domain mediates downstream signaling during T-cell activation.

In some embodiments, a donor cell or acceptor cell comprises a second generation CAR or a nucleic acid encoding a second generation CAR. In some embodiments, a membrane-associated agent or a cargo molecule comprises a second generation CAR or a nucleic acid encoding a second generation CAR. In some embodiments a second generation CAR comprises an antigen binding domain, a transmembrane domain, and two signaling domains. In some embodiments a signaling domain mediates downstream signaling during T-cell activation. In some embodiments a signaling domain is a costimulatory domain. In some embodiments, a costimulatory domain enhances cytokine production, CAR T-cell proliferation, and or CAR T-cell persistence during T cell activation.

In some embodiments, a donor cell or acceptor cell comprises a third generation CAR or a nucleic acid encoding a third generation CAR. In some embodiments, a membrane-associated agent or a cargo molecule comprises a third generation CAR or a nucleic acid encoding a third generation CAR. In some embodiments, a third generation CAR comprises an antigen binding domain, a transmembrane domain, and at least three signaling domains. In some embodiments a signaling domain mediates downstream signaling during T-cell activation. In some embodiments a signaling domain is a costimulatory domain. In some embodiments, a costimulatory domain enhances cytokine production, CAR T-cell proliferation, and or CAR T-cell persistence during T cell activation. In some embodiments, a third generation CAR comprises at least two costimulatory domains. In some embodiments, the at least two costimulatory domains are not the same.

In some embodiments, a donor cell or acceptor cell comprises a fourth generation CAR or a nucleic acid encoding a fourth generation CAR. In some embodiments, a membrane-associated agent or a cargo molecule comprises a fourth generation CAR or a nucleic acid encoding a fourth generation CAR. In some embodiments a fourth generation CAR comprises an antigen binding domain, a transmembrane domain, and at least two, three, or four signaling domains. In some embodiments a signaling domain mediates downstream signaling during T-cell activation. In some embodiments a signaling domain is a costimulatory domain. In some embodiments, a costimulatory domain enhances cytokine production, CAR T-cell proliferation, and or CAR T-cell persistence during T cell activation. In some embodiments, the CAR has reduced killing capacity (e.g., by at least about 10%, 20%, 30%, 40%, 50%, 60%, 70%, 80%, 90%, 95%, 96%, 97%, 98%, 99%, or 100%) compared to an otherwise similar CAR. In some embodiments, the CAR induces reduced cytokine release (e.g., by at least about 10%, 20%, 30%, 40%, 50%, 60%, 70%, 80%, 90%, 95%, 96%, 97%, 98%, 99%, or 100%) compared to an otherwise similar CAR.

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments a cytokine gene encodes a pro-inflammatory cytokine. In some embodiments a cytokine gene encodes IL-1, IL-2, IL-9, IL-12, IL-18, TNF, or IFN-gamma, or functional fragment thereof. In some embodiments a domain which upon successful signaling of the CAR induces expression of a cytokine gene is or comprises a transcription factor or functional domain or fragment thereof. In some embodiments a domain which upon successful signaling of the CAR induces expression of a cytokine gene is or comprises a transcription factor or functional domain or fragment thereof. In some embodiments a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, a membrane-associated agent comprises a CAR that specifically binds to a peptide presented on an MHC class I protein. In some embodiments, a membrane-associated agent comprises a CAR that specifically binds to a peptide comprising the amino acid sequence SIINFEKL.

In some embodiments, a membrane-associated agent comprises a CAR that specifically binds to myelin oligodendrocyte glycoprotein (MOG).

In some embodiments, a membrane-associated agent comprises a CAR that comprises a streptavidin moiety. In some embodiments, a membrane-associated agent comprises a CAR that comprises a biotin moiety.

### CAR Antigen Binding Domains

In some embodiments, a CAR (e.g., comprised in a donor cell or acceptor cell, e.g., in a membrane-associated agent, or cargo molecule) comprises an antigen binding domain (a CAR antigen binding domain). In some embodiments, the CAR antigen binding domain binds a cancer associated antigen. In some embodiments, the antigen binding domain is extracellular. In some embodiments, the antigen binding domain is comprised in a docking moiety as described herein (e.g., a first docking moiety of a membrane-associated agent). In some embodiments, a CAR antigen binding domain is or comprises an antibody molecule (e.g., an antibody or a functional fragment, variant, or antigen-binding portion thereof). In some embodiments, a CAR antigen binding domain is or comprises an scFv or Fab. In some embodiments, a CAR antigen binding domain is or comprises a full length antibody (e.g., an antibody comprising two heavy chains and two light chains). In some embodiments a CAR antigen binding domain (e.g., an antigen binding domain that comprises an scFv or Fab fragment) binds an antigen chosen from: T-cell alpha chain; T-cell β chain; T-cell γ chain; T-cell δ chain; CCR7; CD3; CD4; CD5; CD7; CD8; CD11b; CD11c; CD16; CD19; CD20; CD21; CD22; CD25; CD28; CD34; CD35; CD40; CD45RA; CD45RO; CD52; CD56; CD62L; CD68; CD80; CD95; CD117; CD127; CD133; CD137 (4-1 BB); CD163; F4/80; IL-4Ra; Sca-1; CTLA-4; GITR GARP; LAP; granzyme B; LFA-1; or transferrin receptor.

In some embodiments the antigen bound by the CAR is a molecule (typically a protein, carbohydrate or lipid) that is expressed on the surface of an acceptor cell, either entirely or as a fragment (e.g., MHC/peptide), and which is useful for preferential binding of a pharmacological agent to the acceptor cell.

In some embodiments, an antigen binding domain binds to a cell surface antigen of a target cell (e.g., an acceptor cell). In some embodiments, a cell surface antigen is characteristic of one type of cell. In some embodiments, a cell surface antigen is characteristic of more than one type of cell. In some embodiments, a CAR antigen binding domain binds to a cell surface receptor, a membrane transport protein (e.g., an active or passive transport protein such as, for example, an ion channel protein, a pore-forming protein, etc.), a transmembrane receptor, a membrane enzyme, and/or a cell adhesion protein. In some embodiments, a CAR antigen binding domain binds to a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, a CAR antigen binding domain binds to a T-cell receptor (TCR). In some embodiments, a T-cell receptor may be AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p380); MAPK12 (p38γ); MAPK13 (p386); MAPK14 (p38a); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments a CAR antigen binding domain binds an antigen characteristic of a neoplastic cell, e.g., cancer. In some embodiments an antigen characteristic of a cancer is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (SIPIR), NMDA channel, transmembrane protein, multispan transmembrane protein, T-cell receptor motifs; T-cell alpha chains; T-cell β chains; T-cell γ chains; T-cell δ chains; CCR7; CD3; CD4; CD5; CD7; CD8; CD11b; CD11c; CD16; CD19; CD20; CD21; CD22; CD25; CD28; CD34; CD35; CD40; CD45RA; CD45RO; CD52; CD56; CD62L; CD68; CD80; CD95; CD117; CD127; CD133; CD137 (4-1 BB); CD163; F4/80; IL-4Ra; Sca-1; CTLA-4; GITR; GARP; LAP; granzyme B; LFA-1; transferrin receptor; NKp46, perforin, CD4+; Th1; Th2; Th17; Th40; Th22; Th9; Tfh, Canonical Treg. FoxP3+; Trl; Th3; Treg17; T_{RE}G; CDCP1, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), LiCAM, LeY, MSLN, IL13Rα1, L1-CAM, Tn Ag, prostate specific membrane antigen (PSMA), RORI, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLACl, GloboH, NY-BR-1, UPK2, HAVCR1, ADRB3, PANX3, GPR20, LY6K, OR51E2, TARP, WTi, NY-ESO-1, LAGE-1a, MAGE-A1, legumain, HPV E6, E7, ETV6-AML, sperm protein 17, XAGEl, Tie 2, MAD-CT-1, MAD-CT-2, Fos-related antigen 1, p53, p53 mutant, prostein, survivin, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIRI, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, or an antigenic fragment or antigenic portion thereof. In other embodiments, the antigen is not characteristic of cancer cells. In some embodiments, the antigen is a marker expressed by both normal cells and cancer cells, e.g., a lineage marker, e.g., CD19 on B cells.

In some embodiments a CAR antigen binding domain binds an antigen characteristic of an infectious disease (e.g. a viral infection or a bacterial infection). In some embodiments an antigen is characteristic of an infectious disease selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Eptstein-Barr virus, CMV, human papillomavirus. In some embodiments an antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor. In some embodiments, a CAR antigen binding domain binds an antigen characteristic of an infectious disease, wherein the antigen is selected from HIV Env, gpl20, or CD4-induced epitope on HIV-1 Env. See, e.g., WO2015/077789, the contents of which are herein incorporated by reference. In some embodiments, a CAR antigen binding domain comprises CD4 or an HIV binding fragment thereof. In other embodiments, the antigen is not characteristic of an infectious disease.

In some embodiments a CAR antigen binding domain binds an antigen characteristic of an autoimmune or inflammatory disorder. In some embodiments the antigen is characteristic of an autoimmune or inflammatory disorder selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments an antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor. In some embodiments, a CAR antigen binding domain binds to a ligand expressed on B cells, plasma cells, or plasmablasts. In some embodiments, a CAR antigen binding domain binds an antigen characteristic of an autoimmune or inflammatory disorder, wherein the antigen is selected from CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See US 2003/0077249; WO 2017/058753; WO 2017/058850, the contents of which are herein incorporated by reference. In other embodiments, the antigen is not characteristic of an autoimmune or inflammatory disorder.

### CAR Transmembrane Domains

In some embodiments, a CAR (e.g., comprised in a donor cell or acceptor cell, e.g., in a membrane-associated agent, or cargo molecule) comprises a transmembrane domain (a “CAR transmembrane domain”). In some embodiments, a membrane-associated moiety of a membrane-associated agent comprises a CAR transmembrane domain. In some embodiments a CAR comprises at least a transmembrane region of the alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments a CAR comprises at least a transmembrane region of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof. In some embodiments, the transmembrane domain comprises a hydrophobic alpha helix. In some embodiments, the transmembrane domain comprises a CD28 transmembrane domain. In some embodiments, the transmembrane domain comprises a CD8 transmembrane domain. In some embodiments, the transmembrane domain comprises a CD3 zeta transmembrane domain. In some embodiments, the intracellular moiety comprises a CD3 zeta intracellular domain, or a functional fragment or variant thereof.

### CAR Intracellular Domains

In some embodiments, a CAR (e.g., comprised in a donor cell or acceptor cell, e.g., in a membrane-associated agent, or cargo molecule) comprises a signaling domain (a “CAR signaling domain”), e.g., in an intracellular domain. In some embodiments, a CAR comprises a functional signaling domain. In some embodiments, a CAR comprises a nonfunctional signaling domain. In some embodiments, a CAR does not comprise a signaling domain. In some embodiments, a CAR comprises a signaling domain that induces or promotes activation of a cell (e.g., a donor cell or an acceptor cell), e.g., a T cell or an NK cell. In some embodiments, activation of the cell comprises upregulation of the CAR by the cell, e.g., the T cell or NK cell. In some embodiments a CAR comprises a signaling domain of one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thyl; CD96; CD160; CD200; CD300a/LMIRI; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), or functional variant thereof. In some embodiments, a CAR as described herein does not substantially induce cytotoxic signaling, or induces reduced cytotoxicity signaling relative to a reference CAR (e.g., by at least about 10%, 20%, 30%, 40%, 50%, 60%, 70%, 80%, 90%, 95%, 96%, 97%, 98%, 99%, or 100%). In some embodiments, the CAR comprises an intracellular domain without a signaling domain. In some embodiments, the intracellular domain comprises an antibody molecule (e.g., an scFv), e.g., capable of binding to a cargo molecule, e.g., as described herein.

In some embodiments a CAR comprises a signaling domain which is a costimulatory domain. In some embodiments a CAR comprises a second costimulatory domain. In some embodiments a CAR comprises at least two costimulatory domains. In some embodiments a CAR comprises at least three costimulatory domains. In some embodiments a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83.

In some embodiments, a CAR comprises a CD3 zeta domain or an immunoreceptor tyrosine-based activation motif (ITAM), or functional variant thereof. In some embodiments, a CAR comprises a (i) a CD3 zeta domain, or an immunoreceptor tyrosine-based activation motif (ITAM), or functional variant thereof, and (ii) a CD28 domain, or a CD8 domain, or a 4-1BB domain, or functional variant thereof. In some embodiments, a CAR comprises a (i) a CD3 zeta domain, or an immunoreceptor tyrosine-based activation motif (ITAM), or functional variant thereof, (ii) a CD28 domain, CD8 domain, or a functional fragment or variant thereof, and (iii) a 4-1BB domain, or a CD134 domain, or functional variant thereof. In some embodiments, a CAR comprises a (i) a CD3 zeta domain, or an immunoreceptor tyrosine-based activation motif (ITAM), or functional variant thereof, (ii) a CD28 domain, CD8 domain, or a functional fragment or variant thereof, (iii) a 4-1BB domain, or a CD134 domain, or functional variant thereof, and (iv) a cytokine or costimulatory ligand transgene.

### CAR Spacers

In some embodiments a CAR (e.g., comprised in a donor cell or acceptor cell, e.g., in a membrane-associated agent, or cargo molecule) comprises one or more spacers. In some embodiments a CAR comprises a spacer between the antigen binding domain and the transmembrane domain. In some embodiments a CAR comprises a spacer between a transmembrane domain and an intracellular signaling domain.

In addition to the CARs described herein, various chimeric antigen receptors and nucleotide sequences encoding the same are known in the art and would be suitable for transfer from a donor cell to an acceptor cell, e.g., as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

In some embodiments, a membrane-associated agent comprising a CAR, e.g., comprised in a donor cell, is transferred to an acceptor cell. In some embodiments, an acceptor cell may include, but may not be limited to, one or more of a monocyte, macrophage, neutrophil, dendritic cell, eosinophil, mast cell, platelet, large granular lymphocyte, Langerhans' cell, natural killer (NK) cell, T-lymphocyte (e.g., T-cell), a Gamma delta T cell, B-lymphocyte (e.g., B-cell) and may be from any organism including but not limited to humans, mice, rats, rabbits, and monkeys.

### Antibody Molecules

In some embodiments, a CAR (e.g., in a membrane-associated agent) comprises (e.g., in its extracellular moiety, e.g., in a docking moiety) an antibody molecule (e.g., an antibody or a functional fragment or variant thereof). In some embodiments, the CAR comprises a full-length antibody (e.g., an antibody comprising two heavy chains and two light chains). In embodiments, the antibody is attached to a moiety (e.g., a biotin moiety) that binds to an extracellular region of the CAR (e.g., a streptavidin moiety). In some embodiments, the CAR comprises an antigen-binding fragment of an antibody. Non-limiting examples of antigen-binding fragments include: (i) a Fab fragment, a monovalent fragment consisting of the VL, VH, CL and CHi domains; (ii) a F(ab′)2 fragment, a bivalent fragment comprising two Fab fragments linked by a disulfide bridge at the hinge region; (iii) a Fd fragment consisting of the VH and CHi domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including several conventional techniques known to those with skill in the art, and the fragments can be screened for utility in the same manner as are intact antibodies.

The VH and VL regions of an antibody molecule can be subdivided into regions of hypervariability, termed “complementarity determining regions” (CDR), interspersed with regions that are more conserved, termed “framework regions” (FR or FW). The terms “complementarity determining region,” and “CDR,” as used herein refer to the sequences of amino acids within antibody variable regions which confer antigen specificity and binding affinity. As used herein, the terms “framework,” “FW” and “FR” are used interchangeably.

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). In an embodiment, the following definitions are used: AbM definition of CDR1 of the heavy chain variable domain and Kabat definitions for the other CDRs. In an embodiment, Kabat definitions are used for all CDRs. In addition, embodiments described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, and FR4.

In some embodiments, the antibody molecule is a single chain antibody. A single-chain antibody (scFv) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target protein. The antibody molecules disclosed herein can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to some aspects, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are also contemplated.

In an embodiment, the antibody molecule is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. In an embodiment, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by any suitable recombinant DNA technique. Several are known in the art (see Robinson et al., International Patent Application Publication No. WO1987/002671; Akira, et al., European Patent Application Publication No. 184,187; Taniguchi, M., European Patent Application Publication No. 171,496; Morrison et al., European Patent Application Publication No. 173,494; Neuberger et al., International Patent Application Publication No. WO 86/01533; Cabilly et al. U.S. Pat. No. 4,816,567; Cabilly et al., European Patent Application Publication No. 125,023; Better et al. (1988 Science 240:1041-1043); Liu et al. (1987) PNAS 84:3439-3443; Liu et al., 1987, J Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to lipopolysaccharide. In an embodiment, the donor will be a rodent antibody, e.g., a rat or mouse antibody, and the recipient will be a human framework or a human consensus framework. In some embodiments, the donor immunoglobulin is a non-human (e.g., rodent). The acceptor framework is typically a naturally-occurring (e.g., a human) framework or a consensus framework, or a sequence about 85% or higher, e.g., 90%, 95%, 99% or higher identical thereto.

An antibody can be humanized by any suitable method, and several such methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare humanized antibodies (UK Patent Application GB 2188638A, filed on Mar. 26, 1987; Winter U.S. Pat. No. 5,225,539), the contents of which is expressly incorporated by reference.

In an embodiment, the antibody molecule has a heavy chain constant region chosen from, e.g., the heavy chain constant regions of IgG1, IgG2 (e.g., IgG2a), IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody molecule (e.g., to increase or decrease one or more of: Fc receptor binding, antibody glycosylation, the number of cysteine residues, effector cell function, and/or complement function). In an embodiment, the antibody molecule has effector function and can fix complement. In another embodiment, the antibody molecule does not recruit effector cells or fix complement. In certain embodiments, the antibody molecule has reduced or no ability to bind an Fc receptor. For example, it may be an isotype or subtype, fragment or other mutant, which does not support binding to an Fc receptor, e.g., it has a mutagenized or deleted Fc receptor binding region.

In an embodiment, a constant region of the antibody molecule is altered. Methods for altering an antibody constant region are known in the art. Antibody molecules with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Amino acid mutations which stabilize antibody structure, such as S228P (EU nomenclature, S241P in Kabat nomenclature) in human IgG4 are also contemplated. Similar type of alterations could be described which if applied to the murine, or other species immunoglobulin would reduce or eliminate these functions.

### CARs as Membrane-Associated Agents

In some embodiments, a membrane-associated agent comprises a CAR. In some embodiments, a membrane-associated agent comprises an antibody molecule (e.g., an antibody or a functional fragment, variant, or antigen-binding fragment thereof, e.g., an scFv), e.g., which binds specifically to a moiety on the surface of a target cell (e.g., an acceptor cell). In some embodiments, a membrane-associated agent comprises a CD3 zeta molecule, or a functional fragment or variant thereof.

In some embodiments, a membrane-associated agent comprises a docking moiety (e.g., a first docking moiety as described herein). In embodiments, the docking moiety comprises an extracellular portion (e.g., the extracellular domain) of a CAR. In embodiments, the docking moiety comprises an antibody molecule (e.g., an antibody or a functional fragment, variant, or antigen-binding fragment thereof, e.g., an scFv), e.g., which binds specifically to a moiety (e.g., a second docking moiety) on the surface of a target cell (e.g., an acceptor cell). In embodiments, the docking moiety comprises a moiety that is associated with the antibody molecule, e.g., is covalently or noncovalently bound to the antibody molecule. In embodiments, the portion of the docking moiety associated with the antibody molecule comprises an avidin, e.g., a streptavidin (e.g., comprises an mSA2 domain) and the antibody molecule is biotinylated. In some embodiments, the antibody is an abiotinylated antibody attached to an mSA2 domain. In an embodiment, the antibody is an anti-CD19 antibody. In an embodiment, the antibody is an anti-HLA antibody (e.g., an anti-HLA-G antibody).

In some embodiments, a membrane-associated agent comprises an intracellular moiety. In embodiments, the intracellular moiety comprises an intracellular portion (e.g., the intracellular domain) of a CD3 zeta molecule, or a functional fragment or variant thereof (e.g., a CD3 zeta mutant, e.g., comprising one or more (e.g., 1, 2, 3, 4, 5, or 6) mutations of tyrosine residues (e.g., to phenylalanine). In an embodiment, the intracellular moiety comprises a CD3 zeta YFx6 domain, e.g., as described herein, e.g., having an amino acid sequence encoded by the nucleic acid sequence of SEQ ID NO: 1, or an amino acid sequence having at least 75%, 80%, 85%, 90%, 95%, 96%, 97%, 98%, or 99% identity thereto.

>CD3z YFx6-EGFP (uppercase sequence corresponds to CD3 zeta YFx6 domain)
(SEQ ID NO: 1)
gccaccATGAAGTGGAAAGTGTCTGTTCTCGCCTGCATCCTCCACGTGCGGTTCCCAGGAGCAG
AGGCACAGAGCTTTGGTCTGCTGGACCCCAAACTCTGCTACTTGCTAGATGGAATCCTCTTC
ATCTACGGAGTCATCATCACAGCCCTGTACCTGAGAGCAAAATTCAGCAGGAGTGCAGAGA
CTGCTGCCAACCTGCAGGACCCCAACCAGCTCTTCAATGAGCTCAATCTAGGGCGAAGAGA
GGAATTTGACGTCTTGGAGAAGAAGCGGGCTCGGGACCCAGAGATGGGAGGCAAACAGCA
GAGGAGGAGGAACCCCCAGGAAGGCGTATTCAATGCACTGCAGAAAGACAAGATGGCAGA
AGCCTTCAGTGAGATCGGCACAAAAGGCGAGAGGCGGAGAGGCAAGGGGCACGATGGCCT
TTTCCAGGGTCTCAGCACTGCCACCAAGGACACCTTTGATGCCCTGCATATGCAGACCCTGG
CCCCTCGCccacccgtcgccaccatggtgagcaagggcgaggagctgttcaccggggtggtgcccatcctggtcgagctggacggcgacgta
aacggccacaagttcagcgtgtccggcgagggcgagggcgatgccacctacggcaagctgaccctgaagttcatctgcaccaccggcaagctgc
ccgtgccctggcccaccctcgtgaccaccctgacctacggcgtgcagtgcttcagccgctaccccgaccacatgaagcagcacgacttcttcaa
gtccgccatgcccgaaggctacgtccaggagcgcaccatcttcttcaaggacgacggcaactacaagacccgcgccgaggtgaagttcgagggc
gacaccctggtgaaccgcatcgagctgaagggcatcgacttcaaggaggacggcaacatcctggggcacaagctggagtacaactacaacagcc
acaacgtctatatcatggccgacaagcagaagaacggcatcaaggtgaacttcaagatccgccacaacatcgaggacggcagcgtgcagctcgc
cgaccactaccagcagaacacccccatcggcgacggccccgtgctgctgcccgacaaccactacctgagcacccagtccgccctgagcaaagac
cccaacgagaagcgcgatcacatggtcctgctggagttcgtgaccgccgccgggatcactctcggcatggacgagctgtataagtga

In an embodiment, a membrane-associated agent comprises a wild-type CD3 zeta molecule, e.g., comprising the amino acid sequence MKWKVSVLACILHVRFPGAEAQSFGLLDPKLCYLLDGILFIYGVIITALYLRAKFSRSAETAANLQ DPNQLYNELNLGRREEYDVLEKKRARDPEMGGKQQRRRNPQEGVYNALQKDKMAEAYSEIGT KGERRRGKGHDGLYQGLSTATKDTYDALHMQTLAPR (SEQ ID NO: 39), or an amino acid sequence having at least 75%, 80%, 85%, 90%, 95%, 96%, 97%, 98%, or 99% sequence identity thereto.

In embodiments, the intracellular moiety comprises a marker (e.g., a fluorescent marker, e.g., a fluorescent protein, e.g., GFP, RFP, YFP, or mCherry).

In some embodiments, a membrane-associated agent comprises a membrane-associated moiety (e.g., a transmembrane domain). In embodiments, the membrane-associated moiety comprises a membrane-associated portion (e.g., the transmembrane domain) of CD28, or a functional fragment or variant thereof. In embodiments, the membrane-associated moiety comprises a membrane-associated portion (e.g., the transmembrane domain) of CD8, or a functional fragment or variant thereof. In embodiments, the membrane-associated moiety comprises a membrane-associated portion (e.g., the transmembrane domain) of a CD3 zeta molecule, or a functional fragment or variant thereof.

In some embodiments, a membrane-associated agent is associated with (e.g., is covalently bound to, non-covalently bound to, is present in a membrane with, or is colocalized in a membrane with) a cargo molecule, e.g., as described herein. In embodiments, the cargo molecule is not bound to the membrane-associated agent, but is attached to the cell membrane (e.g., comprises a transmembrane domain and/or is tethered to the membrane, e.g., via an inner leaflet tether, e.g., a palmitoylation/myristoylation domain of Lck, e.g., as described herein). In embodiments, the cargo molecule and the membrane-associated agent are present in the same lipid raft in the membrane. In some embodiments, a membrane-associated agent is present in the cytoplasm of the cell.

In embodiments, the cargo molecule is covalently attached to the membrane-associated agent (e.g., the CAR). In embodiments, the cargo molecule and at least a portion of the membrane-associated agent are a fusion protein. In an embodiment, the cargo molecule is covalently bound to the intracellular moiety of the membrane-associated agent. In an embodiment, the cargo molecule is covalently bound to a CD3 zeta molecule, e.g., at the C-terminus of the CD3 zeta molecule.

In embodiments, the cargo molecule is non-covalently bound to the membrane-associated agent (e.g., the CAR). In an embodiment, the cargo molecule is non-covalently bound to the intracellular moiety of the membrane-associated agent. In an embodiment, the cargo molecule is non-covalently bound to a CD3 zeta molecule. In an embodiment, the cargo molecule comprises a ZAP70 molecule, or a functional fragment or variant thereof (e.g., the SH2 domains of a ZAP70 molecule). In an embodiment, the ZAP70 molecule is a human ZAP70 molecule.

In some embodiments, the membrane-associated agent comprises a nucleic acid binding domain (e.g., in the intracellular moiety), e.g., wherein the cargo molecule is a nucleic acid molecule (e.g., a DNA or RNA), e.g., as described herein. In embodiments, the membrane-associated agent comprises a DNA binding domain, e.g., in the intracellular moiety. In embodiments, the membrane-associated agent comprises an RNA binding domain, e.g., in the intracellular moiety. In embodiments, the RNA binding domain comprises an RNA binding domain from a viral protein (e.g., a viral coat protein, e.g., a bacteriophage coat protein, e.g., an MS2 coat protein). In embodiments, the nucleic acid binding domain is associated with (e.g., covalently or non-covalently bound to) a domain or subunit of the membrane-associated agent. In embodiments, the nucleic acid binding domain is covalently attached to a CD3 zeta molecule, or a functional fragment or variant thereof, in the membrane-associated agent. In embodiments, the nucleic acid binding domain is bound to a ZAP70 molecule, or a functional fragment or variant thereof (e.g., the SH2 domains of a ZAP70 molecule), which in turn binds non-covalently to a CD3 zeta molecule, or a functional fragment or variant thereof, in the membrane-associated agent.

In some embodiments, a membrane-associated agent (e.g., comprising a CAR) induces or promotes activation of the donor cell (e.g., a T cell or NK cell). In embodiments, activation of the donor cell comprises upregulation of the membrane-associated agent (e.g., the CAR) by the donor cell.

### Modifications to Exogenous Membrane-Associated Agents

In some embodiments a membrane-associated agent can be altered to reduce immunoreactivity. For instance, a membrane-associated agent may be decorated with molecules that reduce immune interactions, such as PEG (DOI: 10.1128/JVI.78.2.912-921.2004). Thus, in some embodiments, the membrane-associated agent comprises PEG, e.g., is a PEGylated polypeptide. Amino acid residues in the membrane-associated agent that are targeted by the immune system may be altered to be unrecognized by the immune system (doi: 10.1016/j.virol.2014.01.027, doi:10.1371/journal.pone.0046667). In some embodiments the protein sequence of the membrane-associated agent is altered to resemble amino acid sequences found in humans (humanized). In some embodiments the protein sequence of the membrane-associated agent is changed to a protein sequence that binds MHC complexes less strongly. In some embodiments, the membrane-associated agent is derived, at least in part, from viruses or organisms that do not infect humans (and which humans have not been vaccinated against), increasing the likelihood that a patient's immune system is naïve to the membrane-associated agent (e.g., there is a negligible humoral or cell-mediated adaptive immune response towards the membrane-associated agent) (doi: 10.1006/mthe.2002.0550, doi:10.1371/journal.ppat.1005641, doi:10.1038/gt.2011.209, DOI 10.1182/blood-2014-02-558163). In some embodiments, glycosylation of the membrane-associated agent may be changed to alter immune interactions or reduce immunoreactivity. Without wishing to be bound by theory, in some embodiments, a membrane-associated agent derived from a virus or organism that does not infect humans does not have a natural target cell moiety (e.g., to which it binds) in patients, and thus has high specificity.

In some embodiments, a membrane-associated agent comprises one or more (e.g., one, two, three, or more) cleavage sites recognized by a protease. In some embodiments, the protease is not present in a donor cell, or a source cell from which the donor cell is derived. In some embodiments, the protease is present in an acceptor cell or source cell from which the acceptor cell is derived. Without wishing to be bound by theory, a membrane-associated agent comprising a cleavage site for a protease not present in a donor cell may be safely carried, e.g., without being cleaved, by a donor cell. Only upon transfer to a cell containing the appropriate protease (e.g., an acceptor cell) would the membrane-associated agent be cleaved. Exemplary cleavage sites include, but are not limited to, a TEV protease cleavage site and a Rhomboid, veinlet-like 2 (RHBDL2) cleavage site.

In some embodiments, the membrane-associated agent comprises a cleavage site in the membrane-associated moiety. In some embodiments, the membrane-associated agent comprises a cleavage site in the extracellular moiety. In some embodiments, the membrane-associated agent comprises a cleavage site in the intracellular moiety. In some embodiments, the membrane-associated agent comprises a protein that is inactive or unable to activate its function until cleaved from the membrane-associated agent. For example, in some embodiments the intracellular moiety comprises a transcription factor and a cleavage site, wherein upon cleavage the transcription factor is free to translocate to the nucleus and alter transcription. As a further example, in some embodiments, the intracellular moiety comprises a zymogen that is transformed into an active enzyme by cleavage.

### Membrane-Associated Moieties

A membrane-associated moiety associates with (e.g., is localized in and/or on) or is capable of associating with a membrane (e.g., a cell membrane). Generally, the membrane-associated moiety localizes a membrane-associated agent to a membrane.

In some embodiments a membrane-associated moiety is or comprises a transmembrane protein or the transmembrane domain of a transmembrane protein. In some embodiments, a membrane-associated moiety comprises a transmembrane domain from a TCR protein (e.g., a TCR alpha molecule and/or a TCR beta molecule, or a TCR gamma molecule and/or a TCR delta molecule). In some embodiments, a membrane-associated moiety comprises a transmembrane domain from CD28, CD8, or CD3 zeta. In some embodiments, a membrane-associated moiety comprises a lipidation modification sequence, e.g., a N-myristoylation, N-palmitoylation, or S-palmitoylation sequence, or a hydrophobic signal sequence suitable for addition of Glycosylphosphatidylinositol (GPI), e.g., comprises a myristoyl, palmitoyl, or GPI modification. In some embodiments, a membrane-associated moiety is associated with an interior (e.g., cytosolic) portion of a membrane lipid bilayer. In some embodiments a membrane-associated moiety is associated with an exterior portion of a membrane lipid bilayer (e.g., with a cell surface or with a surface of a donor cell, acceptor cell, or a membrane-enclosed preparation as described herein). In some embodiments, a membrane-associated moiety is associated with an exterior portion of a membrane lipid bilayer and is or comprises a cell surface protein. In some embodiments a membrane-associated moiety is a naturally occurring protein. In some embodiments a membrane-associated moiety is an engineered and/or synthetic protein. In some embodiments a membrane-associated moiety is a therapeutic agent. In some embodiments, a membrane-associated moiety is operably associated or linked (e.g., tethered) to one or more of an intracellular moiety, an extracellular moiety, or a cargo molecule.

In some embodiments, a membrane-associated moiety is or comprises a lipidation modification sequence. A lipidation modification sequence comprises an amino acid sequence recognized by and/or modified by a fatty acid transferase enzyme present in a cell, e.g., a source cell or donor cell. A membrane-associated moiety comprising a lipidation modification sequence may have a lipid anchor attached that associates, e.g., anchors, the membrane-associated moiety to a membrane. In some embodiments, a lipid anchor may be attached to the terminus of a polypeptide (e.g., of a membrane-associated agent, e.g., the membrane-associated moiety). Exemplary lipid anchors include, but are not limited to, myristoyl, palmitoyl, farnesyl, glycosylphosphatidylinositol (GPI), or CAAX domain. Exemplary lipidation modification sequences are known to those of skill in the art and include but are not limited to myristolyation or palmitoylation (MYR/PA)-binding sequence (e.g., a MYR/PA sequence from an LCK tyrosine kinase).

In some embodiments, a membrane-associated moiety is or comprises a membrane protein, e.g., a naturally occurring or synthetic membrane protein (e.g., a TCR protein, e.g., a TCR alpha molecule and/or a TCR beta molecule, a TCR gamma molecule and/or a TCR delta molecule, CD28, CD8, or CD3 zeta), or a portion thereof. In some embodiments, a membrane-associated moiety is or comprises a membrane protein described herein or a portion thereof.

In some embodiments, a membrane protein relevant to the present disclosure is an integral membrane protein; in some embodiments, a membrane protein is a peripheral membrane protein. In other embodiments, a membrane protein is temporarily associated with a membrane. In some embodiments, a membrane protein is a protein that is associated with, and/or wholly or partially spans (e.g., a transmembrane protein) a target cell's membrane. In some embodiments, a membrane protein is an integral monotopic protein (i.e., associated with only one side of a membrane). In some embodiments, a membrane protein is or becomes associated with (e.g., is partly or wholly present on) an outer surface of a target cell's membrane. In some embodiments, a membrane protein is or becomes associated with (e.g., is partly or wholly present on) an inner surface of a target cell's membrane.

In some embodiments, a membrane protein relevant to the present disclosure is a therapeutic membrane protein. In some embodiments, a membrane protein relevant to the present disclosure is or comprises a receptor (e.g., a cell surface receptor and/or a transmembrane receptor), a cell surface ligand, a membrane transport protein (e.g., an active or passive transport protein such as, for example, an ion channel protein, a pore-forming protein [e.g., a toxin protein], etc), a membrane enzyme, and/or a cell adhesion protein).

In some embodiments, a membrane protein relevant to the present disclosure comprises a sequence of a naturally-occurring membrane protein. In some embodiments, a membrane protein relevant to the present disclosure is or comprises a variant or modified version of a naturally-occurring membrane protein. In some embodiments, a membrane protein relevant to the present disclosure is or comprises an engineered membrane protein. In some embodiments, a membrane protein relevant to the present disclosure is or comprises a fusion protein.

### Extracellular Moieties

An extracellular moiety is an optional part of a membrane-associated agent positioned on the exterior (e.g., non-lumen or non-cytosolic side) of a membrane (e.g., a cell membrane). In some embodiments, an extracellular moiety comprises one or more specificity portions, one or more accessory portions, or both. In some embodiments, an extracellular moiety comprises an extracellular domain, or a portion thereof, from a TCR protein (e.g., a TCR alpha molecule and/or a TCR beta molecule, or a TCR gamma molecule and/or a TCR delta molecule). In embodiments, the extracellular moiety is attached to a binding moiety (e.g., an avidin moiety, e.g., a streptavidin moiety) that binds to a binding molecule of interest (e.g., an antibody molecule) attached to a cognate binding moiety that binds to the binding moiety of the extracellular moiety (e.g., a biotin moiety). In some embodiments, an extracellular moiety comprises an antibody molecule (e.g., an antibody or a functional fragment thereof, e.g., an scFv), e.g., as described herein. In some embodiments, the antibody molecule binds specifically to an epitope on a molecule on the surface of a target cell (e.g., an acceptor cell), e.g., an epitope on a docking moiety of an acceptor cell.

In some embodiments, an extracellular moiety comprises a specificity portion which may comprise a targeting domain or a transfer promoting domain. In some embodiments, a targeting domain specifically (e.g., under conditions of exposure, e.g., of donor cell contact/proximity to an acceptor cell) associates or interacts with a target cell moiety. In some embodiments, a targeting domain specifically binds to a target cell moiety present on a target cell. In some embodiments, a targeting domain is or comprises a domain of a membrane-associated agent e.g., is covalently linked to a membrane-associated agent, e.g., is part of a membrane-associated agent polypeptide. In some embodiments, a targeting domain is a separate entity from any exogenous membrane-associated agent, e.g., is not covalently linked to a membrane-associated agent, e.g., is not part of a membrane-associated agent polypeptide. In some embodiments, the targeting domain facilitates contact between a donor cell and a target cell, e.g., acceptor cell, e.g., by binding a target cell moiety on the target cell or being bound by a target cell moiety on the target cell. In some embodiments, an extracellular moiety, e.g., a specificity portion, e.g., a targeting domain, comprises a membrane protein or a portion thereof (e.g., a membrane protein described herein). Exemplary specificity portions (e.g., targeting domains) include, but are not limited to, an antibody molecule, e.g., an antibody or functional fragment thereof (e.g., a Fab, F(ab′)2, Fab′, scFv, or di-scFv), a streptavidin domain (e.g., associated with a biotinylated agent, e.g., a biotinylated antibody), a receptor (e.g., a surface receptor) (e.g., that specifically binds a ligand on the acceptor cell), a ligand (e.g., a ligand that binds a target cell moiety, e.g., receptor, on an acceptor cell), a cell surface protein, a sugar, or a lipid.

In some embodiments, the transfer promoting domain promotes transfer of the membrane-associated agent from a membrane of a first cell (e.g., donor cell) to a membrane of a second cell (e.g., acceptor cell). Exemplary specificity portions, e.g., transfer promoting domains, include but are not limited to the extracellular domains of one or more TCR proteins (e.g., a TCR alpha molecule and/or a TCR beta molecule, or a TCR gamma molecule and/or a TCR delta molecule).

In some embodiments, an accessory portion provides an ancillary function to the membrane-associated agent, e.g., unrelated to targeting the donor cell to the acceptor cell or with promoting transfer. In some embodiments, an accessory portion comprises one or more of a tag (e.g., a label (e.g., a fluorescent or radio label) or a cleavage site), a reporter agent, or a marker.

### Intracellular Moieties

An intracellular moiety is an optional part of a membrane-associated agent positioned on the interior (e.g., lumen or cytosolic side) of a membrane (e.g., a cell membrane). In some embodiments, an intracellular moiety comprises one or more functional portions, one or more accessory portions, or both. In some embodiments, an intracellular moiety comprises an intracellular domain from a TCR protein (e.g., a TCR alpha molecule and/or a TCR beta molecule, or a TCR gamma molecule and/or a TCR delta molecule). In some embodiments, an intracellular moiety comprises a CD3 zeta molecule.

In some embodiments, an intracellular moiety comprises a functional portion which modulates a biological function in the acceptor cell (e.g., and optionally does not modulate or modulates to a lesser extent the biological process in the donor cell). Exemplary modulation includes but is not limited to: altering (e.g., decreasing or increasing) expression of a gene, epigenetic modification, increasing or decreasing activity of an intra- or inter-cell signaling pathway, altering the stability (e.g., degradation and/or half-life) of one or more cell component (e.g., signaling molecule or protein), altering secretion of a biological effector, altering cellular metabolism, inducing or inhibiting cellular migration, inducing or inhibiting apoptosis, or altering potency or the cell identity/differentiation of the cell. In some embodiments, the intracellular moiety comprises a functional portion that modulates a biological function in the donor cell (e.g., promoting or inducing activation of a donor cell that is a T cell, or is derived from a T cell).

In some embodiments, the biological function is chosen from:
(i) modulating, e.g., increasing or decreasing a level or activity of, a molecule (e.g., a protein, nucleic acid, or metabolite, drug, or toxin) in an acceptor cell or a plurality of acceptor cells;
(ii) modulating, e.g., increasing or decreasing, enzyme activity in an acceptor cell or a plurality of acceptor cells;
(iii) modulating, e.g., increasing or decreasing, a genetic or an epigenetic event in the acceptor cell or the plurality of acceptor cells;
(iv) modulating, e.g., promoting or inhibiting, acceptor cell differentiation or the differentiation of a plurality of acceptor cells;
(v) modulating acceptor cell reprogramming; or
(vi) modulating, e.g., activating or inhibiting, a signaling pathway in an acceptor cell or a plurality of acceptor cells;
(vii) modulating, e.g., increasing, decreasing, or redistributing, cell adhesion or trafficking;
(viii) introducing a genetic alteration (e.g., a substitution, insertion, or deletion) into an acceptor cell or plurality of acceptor cells, e.g., inserting an exogenous nucleic acid (e.g., encoding a gene) or mutating (e.g., knocking out) an endogenous gene.

In some embodiments, the intracellular moiety, e.g., functional portion, comprises one or more of an antibody or functional fragment thereof (e.g., a Fab, F(ab′)2, Fab′, scFv, or di-scFv), a reporter agent (e.g., a fluorescent tag), a signaling protein, an enzyme (or functional portion thereof), a transcription factor, an epigenetic remodeling agent, a protein binding domain, an RNA-binding protein or domain, a hydrophobic domain, a lipid raft targeting domain, or drug-binding domain. In some embodiments, the intracellular moiety, e.g., functional portion, comprises EGFP, β-galactosidase, β-lactamase, Cre recombinase, a CRISPR/Cas protein (e.g., Cas9), and optionally a guide RNA, or a functional portion or variant of any thereof. In some embodiments, the intracellular moiety, e.g., functional portion, comprises a nucleic acid binding domain, e.g., an RNA binding protein or domain, e.g., an mRNA binding protein or domain.

In some embodiments, the intracellular moiety, e.g., functional portion, comprises an agent that binds to another agent (e.g., comprises a protein that binds to a cargo molecule). In some embodiments, the intracellular moiety, e.g., functional portion, comprises MS2 coat protein (e.g., bound to an mRNA), an scFv (e.g., bound to a protein or an organelle), one part of a protein binding pair (e.g., bound to the other partner of the protein binding pair), streptavidin (e.g., bound to biotin or a biotin-conjugated agent), an organelle-specific integral membrane protein (e.g., bound or associated with an organelle), a CRISPR protein (e.g., a Cas9, Cas12, or MAD7 protein) (e.g., bound or associated with a guide sequence, e.g., gRNA), or a poly-A binding protein (e.g., bound or associated with an mRNA).

In some embodiments, an accessory portion provides an ancillary function to the membrane-associated agent, e.g., unrelated to the functional portion's one or more functions. In some embodiments, an accessory portion comprises one or more of a tag (e.g., a label (e.g., a fluorescent or radio label) or a cleavage site), a reporter agent, or a marker. In some embodiments, the intracellular moiety, e.g., accessory portion, comprises a Lumio tag, a TEV protease cleavage site, or a rhomboid protease cleavage site, e.g., RHBDL2.

In some embodiments, an intracellular moiety comprises a cleavage site. In some embodiments, a target cell (e.g., acceptor cell) comprises a protease that recognizes an intracellular moiety cleavage site, but the donor cell do not comprise a protease that recognizes the intracellular moiety cleavage site. Without wishing to be bound by theory, it is thought that this will allow a membrane-associated agent comprising a cleavable intracellular moiety to be delivered by a donor cell without being cleaved, wherein upon arrival in the target cell (e.g. acceptor cell) a protease will recognize the site and cleave the intracellular moiety or a portion thereof. In some embodiments, cleavage of the intracellular moiety activates a function of the intracellular moiety or a portion thereof (e.g., an enzymatic or signaling function). In some embodiments, cleavage of the intracellular moiety causes the intracellular moiety or a portion thereof to dissociate from the membrane-associated agent and/or the membrane, e.g., and enables it to fulfill its function. For example, an intracellular moiety may comprise a cleavable Cre recombinase or a transcription factor, where the Cre recombinase or transcription factor require translocation to the nucleus to function and cleavage enables said translocation.

### Cargo Molecules

In some embodiments, a donor cell described herein includes a cargo molecule. In some embodiments, a cargo molecule is an agent that may be transferred from a first membrane (e.g., a donor cell) to a second membrane (e.g., an acceptor cell membrane). Transfer of a cargo molecule may be promoted by a membrane-associated agent. A cargo molecule may be non-covalently associated with the membrane-associated agent, covalently associated via a non-peptide bond, or not associated with the membrane-associated agent (e.g., and separately associated with the membrane, e.g., of the donor cell or acceptor cell).

In some embodiments a cargo molecule comprises one or more a protein, e.g., an enzyme, a transmembrane protein, a receptor, or an antibody; a nucleic acid, e.g., a circular or linear nucleic acid, e.g., DNA, a chromosome (e.g. a human artificial chromosome), or RNA, e.g., mRNA, siRNA, miRNA, piRNA, or lncRNA; a lipid; or a small molecule (e.g., a signaling molecule (e.g., a second messenger) or a drug molecule). In some embodiments, a cargo is or comprises an organelle.

In some embodiments, the cargo molecule may be or may encode a therapeutic protein. In some embodiments, a donor cell described herein includes cargo molecules that are or comprise a plurality of therapeutic agents. In some embodiments, a cargo molecule may be a therapeutic agent that is exogenous or endogenous relative to the donor cell, acceptor cell, or source cell from which the aforementioned were derived.

In some embodiments, a donor cell or acceptor cell comprises a plurality of different cargo molecules. In some embodiments, a donor cell or acceptor cell comprises 1, 2, 3, 4, 5, 6, 7, 8, 9, 10, or more different cargo molecules (and optionally no more than 20, 15, or 10 different cargo molecules). For example, a donor cell may comprise a first cargo molecule comprising a first therapeutic agent and a second cargo molecule comprising a second different therapeutic agent.

In some embodiments a donor cell comprises a cargo molecule associated with the donor cell lipid bilayer. In some embodiments a donor cell comprises a cargo molecule disposed within the cytosol or lumen of the donor cell. In some embodiments, a donor cell comprises a cargo molecule associated with the donor cell lipid bilayer and a cargo molecule disposed within the cytosol or lumen of the donor cell.

In some embodiments, a cargo molecule is not expressed naturally in a donor cell, or source cell from which the donor cell is derived. In some embodiments, a cargo molecule is expressed naturally in the donor cell, or source cell from which the donor cell is derived. In some embodiments, a cargo molecule is a mutant of a wild type nucleic acid or protein expressed naturally in a donor cell, or source cell from which the donor cell is derived, or the cargo molecule is a wild type variant of a mutant cargo molecule expressed naturally in a donor cell, or source cell from which the donor cell is derived.

In some embodiments, a cargo molecule is expressed from an exogenous nucleic acid molecule introduced into a donor cell or an exogenous nucleic acid sequence inserted into the genome of a donor cell. In some embodiments, the cargo molecule is encoded in the exogenous nucleic acid molecule or the exogenous insert sequence under the control of a promoter (e.g., a lineage-restricted, tissue-specific, or cell type-specific promoter). In some embodiments, the promoter is activated by the membrane-associated moiety (e.g., expression of the cargo molecule is increased by at least 2, 3, 4, 5, 6, 7, 8, 9, 10, 20, 30, 40, 50, 60, 70, 80, 90, 100, 200, 300, 400, 500, 600, 700, 800, 900, or 100-fold, or more, when the membrane-associated moiety is present in the donor cell). In some embodiments, the promoter is repressed by the membrane-associated moiety (e.g., expression of the cargo molecule is decreased by at least 2, 3, 4, 5, 6, 7, 8, 9, 10, 20, 30, 40, 50, 60, 70, 80, 90, 100, 200, 300, 400, 500, 600, 700, 800, 900, or 100-fold, or more, when the membrane-associated moiety is present in the donor cell). In an embodiment, the cargo molecule is under the control of an NFAT promoter. In an embodiment, an NFAT promoter is activated by a CD3-zeta domain on the membrane-associated moiety (e.g., expression of the cargo molecule is increased by at least 2, 3, 4, 5, 6, 7, 8, 9, 10, 20, 30, 40, 50, 60, 70, 80, 90, 100, 200, 300, 400, 500, 600, 700, 800, 900, or 100-fold, or more, when the CD3-zeta domain is present in the donor cell). In an embodiment, the cargo molecule is under the control of a FoxP3 promoter.

In some embodiments, a cargo molecule is secreted from a donor cell, e.g., upon binding of a membrane-associated agent of the donor cell to a second docking moiety on an acceptor cell. In certain embodiments, the cargo molecule is present at a higher concentration in the solution surrounding the donor cell relative to solution farther away from the donor cell, e.g., due to the secretion of the cargo molecule by the donor cell. In certain embodiments, the cargo molecule modulates (e.g., activates or inhibits) a downstream biological activity of the acceptor cell. In an embodiment, the secreted cargo molecule comprises insulin.

In some embodiments, a cargo molecule is loaded into a donor cell via expression in a source cell from which the donor cell is derived (e.g. expression from DNA introduced via transfection, transduction, or electroporation). In some embodiments, a cargo molecule is expressed from DNA integrated into the genome of the source cell from which the donor cell is derived or maintained episosomally in the source cell from which the donor cell is derived. In some embodiments, expression of a cargo molecule is constitutive in the source cell from which the donor cell is derived. In some embodiments, expression of a cargo molecule in the source cell from which the donor cell is derived is induced. In some embodiments, expression of the cargo molecule is induced in the source cell from which the donor cell is derived immediately prior to generating the donor cell. In some embodiments, expression of a cargo molecule in the source cell from which the donor cell is derived is induced at the same time as expression of the membrane-associated agent in the cell from which the donor cell is derived.

In some embodiments, a cargo molecule is loaded into a donor cell via electroporation into the donor cell itself or into a source cell from which the donor cell is derived. In some embodiments, a cargo molecule is loaded into a donor cell via transfection into the donor cell itself or into a source cell from which the donor cell is derived.

In some embodiments, the cargo molecule may include one or more nucleic acid sequences, one or more polypeptides, a combination of nucleic acid sequences and/or polypeptides, one or more organelles, and any combination thereof. In some embodiments, the cargo molecule may include one or more cellular components. In some embodiments, the cargo molecule includes one or more cytosolic and/or nuclear components.

In some embodiments, the cargo molecule includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences.

In some embodiments, the cargo molecule may include a nucleic acid. For example, the cargo molecule may comprise RNA to enhance expression of an endogenous protein (e.g., in some embodiments, endogenous relative to the source cell, and in some embodiments, endogenous relative to the target cell), or a siRNA or miRNA that inhibits protein expression of an endogenous protein. For example, the endogenous protein may modulate structure or function in the target cells (e.g., acceptor cells). In some embodiments, the cargo molecule may include a nucleic acid encoding an engineered protein that modulates structure or function in the target cells (e.g., acceptor cells). In some embodiments, the cargo molecule is a nucleic acid that targets a transcriptional activator that modulate structure or function in the target cells (e.g., acceptor cells).

In some embodiments, the cargo molecule comprises a self-replicating RNA, e.g., as described herein. In some embodiments, the self-replicating RNA is single stranded RNA and/or linear RNA. In some embodiments, the self-replicating RNA encodes one or more proteins, e.g., a protein described herein, e.g., a membrane protein or a secreted protein. In some embodiments, the self-replicating RNA comprises a partial or complete genome from arterivirus or alphavirus, or a variant thereof.

In some embodiments, the cargo molecule can comprise an RNA that can be delivered into a target cell (e.g., an acceptor cell), and RNA is replicated inside the target cell (e.g., acceptor cell). Replication of the self-replicating RNA can involve RNA replication machinery that is exogenous to the target cell (e.g., acceptor cell), and/or RNA replication machinery that is endogenous to the target cell (e.g., acceptor cell).

In some embodiments, the self-replicating RNA comprises a viral genome, or a self-replicating portion or analog thereof. In some embodiments, the self-replicating RNA is from a positive-sense single-stranded RNA virus. In some embodiments, the self-replicating RNA comprises a partial or complete arterivirus genome, or a variant thereof. In some embodiments, the arterivirus comprises Equine arteritis virus (EAV), Porcine respiratory and reproductive syndrome virus (PRRSV), Lactate dehydrogenase elevating virus (LDV), and Simian hemorrhagic fever virus (SHFV). In some embodiments, the self-replicating RNA comprises a partial or complete alphavirus genome, or a variant thereof. In some embodiments, the alphavirus belongs to the VEEV/EEEV group (e.g., Venezuelan equine encephalitis virus), the SF group, or the SIN group.

In some embodiments, the donor cell that comprises the self-replicating RNA further comprises: (i) one or more proteins that promote replication of the RNA, or (ii) a nucleic acid encoding one or more proteins that promote replication of the RNA, e.g., as part of the self-replicating RNA or in a separate nucleic acid molecule.

In some embodiments, the self-replicating RNA lacks at least one functional gene encoding one or more viral structural protein relative to the corresponding wild-type genome. For instance, in some embodiments the self-replicating RNA fully lacks one or more genes for viral structural proteins or comprises a non-functional mutant gene for a viral structural protein. In some embodiments, the self-replicating RNA does not comprise any genes for viral structural proteins.

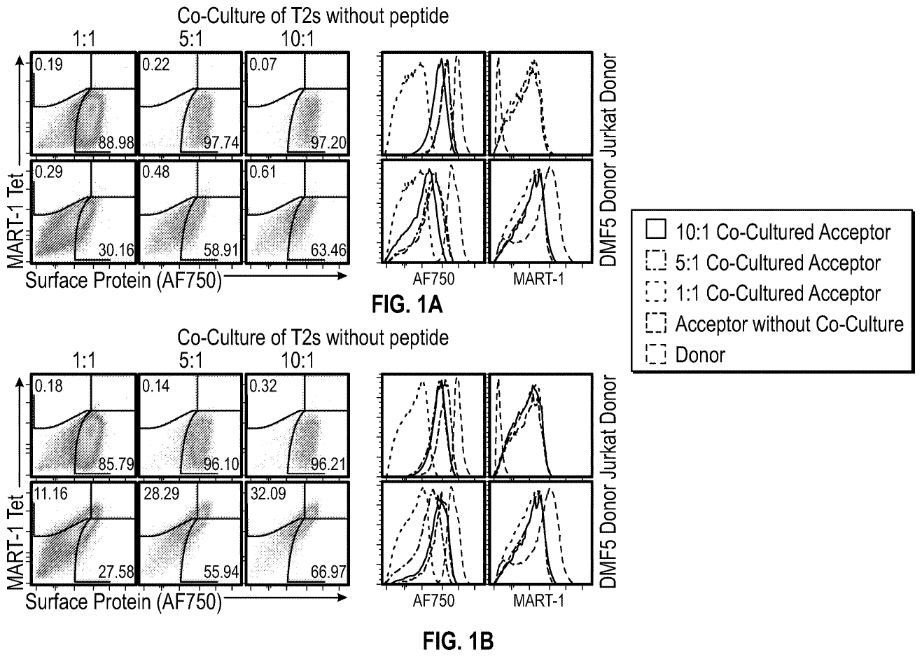

In some embodiments, the self-replicating RNA comprises a viral capsid enhancer, e.g., as described in International Application WO2018/106615, which is hereby incorporated by reference in its entirety. In some embodiments, the viral capsid enhancer is an RNA structure that increases translation of a coding sequence in cis, e.g., by allowing eIF2alpha independent translation of the coding sequence. In some embodiments, a host cell has impaired translation, e.g., due to PKR-mediated phosphorylation of eIF2alpha. In embodiments, the viral capsid enhancer comprises a Downstream Loop (DLP) from a viral capsid protein, or a variant of the DLP. In some embodiments, the viral capsid enhancer is from a virus belonging to the Togaviridae family, e.g., the Alphavirus genus of the Togaviridae family. In some embodiments, the viral capsid enhancer has a sequence of SEQ ID NO: 1 of WO2018/106615 (which sequence is herein incorporated by reference in its entirety), or a sequence having at least 70%, 80%, 85%, 90%, 95%, or 99% identity thereto. In some embodiments, the sequence has the same secondary structure shown in FIG. 1 of WO2018/106615.

In some embodiments, the self-replicating RNA comprises one or more arterivirus sequences, e.g., as described in International Application WO2017/180770, which is hereby incorporated by reference in its entirety. In some embodiments, the self-replicating RNA comprises ORF7 (or a functional fragment or variant thereof) and/or the self-replicating RNA lacks a functional ORF2a (e.g., fully lacks ORF2a, or comprises a non-functional mutant of ORF2a) of an arterivirus. In some embodiments, the self-replicating RNA lacks a functional ORF2b, ORF3, ORF4, ORF5a, ORF5, or ORF6 or any combination thereof (e.g., fully lacks the sequence(s) or comprises a non-functional mutant of the sequence(s)). In some embodiments, the self-replicating RNA lacks a portion of one or more of ORF2a, ORF2b, ORF3, ORF4, ORF5a, ORF5, or ORF6. In some embodiments, the self-replicating RNA comprises one or more subgenomic (sg) promoters, e.g., situated at a non-native site. In some embodiments, the promoter comprises sg promoter 1, sg promoter 2, sg promoter 3, sg promoter 4, sg promoter 5, sg promoter 6, sg promoter 7, or a functional fragment or variant thereof. In some embodiments, the self-replicating RNA comprises one or more transcriptional termination signals, e.g., T7 transcriptional termination signals, e.g., a mutant T7 transcription termination signal, e.g., a mutant T7 transcription termination signal comprising one or more of (e.g., any two of, or all of) T9001G, T3185A, or G3188A.

In some embodiments, the self-replicating RNA comprises a 5′ UTR, e.g., a mutant alphavirus 5′ UTR, e.g., as described in International Application WO2018/075235, which is hereby incorporated by reference in its entirety. In some embodiments, the mutant alphavirus 5′ UTR comprises one or more nucleotide substitutions at position 1, 2, 4, or a combination thereof. In some embodiments, the mutant alphavirus 5′ UTR comprises a U->G substitution at position 2.

In some embodiments, the cargo molecule includes a protein, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, a homeodomain protein, a scavenger receptor, a scavenger receptor ligand, a Ran GTPase, RanQ69L, and any combination thereof. In some embodiments the protein targets a protein in the acceptor cell for degradation. In some embodiments the protein targets a protein in the acceptor cell for degradation by localizing the protein to the proteasome. In some embodiments, the protein is a wild-type protein. In some embodiments, the protein is a mutant protein. In some embodiments the protein is a fusion or chimeric protein.

In some embodiments, the cargo molecule comprises a gene modifying polypeptide, recombinase, retrotransposase, or protein comprising a CRISPR/Cas domain. Exemplary polypeptides of these types are described, e.g., in PCT Publication Nos. WO/2020/047124, WO/2021/016075, WO/2021/102390, WO/2021/178720, WO/2021/178709, WO/2021/178717, or WO/2021/178898, each of which is incorporated by reference herein in its entirety.

In some embodiments, the cargo molecule comprises a membrane protein. In certain embodiments, the membrane protein comprises a membrane protein payload agent (e.g., as described in PCT Publication No. WO/2020/102578; incorporated herein by reference in its entirety).

In some embodiments, the cargo molecule comprises a protein comprising a nuclear localization signal.

In some embodiments, the cargo molecule comprises an exogenous agent as described, e.g., in PCT Publication No. WO/2019/222403; incorporated herein by reference in its entirety. In certain embodiments, the exogenous agent comprises a polypeptide (e.g., an enzyme, transport protein, transcription factor, antibody, cytokine, hormone, chimeric antigen receptor, nucleic acid binding protein, cell death protein, proteasome targeting protein, or endonuclease). In certain embodiments, the protein is a fusion protein or a chimeric protein. In certain embodiments, the exogenous agent comprises a cytosolic protein, a membrane protein, a secreted protein, a nuclear protein, or an organellar protein (e.g., a mitochondrial protein). In certain embodiments, the exogenous agent comprises a nucleic acid, e.g., an RNA (e.g., an mRNA, tRNA, siRNA, miRNA gRNA, or shRNA). In certain embodiments, the exogenous agent is a therapeutic agent (e.g., an agent for treating a disease or disorder, e.g., as described herein). In certain embodiments, the disease or disorder is a cancer, infectious disease, or immune disorder.

In some embodiments, the cargo molecule comprises an exogenous agent as described, e.g., in PCT Publication No. WO/2020/014209; incorporated herein by reference in its entirety. In certain embodiments, the exogenous agent comprises a cytosolic protein, a membrane protein, a secreted protein, a nuclear protein, or an organellar protein (e.g., a mitochondrial protein). In certain embodiments, the exogenous agent is chosen from: PAL, OTC, CPS1, NAGS, BCKDHA, BCKDHB, DBT, DLD, MUT, MMAA, MMAB, MMACHC, MMADHC, MCEE, PCCA, PCCB, UGT1A1, ASS1, PAH, ATP8B1, ABCB11, ABCB4, TJP2, IVD, GCDH, ETFA, ETFB, ETFDH, ASL, D2HGDH, HMGCL, MCCC1, MCCC2, ABCD4, HCFC1, LMBRD1, ARG1, SLC25A15, SLC25A13, ALAD, CPOX, HMBS, PPOX, BTD, HLCS, PC, SLC7A7, CPT2, ACADM, ACADS, ACADVL, AGL, G6PC, GBE1, PHKA1, PHKA2, PHKB, PHKG2, SLC37A4, PMM2, CBS, FAH, TAT, GALT, GALK1, GALE, G6PD, SLC3A1, SLC7A9, MTHFR, MTR, MTRR, ATP7B, HPRT1, HJV, HAMP, JAG1, TTR, AGXT, LIPA, SERPINGI, HSD17B4, UROD, HFE, LPL, GRHPR, HOGA1, or LDLR. In certain embodiments, the exogenous agent comprises a protein listed in Table 5 of PCT Publication No. WO/2020/014209.

In some embodiments, the cargo molecule is or comprises a receptor, a ligand, or a functional portion of either thereof. In some embodiments, the receptor is a receptor described herein, e.g., an RTK or scavenger receptor.

In some embodiments, the cargo molecule is or comprises a cancer driver, e.g., a protein or gene product encoded by a cancer driver gene as described in Bailey et al. Cell. 2018 Apr. 5; 173(2):371-385, the list of which is hereby incorporated by reference.

In some embodiments, the cargo molecule is or comprises a Cluster of Differentiation protein or a functional portion or variant thereof.

In some embodiments a cargo molecule is a protein (or a nucleic acid that encodes it) that is naturally found on a membrane surface of a cell (e.g., on a surface of a plasma membrane).

Exemplary membrane proteins (and/or nucleic acids encoding them) include any described herein, and can be found, for example, in U.S. Patent Publication No. 2016/0289674, the contents of which are hereby incorporated by reference. In some embodiments, a cargo molecule (and/or a nucleic acid that encodes it) has a sequence as set forth in any one of SEQ ID NOs: 8144-16131 of U.S. Patent Publication No. 2016/0289674, or in a functional fragment thereof. In some embodiments, a cargo molecule is a plasma membrane protein (nucleic acid encoding it) as set forth in any one of SEQ ID NOs: 8144-16131 of U.S. Patent Publication No. 2016/0289674, or a fragment, variant, or homolog thereof (or nucleic acid that encodes it) of a plasma membrane protein of.

In some embodiments, a membrane protein relevant to the present disclosure is a therapeutic membrane protein. In some embodiments, a membrane protein relevant to the present disclosure is or comprises a cell surface receptor, a membrane transport protein (e.g., an active or passive transport protein such as, for example, an ion channel protein, a pore-forming protein [e.g., a toxin protein], etc), a membrane enzyme, and/or a cell adhesion protein).

In some embodiments a membrane protein is a single spanning membrane protein. In some embodiments a single-spanning membrane protein may assume a final topology with a cytoplasmic N- and an exoplasmic C-terminus (N_{cyt}/Cₑₓₒ) or with the opposite orientation (Nₑₓₒ/C_{cyt}).

In some embodiments a membrane protein is a Type I membrane protein comprising an N-terminal cleavable signal sequence and stop-transfer sequence (Nₑₓₒ/C_{cyt}). In some embodiments a signal is at the C terminus. In some embodiments the N-terminal cleavable signal sequence targets nascent peptide to the ER. In some embodiments an N-terminal cleavable signal sequence comprises a hydrophobic stretch of typically 7-15 predominantly apolar residues. In some embodiments a Type I membrane protein comprises a stop-transfer sequence which halts the further translocation of the polypeptide and acts as a transmembrane anchor. In some embodiments a stop transfer sequence comprises an amino acid sequence of about 20 hydrophobic residues. In some embodiments the N-terminus of the Type I membrane protein is extracellular and the C-terminus is cytoplasmic. In some embodiments a Type I membrane protein may be a glycophorin or an LDL receptor.

In some embodiments a membrane protein is a Type II membrane protein comprising a signal-anchor sequence (N_{cyt}/Cₑₓₒ). In some embodiments a signal is at the C terminus. In some embodiments a signal-anchor sequence is responsible for both insertion and anchoring of a Type II membrane protein. In some embodiments a signal-anchor sequence comprises about 18-25 predominantly apolar residues. In some embodiments a signal-anchor sequence lacks a signal peptidase cleavage site. In some embodiments a signal-anchor sequence may be positioned internally within a polypeptide chain. In some embodiments a signal-anchor sequence induces translocation of the C-terminal end of a protein across a cell membrane. In some embodiments the C-terminus of the Type II membrane protein is extracellular and the N-terminus is cytoplasmic. In some embodiments a Type II membrane protein may be a transferrin receptor or a galactosyl transferase receptor.

In some embodiments a membrane protein is a Type III membrane protein comprising a reverse signal-anchor sequence (Nₑₓₒ/C_{cyt}). In some embodiments a signal is at the N terminus. In some embodiments a reverse signal-anchor sequence is responsible for both insertion and anchoring of a Type III membrane protein. In some embodiments a reverse signal-anchor sequence comprises about 18-25 predominantly apolar residues. In some embodiments a signal-anchor sequence lacks a signal peptidase cleavage site. In some embodiments a signal-anchor sequence may be positioned internally within a polypeptide chain. In some embodiments a signal-anchor sequence induces translocation of the N-terminal end of a protein across a cell membrane. In some embodiments the N-terminus of the Type III membrane protein is extracellular and the C-terminus is cytoplasmic. In some embodiments a Type I membrane protein may be a synaptogamin, neuregulin, or cytochrome P-450.

In some embodiments, Type I, Type II, or Type III membrane proteins are inserted into a cell membrane via a cellular pathway comprising SRP, SRP receptor and Sec61 translocon.

In some embodiments a membrane protein is predominantly exposed to cytosol and anchored to a membrane by a C-terminal signal sequence, but which does not interact with an SRP. In some embodiments a protein is cytochrome b₅, or a SNARE protein (e.g., synaptobrevin).

In some embodiments a cargo molecule comprises a signal sequence which localizes the cargo molecule to the cell membrane. In some embodiments a cargo molecule is a nucleic acid wherein the nucleic acid encodes a signal sequence which localizes a protein encoded by the nucleic acid to the cell membrane.

In some embodiments, the cargo molecule includes a small molecule, e.g., ions (e.g. Ca²⁺, Cl⁻, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the cargo molecule includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

In some embodiments, the cargo molecule includes one or more organelles, e.g., chondrisomes, mitochondria, lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granule, networks of organelles, and any combination thereof.

In some embodiments, the cargo molecule is enriched at the donor cell or acceptor cell cell membrane. In some embodiments, the cargo molecule is enriched by targeting to the membrane via a peptide signal sequence. In some embodiments, the cargo molecule is enriched by binding with a membrane associated protein, lipid, or small molecule. In some embodiments, the cargo molecule is enriched by dimerizing with a membrane associated protein, lipid, or small molecule. In some embodiments the cargo molecule is chimeric (e.g. a chimeric protein, or nucleic acid) and comprises a domain that mediates binding or dimerization with a membrane associated protein, lipid, or small molecule. Membrane-associated proteins of interest include, but are not limited to, membrane proteins described herein or any protein having a domain that stably associates, e.g., binds to, integrates into, etc., a cell membrane (i.e., a membrane-association domain), where such domains may include myristoylated domains, farnesylated domains, transmembrane domains, and the like. Specific membrane-associated proteins of interest include, but are not limited to: myristoylated proteins, e.g., p 60 v-src and the like; farnesylated proteins, e.g., Ras, Rheb and CENP-E,F, proteins binding specific lipid bilayer components e.g. AnnexinV, by binding to phosphatidyl-serine, a lipid component of the cell membrane bilayer and the like; membrane anchor proteins; transmembrane proteins, e.g., transferrin receptors and portions thereof; and membrane fusion proteins.

In some embodiments, the cargo molecule is present in a donor cell or acceptor cell at a higher level (e.g., a higher copy number) than a membrane-associated agent. In some embodiments, the cargo molecule is present in an acceptor cell at a higher level than a membrane-associated agent or one or more components of thereof (e.g., one, two, or all of membrane-associated moiety, extracellular moiety, or intracellular moiety). In some embodiments, the acceptor cell comprises the cargo molecule but does not comprise the membrane-associated agent or one, two, or all of the membrane-associated moiety, intracellular moiety, or extracellular moiety. In some embodiments, the acceptor cell comprises the cargo molecule and comprises only residual levels of the membrane-associated agent or one, two, or all of the membrane-associated, intracellular, or extracellular moiety (e.g., less than 30, 25, 20, 18, 16, 14, 12, 10, 9, 8, 7, 6, 5, 4, 3, 2, or 1% of cells in a sample of acceptor cells comprise exogenous membrane-associated agent or one, two, or all of the membrane-associated moiety, extracellular moiety, or intracellular moiety).

In some embodiments, the cargo molecule is operably associated or linked (e.g., tethered) to the membrane-associated agent (e.g., the membrane-associated moiety, extracellular moiety, or intracellular moiety) when in the donor cell, but may separate from the membrane-associated agent when in the target cell, e.g., acceptor cell. In some embodiments, a cargo molecule can be non-covalently associated with a membrane-associated agent in the donor cell and can dissociate from the membrane-associated agent in the target cell (e.g., acceptor cell). In some embodiments, a cargo molecule can be covalently associated with a membrane-associated agent in the donor cell and the covalent association can be broken in the target cell (e.g., acceptor cell). Without wishing to be bound by theory, a difference in one or more conditions between the donor cell and the target cell (e.g., acceptor cell) may promote separation of the cargo molecule from the membrane-associated agent. Such differences in condition include but are not limited to differences in: pH, level of a signaling molecule, level or activity of an enzyme, expression of a gene, or the presence or level of an exogenous agent (e.g., a chemical or synthetic agent).

In some embodiments, the cargo molecule is operably associated or linked (e.g., tethered) to a membrane (e.g., the cell membrane) when in the donor cell, but may separate from the membrane when in the target cell, e.g., acceptor cell. In some embodiments, a cargo molecule can be non-covalently associated with a membrane (e.g., the cell membrane) in the donor cell and can dissociate from the membrane-associated agent in the target cell (e.g., acceptor cell). In some embodiments, a cargo molecule can be covalently associated with a membrane (e.g., the cell membrane) in the donor cell and the covalent association can be broken in the target cell (e.g., acceptor cell). Without wishing to be bound by theory, a difference in one or more conditions between the donor cell and the target cell (e.g., acceptor cell) may promote separation of the cargo molecule from the membrane (e.g., the cell membrane). Such differences in condition include but are not limited to differences in: pH, level of a signaling molecule, level or activity of an enzyme, expression of a gene, or the presence or level of an exogenous agent (e.g., a chemical or synthetic agent). In some embodiments, the difference in condition comprises a difference in the presence, level, or activity of a nuclease or protease (e.g., which recognizes a cleavable sequence in a cargo molecule). In some embodiments, a target cell (e.g., acceptor cell) comprises a nuclease or protease that recognizes a cleavable sequence in the cargo molecule, e.g., wherein cleavage frees the cargo molecule from its association with the membrane. In some embodiments, a donor cell, or source cell from which the aforementioned are derived does not comprise a nuclease or protease that recognizes a cleavable sequence in the cargo molecule. Suitable proteases and protease cleavable tags include, but are not limited to, any described herein.

### Signal Sequences

In some embodiments, a cargo molecule is a protein (or nucleic acid encoding it) that includes or included a signal sequence directing the protein to a particular site or location (e.g., to the cell surface). Those skilled in the art will appreciate that, in certain instances, a cell uses “sorting signals” which are amino acid motifs that are at least temporarily part of a protein (e.g., when initially produced), to target the protein to particular subcellular location (e.g., to a particular organelle or surface membrane of a target cell). In some embodiments a sorting signal is a signal sequence, a signal peptide, or a leader sequence, which directs a protein to an organelle called the endoplasmic reticulum (ER); in some such embodiments, the protein is then delivered to the plasma membrane. See US20160289674A1. In some such embodiments, the protein is then secreted. In some such embodiments, the protein is then trafficked to the lysosome. In some such embodiments, the protein is then trafficked to the Golgi apparatus. In some such embodiments, the protein is then trafficked to a secretory vesicle, and may then be secreted from the cell. In some such embodiments, the protein is then trafficked to an endosome.

In some embodiments, protein targeting to the ER is cotranslational. In some embodiments protein translocation and membrane insertion are coupled to protein synthesis. In some embodiments a signal sequence may be hydrophobic. In some embodiments a signal sequence may be partially hydrophobic. In some embodiments a signal sequence is recognized by a signal recognition particle (SRP). In some embodiments the SRP recognizing a signal sequence as it emerges from a ribosome. In some embodiments, a nascent peptide chain-ribosome complex is targeted to the ER by binding to an SRP receptor. In some embodiments a signal sequence interacts with an Sec61α subunit of a translocon and initiates translocation of a membrane protein or partial chain of said membrane protein.

In some embodiments, a cargo molecule comprises an in-frame fusion of a protein of interest to the coding sequence of a transmembrane protein, or an in-frame fusion of a protein of interest to the transmembrane domain or membrane-anchoring domain of a protein (e.g. fusion to the transferrin receptor membrane anchor domain). See, e.g., Winndard, P, et al. Development of novel chimeric transmembrane proteins for multimodality imaging of cancer cells, Cancer Biology & Therapy. 12:1889-1899 (2007).

In some embodiments a sorting signal or signal peptide is appended to the N or C terminus of a protein (e.g., membrane protein or secreted protein). See Goder, V. & Spiess, M., Topogenesis of membrane proteins: determinants and dynamics. FEBS Letters. 504(3): 87-93 (2001). In some embodiments the protein is a natural protein. In some embodiments the membrane protein is a synthetic protein.

In some embodiments, a signal emerges from a ribosome only after translation of a transcript has reached a stop codon. In some embodiments insertion of a membrane protein is post-translational.

In some embodiments a signal sequence is selected from Table 1. In some embodiments a signal sequence comprises a sequence selected from Table 1. In some embodiments a signal sequence of Table 1 may be appended to the N-terminus of a protein, e.g., a membrane protein or secreted protein. In some embodiments a signal sequence of Table 1 may be appended to the C-terminus of a protein, e.g., a membrane protein or secreted protein. A person of ordinary skill will appreciate that the signal sequences below are not limited for use with their respective naturally associated proteins. In some embodiments, the nucleic acid includes one or more regulatory elements that direct expression of sequences encoding the membrane protein by the target cell.

TABLE 1
Exemplary signal sequences.

| | | Naturally | Location |
|---|---|---|---|
| SEQ ID | | Associated | Associated Protein |
| NO: | Signal Sequence | Protein | is Directed |
| 2 | MRVKEKYQHL WRWGWKWGTM | HIV-1 gp41 | Plasma membrane |
| | LLGILMICSA TE | | |
| 3 | CAAL | p21ras | Plasma membrane |
| 4 | KKKKKK | p21ras | Plasma membrane |
| 5 | RRRRR | p21ras | Plasma membrane |
| 6 | MRLLLALLGV LLSVPGPPVL S | FGFR4 | Plasma membrane |
| 7 | CSIMNLMCGS TC | ROP7 GTPase | Plasma membrane |
| 8 | GHKSEEKREK MKRTLLKDWK | RGS2 | Plasma membrane |
| | TRLSYFLQNS STPGKPKTGK KSKQQ | | |
| 9 | RSTLKLTTLQ CQYSTVMD | LHR | Plasma membrane\ |
| | | | basolateral cell |
| | | | surface |
| 10 | RQGLHNMEDV YELIENSH | TSHR | Plasma membrane\ |
| | | | basolateral cell |
| | | | surface |
| 11 | MDCRKMARFS YSVIWIMAIS | TDGF1 | Plasma membrane |
| | KVFELGLVAG | | |
| 12 | MPAWGALFLL WATAEA | (GP)IX | Plasma membrane |
| 13 | RDYR | VPAC2 | Plasma membrane |
| 14 | KMALRVALNN KQSGQITVKT | Toc 159 | Plasma membrane |
| | SSSDHLSLAI AGLVPIALSI | | |
| | YQKFKPGVSP SYSIY | | |
| 15 | MGSKIVQVFL MLALFATSAL A | Classical | Plasma membrane |
| | | arabinogalactan | |
| | | protein 4 | |
| 16 | MNSKAMQALI FLGFLATSCL A | Classical | Plasma membrane |
| | | arabinogalactan | |
| | | protein 2 | |
| 17 | MGAAASIQTT VN | LIR | Plasma membrane |
| | SVM | GTP-binding protein | Plasma membrane |
| | | Rheb | |
| 18 | FALLGTHGAS G | CD147 | Plasma membrane |
| 19 | RRRTFLK | PlcH | Plasma membrane |
| 20 | MGGKWSKSSV | Nef | Plasma membrane |
| 21 | DDPERE | Nef | Plasma membrane |
| 22 | EEANTGENNS LLHPMS | HIV-1 NA7 | Plasma membrane |
| 23 | SRRGLV | DmsA | Plasma membrane |
| 24 | SRRRFL | TorA/TorA-MalE | Plasma membrane |
| 25 | SRRQFI | SufI | Plasma membrane\ |
| | | | periplasm |
| 26 | QRRDFL | YacK | Plasma membrane\ |
| | | | periplasm |
| 27 | MNKIYSIKYS AATGGLIAVS | Pet (Serine protease | Plasma membrane |
| | ELAKKVICKT NRKISAALLS | pet autotransporter) | |
| | LAVISYTNII YA | | |
| 28 | MNPNQKIITI GSICMVIGIV | Influenza A | Plasma Membrane |
| | SLMLQIGNII SIWVSHSIQT | Neuraminidase | |
| 29 | LRCLACSCFR TPVWPR | prRDH | Plasma Membrane |
| 30 | MGCGCSSHPE | Lck | Plasma Membrane |
| 31 | MPFVNKQFN | BoNT/A-LC | Plasma Membrane |
| 32 | DEQNAKNAAQ DRNSNKSSKG | Yck2p | Plasma Membrane |
| | FFSKLGCC | | |
| 33 | MLCCMRRTKQ | GAP-43 | Plasma Membrane |
| 34 | VTNGSTYILV PLSH | FSHR | Plasma Membrane |
| 35 | AETENFV | M3 mAChR | Plasma Membrane |
| 36 | RARHRRNVDR VSIGSYRT | pIgR | Plasma Membrane |
| 37 | YEDQ | RhBG | Plasma Membrane |
| 38 | LLVTSLLLCELPHPAFL IP | GM-CSF Receptor | Plasma membrane |
| | | (GM_CSFR) | |

### Exemplary Construct Sequences

In some embodiments, a membrane-associated agent comprises an amino acid sequence as listed in Table 2 below (or an amino acid sequence having at least 75%, 80%, 85%, 90%, 95%, 96%, 97%, 98%, or 99% sequence identity thereto), or a functional fragment thereof (e.g., a subsequence indicated by underlining or the absence thereof, or a subsequence indicated by bolded italics or the absence thereof). In some embodiments, a membrane-associated agent is encoded by a nucleic acid sequence as listed in Table 3 below (or an amino acid sequence having at least 75%, 80%, 85%, 90%, 95%, 96%, 97%, 98%, or 99% sequence identity thereto), or a functional fragment thereof.

TABLE 2
Exemplary Amino Acid Sequences

| Name | Sequence |
|---|---|
| MCP protein | NASNFTQFVLVDNGGTGDVTVAPSNFANGIAEWISSNSRSQAYKVTCSV |
| sequence | RQSSAQNRKYTIKVEVPKGAWRSYLNMELTIPIFATNSDCELIVKAMQGL |
| | LKDGNPIPSAIAANSGIYAMASNFTQFVLVDNGGTGDVTVAPSNFANGIA |
| | EWISSNSRSQAYKVTCSVRQSSAQNRKYTIKVEVPKGAWRSYLNMELTIP |
| | IFATNSDCELIVKAMQGLLKDGNPIPSAIAANSGIYA |
| CD19CAR-MCP | MALPVTALLLPLALLLHAARPDIQMTQTTSSLSASLGDRVTISCRASQDIS |
| (MCP sequence | KYLNWYQQKPDGTVKLLIYHTSRLHSGVPSRFSGSGSGTDYSLTISNLEQ |
| underlined) | EDIATYFCQQGNTLPYTFGGGTKLEITGGGGSGGGGSGGGGSEVKLQESG |
| | PGLVAPSQSLSVTCTVSGVSLPDYGVSWIRQPPRKGLEWLGVIWGSETTY |
| | YNSALKSRLTIIKDNSKSQVFLKMNSLQTDDTAIYYCAKHYYYGGSYAM |
| | DYWGQGTSVTVSSTTTPAPRPPTPAPTIASQPLSLRPEACRPAAGGAVHTR |
| | GLDFACDIYIWAPLAGTCGVLLLSLVITLYCNHRNRRRRVKFSRSADAPA |
| | YQQGQNQLYNELNLGRREEYDVLDKRRGRDPEMGGKPRRKNPQEGLYN |
| | ELQKDKMAEAYSEIGMKGERRRGKGHDGLYQGLSTATKDTYDALHMQ |
| | ALPPRGSGGDYKDDDDKGSNASNFTQFVLVDNGGTGDVTVAPSNFANGI |
| | AEWISSNSRSQAYKVTCSVRQSSAQNRKYTIKVEVPKGAWRSYLNMELT |
| | IPIFATNSDCELIVKAMQGLLKDGNPIPSAIAANSGIYAMASNFTQFVLVD |
| | NGGTGDVTVAPSNFANGIAEWISSNSRSQAYKVTCSVRQSSAQNRKYTIK |
| | VEVPKGAWRSYLNMELTIPIFATNSDCELIVKAMQGLLKDGNPIPSAIAA |
| | NSGIYA* |
| EGFP-MS2 | MVSKGEELFTGVVPILVELDGDVNGHKFSVSGEGEGDATYGKLTLKFICT |
| | TGKLPVPWPTLVTTLTYGVQCFSRYPDHMKQHDFFKSAMPEGYVQERTI |
| | FFKDDGNYKTRAEVKFEGDTLVNRIELKGIDFKEDGNILGHKLEYNYNSH |
| | NVYIMADKQKNGIKVNFKIRHNIEDGSVQLADHYQQNTPIGDGPVLLPD |
| | NHYLSTQSALSKDPNEKRDHMVLLEFVTAAGITLGMDELYK |
| CD3z YFx6-EGFP | MKWKVSVLACILHVRFPGAEAQSFGLLDPKLCYLLDGILFIYGVIITALYL |
| (Y to F substitutions | RAKFSRSAETAANLQDPNQLFNELNLGRREEFDVLEKKRARDPEMGGKQ |
| indicated with | QRRRNPQEGVFNALQKDKMAEAFSEIGTKGERRRGKGHDGLFQGLSTA |
| bolded underlining, | TKDTFDALHMQTLAPRPPVATMVSKGEELFTGVVPILVELDGDVNGHKF |
| EGFP indicated with | SVSGEGEGDATYGKLTLKFICTTGKLPVPWPTLVTTLTYGVQCFSRYPDH |
| underlining) | MKQHDFFKSAMPEGYVQERTIFFKDDGNYKTRAEVKFEGDTLVNRIELK |
| | GIDFKEDGNILGHKLEYNYNSHNVYIMADKQKNGIKVNFKIRHNIEDGSV |
| | QLADHYQQNTPIGDGPVLLPDNHYLSTQSALSKDPNEKRDHMVLLEFVT |
| | AAGITLGMDELYK* |
| Lck palmitoylation/ | MGCVCSSNPE |
| myristoylation domain | |
| PM-EGFP (EGFP | MGCVCSSNPELPVATMVSKGEELFTGVVPILVELDGDVNGHKFSVSGEG |
| indicated with | EGDATYGKLTLKFICTTGKLPVPWPTLVTTLTYGVQCFSRYPDHMKQHD |
| underlining) | FFKSAMPEGYVQERTIFFKDDGNYKTRAEVKFEGDTLVNRIELKGIDFKE |
| | DGNILGHKLEYNYNSHNVYIMADKQKNGIKVNFKIRHNIEDGSVQLADH |
| | YQQNTPIGDGPVLLPDNHYLSTQSALSKDPNEKRDHMVLLEFVTAAGITL |
| | GMDELYK* |
| CD3z-EGFP (EGFP | MKWKALFTAAILQAQLPITEAQSFGLLDPKLCYLLDGILFIYGVILTALFL |
| indicated with | RVKFSRSADAPAYQQGQNQLYNELNLGRREEYDVLDKRRGRDPEMGGK |
| underlining) | PRRKNPQEGLYNELQKDKMAEAYSEIGMKGERRRGKGHDGLYQGLSTA |
| | TKDTYDALHMQALPPRPPVATMVSKGEELFTGVVPILVELDGDVNGHKF |
| | SVSGEGEGDATYGKLTLKFICTTGKLPVPWPTLVTTLTYGVQCFSRYPDH |
| | MKQHDFFKSAMPEGYVQERTIFFKDDGNYKTRAEVKFEGDTLVNRIELK |
| | GIDFKEDGNILGHKLEYNYNSHNVYIMADKQKNGIKVNFKIRHNIEDGSV |
| | QLADHYQQNTPIGDGPVLLPDNHYLSTQSALSKDPNEKRDHMVLLEFVT |
| | AAGITLGMDELYK* |
| EGFP-hZAP70 SH2 | MVSKGEELFTGVVPILVELDGDVNGHKFSVSGEGEGDATYGKLTLKFICT |
| (EGFP indicated | TGKLPVPWPTLVTTLTYGVQCFSRYPDHMKQHDFFKSAMPEGYVQERTI |
| with underlining) | FFKDDGNYKTRAEVKFEGDTLVNRIELKGIDFKEDGNILGHKLEYNYNSH |
| | NVYIMADKQKNGIKVNFKIRHNIEDGSVQLADHYQQNTPIGDGPVLLPD |
| | NHYLSTQSALSKDPNEKRDHMVLLEFVTAAGITLGMDELYKGPGSASGE |
| | GLPGSAGPGMPDPAAHLPFFYGSISRAEAEEHLKLAGMADGLFLLRQCLR |
| | SLGGYVLSLVHDVRFHHFPIERQLNGTYAIAGGKAHCGPAELCEFYSRDP |
| | DGLPCNLRKPCNRPSGLEPQPGVFDCLRDAMVRDYVRQTWKLEGEALE |
| | QAIISQAPQVEKLIATTAHERMPWYHSSLTREEAERKLYSGAQTDGKFLL |
| | RPRKEQGTYALSLIYGKTVYHYLISQDKAGKYCIPEGTKFDTLWQLVEYL |
| | KLKADGLIYCLKEACPNSSA* |
| MCP-hZAP70_SH2 | MGNASNFTQFVLVDNGGTGDVTVAPSNFANGIAEWISSNSRSQAYKVTC |
| (MCP indicated with | SVRQSSAQNRKYTIKVEVPKGAWRSYLNMELTIPIFATNSDCELIVKAMQ |
| underlining) | GLLKDGNPIPSAIAANSGIYAMASNFTQFVLVDNGGTGDVTVAPSNFANG |
| | IAEWISSNSRSQAYKVTCSVRQSSAQNRKYTIKVEVPKGAWRSYLNMEL |
| | TIPIFATNSDCELIVKAMQGLLKDGNPIPSAIAANSGIYAGPGSASGEGLPG |
| | SAGPGMPDPAAHLPFFYGSISRAEAEEHLKLAGMADGLFLLRQCLRSLGG |
| | YVLSLVHDVRFHHFPIERQLNGTYAIAGGKAHCGPAELCEFYSRDPDGLP |
| | CNLRKPCNRPSGLEPQPGVFDCLRDAMVRDYVRQTWKLEGEALEQAIIS |
| | QAPQVEKLIATTAHERMPWYHSSLTREEAERKLYSGAQTDGKFLLRPRK |
| | EQGTYALSLIYGKTVYHYLISQDKAGKYCIPEGTKFDTLWQLVEYLKLK |
| | ADGLIYCLKEACPNSSA* |
| hCD3z-MCP (MCP | MKWKALFTAAILQAQLPITEAQSFGLLDPKLCYLLDGILFIYGVILTALFL |
| indicated with | RVKFSRSADAPAYQQGQNQLYNELNLGRREEYDVLDKRRGRDPEMGGK |
| underlining) | PRRKNPQEGLYNELQKDKMAEAYSEIGMKGERRRGKGHDGLYQGLSTA |
| | TKDTYDALHMQALPPRGSGGDYKDDDDKGSNASNFTQFVLVDNGGTGD |
| | VTVAPSNFANGIAEWISSNSRSQAYKVTCSVRQSSAQNRKYTIKVEVPKG |
| | AWRSYLNMELTIPIFATNSDCELIVKAMQGLLKDGNPIPSAIAANSGIYAM |
| | ASNFTQFVLVDNGGTGDVTVAPSNFANGIAEWISSNSRSQAYKVTCSVR |
| | QSSAQNRKYTIKVEVPKGAWRSYLNMELTIPIFATNSDCELIVKAMQGLL |
| | KDGNPIPSAIAANSGIYA* |
| OT1 TCR | MSNTVLADSAWGITLLSWVTVFLLGTSSADSGVVQSPRHIIKEKGGRSVL |
| | TCIPISGHSNVVWYQQTLGKELKFLIQHYEKVERDKGFLPSRFSVQQFDD |
| | YHSEMNMSALELEDSAMYFCASSRANYEQYFGPGTRLTVLEDLRNVTPP |
| | KVSLFEPSKAEIANKQKATLVCLARGFFPDHVELSWWVNGKEVHSGVST |
| | DPQAYKESNYSYCLSSRLRVSATFWHNPRNHFRCQVQFHGLSEEDKWPE |
| | GSPKPVTQNISAEAWGRADCGITSASYHQGVLSATILYEILLGKATLYAV |
| | LVSGLVLMAMVKKKNSATNFSLLKQAGDVEENPGPMDKILTASFLLLGL |
| | HLAGVNGQQQEKRDQQQVRQSPQSLTVWEGETAILNCSYEDSTFNYFPW |
| | YQQFPGEGPALLISIRSVSDKKEDGRFTIFFNKREKKLSLHITDSQPGDSAT |
| | YFCAASDNYQLIWGSGTKLIIKPDIQNPEPAVYQLKDPRSQDSTLCLFTDF |
| | DSQINVPKTMESGTFITDKTVLDMKAMDSKSNGAIAWSNQTSFTCQDIFK |
| | ETNATYPSSDVPCDATLTEKSFETDMNLNFQNLSVMGLRILLLKVAGFNL |
| | LMTLRLWSS* |
| mCD3z-EGFP | MKWKVSVLACILHVRFPGAEAQSFGLLDPKLCYLLDGILFIYGVIITALYL |
| (EGFP indicated | RAKFSRSAETAANLQDPNQLYNELNLGRREEYDVLEKKRARDPEMGGK |
| with underlining) | QQRRRNPQEGVYNALQKDKMAEAYSEIGTKGERRRGKGHDGLYQGLST |
| | ATKDTYDALHMQTLAPRPPVATMVSKGEELFTGVVPILVELDGDVNGHK |
| | FSVSGEGEGDATYGKLTLKFICTTGKLPVPWPTLVTTLTYGVQCFSRYPD |
| | HMKQHDFFKSAMPEGYVQERTIFFKDDGNYKTRAEVKFEGDTLVNRIEL |
| | KGIDFKEDGNILGHKLEYNYNSHNVYIMADKQKNGIKVNFKIRHNIEDGS |
| | VQLADHYQQNTPIGDGPVLLPDNHYLSTQSALSKDPNEKRDHMVLLEFV |
| | TAAGITLGMDELYK* |
| EGFP- | MVSKGEELFTGVVPILVELDGDVNGHKFSVSGEGEGDATYGKLTLKFICT |
| mZAP70_SH2 | TGKLPVPWPTLVTTLTYGVQCFSRYPDHMKQHDFFKSAMPEGYVQERTI |
| (EGFP indicated | FFKDDGNYKTRAEVKFEGDTLVNRIELKGIDFKEDGNILGHKLEYNYNSH |
| with underlining) | NVYIMADKQKNGIKVNFKIRHNIEDGSVQLADHYQQNTPIGDGPVLLPD |
| | NHYLSTQSALSKDPNEKRDHMVLLEFVTAAGITLGMDELYKGPGSASGE |
| | GLPGSAGPGMPDPAAHLPFFYGSISRAEAEEHLKLAGMADGLFLLRQCLR |
| | SLGGYVLSLVHDVRFHHFPIERQLNGTYAIAGGKAHCGPAELCQFYSQDP |
| | DGLPCNLRKPCNRPPGLEPQPGVFDCLRDAMVCDYVRQTWKLEGDALE |
| | QAIISQAPQVEKLIATTAHERMPWYHSSLTREEAERKLYSGQQTDGKFLL |
| | RPRKEQGTYALSLVYGKTVYHYLISQDKAGKYCIPEGTKFDTLWQLVEY |
| | LKLKADGLIYRLKEVCPNSSA* |
| Cre-mZAP70 _SH2 | MANLLTVHQNLPALPVDATSDEVRKNLMDMFRDRQAFSEHTWKMLLS |
| (Cre indicated with | VCRSWAAWCKLNNRKWFPAEPEDVRDYLLYLQARGLAVKTIQQHLGQL |
| underlining) | NMLHRRSGLPRPSDSNAVSLVMRRIRKENVDAGERAKQALAFERTDFDQ |
| | VRSLMENSDRCQDIRNLAFLGIAYNTLLRIAEIARIRVKDISRTDGGRMLI |
| | HIGRTKTLVSTAGVEKALSLGVTKLVERWISVSGVADDPNNYLFCRVRK |
| | NGVAAPSATSQLSTRALEGIFEATHRLIYGAKDDSGQRYLAWSGHSARV |
| | GAARDMARAGVSIPEIMQAGGWTNVNIVMNYIRNLDSETGAMVRLLED |
| | GDGPVGSENLYFQSGGGSMPDPAAHLPFFYGSISRAEAEEHLKLAGMAD |
| | GLFLLRQCLRSLGGYVLSLVHDVRFHHFPIERQLNGTYAIAGGKAHCGPA |
| | ELCQFYSQDPDGLPCNLRKPCNRPPGLEPQPGVFDCLRDAMVCDYVRQT |
| | WKLEGDALEQAIISQAPQVEKLIATTAHERMPWYHSSLTREEAERKLYSG |
| | QQTDGKFLLRPRKEQGTYALSLVYGKTVYHYLISQDKAGKYCIPEGTKF |
| | DTLWQLVEYLKLKADGLIYRLKEVCPNSSA* |
| MCP-mZAP70_SH2 | MGNASNFTQFVLVDNGGTGDVTVAPSNFANGIAEWISSNSRSQAYKVTC |
| (MCP indicated with | SVRQSSAQNRKYTIKVEVPKGAWRSYLNMELTIPIFATNSDCELIVKAMQ |
| underlining) | GLLKDGNPIPSAIAANSGIYAMASNFTQFVLVDNGGTGDVTVAPSNFANG |
| | IAEWISSNSRSQAYKVTCSVRQSSAQNRKYTIKVEVPKGAWRSYLNMEL |
| | TIPIFATNSDCELIVKAMQGLLKDGNPIPSAIAANSGIYAGPGSASGEGLPG |
| | SAGPGMPDPAAHLPFFYGSISRAEAEEHLKLAGMADGLFLLRQCLRSLGG |
| | YVLSLVHDVRFHHFPIERQLNGTYAIAGGKAHCGPAELCQFYSQDPDGLP |
| | CNLRKPCNRPPGLEPQPGVFDCLRDAMVCDYVRQTWKLEGDALEQAIIS |
| | QAPQVEKLIATTAHERMPWYHSSLTREEAERKLYSGQQTDGKFLLRPRK |
| | EQGTYALSLVYGKTVYHYLISQDKAGKYCIPEGTKFDTLWQLVEYLKLK |
| | ADGLIYRLKEVCPNSSA* |
| Lck-Scarlet-I | MGCVCSSNPELPVATMVSKGEAVIKEFMRFKVHMEGSMNGHEFEIEGEG |
| (Scarlet-I sequence | EGRPYEGTQTAKLKVTKGGPLPFSWDILSPQFMYGSRAFIKHPADIPDYY |
| indicated with | KQSFPEGFKWERVMNFEDGGAVTVTQDTSLEDGTLIYKVKLRGTNFPPD |
| underlining) | GPVMQKKTMGWEASTERLYPEDGVLKGDIKMALRLKDGGRYLADFKTT |
| | YKAKKPVQMPGAYNVDRKLDITSHNEDYTVVEQYERSEGRHSTGGMDE |
| | LYK* |
| CD19CAR-CD3z- | MALPVTALLLPLALLLHAARPDIQMTQTTSSLSASLGDRVTISCRASQDIS |
| mCherry (CD19 | KYLNWYQQKPDGTVKLLIYHTSRLHSGVPSRFSGSGSGTDYSLTISNLEQ |
| CAR indicated by | EDIATYFCQQGNTLPYTFGGGTKLEITGGGGSGGGGSGGGGSEVKLQESG |
| underlining; | PGLVAPSQSLSVTCTVSGVSLPDYGVSWIRQPPRKGLEWLGVIWGSETTY |
| mCherry indicated | YNSALKSRLTIIKDNSKSQVFLKMNSLQTDDTAIYYCAKHYYYGGSYAM |
| by bolded italics) | DYWGQGTSVTVSSTTTPAPRPPTPAPTIASQPLSLRPEACRPAAGGAVHTR |
| | GLDFACDIYIWAPLAGTCGVLLLSLVITLYCNHRNRRRRVKFSRSADAPA |
| | YQQGQNQLYNELNLGRREEYDVLDKRRGRDPEMGGKPRRKNPQEGLYN |
| | ELQKDKMAEAYSEIGMKGERRRGKGHDGLYQGLSTATKDTYDALHMQ |
| | ALPPRPPVATMVSKGEEDNMAIIKEFMRFKVHMEGSVNGHEFEIEGEGE |
| | GRPYEGTQTAKLKVTKGGPLPFAWDILSPQFMYGSKAYVKHPADIPDYLK |
| | LSFPEGFKWERVMNFEDGGVVTVTQDSSLQDGEFIYKVKLRGTNFPSDG |
| | PVMQKKTMGWEASSERMYPEDGALKGEIKQRLKLKDGGHYDAEVKTTY |
| | KAKKPVQLPGAYNVNIKLDITSHNEDYTIVEQYERAEGRHSTGGMDELYK |
| | * |
| mSA2-CAR-CD3z- | METDTLLLWVLLLWVPGSTGGAEAGITGTWYNQHGSTFTVTAGADGNL |
| mCherry (mSA2 | TGQYENRAQGTGCQNSPYTLTGRYNGTKLEWRVEWNNSTENCHSRTEW |
| CAR indicated by | RGQYQGGAEARINTQWNLTYEGGSGPATEQGQDTFTKVKPSAASGSTTT |
| underlining; | PAPRPPTPAPTIASQPLSLRPEACRPAAGGAVHTRGLDFACDIYIWAPLAG |
| mCherry indicated | TCGVLLLSLVITLYCNHRNRRRRVKFSRSADAPAYQQGQNQLYNELNLG |
| by bolded italics) | RREEYDVLDKRRGRDPEMGGKPRRKNPQEGLYNELQKDKMAEAYSEIG |
| | MKGERRRGKGHDGLYQGLSTATKDTYDALHMQALPPRPPVATMVSKGE |
| | EDNMAIIKEFMRFKVHMEGSVNGHEFEIEGEGEGRPYEGTQTAKLKVT |
| | KGGPLPFAWDILSPQFMYGSKAYVKHPADIPDYLKLSFPEGFKWERVMN |
| | FEDGGVVTVTQDSSLQDGEFIYKVKLRGTNFPSDGPVMQKKTMGWEASS |
| | ERMYPEDGALKGEIKQRLKLKDGGHYDAEVKTTYKAKKPVQLPGAYNVN |
| | IKLDITSHNEDYTIVEQYERAEGRHSTGGMDELYK* |

TABLE 3
Exemplary Nucleic Acid Sequences Encoding Membrane-Associated Agents

| Name | Sequence |
|---|---|
| MCP-ZAP70_ | gccaccatgggcaacgcttctaactttactcagttcgttctcgtcgacaatggcggaactggcgacgtgactgtcgcc |
| SH2 | ccaagcaacttcgctaacgggatcgctgaatggatcagctctaactcgcgttcacaggcttacaaagtaacctgtagc |
| | gttcgtcagagctctgcgcagaatcgcaaatacaccatcaaagtcgaggtgcctaaaggcgcctggcgttcgtactt |
| | aaatatggaactaaccattccaattttcgccacgaattccgactgcgagcttattgttaaggcaatgcaaggtctcctaa |
| | aagatggaaacccgattccctcagcaatcgcagcaaactccggcatctacgccatggccagcaacttcacccagttc |
| | gtgctggtggacaacggcggcaccggcgacgtgaccgtggcccccagcaacttcgccaacggcatcgccgagtg |
| | gatcagcagcaacagcagaagccaggcctacaaggtgacctgcagcgtgagacagagcagcgcccagaacaga |
| | aagtacaccatcaaggtggaggtgcccaagggcgcctggagaagctacctgaacatggagctgaccatccccatc |
| | ttcgccaccaacagcgactgcgagctgatcgtgaaggccatgcagggcctgctgaaggacggcaaccccatcccc |
| | agcgccatcgccgccaacagcggcatctacgccggaccaggatcggcgtctggggaaggactgccaggatcag |
| | ccggaccaggaatgccagaccccgcggcgcacctgcccttcttctacggcagcatctcgcgtgccgaggccgag |
| | gagcacctgaagctggcgggcatggcggacgggctcttcctgctgcgccagtgcctgcgctcgctgggcggctat |
| | gtgctgtcgctcgtgcacgatgtgcgcttccaccactttcccatcgagcgccagctcaacggcacctacgccattgcc |
| | ggcggcaaagcgcactgtggaccggcagagctctgcgagttctactcgcgcgaccccgacgggctgccctgcaa |
| | cctgcgcaagccgtgcaaccggccgtcgggcctcgagccgcagccgggggtcttcgactgcctgcgagacgcca |
| | tggtgcgtgactacgtgcgccagacgtggaagctggagggcgaggccctggagcaggccatcatcagccaggcc |
| | ccgcaggtggagaagctcattgctacgacggcccacgagcggatgccctggtaccacagcagcctgacgcgtga |
| | ggaggccgagcgcaaactttactctggggcgcagaccgacggcaagttcctgctgaggccgcggaaggagcag |
| | ggcacatacgccctgtccctcatctatgggaagacggtgtaccactacctcatcagccaagacaaggcgggcaagt |
| | actgcattcccgagggcaccaagtttgacacgctctggcagctggtggagtatctgaagctgaaggcggacgggct |
| | catctactgcctgaaggaggcctgccccaacagcagtgccccagggcaccatcatcatcatcattaa |
| hCD3z-MCP | gccaccATGAAGTGGAAGGCGCTTTTCACCGCGGCCATCCTGCAGGCAC |
| | AGTTGCCGATTACAGAGGCACAGAGCTTTGGCCTGCTGGAcCCCAAAC |
| | TCTGCTACCTGCTGGATGGAATCCTCTTCATCTATGGTGTCATTCTCAC |
| | TGCCTTGTTCCTGAGAGTGAAGTTCAGCAGGAGCGCAGACGCCCCCGC |
| | GTACCAGCAGGGCCAGAACCAGCTCTATAACGAGCTCAATCTAGGAC |
| | GAAGAGAGGAGTACGATGTTTTGGACAAGAGACGTGGCCGGGACCCT |
| | GAGATGGGGGGAAAGCCGAGAAGGAAGAACCCTCAGGAAGGCCTGT |
| | AtAATGAACTGCAGAAAGATAAGATGGCGGAGGCCTACAGTGAGATT |
| | GGGATGAAAGGCGAGCGCCGGAGGGGCAAGGGGCACGATGGCCTTT |
| | ACCAGGGTCTCAGTACAGCCACCAAGGACACCTACGACGCCCTTCAC |
| | ATGCAGGCCCTGCCCCCTCGCggtagcggtggtGATTACAAGGACGACGATG |
| | ACAAGggtagcaacgcttctaactttactcagttcgttctcgtcgacaatggcggaactggcgacgtgactgtcg |
| | ccccaagcaacttcgctaacgggatcgctgaatggatcagctctaactcgcgttcacaggcttacaaagtaacctgta |
| | gcgttcgtcagagctctgcgcagaatcgcaaatacaccatcaaagtcgaggtgcctaaaggcgcctggcgttcgta |
| | cttaaatatggaactaaccattccaattttcgccacgaattccgactgcgagcttattgttaaggcaatgcaaggtctcc |
| | taaaagatggaaacccgattccctcagcaatcgcagcaaactccggcatctacgccatggccagcaacttcacccag |
| | ttcgtgctggtggacaacggcggcaccggcgacgtgaccgtggcccccagcaacttcgccaacggcatcgccga |
| | gtggatcagcagcaacagcagaagccaggcctacaaggtgacctgcagcgtgagacagagcagcgcccagaac |
| | agaaagtacaccatcaaggtggaggtgcccaagggcgcctggagaagctacctgaacatggagctgaccatccc |
| | catcttcgccaccaacagcgactgcgagctgatcgtgaaggccatgcagggcctgctgaaggacggcaaccccat |
| | ccccagcgccatcgccgccaacagcggcatctacgccTAG |
| EGFP-MS2 mRNA | Gccaccatggtgagcaagggcgaggagctgttcaccggggtggtgcccatcctggtcgagctggacggcgacgt |
| | aaacggccacaagttcagcgtgtccggcgagggcgagggcgatgccacctacggcaagctgaccctgaagttcat |
| | ctgcaccaccggcaagctgcccgtgccctggcccaccctcgtgaccaccctgacctacggcgtgcagtgcttcagc |
| | cgctaccccgaccacatgaagcagcacgacttcttcaagtccgccatgcccgaaggctacgtccaggagcgcacc |
| | atcttcttcaaggacgacggcaactacaagacccgcgccgaggtgaagttcgagggcgacaccctggtgaaccgc |
| | atcgagctgaagggcatcgacttcaaggaggacggcaacatcctggggcacaagctggagtacaactacaacag |
| | ccacaacgtctatatcatggccgacaagcagaagaacggcatcaaggtgaacttcaagatccgccacaacatcgag |
| | gacggcagcgtgcagctcgccgaccactaccagcagaacacccccatcggcgacggccccgtgctgctgcccg |
| | acaaccactacctgagcacccagtccgccctgagcaaagaccccaacgagaagcgcgatcacatggtcctgctgg |
| | agttcgtgaccgccgccgggatcactctcggcatggacgagctgtataagtaaagcgccgcgtgactacggtactta |
| | ttgccaagaaaGCACGAGCATCAGCCGTGCctccaggtcgaatcttcaaaCGACGACGA |
| | TCACGCGTCGctccagtattccagggttcatc |
| PM-EGFP | Gccaccatgggctgcgtgtgcagcagcaaccccgagctaccggtcgccaccatggtgagcaagggcgaggagc |
| | tgttcaccggggtggtgcccatcctggtcgagctggacggcgacgtaaacggccacaagttcagcgtgtccggcg |
| | agggcgagggcgatgccacctacggcaagctgaccctgaagttcatctgcaccaccggcaagctgcccgtgccct |
| | ggcccaccctcgtgaccaccctgacctacggcgtgcagtgcttcagccgctaccccgaccacatgaagcagcacg |
| | acttcttcaagtccgccatgcccgaaggctacgtccaggagcgcaccatcttcttcaaggacgacggcaactacaag |
| | acccgcgccgaggtgaagttcgagggcgacaccctggtgaaccgcatcgagctgaagggcatcgacttcaagga |
| | ggacggcaacatcctggggcacaagctggagtacaactacaacagccacaacgtctatatcatggccgacaagca |
| | gaagaacggcatcaaggtgaacttcaagatccgccacaacatcgaggacggcagcgtgcagctcgccgaccact |
| | accagcagaacacccccatcggcgacggccccgtgctgctgcccgacaaccactacctgagcacccagtccgcc |
| | ctgagcaaagaccccaacgagaagcgcgatcacatggtcctgctggagttcgtgaccgccgccgggatcactctc |
| | ggcatggacgagctgtacaagtaa |
| CD3z-EGFP | gccaccATGAAGTGGAAGGCGCTTTTCACCGCGGCCATCCTGCAGGCAC |
| | AGTTGCCGATTACAGAGGCACAGAGCTTTGGCCTGCTGGAcCCCAAAC |
| | TCTGCTACCTGCTGGATGGAATCCTCTTCATCTATGGTGTCATTCTCAC |
| | TGCCTTGTTCCTGAGAGTGAAGTTCAGCAGGAGCGCAGACGCCCCCGC |
| | GTACCAGCAGGGCCAGAACCAGCTCTATAACGAGCTCAATCTAGGAC |
| | GAAGAGAGGAGTACGATGTTTTGGACAAGAGACGTGGCCGGGACCCT |
| | GAGATGGGGGGAAAGCCGAGAAGGAAGAACCCTCAGGAAGGCCTGT |
| | AtAATGAACTGCAGAAAGATAAGATGGCGGAGGCCTACAGTGAGATT |
| | GGGATGAAAGGCGAGCGCCGGAGGGGCAAGGGGCACGATGGCCTTT |
| | ACCAGGGTCTCAGTACAGCCACCAAGGACACCTACGACGCCCTTCAC |
| | ATGCAGGCCCTGCCCCCTCGCccacccgtcgccaccatggtgagcaagggcgaggagctgttc |
| | accggggtggtgcccatcctggtcgagctggacggcgacgtaaacggccacaagttcagcgtgtccggcgaggg |
| | cgagggcgatgccacctacggcaagctgaccctgaagttcatctgcaccaccggcaagctgcccgtgccctggcc |
| | caccctcgtgaccaccctgacctacggcgtgcagtgcttcagccgctaccccgaccacatgaagcagcacgacttc |
| | ttcaagtccgccatgcccgaaggctacgtccaggagcgcaccatcttcttcaaggacgacggcaactacaagaccc |
| | gcgccgaggtgaagttcgagggcgacaccctggtgaaccgcatcgagctgaagggcatcgacttcaaggaggac |
| | ggcaacatcctggggcacaagctggagtacaactacaacagccacaacgtctatatcatggccgacaagcagaag |
| | aacggcatcaaggtgaacttcaagatccgccacaacatcgaggacggcagcgtgcagctcgccgaccactacca |
| | gcagaacacccccatcggcgacggccccgtgctgctgcccgacaaccactacctgagcacccagtccgccctga |
| | gcaaagaccccaacgagaagcgcgatcacatggtcctgctggagttcgtgaccgccgccgggatcactctcggca |
| | tggacgagctgtataagtga |
| EGFP-ZAP70_ | gccaccatggtgagcaagggcgaggagctgttcaccggggtggtgcccatcctggtcgagctggacggcgacgt |
| SH2 | aaacggccacaagttcagcgtgtccggcgagggcgagggcgatgccacctacggcaagctgaccctgaagttcat |
| | ctgcaccaccggcaagctgcccgtgccctggcccaccctcgtgaccaccctgacctacggcgtgcagtgcttcagc |
| | cgctaccccgaccacatgaagcagcacgacttcttcaagtccgccatgcccgaaggctacgtccaggagcgcacc |
| | atcttcttcaaggacgacggcaactacaagacccgcgccgaggtgaagttcgagggcgacaccctggtgaaccgc |
| | atcgagctgaagggcatcgacttcaaggaggacggcaacatcctggggcacaagctggagtacaactacaacag |
| | ccacaacgtctatatcatggccgacaagcagaagaacggcatcaaggtgaacttcaagatccgccacaacatcgag |
| | gacggcagcgtgcagctcgccgaccactaccagcagaacacccccatcggcgacggccccgtgctgctgcccg |
| | acaaccactacctgagcacccagtccgccctgagcaaagaccccaacgagaagcgcgatcacatggtcctgctgg |
| | agttcgtgaccgccgccgggatcactctcggcatggacgagctgtataagggaccaggatcggcgtctggggaag |
| | gactgccaggatcagccggaccaggaATGCCCGATCCCGCGGCGCAtCTGCCATTCT |
| | TCTATGGCAGCATCTCGCGGGCTGAGGCCGAGGAGCACCTGAAGCTG |
| | GCAGGCATGGCCGACGGGCTGTTCCTCCTGCGCCAGTGTTTGCGCTCC |
| | CTGGGCGGCTACGTGCTGTCGTTGGTGCACGACGTGCGCTTCCACCAT |
| | TTCCCCATCGAGCGCCAACTCAACGGCACGTACGCCATCGCGGGCGG |
| | GAAGGCGCACTGCGGCCCGGCCGAGCTCTGCCAGTTCTACTCTCAGGA |
| | CCCCGATGGGCTGCCCTGCAACCTGCGTAAGCCGTGTAACCGGCCGCC |
| | CGGACTGGAGCCACAGCCCGGGGTCTTCGACTGCCTGCGTGATGCTAT |
| | GGTGTGCGACTACGTGCGCCAGACCTGGAAGCTGGAGGGCGATGCAC |
| | TAGAGCAGGCCATCATCAGCCAGGCCCCACAGGTGGAGAAGCTCATT |
| | GCTACCACAGCCCACGAGCGAATGCCCTGGTATCACAGCAGCCTGAC |
| | TCGTGAGGAGGCCGAGCGCAAACTCTATTCCGGCCAGCAGACCGACG |
| | GCAAGTTCCTGCTGAGGCCCCGGAAGGAGCAGGGCACATATGCACTG |
| | TCCCTGGTCTATGGGAAAACTGTATACCACTATCTCATCAGCCAGGAC |
| | AAGGCTGGCAAGTACTGCATCCCCGAAGGCACCAAGTTTGACACGCT |
| | CTGGCAGCTGGTGGAGTACCTGAAGCTGAAGGCGGATGGACTAATTT |
| | ACCGCTTGAAGGAGGTCTGTCCCAACAGCAGCGCCccagggcaccatcatcatca |
| | tcattaa |
| OT1 TCR | atgtctaacactgtcctcgctgattctgcctggggcatcaccctgctatcttgggttactgtctttctcttgggaacaag |
| | ttcagcagattctggggttgtccagtctccaagacacataatcaaagaaaagggaggaaggtccgttctgacgtgtattc |
| | ccatctctggacatagcaatgtggtctggtaccagcagactctggggaaggaattaaagttccttattcagcattatga |
| | aaaggtggagagagacaaaggattcctacccagcagattctcagtccaacagtttgatgactatcactctgaaatgaa |
| | catgagtgccttggaactggaggactctgctatgtacttctgtgccagctctcgggccaattatgaacagtacttcggt |
| | cccggcaccaggctcacggttttagaggatctgagaaatgtgactccacccaaggtctccttgtttgagccatcaaaa |
| | gcagagattgcaaacaaacaaaaggctaccctcgtgtgcttggccaggggcttcttccctgaccacgtggagctga |
| | gctggtgggtgaatggcaaggaggtccacagtggggtcagcacggaccctcaggcctacaaggagagcaattata |
| | gctactgcctgagcagccgcctgagggtctctgctaccttctggcacaatcctcgaaaccacttccgctgccaagtgc |
| | agttccatgggctttcagaggaggacaagtggccagagggctcacccaaacctgtcacacagaacatcagtgcag |
| | aggcctggggccgagcagactgtggaatcacttcagcatcctatcatcagggggttctgtctgcaaccatcctctatg |
| | agatcctactggggaaggccaccctatatgctgtgctggtcagtggcctggtgctgatggccatggtcaagaaaaaa |
| | aattccgccacgaacttctctctgttaaagcaagcaggagacgtggaagaaaaccccggtcctatggacaagatcct |
| | gacagcatcgtttttactcctaggccttcacctagctggggtgaatggccagcagcaggagaaacgtgaccagcag |
| | caggtgagacaaagtccccaatctctgacagtctgggaaggagagaccgcaattctgaactgcagttatgaggaca |
| | gcacttttaactacttcccatggtaccagcagttccctggggaaggccctgcactcctgatatccatacgttcagtgtcc |
| | gataaaaaggaagatggacgattcacaatcttcttcaataaaagggagaaaaagctctccttgcacatcacagactct |
| | cagcctggagactcagctacctacttctgtgcagcaagtgacaactatcagttgatctggggctctgggaccaagcta |
| | attataaagccagacatccagaacccagaacctgctgtgtaccagttaaaagatcctcggtctcaggacagcaccct |
| | ctgcctgttcaccgactttgactcccaaatcaatgtgccgaaaaccatggaatctggaacgttcatcactgacaaaact |
| | gtgctggacatgaaagctatggattccaagagcaatggggccattgcctggagcaaccagacaagcttcacctgcc |
| | aagatatcttcaaagagaccaacgccacctaccccagttcagacgttccctgtgatgccacgttgactgagaaaagct |
| | ttgaaacagatatgaacctaaactttcaaaacctgtcagttatgggactccgaatcctcctgctgaaagtagccggattt |
| | aacctgctcatgacgctgaggctgtggtcctcctaa |
| mCD3z-EGFP | gccaccatgaagtggaaagtgtctgttctcgcctgcatcctccacgtgcggttcccaggagcagaggcacagagctt |
| | tggtctgctggaccccaaactctgctacttgctagatggaatcctcttcatctacggagtcatcatcacagccctgtacc |
| | tgagagcaaaattcagcaggagtgcagagactgctgccaacctgcaggaccccaaccagctctacaatgagctca |
| | atctagggcgaagagaggaatatgacgtcttggagaagaagcgggctcgggacccagagatgggaggcaaaca |
| | gcagaggaggaggaacccccaggaaggcgtatacaatgcactgcagaaagacaagatggcagaagcctacagt |
| | gagatcggcacaaaaggcgagaggcggagaggcaaggggcacgatggcctttaccagggtctcagcactgcca |
| | ccaaggacacctatgatgccctgcatatgcagaccctggcccctcgcccacccgtcgccaccatggtgagcaagg |
| | gcgaggagctgttcaccggggtggtgcccatcctggtcgagctggacggcgacgtaaacggccacaagttcagc |
| | gtgtccggcgagggcgagggcgatgccacctacggcaagctgaccctgaagttcatctgcaccaccggcaagct |
| | gcccgtgccctggcccaccctcgtgaccaccctgacctacggcgtgcagtgcttcagccgctaccccgaccacatg |
| | aagcagcacgacttcttcaagtccgccatgcccgaaggctacgtccaggagcgcaccatcttcttcaaggacgacg |
| | gcaactacaagacccgcgccgaggtgaagttcgagggcgacaccctggtgaaccgcatcgagctgaagggcatc |
| | gacttcaaggaggacggcaacatcctggggcacaagctggagtacaactacaacagccacaacgtctatatcatgg |
| | ccgacaagcagaagaacggcatcaaggtgaacttcaagatccgccacaacatcgaggacggcagcgtgcagctc |
| | gccgaccactaccagcagaacacccccatcggcgacggccccgtgctgctgcccgacaaccactacctgagcac |
| | ccagtccgccctgagcaaagaccccaacgagaagcgcgatcacatggtcctgctggagttcgtgaccgccgccg |
| | ggatcactctcggcatggacgagctgtataagtga |
| Lck-Scarlet-I | TCCGGAgccaccatgggctgcgtgtgcagcagcaaccccgagctacccgtcgccaccatggtgagcaaggg |
| | cgaggcagtgatcaaggagttcatgcggttcaaggtgcacatggagggctccatgaacggccacgagttcgagat |
| | cgagggcgagggcgagggccgcccctacgagggcacccagaccgccaagctgaaggtgaccaagggtggcc |
| | ccctgcccttctcctgggacatcctgtcccctcagttcatgtacggctccagggccttcatcaagcaccccgccgaca |
| | tccccgactactataagcagtccttccccgagggcttcaagtgggagcgcgtgatgaacttcgaggacggcggcgc |
| | cgtgaccgtgacccaggacacctccctggaggacggcaccctgatctacaaggtgaagctccgcggcaccaactt |
| | ccctcctgacggccccgtaatgcagaagaagacaatgggctgggaagcgtccaccgagcggttgtaccccgagg |
| | acggcgtgctgaagggcgacattaagatggccctgcgcctgaaggacggcggccgctacctggcggacttcaag |
| | accacctacaaggccaagaagcccgtgcagatgcccggcgcctacaacgtcgaccgcaagttggacatcacctcc |
| | cacaacgaggactacaccgtggtggaacagtacgaacgctccgagggccgccactccaccggcggcatggacg |
| | agctgtataagtaaGGATCC |
| CD19CAR-CD3z- | TCCGGAgccaccatggccttaccagtgaccgccttgctcctgccgctggccttgctgctccacgccgccaggc |
| mCherry | cggacatccagatgacacagactacatcctccctgtctgcctctctgggagacagagtcaccatcagttgcagggca |
| | agtcaggacattagtaaatatttaaattggtatcagcagaaaccagatggaactgttaaactcctgatctaccatacatc |
| | aagattacactcaggagtcccatcaaggttcagtggcagtgggtctggaacagattattctctcaccattagcaacctg |
| | gagcaagaagatattgccacttacttttgccaacagggtaatacgcttccgtacacgttcggaggggggaccaagct |
| | ggagatcacaggtggcggtggctcgggcggtggtgggtcgggtggcggcggatctgaggtgaaactgcaggagt |
| | caggacctggcctggtggcgccctcacagagcctgtccgtcacatgcactgtctcaggggtctcattacccgactat |
| | ggtgtaagctggattcgccagcctccacgaaagggtctggagtggctgggagtaatatggggtagtgaaaccacat |
| | actataattcagctctcaaatccagactgaccatcatcaaggacaactccaagagccaagttttcttaaaaatgaacag |
| | tctgcaaactgatgacacagccatttactactgtgccaaacattattactacggtggtagctatgctatggactactggg |
| | gccaaggaacctcagtcaccgtctcctcaaccacgacgccagcgccgcgaccaccaacaccggcgcccaccatc |
| | gcgtcgcagcccctgtccctgcgcccagaggcgtgccggccagcggggggggcgcagtgcacacgaggggg |
| | ctggacttcgcctgtgatatctacatctgggcgcccttggccgggacttgtggggtccttctcctgtcactggttatcac |
| | cctttactgcaaccacaggaaccgaagacgtCGGGTGAAGTTCAGCAGAAGCGCCGACG |
| | CCCCTGCCTACCAGCAGGGCCAGAATCAGCTGTACAACGAGCTGAAC |
| | CTGGGCAGAAGGGAAGAGTACGACGTCCTGGATAAGCGGAGAGGCC |
| | GGGACCCTGAGATGGGCGGCAAGCCTCGGCGGAAGAACCCCCAGGAA |
| | GGCCTGTATAACGAACTGCAGAAAGACAAGATGGCCGAGGCCTACAG |
| | CGAGATCGGCATGAAGGGCGAGCGGAGGCGGGGCAAGGGCCACGAC |
| | GGCCTGTATCAGGGCCTGTCCACCGCCACCAAGGATACCTACGACGCC |
| | CTGCACATGCAGGCCCTGCCCCCAAGGccaccggtcgccaccATGGTGAGCAA |
| | GGGCGAGGAGGATAACATGGCCATCATCAAGGAGTTCATGCGCTTCA |
| | AGGTGCACATGGAGGGCTCCGTGAACGGCCACGAGTTCGAGATCGAG |
| | GGCGAGGGCGAGGGCCGCCCCTACGAGGGCACCCAGACCGCCAAGCT |
| | GAAGGTGACCAAGGGTGGCCCCCTGCCCTTCGCCTGGGACATCCTGTC |
| | CCCTCAGTTCATGTACGGCTCCAAGGCCTACGTGAAGCACCCCGCCGA |
| | CATCCCCGACTACTTGAAGCTGTCCTTCCCCGAGGGCTTCAAGTGGGA |
| | GCGCGTGATGAACTTCGAGGACGGCGGCGTGGTGACCGTGACCCAGG |
| | ACTCCTCCCTGCAGGACGGCGAGTTCATCTACAAGGTGAAGCTGCGCG |
| | GCACCAACTTCCCCTCCGACGGCCCCGTAATGCAGAAGAAGACCATG |
| | GGCTGGGAGGCCTCCTCCGAGCGGATGTACCCCGAGGACGGCGCCCT |
| | GAAGGGCGAGATCAAGCAGAGGCTGAAGCTGAAGGACGGCGGCCAC |
| | TACGACGCTGAGGTCAAGACCACCTACAAGGCCAAGAAGCCCGTGCA |
| | GCTGCCCGGCGCCTACAACGTCAACATCAAGTTGGACATCACCTCCCA |
| | CAACGAGGACTACACCATCGTGGAACAGTACGAACGCGCCGAGGGCC |
| | GCCACTCCACCGGCGGCATGGACGAGCTGTACAAGTAGGGATCC |
| mSA2 CAR-CD3z- | TCCGGAgccaccATGGAGACAGACACACTCCTGCTATGGGTGCTGCTGCT |
| mCherry | CTGGGTTCCAGGTTCCACAGGTGGCGCAGAGGCGGGTATCACCGGGA |
| | CATGGTACAACCAACACGGAAGCACATTTACAGTCACCGCTGGAGCA |
| | GACGGGAATCTGACCGGACAGTACGAGAACAGGGCTCAGGGGACAG |
| | GTTGTCAGAACAGTCCGTATACTCTGACTGGGAGGTACAATGGCACG |
| | AAGCTGGAGTGGCGAGTCGAGTGGAATAATTCCACGGAAAACTGTCA |
| | CAGTAGAACAGAGTGGAGGGGACAGTACCAGGGGGGAGCAGAGGCC |
| | CGGATCAACACCCAATGGAACTTGACATATGAAGGCGGGTCAGGCCC |
| | CGCGACAGAGCAAGGACAGGATACATTCACGAAGGTCAAGCCAAGCG |
| | CAGCCTCTGGCTCTaccacgacgccagcgccgcgaccaccaacaccggcgcccaccatcgcgtcgc |
| | agcccctgtccctgcgcccagaggcgtgccggccagcggggggggcgcagtgcacacgagggggctggactt |
| | cgcctgtgatatctacatctgggcgcccttggccgggacttgtggggtccttctcctgtcactggttatcaccctttactg |
| | caaccacaggaaccgaagacgtCGGGTGAAGTTCAGCAGAAGCGCCGACGCCCCT |
| | GCCTACCAGCAGGGCCAGAATCAGCTGTACAACGAGCTGAACCTGGG |
| | CAGAAGGGAAGAGTACGACGTCCTGGATAAGCGGAGAGGCCGGGAC |
| | CCTGAGATGGGCGGCAAGCCTCGGCGGAAGAACCCCCAGGAAGGCCT |
| | GTATAACGAACTGCAGAAAGACAAGATGGCCGAGGCCTACAGCGAGA |
| | TCGGCATGAAGGGCGAGCGGAGGCGGGGCAAGGGCCACGACGGCCT |
| | GTATCAGGGCCTGTCCACCGCCACCAAGGATACCTACGACGCCCTGCA |
| | CATGCAGGCCCTGCCCCCAAGGccaccggtcgccaccATGGTGAGCAAGGGC |
| | GAGGAGGATAACATGGCCATCATCAAGGAGTTCATGCGCTTCAAGGT |
| | GCACATGGAGGGCTCCGTGAACGGCCACGAGTTCGAGATCGAGGGCG |
| | AGGGCGAGGGCCGCCCCTACGAGGGCACCCAGACCGCCAAGCTGAAG |
| | GTGACCAAGGGTGGCCCCCTGCCCTTCGCCTGGGACATCCTGTCCCCT |
| | CAGTTCATGTACGGCTCCAAGGCCTACGTGAAGCACCCCGCCGACATC |
| | CCCGACTACTTGAAGCTGTCCTTCCCCGAGGGCTTCAAGTGGGAGCGC |
| | GTGATGAACTTCGAGGACGGCGGCGTGGTGACCGTGACCCAGGACTC |
| | CTCCCTGCAGGACGGCGAGTTCATCTACAAGGTGAAGCTGCGCGGCA |
| | CCAACTTCCCCTCCGACGGCCCCGTAATGCAGAAGAAGACCATGGGC |
| | TGGGAGGCCTCCTCCGAGCGGATGTACCCCGAGGACGGCGCCCTGAA |
| | GGGCGAGATCAAGCAGAGGCTGAAGCTGAAGGACGGCGGCCACTAC |
| | GACGCTGAGGTCAAGACCACCTACAAGGCCAAGAAGCCCGTGCAGCT |
| | GCCCGGCGCCTACAACGTCAACATCAAGTTGGACATCACCTCCCACAA |
| | CGAGGACTACACCATCGTGGAACAGTACGAACGCGCCGAGGGCCGCC |
| | ACTCCACCGGCGGCATGGACGAGCTGTACAAGTAGGGATCC |
| MCP-ZAP70_ | gccaccatgggcaacgcttctaactttactcagttcgttctcgtcgacaatggcggaactggcgacgtgactgtcgcc |
| SH2 | ccaagcaacttcgctaacgggatcgctgaatggatcagctctaactcgcgttcacaggcttacaaagtaacctgtagc |
| | gttcgtcagagctctgcgcagaatcgcaaatacaccatcaaagtcgaggtgcctaaaggcgcctggcgttcgtactt |
| | aaatatggaactaaccattccaattttcgccacgaattccgactgcgagcttattgttaaggcaatgcaaggtctcctaa |
| | aagatggaaacccgattccctcagcaatcgcagcaaactccggcatctacgccatggccagcaacttcacccagttc |
| | gtgctggtggacaacggcggcaccggcgacgtgaccgtggcccccagcaacttcgccaacggcatcgccgagtg |
| | gatcagcagcaacagcagaagccaggcctacaaggtgacctgcagcgtgagacagagcagcgcccagaacaga |
| | aagtacaccatcaaggtggaggtgcccaagggcgcctggagaagctacctgaacatggagctgaccatccccatc |
| | ttcgccaccaacagcgactgcgagctgatcgtgaaggccatgcagggcctgctgaaggacggcaaccccatcccc |
| | agcgccatcgccgccaacagcggcatctacgccggaccaggatcggcgtctggggaaggactgccaggatcag |
| | ccggaccaggaatgccagaccccgcggcgcacctgcccttcttctacggcagcatctcgcgtgccgaggccgag |
| | gagcacctgaagctggcgggcatggcggacgggctcttcctgctgcgccagtgcctgcgctcgctgggcggctat |
| | gtgctgtcgctcgtgcacgatgtgcgcttccaccactttcccatcgagcgccagctcaacggcacctacgccattgcc |
| | ggcggcaaagcgcactgtggaccggcagagctctgcgagttctactcgcgcgaccccgacgggctgccctgcaa |
| | cctgcgcaagccgtgcaaccggccgtcgggcctcgagccgcagccgggggtcttcgactgcctgcgagacgcca |
| | tggtgcgtgactacgtgcgccagacgtggaagctggagggcgaggccctggagcaggccatcatcagccaggcc |
| | ccgcaggtggagaagctcattgctacgacggcccacgagcggatgccctggtaccacagcagcctgacgcgtga |
| | ggaggccgagcgcaaactttactctggggcgcagaccgacggcaagttcctgctgaggccgcggaaggagcag |
| | ggcacatacgccctgtccctcatctatgggaagacggtgtaccactacctcatcagccaagacaaggcgggcaagt |
| | actgcattcccgagggcaccaagtttgacacgctctggcagctggtggagtatctgaagctgaaggcggacgggct |
| | catctactgcctgaaggaggcctgccccaacagcagtgccccagggcaccatcatcatcatcattaa |
| MCP DNA | aacgcttctaactttactcagttcgttctcgtcgacaatggcggaactggcgacgtgactgtcgccccaagcaacttcg |
| sequence | ctaacgggatcgctgaatggatcagctctaactcgcgttcacaggcttacaaagtaacctgtagcgttcgtcagagct |
| | ctgcgcagaatcgcaaatacaccatcaaagtcgaggtgcctaaaggcgcctggcgttcgtacttaaatatggaacta |
| | accattccaattttcgccacgaattccgactgcgagcttattgttaaggcaatgcaaggtctcctaaaagatggaaacc |
| | cgattccctcagcaatcgcagcaaactccggcatctacgccatggccagcaacttcacccagttcgtgctggtggac |
| | aacggcggcaccggcgacgtgaccgtggcccccagcaacttcgccaacggcatcgccgagtggatcagcagca |
| | acagcagaagccaggcctacaaggtgacctgcagcgtgagacagagcagcgcccagaacagaaagtacaccat |
| | caaggtggaggtgcccaagggcgcctggagaagctacctgaacatggagctgaccatccccatcttcgccaccaa |
| | cagcgactgcgagctgatcgtgaaggccatgcagggcctgctgaaggacggcaaccccatccccagcgccatcg |
| | ccgccaacagcggcatctacgccTAG |
| hCD3z-MCP | gccaccATGAAGTGGAAGGCGCTTTTCACCGCGGCCATCCTGCAGGCAC |
| | AGTTGCCGATTACAGAGGCACAGAGCTTTGGCCTGCTGGAcCCCAAAC |
| | TCTGCTACCTGCTGGATGGAATCCTCTTCATCTATGGTGTCATTCTCAC |
| | TGCCTTGTTCCTGAGAGTGAAGTTCAGCAGGAGCGCAGACGCCCCCGC |
| | GTACCAGCAGGGCCAGAACCAGCTCTATAACGAGCTCAATCTAGGAC |
| | GAAGAGAGGAGTACGATGTTTTGGACAAGAGACGTGGCCGGGACCCT |
| | GAGATGGGGGGAAAGCCGAGAAGGAAGAACCCTCAGGAAGGCCTGT |
| | AtAATGAACTGCAGAAAGATAAGATGGCGGAGGCCTACAGTGAGATT |
| | GGGATGAAAGGCGAGCGCCGGAGGGGCAAGGGGCACGATGGCCTTT |
| | ACCAGGGTCTCAGTACAGCCACCAAGGACACCTACGACGCCCTTCAC |
| | ATGCAGGCCCTGCCCCCTCGCggtagcggtggtGATTACAAGGACGACGATG |
| | ACAAGggtagcaacgcttctaactttactcagttcgttctcgtcgacaatggcggaactggcgacgtgactgtcg |
| | ccccaagcaacttcgctaacgggatcgctgaatggatcagctctaactcgcgttcacaggcttacaaagtaacctgta |
| | gcgttcgtcagagctctgcgcagaatcgcaaatacaccatcaaagtcgaggtgcctaaaggcgcctggcgttcgta |
| | cttaaatatggaactaaccattccaattttcgccacgaattccgactgcgagcttattgttaaggcaatgcaaggtctcct |
| | aaaagatggaaacccgattccctcagcaatcgcagcaaactccggcatctacgccatggccagcaacttcacccag |
| | ttcgtgctggtggacaacggcggcaccggcgacgtgaccgtggcccccagcaacttcgccaacggcatcgccga |
| | gtggatcagcagcaacagcagaagccaggcctacaaggtgacctgcagcgtgagacagagcagcgcccagaac |
| | agaaagtacaccatcaaggtggaggtgcccaagggcgcctggagaagctacctgaacatggagctgaccatccc |
| | catcttcgccaccaacagcgactgcgagctgatcgtgaaggccatgcagggcctgctgaaggacggcaaccccat |
| | ccccagcgccatcgccgccaacagcggcatctacgccTAG |
| EGFP-MS2 mRNA | gccaccatggtgagcaagggcgaggagctgttcaccggggtggtgcccatcctggtcgagctggacggcgacgt |
| | aaacggccacaagttcagcgtgtccggcgagggcgagggcgatgccacctacggcaagctgaccctgaagttcat |
| | ctgcaccaccggcaagctgcccgtgccctggcccaccctcgtgaccaccctgacctacggcgtgcagtgcttcagc |
| | cgctaccccgaccacatgaagcagcacgacttcttcaagtccgccatgcccgaaggctacgtccaggagcgcacc |
| | atcttcttcaaggacgacggcaactacaagacccgcgccgaggtgaagttcgagggcgacaccctggtgaaccgc |
| | atcgagctgaagggcatcgacttcaaggaggacggcaacatcctggggcacaagctggagtacaactacaacag |
| | ccacaacgtctatatcatggccgacaagcagaagaacggcatcaaggtgaacttcaagatccgccacaacatcgag |
| | gacggcagcgtgcagctcgccgaccactaccagcagaacacccccatcggcgacggccccgtgctgctgcccg |
| | acaaccactacctgagcacccagtccgccctgagcaaagaccccaacgagaagcgcgatcacatggtcctgctgg |
| | agttcgtgaccgccgccgggatcactctcggcatggacgagctgtataagtaaagcgccgcgtgactacggtactta |
| | ttgccaagaaaGCACGAGCATCAGCCGTGCctccaggtcgaatcttcaaaCGACGACGA |
| | TCACGCGTCGctccagtattccagggttcatc |
| EGFP-MS2 | atggtgagcaagggcgaggagctgttcaccggggtggtgcccatcctggtcgagctggacggcgacgtaaacgg |
| | ccacaagttcagcgtgtccggcgagggcgagggcgatgccacctacggcaagctgaccctgaagttcatctgcac |
| | caccggcaagctgcccgtgccctggcccaccctcgtgaccaccctgacctacggcgtgcagtgcttcagccgctac |
| | cccgaccacatgaagcagcacgacttcttcaagtccgccatgcccgaaggctacgtccaggagcgcaccatcttctt |
| | caaggacgacggcaactacaagacccgcgccgaggtgaagttcgagggcgacaccctggtgaaccgcatcgag |
| | ctgaagggcatcgacttcaaggaggacggcaacatcctggggcacaagctggagtacaactacaacagccacaa |
| | cgtctatatcatggccgacaagcagaagaacggcatcaaggtgaacttcaagatccgccacaacatcgaggacgg |
| | cagcgtgcagctcgccgaccactaccagcagaacacccccatcggcgacggccccgtgctgctgcccgacaacc |
| | actacctgagcacccagtccgccctgagcaaagaccccaacgagaagcgcgatcacatggtcctgctggagttcgt |
| | gaccgccgccgggatcactctcggcatggacgagctgtataagtaaagcgccgcgtgactacggtacttattgcca |
| | agaaaGCACGAGCATCAGCCGTGCctccaggtcgaatcttcaaaCGACGACGATCAC |
| | GCGTCGctccagtattccagggttcatc |
| CD19CAR-CD3z- | TCCGGAgccaccatggccttaccagtgaccgccttgctcctgccgctggccttgctgctccacgccgccaggc |
| mCherry | cggacatccagatgacacagactacatcctccctgtctgcctctctgggagacagagtcaccatcagttgcagggca |
| | agtcaggacattagtaaatatttaaattggtatcagcagaaaccagatggaactgttaaactcctgatctaccatacatc |
| | aagattacactcaggagtcccatcaaggttcagtggcagtgggtctggaacagattattctctcaccattagcaacctg |
| | gagcaagaagatattgccacttacttttgccaacagggtaatacgcttccgtacacgttcggaggggggaccaagct |
| | ggagatcacaggtggcggtggctcgggcggtggtgggtcgggtggcggcggatctgaggtgaaactgcaggagt |
| | caggacctggcctggtggcgccctcacagagcctgtccgtcacatgcactgtctcaggggtctcattacccgactat |
| | ggtgtaagctggattcgccagcctccacgaaagggtctggagtggctgggagtaatatggggtagtgaaaccacat |
| | actataattcagctctcaaatccagactgaccatcatcaaggacaactccaagagccaagttttcttaaaaatgaacag |
| | tctgcaaactgatgacacagccatttactactgtgccaaacattattactacggtggtagctatgctatggactactggg |
| | gccaaggaacctcagtcaccgtctcctcaaccacgacgccagcgccgcgaccaccaacaccggcgcccaccatc |
| | gcgtcgcagcccctgtccctgcgcccagaggcgtgccggccagcggcggggggcgcagtgcacacgaggggg |
| | ctggacttcgcctgtgatatctacatctgggcgcccttggccgggacttgtggggtccttctcctgtcactggttatcac |
| | cctttactgcaaccacaggaaccgaagacgtCGGGTGAAGTTCAGCAGAAGCGCCGACG |
| | CCCCTGCCTACCAGCAGGGCCAGAATCAGCTGTACAACGAGCTGAAC |
| | CTGGGCAGAAGGGAAGAGTACGACGTCCTGGATAAGCGGAGAGGCC |
| | GGGACCCTGAGATGGGCGGCAAGCCTCGGCGGAAGAACCCCCAGGAA |
| | GGCCTGTATAACGAACTGCAGAAAGACAAGATGGCCGAGGCCTACAG |
| | CGAGATCGGCATGAAGGGCGAGCGGAGGCGGGGCAAGGGCCACGAC |
| | GGCCTGTATCAGGGCCTGTCCACCGCCACCAAGGATACCTACGACGCC |
| | CTGCACATGCAGGCCCTGCCCCCAAGGccaccggtcgccaccATGGTGAGCAA |
| | GGGCGAGGAGGATAACATGGCCATCATCAAGGAGTTCATGCGCTTCA |
| | AGGTGCACATGGAGGGCTCCGTGAACGGCCACGAGTTCGAGATCGAG |
| | GGCGAGGGCGAGGGCCGCCCCTACGAGGGCACCCAGACCGCCAAGCT |
| | GAAGGTGACCAAGGGTGGCCCCCTGCCCTTCGCCTGGGACATCCTGTC |
| | CCCTCAGTTCATGTACGGCTCCAAGGCCTACGTGAAGCACCCCGCCGA |
| | CATCCCCGACTACTTGAAGCTGTCCTTCCCCGAGGGCTTCAAGTGGGA |
| | GCGCGTGATGAACTTCGAGGACGGCGGCGTGGTGACCGTGACCCAGG |
| | ACTCCTCCCTGCAGGACGGCGAGTTCATCTACAAGGTGAAGCTGCGCG |
| | GCACCAACTTCCCCTCCGACGGCCCCGTAATGCAGAAGAAGACCATG |
| | GGCTGGGAGGCCTCCTCCGAGCGGATGTACCCCGAGGACGGCGCCCT |
| | GAAGGGCGAGATCAAGCAGAGGCTGAAGCTGAAGGACGGCGGCCAC |
| | TACGACGCTGAGGTCAAGACCACCTACAAGGCCAAGAAGCCCGTGCA |
| | GCTGCCCGGCGCCTACAACGTCAACATCAAGTTGGACATCACCTCCCA |
| | CAACGAGGACTACACCATCGTGGAACAGTACGAACGCGCCGAGGGCC |
| | GCCACTCCACCGGCGGCATGGACGAGCTGTACAAGTAGGGATCC |
| EGFP-ZAP70_ | gccaccatggtgagcaagggcgaggagctgttcaccggggtggtgcccatcctggtcgagctggacggcgacgt |
| SH2 | aaacggccacaagttcagcgtgtccggcgagggcgagggcgatgccacctacggcaagctgaccctgaagttcat |
| | ctgcaccaccggcaagctgcccgtgccctggcccaccctcgtgaccaccctgacctacggcgtgcagtgcttcagc |
| | cgctaccccgaccacatgaagcagcacgacttcttcaagtccgccatgcccgaaggctacgtccaggagcgcacc |
| | atcttcttcaaggacgacggcaactacaagacccgcgccgaggtgaagttcgagggcgacaccctggtgaaccgc |
| | atcgagctgaagggcatcgacttcaaggaggacggcaacatcctggggcacaagctggagtacaactacaacag |
| | ccacaacgtctatatcatggccgacaagcagaagaacggcatcaaggtgaacttcaagatccgccacaacatcgag |
| | gacggcagcgtgcagctcgccgaccactaccagcagaacacccccatcggcgacggccccgtgctgctgcccg |
| | acaaccactacctgagcacccagtccgccctgagcaaagaccccaacgagaagcgcgatcacatggtcctgctgg |
| | agttcgtgaccgccgccgggatcactctcggcatggacgagctgtataagggaccaggatcggcgtctggggaag |
| | gactgccaggatcagccggaccaggaATGCCCGATCCCGCGGCGCAtCTGCCATTCT |
| | TCTATGGCAGCATCTCGCGGGCTGAGGCCGAGGAGCACCTGAAGCTG |
| | GCAGGCATGGCCGACGGGCTGTTCCTCCTGCGCCAGTGTTTGCGCTCC |
| | CTGGGCGGCTACGTGCTGTCGTTGGTGCACGACGTGCGCTTCCACCAT |
| | TTCCCCATCGAGCGCCAACTCAACGGCACGTACGCCATCGCGGGCGG |
| | GAAGGCGCACTGCGGCCCGGCCGAGCTCTGCCAGTTCTACTCTCAGGA |
| | CCCCGATGGGCTGCCCTGCAACCTGCGTAAGCCGTGTAACCGGCCGCC |
| | CGGACTGGAGCCACAGCCCGGGGTCTTCGACTGCCTGCGTGATGCTAT |
| | GGTGTGCGACTACGTGCGCCAGACCTGGAAGCTGGAGGGCGATGCAC |
| | TAGAGCAGGCCATCATCAGCCAGGCCCCACAGGTGGAGAAGCTCATT |
| | GCTACCACAGCCCACGAGCGAATGCCCTGGTATCACAGCAGCCTGAC |
| | TCGTGAGGAGGCCGAGCGCAAACTCTATTCCGGCCAGCAGACCGACG |
| | GCAAGTTCCTGCTGAGGCCCCGGAAGGAGCAGGGCACATATGCACTG |
| | TCCCTGGTCTATGGGAAAACTGTATACCACTATCTCATCAGCCAGGAC |
| | AAGGCTGGCAAGTACTGCATCCCCGAAGGCACCAAGTTTGACACGCT |
| | CTGGCAGCTGGTGGAGTACCTGAAGCTGAAGGCGGATGGACTAATTT |
| | ACCGCTTGAAGGAGGTCTGTCCCAACAGCAGCGCCccagggcaccatcatcatca |
| | tcattaa |
| CD19CAR-CD3z- | TCCGGAgccaccatggccttaccagtgaccgccttgctcctgccgctggccttgctgctccacgccgccaggc |
| mCherry | cggacatccagatgacacagactacatcctccctgtctgcctctctgggagacagagtcaccatcagttgcagggca |
| | agtcaggacattagtaaatatttaaattggtatcagcagaaaccagatggaactgttaaactcctgatctaccatacatc |
| | aagattacactcaggagtcccatcaaggttcagtggcagtgggtctggaacagattattctctcaccattagcaacctg |
| | gagcaagaagatattgccacttacttttgccaacagggtaatacgcttccgtacacgttcggaggggggaccaagct |
| | ggagatcacaggtggcggtggctcgggcggtggtgggtcgggtggcggcggatctgaggtgaaactgcaggagt |
| | caggacctggcctggtggcgccctcacagagcctgtccgtcacatgcactgtctcaggggtctcattacccgactat |
| | ggtgtaagctggattcgccagcctccacgaaagggtctggagtggctgggagtaatatggggtagtgaaaccacat |
| | actataattcagctctcaaatccagactgaccatcatcaaggacaactccaagagccaagttttcttaaaaatgaacag |
| | tctgcaaactgatgacacagccatttactactgtgccaaacattattactacggtggtagctatgctatggactactggg |
| | gccaaggaacctcagtcaccgtctcctcaaccacgacgccagcgccgcgaccaccaacaccggcgcccaccatc |
| | gcgtcgcagcccctgtccctgcgcccagaggcgtgccggccagcggggggggcgcagtgcacacgaggggg |
| | ctggacttcgcctgtgatatctacatctgggcgcccttggccgggacttgtggggtccttctcctgtcactggttatcac |
| | cctttactgcaaccacaggaaccgaagacgtCGGGTGAAGTTCAGCAGAAGCGCCGACG |
| | CCCCTGCCTACCAGCAGGGCCAGAATCAGCTGTACAACGAGCTGAAC |
| | CTGGGCAGAAGGGAAGAGTACGACGTCCTGGATAAGCGGAGAGGCC |
| | GGGACCCTGAGATGGGCGGCAAGCCTCGGCGGAAGAACCCCCAGGAA |
| | GGCCTGTATAACGAACTGCAGAAAGACAAGATGGCCGAGGCCTACAG |
| | CGAGATCGGCATGAAGGGCGAGCGGAGGCGGGGCAAGGGCCACGAC |
| | GGCCTGTATCAGGGCCTGTCCACCGCCACCAAGGATACCTACGACGCC |
| | CTGCACATGCAGGCCCTGCCCCCAAGGccaccggtcgccaccATGGTGAGCAA |
| | GGGCGAGGAGGATAACATGGCCATCATCAAGGAGTTCATGCGCTTCA |
| | AGGTGCACATGGAGGGCTCCGTGAACGGCCACGAGTTCGAGATCGAG |
| | GGCGAGGGCGAGGGCCGCCCCTACGAGGGCACCCAGACCGCCAAGCT |
| | GAAGGTGACCAAGGGTGGCCCCCTGCCCTTCGCCTGGGACATCCTGTC |
| | CCCTCAGTTCATGTACGGCTCCAAGGCCTACGTGAAGCACCCCGCCGA |
| | CATCCCCGACTACTTGAAGCTGTCCTTCCCCGAGGGCTTCAAGTGGGA |
| | GCGCGTGATGAACTTCGAGGACGGCGGCGTGGTGACCGTGACCCAGG |
| | ACTCCTCCCTGCAGGACGGCGAGTTCATCTACAAGGTGAAGCTGCGCG |
| | GCACCAACTTCCCCTCCGACGGCCCCGTAATGCAGAAGAAGACCATG |
| | GGCTGGGAGGCCTCCTCCGAGCGGATGTACCCCGAGGACGGCGCCCT |
| | GAAGGGCGAGATCAAGCAGAGGCTGAAGCTGAAGGACGGCGGCCAC |
| | TACGACGCTGAGGTCAAGACCACCTACAAGGCCAAGAAGCCCGTGCA |
| | GCTGCCCGGCGCCTACAACGTCAACATCAAGTTGGACATCACCTCCCA |
| | CAACGAGGACTACACCATCGTGGAACAGTACGAACGCGCCGAGGGCC |
| | GCCACTCCACCGGCGGCATGGACGAGCTGTACAAGTAGGGATCC |
| EGFP-ZAP70_ | gccaccatggtgagcaagggcgaggagctgttcaccggggtggtgcccatcctggtcgagctggacggcgacgt |
| SH2 | aaacggccacaagttcagcgtgtccggcgagggcgagggcgatgccacctacggcaagctgaccctgaagttcat |
| | ctgcaccaccggcaagctgcccgtgccctggcccaccctcgtgaccaccctgacctacggcgtgcagtgcttcagc |
| | cgctaccccgaccacatgaagcagcacgacttcttcaagtccgccatgcccgaaggctacgtccaggagcgcacc |
| | atcttcttcaaggacgacggcaactacaagacccgcgccgaggtgaagttcgagggcgacaccctggtgaaccgc |
| | atcgagctgaagggcatcgacttcaaggaggacggcaacatcctggggcacaagctggagtacaactacaacag |
| | ccacaacgtctatatcatggccgacaagcagaagaacggcatcaaggtgaacttcaagatccgccacaacatcgag |
| | gacggcagcgtgcagctcgccgaccactaccagcagaacacccccatcggcgacggccccgtgctgctgcccg |
| | acaaccactacctgagcacccagtccgccctgagcaaagaccccaacgagaagcgcgatcacatggtcctgctgg |
| | agttcgtgaccgccgccgggatcactctcggcatggacgagctgtataagggaccaggatcggcgtctggggaag |
| | gactgccaggatcagccggaccaggaATGCCCGATCCCGCGGCGCAtCTGCCATTCT |
| | TCTATGGCAGCATCTCGCGGGCTGAGGCCGAGGAGCACCTGAAGCTG |
| | GCAGGCATGGCCGACGGGCTGTTCCTCCTGCGCCAGTGTTTGCGCTCC |
| | CTGGGCGGCTACGTGCTGTCGTTGGTGCACGACGTGCGCTTCCACCAT |
| | TTCCCCATCGAGCGCCAACTCAACGGCACGTACGCCATCGCGGGCGG |
| | GAAGGCGCACTGCGGCCCGGCCGAGCTCTGCCAGTTCTACTCTCAGGA |
| | CCCCGATGGGCTGCCCTGCAACCTGCGTAAGCCGTGTAACCGGCCGCC |
| | CGGACTGGAGCCACAGCCCGGGGTCTTCGACTGCCTGCGTGATGCTAT |
| | GGTGTGCGACTACGTGCGCCAGACCTGGAAGCTGGAGGGCGATGCAC |
| | TAGAGCAGGCCATCATCAGCCAGGCCCCACAGGTGGAGAAGCTCATT |
| | GCTACCACAGCCCACGAGCGAATGCCCTGGTATCACAGCAGCCTGAC |
| | TCGTGAGGAGGCCGAGCGCAAACTCTATTCCGGCCAGCAGACCGACG |
| | GCAAGTTCCTGCTGAGGCCCCGGAAGGAGCAGGGCACATATGCACTG |
| | TCCCTGGTCTATGGGAAAACTGTATACCACTATCTCATCAGCCAGGAC |
| | AAGGCTGGCAAGTACTGCATCCCCGAAGGCACCAAGTTTGACACGCT |
| | CTGGCAGCTGGTGGAGTACCTGAAGCTGAAGGCGGATGGACTAATTT |
| | ACCGCTTGAAGGAGGTCTGTCCCAACAGCAGCGCCccagggcaccatcatcatca |
| | tcattaa |
| EGFP-MS2 | Atggtgagcaagggcgaggagctgttcaccggggtggtgcccatcctggtcgagctggacggcgacgtaaacg |
| | gccacaagttcagcgtgtccggcgagggcgagggcgatgccacctacggcaagctgaccctgaagttcatctgca |
| | ccaccggcaagctgcccgtgccctggcccaccctcgtgaccaccctgacctacggcgtgcagtgcttcagccgcta |
| | ccccgaccacatgaagcagcacgacttcttcaagtccgccatgcccgaaggctacgtccaggagcgcaccatcttc |
| | ttcaaggacgacggcaactacaagacccgcgccgaggtgaagttcgagggcgacaccctggtgaaccgcatcga |
| | gctgaagggcatcgacttcaaggaggacggcaacatcctggggcacaagctggagtacaactacaacagccaca |
| | acgtctatatcatggccgacaagcagaagaacggcatcaaggtgaacttcaagatccgccacaacatcgaggacg |
| | gcagcgtgcagctcgccgaccactaccagcagaacacccccatcggcgacggccccgtgctgctgcccgacaac |
| | cactacctgagcacccagtccgccctgagcaaagaccccaacgagaagcgcgatcacatggtcctgctggagttc |
| | gtgaccgccgccgggatcactctcggcatggacgagctgtataagtaaagcgccgcgtgactacggtacttattgcc |
| | aagaaaGCACGAGCATCAGCCGTGCctccaggtcgaatcttcaaaCGACGACGATCA |
| | CGCGTCGctccagtattccagggttcatc |
| OT1 TCR | atgtctaacactgtcctcgctgattctgcctggggcatcaccctgctatcttgggttactgtctttctcttgggaacaag |
| | ttcagcagattctggggttgtccagtctccaagacacataatcaaagaaaagggaggaaggtccgttctgacgtgtattc |
| | ccatctctggacatagcaatgtggtctggtaccagcagactctggggaaggaattaaagttccttattcagcattatga |
| | aaaggtggagagagacaaaggattcctacccagcagattctcagtccaacagtttgatgactatcactctgaaatgaa |
| | catgagtgccttggaactggaggactctgctatgtacttctgtgccagctctcgggccaattatgaacagtacttcggt |
| | cccggcaccaggctcacggttttagaggatctgagaaatgtgactccacccaaggtctccttgtttgagccatcaaaa |
| | gcagagattgcaaacaaacaaaaggctaccctcgtgtgcttggccaggggcttcttccctgaccacgtggagctga |
| | gctggtgggtgaatggcaaggaggtccacagtggggtcagcacggaccctcaggcctacaaggagagcaattata |
| | gctactgcctgagcagccgcctgagggtctctgctaccttctggcacaatcctcgaaaccacttccgctgccaagtgc |
| | agttccatgggctttcagaggaggacaagtggccagagggctcacccaaacctgtcacacagaacatcagtgcag |
| | aggcctggggccgagcagactgtggaatcacttcagcatcctatcatcagggggttctgtctgcaaccatcctctatg |
| | agatcctactggggaaggccaccctatatgctgtgctggtcagtggcctggtgctgatggccatggtcaagaaaaaa |
| | aattccgccacgaacttctctctgttaaagcaagcaggagacgtggaagaaaaccccggtcctatggacaagatcct |
| | gacagcatcgtttttactcctaggccttcacctagctggggtgaatggccagcagcaggagaaacgtgaccagcag |
| | caggtgagacaaagtccccaatctctgacagtctgggaaggagagaccgcaattctgaactgcagttatgaggaca |
| | gcacttttaactacttcccatggtaccagcagttccctggggaaggccctgcactcctgatatccatacgttcagtgtcc |
| | gataaaaaggaagatggacgattcacaatcttcttcaataaaagggagaaaaagctctccttgcacatcacagactct |
| | cagcctggagactcagctacctacttctgtgcagcaagtgacaactatcagttgatctggggctctgggaccaagcta |
| | attataaagccagacatccagaacccagaacctgctgtgtaccagttaaaagatcctcggtctcaggacagcaccct |
| | ctgcctgttcaccgactttgactcccaaatcaatgtgccgaaaaccatggaatctggaacgttcatcactgacaaaact |
| | gtgctggacatgaaagctatggattccaagagcaatggggccattgcctggagcaaccagacaagcttcacctgcc |
| | aagatatcttcaaagagaccaacgccacctaccccagttcagacgttccctgtgatgccacgttgactgagaaaagct |
| | ttgaaacagatatgaacctaaactttcaaaacctgtcagttatgggactccgaatcctcctgctgaaagtagccggattt |
| | aacctgctcatgacgctgaggctgtggtcctcctaa |
| mCD3z-EGFP | gccaccatgaagtggaaagtgtctgttctcgcctgcatcctccacgtgcggttcccaggagcagaggcacagagctt |
| | tggtctgctggaccccaaactctgctacttgctagatggaatcctcttcatctacggagtcatcatcacagccctgtacc |
| | tgagagcaaaattcagcaggagtgcagagactgctgccaacctgcaggaccccaaccagctctacaatgagctca |
| | atctagggcgaagagaggaatatgacgtcttggagaagaagcgggctcgggacccagagatgggaggcaaaca |
| | gcagaggaggaggaacccccaggaaggcgtatacaatgcactgcagaaagacaagatggcagaagcctacagt |
| | gagatcggcacaaaaggcgagaggcggagaggcaaggggcacgatggcctttaccagggtctcagcactgcca |
| | ccaaggacacctatgatgccctgcatatgcagaccctggcccctcgcccacccgtcgccaccatggtgagcaagg |
| | gcgaggagctgttcaccggggtggtgcccatcctggtcgagctggacggcgacgtaaacggccacaagttcagc |
| | gtgtccggcgagggcgagggcgatgccacctacggcaagctgaccctgaagttcatctgcaccaccggcaagct |
| | gcccgtgccctggcccaccctcgtgaccaccctgacctacggcgtgcagtgcttcagccgctaccccgaccacatg |
| | aagcagcacgacttcttcaagtccgccatgcccgaaggctacgtccaggagcgcaccatcttcttcaaggacgacg |
| | gcaactacaagacccgcgccgaggtgaagttcgagggcgacaccctggtgaaccgcatcgagctgaagggcatc |
| | gacttcaaggaggacggcaacatcctggggcacaagctggagtacaactacaacagccacaacgtctatatcatgg |
| | ccgacaagcagaagaacggcatcaaggtgaacttcaagatccgccacaacatcgaggacggcagcgtgcagctc |
| | gccgaccactaccagcagaacacccccatcggcgacggccccgtgctgctgcccgacaaccactacctgagcac |
| | ccagtccgccctgagcaaagaccccaacgagaagcgcgatcacatggtcctgctggagttcgtgaccgccgccg |
| | ggatcactctcggcatggacgagctgtataagtga |
| EGFP- | gccaccatggtgagcaagggcgaggagctgttcaccggggtggtgcccatcctggtcgagctggacggcgacgt |
| mZAP70_SH2 | aaacggccacaagttcagcgtgtccggcgagggcgagggcgatgccacctacggcaagctgaccctgaagttcat |
| | ctgcaccaccggcaagctgcccgtgccctggcccaccctcgtgaccaccctgacctacggcgtgcagtgcttcagc |
| | cgctaccccgaccacatgaagcagcacgacttcttcaagtccgccatgcccgaaggctacgtccaggagcgcacc |
| | atcttcttcaaggacgacggcaactacaagacccgcgccgaggtgaagttcgagggcgacaccctggtgaaccgc |
| | atcgagctgaagggcatcgacttcaaggaggacggcaacatcctggggcacaagctggagtacaactacaacag |
| | ccacaacgtctatatcatggccgacaagcagaagaacggcatcaaggtgaacttcaagatccgccacaacatcgag |
| | gacggcagcgtgcagctcgccgaccactaccagcagaacacccccatcggcgacggccccgtgctgctgcccg |
| | acaaccactacctgagcacccagtccgccctgagcaaagaccccaacgagaagcgcgatcacatggtcctgctgg |
| | agttcgtgaccgccgccgggatcactctcggcatggacgagctgtataagggaccaggatcggcgtctggggaag |
| | gactgccaggatcagccggaccaggaATGCCCGATCCCGCGGCGCAtCTGCCATTCT |
| | TCTATGGCAGCATCTCGCGGGCTGAGGCCGAGGAGCACCTGAAGCTG |
| | GCAGGCATGGCCGACGGGCTGTTCCTCCTGCGCCAGTGTTTGCGCTCC |
| | CTGGGCGGCTACGTGCTGTCGTTGGTGCACGACGTGCGCTTCCACCAT |
| | TTCCCCATCGAGCGCCAACTCAACGGCACGTACGCCATCGCGGGCGG |
| | GAAGGCGCACTGCGGCCCGGCCGAGCTCTGCCAGTTCTACTCTCAGGA |
| | CCCCGATGGGCTGCCCTGCAACCTGCGTAAGCCGTGTAACCGGCCGCC |
| | CGGACTGGAGCCACAGCCCGGGGTCTTCGACTGCCTGCGTGATGCTAT |
| | GGTGTGCGACTACGTGCGCCAGACCTGGAAGCTGGAGGGCGATGCAC |
| | TAGAGCAGGCCATCATCAGCCAGGCCCCACAGGTGGAGAAGCTCATT |
| | GCTACCACAGCCCACGAGCGAATGCCCTGGTATCACAGCAGCCTGAC |
| | TCGTGAGGAGGCCGAGCGCAAACTCTATTCCGGCCAGCAGACCGACG |
| | GCAAGTTCCTGCTGAGGCCCCGGAAGGAGCAGGGCACATATGCACTG |
| | TCCCTGGTCTATGGGAAAACTGTATACCACTATCTCATCAGCCAGGAC |
| | AAGGCTGGCAAGTACTGCATCCCCGAAGGCACCAAGTTTGACACGCT |
| | CTGGCAGCTGGTGGAGTACCTGAAGCTGAAGGCGGATGGACTAATTT |
| | ACCGCTTGAAGGAGGTCTGTCCCAACAGCAGCGCCccagggcaccatcatcatca |
| | tcattaa |
| Cre-mZAP70_ | gccaccatggctaatttactgaccgtacaccaaaatttgcctgcattacccgtcgatgcaacgagtgatgaggttcgc |
| SH2 | aagaacctgatggacatgttcagggatcgccaggcgttttctgagcatacctggaaaatgcttctgtccgtttgccggt |
| | cgtgggcggcatggtgcaagttgaataaccggaaatggtttcccgcagaacctgaagatgttcgcgattatcttctata |
| | tcttcaggcgcgcggtctggcagtaaaaactatccagcaacatttgggccagctaaacatgcttcatcgtcggtccgg |
| | gctgccacgaccaagtgacagcaatgctgtttcactggttatgcggcgaatccgaaaagaaaacgttgatgccggtg |
| | aacgtgcaaaacaggctctagcgttcgaacgcactgatttcgaccaggttcgttcactcatggaaaatagcgatcgct |
| | gccaggatatacgtaatctggcatttctggggattgcttataacaccctgttacgtatagccgaaattgccaggatcag |
| | ggttaaagacatctcacgtactgacggtgggagaatgttaatccatattggcagaacgaaaacgctggttagcaccg |
| | caggtgtggagaaggcacttagcctgggggtaactaaactggtcgagcgatggatttccgtctctggtgtagctgat |
| | gatccgaataactacctgttttgccgggtcagaaaaaatggtgttgccgcgccatctgccaccagccagctatcaact |
| | cgcgccctggaagggatttttgaagcaactcatcgattgatttacggcgctaaggatgactctggtcagagatacctg |
| | gcctggtctggacacagtgcccgtgtcggagccgcgcgagatatggcccgcgctggagtttcaataccggagatc |
| | atgcaagctggtggctggaccaatgtaaatattgtcatgaactatatccgtaacctggatagtgaaacaggggcaatg |
| | gtgcgcctgctggaagatggcgatggacccgtcggtagcgaaaacctgtactttcagtccggtggtggtagcATG |
| | CCCGATCCCGCGGCGCACCTGCCATTCTTCTATGGCAGCATCTCGCGG |
| | GCTGAGGCCGAGGAGCACCTGAAGCTGGCAGGCATGGCCGACGGGCT |
| | GTTCCTCCTGCGCCAGTGTTTGCGCTCCCTGGGCGGCTACGTGCTGTC |
| | GTTGGTGCACGACGTGCGCTTCCACCATTTCCCCATCGAGCGCCAACT |
| | CAACGGCACGTACGCCATCGCGGGCGGGAAGGCGCACTGCGGCCCGG |
| | CCGAGCTCTGCCAGTTCTACTCTCAGGACCCCGATGGGCTGCCCTGCA |
| | ACCTGCGTAAGCCGTGTAACCGGCCGCCCGGACTGGAGCCACAGCCC |
| | GGGGTCTTCGACTGCCTGCGTGATGCTATGGTGTGCGACTACGTGCGC |
| | CAGACCTGGAAGCTGGAGGGCGATGCACTAGAGCAGGCCATCATCAG |
| | CCAGGCCCCACAGGTGGAGAAGCTCATTGCTACCACAGCCCACGAGC |
| | GAATGCCCTGGTATCACAGCAGCCTGACTCGTGAGGAGGCCGAGCGC |
| | AAACTCTATTCCGGCCAGCAGACCGACGGCAAGTTCCTGCTGAGGCCC |
| | CGGAAGGAGCAGGGCACATATGCACTGTCCCTGGTCTATGGGAAAAC |
| | TGTATACCACTATCTCATCAGCCAGGACAAGGCTGGCAAGTACTGCAT |
| | CCCCGAAGGCACCAAGTTTGACACGCTCTGGCAGCTGGTGGAGTACCT |
| | GAAGCTGAAGGCGGATGGACTAATTTACCGCTTGAAGGAGGTCTGTC |
| | CCAACAGCAGCGCCccagggcaccatcatcatcatcattaa |
| MCP-mZAP70_ | gccaccatgggcaacgcttctaactttactcagttcgttctcgtcgacaatggcggaactggcgacgtgactgtcgcc |
| SH2 | ccaagcaacttcgctaacgggatcgctgaatggatcagctctaactcgcgttcacaggcttacaaagtaacctgtagc |
| | gttcgtcagagctctgcgcagaatcgcaaatacaccatcaaagtcgaggtgcctaaaggcgcctggcgttcgtactt |
| | aaatatggaactaaccattccaattttcgccacgaattccgactgcgagcttattgttaaggcaatgcaaggtctcctaa |
| | aagatggaaacccgattccctcagcaatcgcagcaaactccggcatctacgccatggccagcaacttcacccagttc |
| | gtgctggtggacaacggcggcaccggcgacgtgaccgtggcccccagcaacttcgccaacggcatcgccgagtg |
| | gatcagcagcaacagcagaagccaggcctacaaggtgacctgcagcgtgagacagagcagcgcccagaacaga |
| | aagtacaccatcaaggtggaggtgcccaagggcgcctggagaagctacctgaacatggagctgaccatccccatc |
| | ttcgccaccaacagcgactgcgagctgatcgtgaaggccatgcagggcctgctgaaggacggcaaccccatcccc |
| | agcgccatcgccgccaacagcggcatctacgccggaccaggatcggcgtctggggaaggactgccaggatcag |
| | ccggaccaggaATGCCCGATCCCGCGGCGCACCTGCCATTCTTCTATGGCAG |
| | CATCTCGCGGGCTGAGGCCGAGGAGCACCTGAAGCTGGCAGGCATGG |
| | CCGACGGGCTGTTCCTCCTGCGCCAGTGTTTGCGCTCCCTGGGCGGCT |
| | ACGTGCTGTCGTTGGTGCACGACGTGCGCTTCCACCATTTCCCCATCG |
| | AGCGCCAACTCAACGGCACGTACGCCATCGCGGGCGGGAAGGCGCAC |
| | TGCGGCCCGGCCGAGCTCTGCCAGTTCTACTCTCAGGACCCCGATGGG |
| | CTGCCCTGCAACCTGCGTAAGCCGTGTAACCGGCCGCCCGGACTGGA |
| | GCCACAGCCCGGGGTCTTCGACTGCCTGCGTGATGCTATGGTGTGCGA |
| | CTACGTGCGCCAGACCTGGAAGCTGGAGGGCGATGCACTAGAGCAGG |
| | CCATCATCAGCCAGGCCCCACAGGTGGAGAAGCTCATTGCTACCACA |
| | GCCCACGAGCGAATGCCCTGGTATCACAGCAGCCTGACTCGTGAGGA |
| | GGCCGAGCGCAAACTCTATTCCGGCCAGCAGACCGACGGCAAGTTCC |
| | TGCTGAGGCCCCGGAAGGAGCAGGGCACATATGCACTGTCCCTGGTC |
| | TATGGGAAAACTGTATACCACTATCTCATCAGCCAGGACAAGGCTGG |
| | CAAGTACTGCATCCCCGAAGGCACCAAGTTTGACACGCTCTGGCAGCT |
| | GGTGGAGTACCTGAAGCTGAAGGCGGATGGACTAATTTACCGCTTGA |
| | AGGAGGTCTGTCCCAACAGCAGCGCCccagggcaccatcatcatcatcattaa |

### Pharmaceutical Compositions

In one aspect, the present disclosure is directed in part to a pharmaceutical composition comprising a donor cell described herein. In some embodiments, a pharmaceutical composition comprises a plurality of donor cells. In some embodiments, the donor cell comprises a membrane-associated agent (e.g., comprising one, two, or all three of an intracellular moiety, a membrane-associated moiety, and/or an extracellular moiety) as described herein. In some embodiments, the donor cell comprises a cargo molecule as described herein. In some embodiments, the membrane-associated agent and the cargo molecule are operably associated or linked. In some embodiments, a pharmaceutical composition described herein comprises one or more pharmaceutically acceptable excipients. In some embodiments, a pharmaceutical composition comprises an acceptor cell or a plurality of acceptor cells. In some embodiments, a pharmaceutical composition comprises a donor cell (e.g., a plurality of donor cells) and an acceptor cell (e.g., a plurality of acceptor cells).

In embodiments, a pharmaceutical composition described herein has one or more of the following characteristics:
the pharmaceutical composition meets a pharmaceutical or good manufacturing practices (GMP) standard;
the pharmaceutical composition was made according to good manufacturing practices (GMP);
the pharmaceutical composition has a pathogen level below a predetermined reference value, e.g., is substantially free of pathogens;
the pharmaceutical composition has a contaminant level (e.g., nuclear DNA) below a predetermined reference value, e.g., is substantially free of contaminants; or
the pharmaceutical composition has low immunogenicity, e.g., as described herein.

### Membrane Transfer Processes

In some embodiments, a cargo molecule and/or a membrane-associated agent is transferred from a donor cell to an acceptor cell by a membrane transfer process. In some embodiments, a membrane transfer process comprises contact between a donor cell (comprising a membrane-associated agent and optionally a cargo molecule) and an acceptor cell. In some embodiments, a membrane transfer process comprises close proximity between a donor cell (comprising a membrane-associated agent and optionally a cargo molecule) and an acceptor cell. In some embodiments, close proximity comprises a distance of no more than 1, 2, 3, 4, 5, 6, 7, 8, 9, 10, 15, 20, 25, 30, 35, 40, 45, or 50 nm, or no more than 1, 2, 3, 4, 5, 6, 7, 8, 9, or 10 μm. In some embodiments, close proximity comprises a distance of no more than 0.1× (i.e., 0.1 times), 0.2×, 0.3×, 0.4×, 0.5×, 0.6×, 0.7×, 0.8×, 0.9×, 1×, 1.1×, 1.2×, 1.3×, 1.4×, 1.5×, 1.75×, 2×, 2.25×, 2.5×, 2.75×, 3×, 3.25×, 3.5×, 3.75×, or 4× the width (e.g., the average diameter) of the donor cell or acceptor cell. In some embodiments, a cargo molecule and/or a membrane-associated agent is transferred from a donor cell to an acceptor cell by a membrane transfer process, e.g., wherein the level of agent delivered via a membrane transfer process is 0.01-0.6, 0.01-0.1, 0.1-0.3, or 0.3-0.6, or at least 1%, 2%, 3%, 4%, 5%, 10%, 20%, 30%, 40%, 50%, 60%, 70%, 80%, 90% or greater than a reference acceptor cell contacted with a similar donor cell not configured to transfer the cargo molecule and/or a membrane-associated agent. In some embodiments, at least 1%, 2%, 3%, 4%, 5%, 10%, 20%, 30%, 40%, 50%, 60%, 70%, 80%, 90% of cargo molecules and/or exogenous membrane-associated agents in a donor cell composition or preparation that enter an acceptor cell enter via a membrane transfer process. In some embodiments, the membrane transfer process comprises a membrane fusion event, a receptor-ligand interaction, a cell bridging event (e.g., an antibody molecule (e.g., a bispecific antibody), a nanotube transfer, or cell to cell contact event. In some embodiments, a membrane transfer process comprises transferring cell membrane, or components thereof, from the donor cell to the acceptor cell. In some embodiments, a membrane transfer process comprises transferring one or more intracellular constituents from the donor cell to the acceptor cell (e.g., cytoplasm, intracellular polypeptides, intracellular nucleic acid molecules, intracellular small molecules, or other intracellular compounds, solutions, or organelles).

### Methods of Use

This disclosure provides, in certain aspects, methods of transferring a cargo molecule and/or exogenous membrane-associated agent from a donor cell to an acceptor cell.

In an aspect, the disclosure provides a method of modifying a cell, comprising contacting the acceptor cell with a donor cell or system described herein, under conditions suitable for transfer of the membrane-associated agent to the acceptor cell, wherein the acceptor cell does not comprise a nucleic acid encoding the membrane-associated agent. In some embodiments, modifying the acceptor cell comprises transferring the membrane-associated agent from the donor cell (e.g., the first donor cell, the second donor cell, or both) to the acceptor cell.

In an aspect, the disclosure provides a method of making a modified cell, comprising: providing an unmodified cell, contacting the unmodified cell with a donor cell, or system described herein, under conditions suitable for transfer of the membrane-associated agent to the unmodified cell, wherein neither the unmodified cell or modified cell comprise a nucleic acid encoding the membrane-associated agent. In some embodiments, the modified cell comprises membrane-associated agent, and wherein the membrane-associated agent was not produced in the acceptor cell. In some embodiments, the method further comprises providing a donor cell (e.g., a first donor cell, second donor cell, both, a third donor cell, or all three) described herein. In some embodiments, the providing comprises contacting a cell with a nucleic acid encoding the membrane-associated agent, thereby providing a donor cell comprising a nucleic acid encoding the membrane-associated agent.

In an aspect, the disclosure provides a method of delivering a cargo molecule to a cell, comprising: providing a donor cell, or system described herein, wherein the donor cell comprises a membrane-associated agent comprising the cargo molecule; providing an acceptor cell that does not comprise a nucleic acid encoding the membrane-associated agent; and contacting the acceptor cell with the donor cell, or system under conditions suitable for transfer of the membrane-associated agent to the acceptor cell. In some embodiments, the method further comprises contacting the donor cell and the acceptor cell with a multispecific molecule, e.g., antibody molecule ((e.g., Fab, F(ab′)2, Fab′, scFv, or di-scFv), e.g., a bispecific antibody molecule), that specifically binds to the donor cell (e.g., specifically binds to the membrane-associated agent) and specifically binds to the acceptor cell, under conditions sufficient to allow the acceptor cell to acquire the membrane-associated agent or cargo molecule.

In some embodiments, acceptor cell acquires a sufficient quantity of the membrane-associated agent and/or cargo molecule to provide a desired function (e.g., relative to a reference cell not receiving the membrane-associated agent and/or cargo molecule). A person of skill in the art will recognize that a sufficient quantity will depend upon the membrane-associated agent and/or cargo molecule in question and the function to be achieved. In some embodiments, a sufficient quantity is a single copy of the membrane-associated agent and/or cargo molecule (e.g., wherein the cargo molecule comprises a virus or nucleic acid that integrates into an acceptor cell genome). In some embodiments, a sufficient quantity comprises at least 1, 2, 3, 4, 5, 6, 7, 8, 9, 10, 20, 30, 40, 50, 60, 70, 80, 90, 95, 96, 97, 98, or 99% resurfacing of the acceptor cell with donor cell derived membrane (e.g., comprising membrane-associated agent and/or cargo molecule).

In some embodiments of any of the above methods, the method further comprises one, two, three, four or all of:
i) expanding the acceptor cell or population comprising acceptor cells;
ii) selecting the acceptor cell or population comprising acceptor cells;
iii) enriching for the acceptor cell or population comprising acceptor cells;
iv) purifying the acceptor cell or population comprising acceptor cells; or
v) formulating the acceptor cell or population comprising acceptor cells.

In some embodiments of any of the above methods, the method further comprises one, two, three, four or all of:
i) expanding the donor cell or population comprising donor cells;
ii) selecting the donor cell or population comprising donor cells;
iii) enriching for the donor cell or population comprising donor cells;
iv) purifying the donor cell or population comprising donor cells; or
v) formulating the donor cell or population comprising donor cells.

In some embodiments, the contacting occurs in vitro or ex-vivo. In some embodiments, the contacting occurs in vivo.

In some embodiments, the donor cell comprises a fusogen (e.g., vsv-g), e.g., as described herein.

In an aspect, the disclosure provides method of modulating, e.g., enhancing or decreasing, a biological function in a subject, a target tissue, or a cell, comprising administering to the subject, or contacting the target tissue or the cell with, a donor cell described herein, an acceptor cell described herein, a system described herein, or a pharmaceutical composition described herein.

In some embodiments, a method of modulating, e.g., increasing or decreasing, a biological function comprises modulating an interaction between a receptor and a ligand or an interleukin and a receptor, e.g., a receptor, ligand, interleukin, or pair thereof, by administering to a subject, or contacting a target tissue or cell with, a donor cell described herein, an acceptor cell described herein, a system described herein, or a pharmaceutical composition described herein. In some embodiments, the method comprises modulating (e.g., increasing or decreasing) the signaling activity of a receptor, ligand, interleukin, or pair thereof described herein (e.g., an RTK, scavenger receptor, or Cluster of Differentiation protein). In some embodiments, the donor cell, acceptor cell, system, or pharmaceutical composition comprises a membrane-associated agent or cargo molecule comprising the receptor, ligand, or interleukin.

In an aspect, the disclosure provides a method of delivering or targeting a function to a subject, comprising administering to the subject a donor cell described herein, an acceptor cell described herein, a system described herein, or a pharmaceutical composition described herein, wherein the donor cell, the acceptor cell or the pharmaceutical composition is administered in an amount and/or time such that the function in the subject is delivered or targeted.

In some embodiments, the subject has a cancer, an inflammatory disorder, autoimmune disease, a chronic disease, inflammation, damaged organ function, an infectious disease, a degenerative disorder, a genetic disease, or an injury.

This disclosure provides, in certain aspects, a method of delivering a donor cell comprising a membrane-associated agent and optionally a cargo molecule as described herein to a human subject, a target tissue, or an acceptor cell, comprising administering to the human subject, or contacting the target tissue or the acceptor cell with, a donor cell described herein, a composition comprising one or more donor cells described herein, or a pharmaceutical composition described herein, thereby administering the donor cell to the subject.

This disclosure provides, in certain aspects, a method of delivering a membrane-associated agent (e.g., comprising or operably associated or linked to a cargo molecule) to a subject, a target tissue, or an acceptor cell, comprising administering to the subject, or contacting the target tissue or the acceptor cell with, a donor cell described herein or a composition or preparation described herein (e.g., a pharmaceutical composition described herein), wherein the donor cell, composition, or preparation is administered in an amount and/or time such that the membrane-associated agent and/or cargo molecule are delivered.

This disclosure provides, in certain aspects, a method of delivering a cargo molecule to a subject, a target tissue, or an acceptor cell, comprising administering to the subject, or contacting the target tissue or the acceptor cell with, a donor cell described herein or a composition or preparation described herein (e.g., a pharmaceutical composition described herein), wherein the donor cell, composition, or preparation is administered in an amount and/or time such that the cargo molecule is delivered.

This disclosure provides, in certain aspects, a method of modulating, e.g., enhancing, a biological function in a subject, a target tissue, or a cell (e.g., an acceptor cell), comprising administering to the subject, or contacting the target tissue or the cell with, a donor cell, composition, or preparation comprising a membrane-associated agent (e.g., comprising or operably associated or linked to a cargo molecule) described herein, e.g., a pharmaceutical composition described herein, thereby modulating the biological function in the subject.

This disclosure provides, in certain aspects, a method of delivering or targeting a membrane protein function to a subject, comprising administering to the subject a donor cell, composition, or preparation described herein that comprises a membrane-associated agent (e.g., comprising or operably associated or linked to a cargo molecule), wherein the donor cell, composition, or preparation is administered in an amount and/or time such that the membrane protein function is delivered or targeted in the subject. In some embodiments, the membrane-associated agent has the membrane protein function to be delivered or targeted. In some embodiments, the cargo molecule has the membrane protein function to be delivered or targeted. In embodiments, the subject has a cancer, an inflammatory disorder, autoimmune disease, a chronic disease, inflammation, damaged organ function, an infectious disease, a degenerative disorder, a genetic disease, or an injury.

The administration of a pharmaceutical composition described herein may be delivered, for example, by way of oral, inhaled, transdermal or parenteral (including intravenous, intratumoral, intraperitoneal, intramuscular, intracavity, and subcutaneous) administration. The donor cells may be administered alone or formulated as a pharmaceutical composition.

The donor cell, composition, or preparation may be administered, in some embodiments, in the form of a unit-dose composition, such as a unit dose oral, parenteral, transdermal or inhaled composition. Such compositions are generally prepared by admixture and are suitably adapted for oral, inhaled, transdermal or parenteral administration, and as such may be in the form of tablets, capsules, oral liquid preparations, powders, granules, lozenges, reconstitutable powders, injectable and infusable solutions or suspensions or suppositories or aerosols.

In some embodiments, delivery of a membrane-associated agent and/or cargo molecule via a donor cell, as described herein, may induce or block cellular differentiation, de-differentiation, or trans-differentiation. The acceptor cell may be a precursor cell. Alternatively, the acceptor cell may be a differentiated cell, and the cell fate alteration includes driving de-differentiation into a pluripotent precursor cell, or blocking such de-differentiation. In situations where a change in cell fate is desired, effective amounts of a donor cell described herein comprising (e.g., as a cargo molecule) a cell fate inductive molecule or signal is introduced into a target cell under conditions such that an alteration in cell fate is induced. In some embodiments, a donor cell described herein is useful to reprogram a subpopulation of cells from a first phenotype to a second phenotype. Such a reprogramming may be temporary or permanent. Optionally, the reprogramming induces a target cell to adopt an intermediate phenotype.

Also provided are methods of reducing cellular differentiation in a target cell population. For example, a target cell population containing one or more precursor cell types is contacted with a donor cell or composition described herein, under conditions such that the composition reduces the differentiation of the precursor cell. In certain embodiments, the target cell population (e.g., a population comprising a plurality of acceptor cells as described herein) contains injured tissue in a mammalian subject or tissue affected by a surgical procedure. The precursor cell is, e.g., a stromal precursor cell, a neural precursor cell, or a mesenchymal precursor cell.

A donor cell or composition thereof described herein, comprising a membrane-associated agent may be used to deliver a cargo molecule (e.g., comprised in, operably associated to or linked to, or not attached to, the membrane-associated agent) to a cell tissue or subject. Delivery of a membrane-associated agent and/or cargo molecule by administration of a donor cell or composition described herein may modify cellular protein expression levels. In certain embodiments, the delivered agent or cargo molecule directs upregulation of (via expression in the cell, delivery in the cell, or induction within the cell) of one or more polypeptides or nucleic acids that provides a functional activity which is substantially absent or reduced in the cell into which the agent or cargo molecule is delivered. For example, the missing functional activity may be enzymatic, structural, signaling or regulatory in nature. In related embodiments, the administered composition directs up-regulation of one or more polypeptides or nucleic acids that increases (e.g., synergistically) a functional activity which is present but substantially deficient in the cell in which the polypeptide or nucleic acid is upregulated. In related embodiments, the administered composition directs down-regulation of one or more polypeptides or nucleic acids that decreases (e.g., synergistically) a functional activity which is present or upregulated in the cell in which the polypeptide or nucleic acid is downregulated. In certain embodiments, the administered agent or cargo molecule directs upregulation of certain functional activities and downregulation of other functional activities.

In embodiments, the donor cell, or composition, or the membrane-associated agent and/or cargo molecule transferred from same, mediates an effect on an acceptor cell, and the effect lasts for at least 1, 2, 3, 4, 5, 6, or 7 days, 2, 3, or 4 weeks, or 1, 2, 3, 6, or 12 months. In some embodiments, the effect lasts for less than 1, 2, 3, 4, 5, 6, or 7 days, 2, 3, or 4 weeks, or 1, 2, 3, 6, or 12 months. In Vivo Uses

The donor cells and compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein). In one embodiment, the subject has cancer. In one embodiment, the subject has an infectious disease.

In some embodiments, the source of the donor cells (e.g., as described herein) is from the same subject that is administered the donor cells or a composition comprising donor cells. In other embodiments, they are different, e.g., are allogeneic. For example, the source of the donor cells and recipient tissue may be autologous (from the same subject) or heterologous (from different subjects). In either case, the donor tissue for donor cells described herein may be a different tissue type than the recipient tissue. For example, the donor tissue may be muscular tissue and the recipient tissue may be connective tissue (e.g., adipose tissue). In other embodiments, the donor tissue and recipient tissue may be of the same or different type, but from different organ systems.

A donor cell or composition described herein may be administered to a subject having a cancer, an autoimmune disease, an infectious disease, a metabolic disease, a neurodegenerative disease, or a genetic disease (e.g., enzyme deficiency). In some embodiments, a tissue of the subject is in need of regeneration.

In some embodiments, a therapeutically effective amount of donor cells or a composition described herein is administered to a subject. In some embodiments, a therapeutically effective amount of a substance (e.g., a donor cell, exogenous membrane-associated agent, and/or cargo molecule) is an amount that is sufficient, when administered to a subject who has or is susceptible to a disease, disorder, and/or condition, to treat, and/or delay the onset of the disease, disorder, and/or condition. For example, in embodiments the effective amount of donor cells, exogenous membrane-associated agents, and/or cargo molecules in a formulation to treat a disease, disorder, and/or condition is the amount that alleviates, ameliorates, relieves, inhibits, delays onset of, reduces severity of and/or reduces incidence of one or more symptoms or features of the disease, disorder, and/or condition.

In some embodiments, a subject is treated with a donor cell, or a composition as described herein. In some embodiments, the treatment partially or completely alleviates, ameliorates, relieves, inhibits, delays onset of, reduces severity of, and/or reduces incidence of one or more symptoms, features, and/or causes of a particular disease, disorder, and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. In some embodiments, treatment partially or completely ameliorates the root cause of the relevant disease, disorder, and/or condition. In some embodiments, the disease, disorder, or condition is selected from cancer, an inflammatory disorder, autoimmune disease, a chronic disease, inflammation, damaged organ function, an infectious disease, a degenerative disorder, a genetic disease, and/or an injury.

In some embodiments, the donor cells, or composition are effective to treat the disease, e.g., cancer. In some embodiments, the donor cells or composition are effective to reduce the number of cancer cells in the subject compared to the number of cancer cells in the subject before administration. In some embodiments, the donor cells or composition are effective to reduce the number of cancer cells in the subject compared to the expected course of disease without treatment. In some embodiments, the subject experiences a complete response or partial response after administration of the donor cells or composition.

In some embodiments, the donor cell is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3:1462 DOI: 10.1038/srep01462). Thus, in some embodiments, the donor cell is co-administered with an inhibitor of sypressyn, e.g., a siRNA or inhibitory antibody.

Donor cells, acceptor cells, and/or cargo molecules can be autologous, allogeneic or xenogeneic to the subject. Donor cells and/or cargo molecules can be autologous, allogeneic or xenogeneic to the target cell, e.g., acceptor cell.

Compositions comprising the donor cells, exogenous membrane-associated agents, and/or cargo molecules described herein may be administered or targeted to the circulatory system, hepatic system, renal system, cardio-pulmonary system, central nervous system, peripheral nervous system, musculoskeletal system, lymphatic system, immune system, sensory nervous systems (sight, hearing, smell, touch, taste), digestive system, endocrine systems (including adipose tissue metabolic regulation), and reproductive system.

In embodiments, a donor cell or composition described herein is delivered ex-vivo to a cell or tissue, e.g., a human cell or tissue. In some embodiments, the donor cell or composition is delivered to an ex vivo tissue that is in an injured state (e.g., from trauma, disease, hypoxia, ischemia or other damage).

In some embodiments, the donor cell or composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the donor cell or composition improves viability, respiration, or other function of the transplant. The donor cell or composition can be delivered to the tissue or organ before, during and/or after transplantation.

In some embodiments, a donor cell or composition described herein is delivered ex-vivo to an acceptor cell or tissue derived from a subject. In some embodiments, the acceptor cell or tissue is readministered to the subject (i.e., the cell or tissue is autologous), e.g., as a donor cell for an acceptor cell within the subject.

The donor cells may transfer a membrane-associated agent (e.g., comprising or operably associated or linked to a cargo molecule) and/or a cargo molecule to an acceptor cell from any mammalian (e.g., human) tissue, e.g., from epithelial, connective, muscular, or nervous tissue or cells, and combinations thereof. The membrane-associated agent and/or cargo molecule can be delivered to any eukaryotic (e.g., mammalian) organ system, for example, from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves)'; reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof.

In embodiments, the donor cell targets an acceptor cell in a tissue, e.g., liver, lungs, heart, spleen, pancreas, gastrointestinal tract, kidney, testes, ovaries, brain, reproductive organs, central nervous system, peripheral nervous system, skeletal muscle, endothelium, inner ear, adipose tissue (e.g., brown adipose tissue or white adipose tissue) or eye, when administered to a subject, e.g., wherein at least 0.1%, 0.5%, 1%, 1.5%, 2%, 2.5%, 3%, 4%, 5%, 10%, 20%, 30%, 40%, 50%, 60%, 70%, 80%, or 90% of the membrane-associated agents and/or cargo molecules in a population of administered donor cells remain present in the target tissue after 24, 48, or 72 hours. In some embodiments, the donor cell targets the acceptor cell via an extracellular moiety described herein (e.g., comprising a target domain described herein), e.g., comprised in a membrane-associated agent described herein.

In embodiments, the donor cell may transfer a membrane-associated agent and/or cargo molecule to an acceptor cell from a source of stem cells or progenitor cells, e.g., bone marrow stromal cells, marrow-derived adult progenitor cells (MAPCs), endothelial progenitor cells (EPC), blast cells, intermediate progenitor cells formed in the subventricular zone, neural stem cells, muscle stem cells, satellite cells, liver stem cells, hematopoietic stem cells, bone marrow stromal cells, epidermal stem cells, embryonic stem cells, mesenchymal stem cells, umbilical cord stem cells, precursor cells, muscle precursor cells, myoblast, cardiomyoblast, neural precursor cells, glial precursor cells, neuronal precursor cells, hepatoblasts.

### Specific Delivery to Target Cells

In some embodiments, a donor cell or composition described herein delivers a cargo molecule (e.g., operably associated with or linked to a membrane-associated agent) preferentially to a target acceptor cell compared to a non-target cell. Accordingly, in certain embodiments, a donor cell described herein has one or both of the following properties: (i) when the plurality of donor cells are contacted with a cell population comprising target acceptor cells and non-target cells, under conditions suitable for transfer of the cargo molecule from a donor cell to an acceptor cell, the cargo molecule is present in at least 2-fold, 5-fold, 10-fold, 20-fold, 50-fold, or 100-fold more in target acceptor cells than in non-target cells, or (ii) the donor cells of the plurality transfer the cargo molecule at a higher rate to a target acceptor cell than with a non-target cell by at least at least 50%.

In some embodiments, a population of donor cells comprising cargo molecules and a first docking moiety deliver more cargo molecules to a population of target acceptor cells that comprises a second docking moiety than to a population of cells that lack the second docking moiety (non-target cells). In some embodiments, after delivery, the cargo molecule is detectably present in about 2-5, 5-10, 10-20, 20-30, 30-40, 40-50, 50-60, 60-70, 70-80, or 80-90 times more target acceptor cells than non-target cells. In some embodiments, after delivery, about 70-80%, 80-90%, 90-95%, or 95-100%, or at least about 70%, 80%, 90%, or 95%, of the cargo molecules that are present in cells other than the donor cells are present in target acceptor cells (rather than to non-target cells). In some embodiments, about 0-5%, 5-10%, 10-20%, or 20-30%, or less than 30%, 20%, 10%, or 5%, of the cargo molecules that are present in cells other than the donor ells are present in non-target cells (rather than to target acceptor cells).

In some embodiments, the population of target acceptor cells comprising the second docking moiety and the population of non-target cells are part of a larger population of cells. In embodiments, the target acceptor cells and non-target cells are mixed together or intermingled in the larger population of cells. In embodiments, the target acceptor cells and non-target cells are separated within the larger population of cells (e.g., the target acceptor cells are present in a first tissue or region within the larger population of cells, and the non-target cells are present in a second tissue or region within the larger population of cells). In some embodiments, the larger population of cells is a biological sample (e.g., a blood sample or biopsy), e.g., obtained from a subject. In some embodiments, the larger population of cells comprises peripheral blood mononuclear cells (PBMCs). In embodiments, the PBMCs comprise B cells, T cells, NK cells, and/or monocytes. In some embodiments, the target acceptor cells are B cells (e.g., B cells expressing CD19). In embodiments, the first docking moiety is comprised in a CAR (e.g., a CAR that binds CD19). In embodiments, the second docking moiety is comprised in CD19, or an epitope thereof (e.g., a fragment or variant of CD19 recognized by a CAR that binds CD19). In some embodiments, the target acceptor cells are T cells. In some embodiments, the target acceptor cells are NK cells. In some embodiments, the target acceptor cells are monocytes.

In some embodiments, the donor cells transfer the cargo molecules to target cells at a rate such that the cargo molecule is delivered to at least 10%, 20%, 30%, 40%, 50%, 60%, 70%, 80%, or 90%, of target cells after 24, 48, or 72 hours. In embodiments, the amount of targeted transfer is about 30%-70%, 35%-65%, 40%-60%, 45%-55%, or 45%-50%. In embodiments, the amount of transfer is about 20%-40%, 25%-35%, or 30%-35.

In some embodiments, the donor cells or composition delivers at least 40%, 45%, 50%, 55%, 60%, 65%, 70%, 75%, 80%, 85%, 90%, 95%, 96%, 97%, 98%, or 99% of the cargo molecule to the target acceptor cell population compared to a reference cell population or to a non-target cell population. In some embodiments, the donor cells or composition transfer at least 40%, 45%, 50%, 55%, 60%, 65%, 70%, 75%, 80%, 85%, 90%, 95%, 96%, 97%, 98%, or 99% more of the cargo molecule to the target acceptor cell population compared to the reference cell population or to the non-target cell population.

### Methods of Treatment

The donor cells, acceptor cells, cargo molecules, exogenous membrane-associated agents, and/or compositions described herein may be used to modulate one or more biological function in a subject, e.g., a patient, e.g., a human patient.

In some embodiments, the biological function is selected from:
modulating, e.g., increasing or decreasing, an interaction between two cells;
modulating, e.g. increasing or decreasing, an immune response;
modulating, e.g. increasing or decreasing, recruitment of cells to a target tissue;
decreasing the growth rate of a cancer; or
reducing the number of cancerous cells in the subject.

In some aspects, provided herein is a method of administering a donor cell to a subject, e.g., a human subject, comprising administering to the subject a donor cell or a composition comprising a plurality of donor cells, a donor cell composition, or a pharmaceutical composition as described herein, thereby administering the donor cell to the subject.

In some aspects, provided herein a method of delivering a cargo molecule (e.g., operably associated with or linked to a membrane-associated agent as described herein) to a subject, comprising administering to the subject a donor cell, or a composition comprising a plurality of donor cells, a donor cell, or a pharmaceutical composition as described herein, wherein the donor cells are administered in an amount and/or time such that the cargo molecule is delivered

In some aspects, provided herein is a method of modulating, e.g., enhancing, a biological function in a subject, comprising administering to the subject a donor cell, or a composition comprising a plurality of donor cells, a donor cell composition, or a pharmaceutical composition as described herein, thereby modulating the biological function in the subject.

In some aspects, provided herein is a method of delivering or targeting a function to a subject, comprising administering to the subject a donor cell or a composition comprising a plurality of donor cells, a donor cell composition, or a pharmaceutical composition as described herein, wherein the donor cells are administered in an amount and/or time such that the function in the subject is delivered or targeted.

In some aspects, provided herein is a method of treating a disease or disorder in a patient comprising administering to the subject a donor cell, or a composition comprising a plurality of donor cells, a donor cell composition, or a pharmaceutical composition as described herein, wherein the donor cells are administered in an amount and/or time such that the disease or disorder is treated.

In some embodiments, the subject has a cancer, an inflammatory disorder, autoimmune disease, a chronic disease, inflammation, damaged organ function, an infectious disease, metabolic disease, degenerative disorder, genetic disease (e.g., a genetic deficiency or a dominant genetic disorder), or an injury. In some embodiments, the subject has an infectious disease and the cargo molecule comprises an antigen for the infectious disease. In some embodiments, the subject has a genetic deficiency and the cargo molecule comprises a protein for which the subject is deficient, or a nucleic acid (e.g., a DNA, a gDNA, a cDNA, an RNA, a pre-mRNA, an mRNA, etc.) encoding the protein, or a DNA encoding the protein, or a chromosome encoding the protein, or a nucleus comprising a nucleic acid encoding the protein. In some embodiments, the subject has a dominant genetic disorder, and the cargo molecule comprises or is associated with a nucleic acid inhibitor (e.g., siRNA or miRNA) of the dominant mutant allele. In some embodiments, the subject has a dominant genetic disorder, and the cargo molecule comprises or is associated with a nucleic acid inhibitor (e.g., siRNA or miRNA) of the dominant mutant allele, and the cargo molecule comprises or is associated with an mRNA encoding a non-mutated allele of the mutated gene that is not targeted by the nucleic acid inhibitor. In some embodiments, the subject is in need of vaccination. In some embodiments, the subject is in need of regeneration, e.g., of an injured site.

In some embodiments, the donor cell, composition, or preparation is administered to the subject at least 1, 2, 3, 4, or 5 times.

In some embodiments, the donor cell, composition, or preparation is administered to the subject systemically (e.g., orally, parenterally, subcutaneously, intravenously, intramuscularly, intraperitoneally) or locally. In some embodiments, the donor cell composition or preparation is administered to the subject such that the donor cell composition or preparation reaches a target tissue selected from liver, lungs, heart, spleen, pancreas, gastrointestinal tract, kidney, testes, ovaries, brain, reproductive organs, central nervous system, peripheral nervous system, skeletal muscle, endothelium, inner ear, or eye. In some embodiments (e.g., wherein the subject has an autoimmune disease), the donor cell composition or preparation is co-administered with an immunosuppressive agent, e.g., a glucocorticoid, cytostatic, antibody, or immunophilin modulator. In some embodiments (e.g., wherein the subject has a cancer or an infectious disease), the donor cell composition or preparation is co-administered with an immunostimulatory agent, e.g., an adjuvant, interleukin, cytokine, or chemokine. In some embodiments, administration of the donor cell composition or preparation results in upregulation or downregulation of a gene in a target cell in the subject, e.g., wherein the donor cell comprises a transcriptional activator or repressor, a translational activator or repressor, or an epigenetic activator or repressor.

In some embodiments, when the plurality of donor cells are contacted with a cell population comprising target acceptor cells and non-target cells, the cargo molecules are present in substantial amounts in at least 2-fold, 5-fold, 10-fold, 20-fold, 50-fold, or 100-fold more target acceptor cells than non-target cells. In some embodiments, the donor cells of the plurality transfer cargo molecules at a higher rate with a target acceptor cell than with a non-target cell by at least at least 50%.

In some embodiments, the disease or disorder is selected from cancer, autoimmune disorder, or infectious disease. In some embodiments, the subject has a cancer. In some embodiments, cargo molecule comprises a neoantigen. In some embodiments, the donor cell or composition thereof is administered to the subject at least 1, 2, 3, 4, or 5 times. In some embodiments, the donor cell or composition thereof is administered to the subject systemically (e.g., orally, parenterally, subcutaneously, intravenously, intramuscularly, intraperitoneally) or locally. In some embodiments, the donor cell or composition thereof is administered to the subject such that the donor cell or composition thereof reaches a target tissue selected from liver, lungs, heart, spleen, pancreas, gastrointestinal tract, kidney, testes, ovaries, brain, reproductive organs, central nervous system, peripheral nervous system, skeletal muscle, endothelium, inner ear, or eye. In some embodiments, the donor cell or composition thereof is co-administered with an immunosuppressive agent, e.g., a glucocorticoid, cytostatic, antibody, or immunophilin modulator. In some embodiments, the donor cell or composition thereof is co-administered with an immunostimulatory agent, e.g., an adjuvant, interleukin, cytokine, or chemokine.

The terms “cancer”, “malignancy”, “neoplasm”, “tumor”, and “carcinoma”, are used herein to refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a tumor may be a disperse tumor or a liquid tumor. In some embodiments, a relevant cancer may be characterized by a hematologic tumor. In general, examples of different types of cancers known in the art include, for example, leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

In some embodiments, the plurality of donor cells has a local, distal, or systemic effect (e.g., via the transfer of cargo molecules to acceptor cells).

In some embodiments, any of the methods disclosed herein, further comprises a step of monitoring adverse events in the organism. In some embodiments, the adverse event includes one or more of cytokine release syndrome, fever, tachycardia, chills, anorexia, nausea, vomiting, myalgia, headaches, capillary leak syndrome, hypotension, pulmonary edema, coagulopathy, renal dysfunction, kidney injury, macrophage-activation syndrome, hemophagocytic lymphohistiocytosis, organ failure, cerebral edema, bystander inflammation from T cell activation, neurologic symptoms, encephalopathy, confusion, hallucination, delirium, obtundation, aphasia, seizures, B-cell aplasia, tumor lysis syndrome, and graft versus host disease.

In some embodiments, the organism is a human. In some embodiments, the human has a disease, disorder, or condition. In some embodiments, presence of the cargo molecule in the target cell (e.g., acceptor cell) improves one or more symptoms of the disease, disorder, or condition.

### Additional Therapeutic Agents

In some embodiments, the donor cell or composition thereof is co-administered with an additional agent, e.g., a therapeutic agent, to a subject, e.g., a recipient, e.g., a recipient described herein. In some embodiments, the co-administered therapeutic agent is an immunosuppressive agent, e.g., a glucocorticoid (e.g., dexamethasone), cytostatic (e.g., methotrexate), antibody (e.g., Muromonab-CD3), or immunophilin modulator (e.g., Ciclosporin or rapamycin). In embodiments, the immunosuppressive agent decreases immune mediated clearance of donor cells, exogenous membrane-associated agents, and/or cargo molecules. In some embodiments the donor cell or composition thereof is co-administered with an immunostimulatory agent, e.g., an adjuvant, an interleukin, a cytokine, or a chemokine.

In some embodiments, the donor cell or composition thereof and the immunosuppressive agent are administered at the same time, e.g., contemporaneously administered. In some embodiments, the donor cell or composition thereof is administered before administration of the immunosuppressive agent. In some embodiments, the donor cell or composition thereof is administered after administration of the immunosuppressive agent.

In some embodiments, the immunosuppressive agent is a small molecule such as ibuprofen, acetaminophen, cyclosporine, tacrolimus, rapamycin, mycophenolate, cyclophosphamide, glucocorticoids, sirolimus, azathriopine, or methotrexate.

In some embodiments, the immunosuppressive agent is an antibody molecule, including but not limited to: muronomab (anti-CD3), Daclizumab (anti-IL12), Basiliximab, Infliximab (Anti-TNFa), or rituximab (Anti-CD20).

In some embodiments, co-administration of the donor cell or composition thereof with the immunosuppressive agent results in enhanced persistence of the donor cell or composition thereof, exogenous membrane-associated agent, and/or cargo molecule in the subject compared to administration of the donor cell or composition thereof alone. In some embodiments, the enhanced persistence of the donor cell or composition thereof, exogenous membrane-associated agent, and/or cargo molecule in the co-administration is at least 10%, 20%, 30%, 40%, 50%, 60%, 70%, 80%, 90% or longer, compared to persistence of the donor cell or composition thereof, exogenous membrane-associated agent, and/or cargo molecule when administered alone. In some embodiments, the enhanced persistence of the donor cell or composition thereof, exogenous membrane-associated agent, and/or cargo molecule in the co-administration is at least 1, 2, 3, 4, 5, 6, 7, 10, 15, 20, 25, or 30 days or longer, compared to survival of the donor cell or composition thereof, exogenous membrane-associated agent, and/or cargo molecule when administered alone.

### Methods of Manufacturing

The disclosure provides, in some aspects, a method of manufacturing a donor cell or a composition thereof, comprising:
a) providing a source cell comprising, e.g., expressing, a membrane-associated agent;
b) producing a donor cell from the source cell, wherein the donor cell comprises a lipid bilayer, a lumen, a membrane-associated agent, and a cargo molecule (e.g., operably associated with or linked to the membrane-associated agent), thereby making a donor cell; and
c) formulating the donor cell, e.g., as a pharmaceutical composition suitable for administration to a subject.

In some aspects, the present disclosure provides a method of manufacturing a donor cell composition, comprising:
a) providing a plurality of donor cells described herein or a donor cell composition described herein; and
b) formulating the donor cells, e.g., as a pharmaceutical composition suitable for administration to a subject.

In some aspects, the present disclosure provides a method of manufacturing a donor cell composition, comprising:
a) providing, e.g., producing, a plurality of donor cells or a donor cell preparation described herein; and
b) assaying a sample of the plurality (e.g., of the preparation) to determine whether one or more (e.g., 2, 3, or more) standards are met. In embodiments, the standard(s) are chosen from:
   donor cells in the sample transfer a membrane-associated agent at a higher rate with a target acceptor cell than with a non-target cell, e.g., by at least at least 1%, 2%, 3%, 4%, 5%, 10%, 20%, 30%, 40%, 50%, 60%, 70%, 80%, 90%;
   donor cells in the sample transfer a membrane-associated agent at a higher rate with a target acceptor cell than other cells, e.g., by at least 10%, 20%, 30%, 40%, 50%, 60%, 70%, 80%, or 90%;
   donor cells in the sample transfer a membrane-associated agent to target acceptor cells at a rate such that a cargo molecule in the donor cell (e.g., operably associated with or linked to the membrane-associated agent) is delivered to at least 10%, 20%, 30%, 40%, 50%, 60%, 70%, 80%, or 90%, of target acceptor cells after 24, 48, or 72 hours;
   the membrane-associated agent is present at a copy number, per donor cell (e.g., on average in the sample), of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies;
   the cargo molecule is present at a copy number, per donor cell (e.g., on average in the sample), of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies;
   the cargo molecule is detectable in donor cells of the sample (e.g., on average in the sample) at a copy number of at least, or no more than, 10, 50, 100, 500, 1,000, 2,000, 5,000, 10,000, 20,000, 50,000, 100,000, 200,000, 500,000 1,000,000, 5,000,000, 10,000,000, 50,000,000, 100,000,000, 500,000,000, or 1,000,000,000 copies;
   the ratio of the copy number of the membrane-associated agent to the copy number of the cargo molecule is between 1,000,000:1 and 100,000:1, 100,000:1 and 10,000:1, 10,000:1 and 1,000:1, 1,000:1 and 100:1, 100:1 and 50:1, 50:1 and 20:1, 20:1 and 10:1, 10:1 and 5:1, 5:1 and 2:1, 2:1 and 1:1, 1:1 and 1:2, 1:2 and 1:5, 1:5 and 1:10, 1:10 and 1:20, 1:20 and 1:50, 1:50 and 1:100, 1:100 and 1:1,000, 1:1,000 and 1:10,000, 1:10,000 and 1:100,000, or 1:100,000 and 1:1,000,000;
   donor cells of the sample are characterized by a lipid composition substantially similar to that of the source cell or wherein one or more of CL, Cer, DAG, HexCer, LPA, LPC, LPE, LPG, LPI, LPS, PA, PC, PE, PG, PI, PS, CE, SM and TAG is within 10%, 15%, 20%, 25%, 30%, 35%, 40%, 45%, 50%, or 75% of the corresponding lipid level in the source cell;
   donor cells of the sample are characterized by a proteomic composition similar to that of the source cell;
   donor cells of the sample are characterized by a ratio of lipids to proteins that is within 10%, 20%, 30%, 40%, or 50% of the corresponding ratio in the source cell;
   donor cells of the sample are characterized by a ratio of proteins to nucleic acids (e.g., DNA) that is within 10%, 20%, 30%, 40%, or 50% of the corresponding ratio in the source cell;
   donor cells of the sample are characterized by a ratio of lipids to nucleic acids (e.g., DNA) that is within 10%, 20%, 30%, 40%, or 50% of the corresponding ratio in the source cell;
   donor cells of the sample are characterized by a half-life in a subject, e.g., in a an experimental animal such as a mouse, that is within 1%, 2%, 3%, 4%, 5%, 10%, 20%, 30%, 40%, 50%, 60%, 70%, 80%, 90%, 100% of the half life of a reference cell;
   donor cells of the sample are characterized by a metabolic activity level that is within 1%, 2%, 3%, 4%, 5%, 10%, 20%, 30%, 40%, 50%, 60%, 70%, 80%, 90%, or 100% of the metabolic activity in a reference cell, e.g., the source cell;
   donor cells of the sample are characterized by a miRNA content level of at least at least 1%, 2%, 3%, 4%, 5%, 10%, 20%, 30%, 40%, 50%, 60%, 70%, 80%, 90%, or greater than that of the source cell;
   the donor cell has a soluble: non-soluble protein ratio is within 1%, 2%, 3%, 4%, 5%, 10%, 20%, 30%, 40%, 50%, 60%, 70%, 80%, 90%, or greater than that of the source cell, e.g., within 1%-2%, 2%-3%, 3%-4%, 4%-5%, 5%-10%, 10%-20%, 20%-30%, 30%-40%, 40%-50%, 50%-60%, 60%-70%, 70%-80%, or 80%-90% of that of the source cell;
   donor cells of the sample are characterized by an LPS level less than 5%, 1%, 0.5%, 0.01%, 0.005%, 0.0001%, 0.00001% or less of the LPS content of the source cell or a reference cell;
   donor cells of the sample are capable of signal transduction, e.g., transmitting an extracellular signal, e.g., by at least 1%, 2%, 3%, 4%, 5%, 10%, 20%, 30%, 40%, 50%, 60%, 70%, 80%, 90%, 100% more than a negative control;
   donor cells of the sample are capable of secreting a protein, e.g., at a rate at least 1%, 2%, 3%, 4%, 5%, 10%, 20%, 30%, 40%, 50%, 60%, 70%, 80%, 90%, or 100% greater than the reference cell; or
   donor cells of the sample are characterized by low immunogenicity, e.g., as described herein; and
c) (optionally) approving the plurality of donor cells or the donor cell composition for release if one or more of the standards is met or (optionally) formulating the plurality of donor cells or the donor cell composition as a drug product if the one or more standards is met.

The present disclosure also provides, in some aspects, a method of manufacturing a donor cell composition, comprising:
a) providing, e.g., producing, a plurality of donor cells described herein or a donor cell composition or preparation described herein; and
b) assaying a sample of the plurality or preparation to determine the presence or level of one or more of the following factors:
   an immunogenic molecule, e.g., an immunogenic protein, e.g., as described herein;
   a pathogen, e.g., a bacterium or virus; or
   a contaminant (e.g., a nuclear structure or component such as nuclear DNA); and
   i) c) (optionally) approving the plurality of donor cells or donor cell composition for release if one or more of the factors is deviates significantly (e.g., by more than a specified amount) from a reference value or (optionally) formulating the plurality of donor cells or the donor cell composition as a drug product if the one or more factors does not significantly deviate (e.g., does not deviate by more than the specified about) from the reference value.

In some aspects, provided herein is a method of manufacturing a donor cell composition, comprising:
i) providing a plurality of donor cell, a donor cell composition, or a pharmaceutical composition as described herein; and
b) assaying one or more donor cells from the plurality to determine whether one or more (e.g., 2, 3, or all) of the following standards are met:
   the donor cell transfers membrane-associated agents and/or cargo molecules (e.g., operably associated with or linked to the membrane-associated agents) at a higher rate with a target acceptor cell than with a non-target cell, e.g., by at least at least 10%;
   the donor cell transfers membrane-associated agents and/or cargo molecules (e.g., operably associated with or linked to the membrane-associated agents) at a higher rate with a target acceptor cell than with other cells, e.g., by at least 50%;
   the donor cell transfers membrane-associated agents and/or cargo molecules (e.g., operably associated with or linked to the membrane-associated agents) to target acceptor cells at a rate such that an agent in the donor cell is delivered to at least 10% of target acceptor cells after 24 hours;
   the membrane-associated agent is present in an acceptor cell after delivery at a copy number of at least 1,000 copies (e.g., at least 1000, 2000, 3000, 4000, 5000, 10,000, 20,000, 30,000, 40,000, 50,000, 100,000, 200,000, 300,000, 400,000, 5000,000, or 1,000,000 copies);
   the cargo molecule is present in an acceptor cell after delivery at a copy number of at least 1,000 copies (e.g., at least 1000, 2000, 3000, 4000, 5000, 10,000, 20,000, 30,000, 40,000, 50,000, 100,000, 200,000, 300,000, 400,000, 5000,000, or 1,000,000 copies);
   the donor cell comprises a membrane-associated agent at a copy number of at least 1,000 copies (e.g., at least 1000, 2000, 3000, 4000, 5000, 10,000, 20,000, 30,000, 40,000, 50,000, 100,000, 200,000, 300,000, 400,000, 5000,000, or 1,000,000 copies);
   the donor cell comprises a cargo molecule at a copy number of at least 1,000 copies (e.g., at least 1000, 2000, 3000, 4000, 5000, 10,000, 20,000, 30,000, 40,000, 50,000, 100,000, 200,000, 300,000, 400,000, 5000,000, or 1,000,000 copies);
   the ratio of the copy number of the membrane-associated agent to the copy number of the cargo molecule is between 1,000,000:1 and 100,000:1, 100,000:1 and 10,000:1, 10,000:1 and 1,000:1, 1,000:1 and 100:1, 100:1 and 50:1, 50:1 and 20:1, 20:1 and 10:1, 10:1 and 5:1, 5:1 and 2:1, 2:1 and 1:1, 1:1 and 1:2, 1:2 and 1:5, 1:5 and 1:10, 1:10 and 1:20, 1:20 and 1:50, 1:50 and 1:100, 1:100 and 1:1,000, 1:1,000 and 1:10,000, 1:10,000 and 1:100,000, or 1:100,000 and 1:1,000,000;
   the donor cell comprises a ratio of lipids to proteins that is within 10%, 20%, 30%, 40%, or 50% of the corresponding ratio in the source cell;
   the donor cell comprises a ratio of proteins to nucleic acids (e.g., DNA) that is within 10%, 20%, 30%, 40%, or 50% of the corresponding ratio in the source cell;
   the donor cell comprises a ratio of lipids to nucleic acids (e.g., DNA) that is within 10%, 20%, 30%, 40%, or 50% of the corresponding ratio in the source cell;
   the donor cell comprises a metabolic activity level that is within 90% of the metabolic activity in a reference cell, e.g., the source cell;
   the donor cell has a miRNA content level of at least 1% than that of the source cell;
   the donor cell has a soluble: non-soluble protein ratio is within 90% of that of the source cell;
   the donor cell has an LPS level less than 5% of the lipid content of donor cells;
   the donor cell and/or compositions or preparations thereof, are capable of signal transduction, e.g., an otherwise similar donor cell in the absence of insulin;
   the donor cell and/or compositions or preparations thereof, are capable of secreting a protein, e.g., at a rate at least 5% greater than a reference cell or
   the donor cell has low immunogenicity, e.g., as described herein; and
   i) c) (optionally) approving the plurality of donor cells or donor cell composition for release if one or more of the standards is met;
      thereby manufacturing a donor cell drug product composition

In some aspects, provided herein is a method of manufacturing a donor cell composition, comprising:
a) providing a plurality of donor cells, a donor cell composition, or a pharmaceutical composition as described herein; and
b) assaying one or more donor cell from the plurality to determine the presence or level of one or more of the following factors:
   an immunogenic molecule, e.g., an immunogenic protein, e.g., as described herein;
   a pathogen, e.g., a bacterium or virus; or
   a contaminant;
   i) c) (optionally) approving the plurality of donor cells or donor cell composition for release if one or more of the factors is below a reference value;
      thereby manufacturing a donor cell drug product composition.

In some embodiments of the methods of making herein, providing a source cell expressing a membrane-associated agent comprises expressing a membrane-associated agent in the source cell or upregulating expression of an endogenous membrane-associated agent in the source cell. In some embodiments, the method comprises inactivating the nucleus of the source cell.

In some embodiments, at least one donor cell of the plurality of donor cells is derived from a source cell.

In embodiments, the donor cell is from a mammalian cell having a modified genome, e.g., to reduce immunogenicity (e.g., by genome editing, e.g., to remove an MHC protein). In embodiments, the method further comprises contacting the source cell of step a) with an immunosuppressive agent, e.g., before or after inactivating the nucleus, e.g., enucleating the cell.

In some embodiments, the donor cell does not comprise Cre or GFP, e.g., EGFP.

In some embodiments of any of the compositions described herein, the composition (e.g., donor cell composition) comprises a donor cell and/or acceptor cell, e.g., as described herein.

### Source Cells

In some embodiments, a donor cell or acceptor cell is derived from a source cell.

In some embodiments, the source cell or target cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeLa, HEK293, HFF-1, MRC-5, WI-38, IMR 90, IMR 91, PER.C6, HT-1080, or BJ cell). In some embodiments, the source cell is other than a 293 cell, HEK cell, human endothelial cell, or a human epithelial cell, monocyte, macrophage, dendritic cell, or stem cell. In some embodiments, the source cell or target cell is a white blood cell or a stem cell. In some embodiments, the source cell or target cell is selected from a neutrophil, a lymphocyte (e.g., a T cell, a B cell, a natural killer cell), a macrophage, a granulocyte, a mesenchymal stem cell, a bone marrow stem cell, an induced pluripotent stem cell, an embryonic stem cell, or a myeloblast.

In some embodiments, the source cell is a cell grown under adherent or suspension conditions. In some embodiments, the source cell is a primary cell, a cultured cell, an immortalized cell, or a cell line. In some embodiments, the source cell is allogeneic, e.g., obtained from a different organism of the same species as the target cell. In some embodiments, the source cell is autologous, e.g., obtained from the same organism as the target cell. In some embodiments, the source cell is heterologous, e.g., obtained from an organism of a different species from the target cell.

In some embodiments, the source cell comprises or further comprises a second agent that is exogenous to the source cell, e.g., a therapeutic agent, e.g., a protein or a nucleic acid (e.g., an RNA, e.g., an mRNA or miRNA). In some embodiments, the second agent is present at least, or no more than, 10, 20, 50, 100, 200, 500, 1,000, 2,000, 5,000, 10,000, 20,000, 50,000, 100,000, 200,000, 500,000 or 1,000,000 copies comprised by the cell (e.g., donor cell or acceptor cell), or is present at an average level of at least, or no more than, 10, 20, 50, 100, 200, 500, 1,000, 2,000, 5,000, 10,000, 20,000, 50,000, 100,000, 200,000, 500,000 or 1,000,000 copies per cell (e.g., donor cell or acceptor cell).

In some embodiments, the cell (e.g., donor cell or acceptor cell) has an altered, e.g., increased or decreased level of one or more endogenous molecules as compared to the source cell, e.g., protein or nucleic acid, e.g., due to treatment of the source cell, e.g., mammalian source cell with a siRNA or gene editing enzyme. In some embodiments, the cell (e.g., donor cell or acceptor cell) comprises at least, or no more than, 10, 20, 50, 100, 200, 500, 1,000, 2,000, 5,000, 10,000, 20,000, 50,000, 100,000, 200,000, 500,000 or 1,000,000 copies of the endogenous molecule, or is present at an average level of at least, or no more than, 10, 20, 50, 100, 200, 500, 1,000, 2,000, 5,000, 10,000, 20,000, 50,000, 100,000, 200,000, 500,000 or 1,000,000 copies of the endogenous molecule per cell (e.g., donor cell or acceptor cell). In some embodiments, the endogenous molecule (e.g., an RNA or protein) is present in the cell (e.g., donor cell or acceptor cell) at a concentration of at least 1, 2, 3, 4, 5, 10, 20, 50, 100, 500, 10³, 5.0×10³, 10⁴, 5.0×10⁴, 10⁵, 5.0×10⁵, 10⁶, 5.0×10⁶, 1.0×10⁷, 5.0×10⁷, or 1.0×10⁸ greater than its concentration in the source cell. In some embodiments, the endogenous molecule (e.g., an RNA or protein) is present in the cell (e.g., donor cell or acceptor cell) at a concentration of at least 1, 2, 3, 4, 5, 10, 20, 50, 100, 500, 10³, 5.0×10³, 10⁴, 5.0×10⁴, 10⁵, 5.0×10⁵, 10⁶, 5.0×10⁶, 1.0×10⁷, 5.0×10⁷, or 1.0×10⁸ less than its concentration in the source cell.

In some embodiments, provided donor cells, acceptor cells, and/or compositions or preparations thereof, comprise less than 0.01%, 0.05%, 0.1%, 0.5%, 1%, 1.5%, 2%, 2.5%, 3%, 4%, 5%, or 10% source cells by protein mass or less than 0.01%, 0.05%, 0.1%, 0.5%, 1%, 1.5%, 2%, 2.5%, 3%, 4%, 5%, or 10% of cells have a functional nucleus. In some embodiments, at least 10%, 20%, 30%, 40%, 50%, 60%, 70%, 80%, 90%, 95%, or 99% of donor cells and/or acceptor cells in a composition or preparation described herein comprise an organelle, e.g., a mitochondrion.

In some embodiments, provided donor cells and/or acceptor cells, and/or compositions or preparations thereof, comprise at least 0.01%-0.05%, 0.05%-0.1%, 0.1%-0.5%, 0.5%-1%, 1%-2%, 2%-3%, 3%-4%, 4%-5%, 5%-10%, 10%-20%, 20%-30%, 30%-40%, 40%-50%, 50%-60%, 60%-70%, 70%-80%, or 80%-90% donor cells and/or acceptor cells, wherein: i) the membrane-associated agent is present at a copy number of at least 1,000 copies per donor cell or acceptor cell, ii) the ratio of the copy number of the membrane-associated agent to the copy number of the cargo molecule per donor cell or acceptor cell is between 1,000,000:1 and 100,000:1, 100,000:1 and 10,000:1, 10,000:1 and 1,000:1, 1,000:1 and 100:1, 100:1 and 50:1, 50:1 and 20:1, 20:1 and 10:1, 10:1 and 5:1, 5:1 and 2:1, 2:1 and 1:1, 1:1 and 1:2, 1:2 and 1:5, 1:5 and 1:10, 1:10 and 1:20, 1:20 and 1:50, 1:50 and 1:100, 1:100 and 1:1,000, 1:1,000 and 1:10,000, 1:10,000 and 1:100,000, or 1:100,000 and 1:1,000,000, or iii) the cargo molecule is present at a copy number of at least 1,000 copies per donor cell and/or acceptor cell.

In embodiments, the source cell is a primary cell, immortalized cell or a cell line. In embodiments, the donor cell or acceptor cell is from a source cell having a modified genome, e.g., having reduced immunogenicity (e.g., by genome editing, e.g., to remove an MHC protein, e.g., MHC complex). In embodiments, the source cell is from a cell culture treated with an immunosuppressive agent. In embodiments, the source cell is substantially non-immunogenic, e.g., using an assay described herein. In embodiments, the source cell comprises an exogenous agent, e.g., a therapeutic agent. In embodiments, the source cell is a recombinant cell.

In some embodiments, the source cell is from a cell culture treated with an anti-inflammatory signal. In some embodiments, a method of making described herein further comprises contacting the source cell with an anti-inflammatory signal, e.g., before or after inactivating the nucleus, e.g., enucleating the cell.

### ENUMERATED EMBODIMENTS

1. A donor cell comprising:
(a) a membrane-associated agent comprising: (i) a membrane-associated moiety, (ii) a first docking moiety, and optionally (iii) an intracellular moiety; and
(b) a cargo molecule (e.g., an exogenous cargo molecule);
wherein the donor cell is deficient in at least one endogenous function, or is T cell receptor (TCR)-deficient, or is MHC-deficient.
   2. The donor cell of embodiment 1, wherein the endogenous function is cytotoxic activity (e.g., wherein the donor cell does not have substantial cytotoxic activity).
   3. The donor cell of embodiment 1 or 2, wherein the donor cell is T cell receptor deficient (TCR deficient).
   4. The donor cell of any of the preceding embodiments, wherein the donor cell is MHC deficient.
   5. The donor cell of any of the preceding embodiments, wherein the cargo molecule comprises a therapeutic agent, e.g., an exogenous therapeutic agent.
   6. The donor cell of any of the preceding embodiments, wherein the cargo molecule is an exogenous cargo molecule that binds to the intracellular moiety.
   7. The donor cell of embodiment 6, wherein the cargo molecule comprises (i) a domain (e.g., a ZAP70 domain) that binds specifically to the intracellular moiety of the membrane-associated agent and (ii) a therapeutic domain.
   8. The donor cell of any of the preceding embodiments, wherein the intracellular moiety does not stimulate T cell effector activity.
   9. The donor cell of any of the preceding embodiments, wherein the first docking moiety (e.g., a CAR, TCR, or antibody molecule) specifically recognizes a moiety that is enriched or characteristically expressed on a target cell of interest (e.g., SIINFEKL, CD19, or Glut2).
   10. The donor cell of any of the preceding embodiments, wherein the first docking moiety (e.g., a CAR, TCR, or antibody molecule) specifically recognizes a peptide comprising the amino acid sequence SIINFEKL, e.g., presented on an MHC class I molecule on an acceptor cell.
   11. The donor cell of any of the preceding embodiments, wherein the first docking moiety (e.g., a CAR, TCR, or antibody molecule) specifically recognizes myelin oligodendrocyte glycoprotein (MOG).
   12. The donor cell of any of the preceding embodiments, wherein the first docking moiety (e.g., a CAR, TCR, or antibody molecule) specifically recognizes a biotin moiety, e.g., a binding moiety comprising a streptavidin moiety.
   13. The donor cell of any of the preceding embodiments, wherein membrane-associated agent comprises a shuttle listed in Table B1.
   14. The donor cell of any of the preceding embodiments, wherein the cargo comprises a cargo molecule listed in Table B1.
   15. The donor cell of any of the preceding embodiments, wherein the donor cell stably expresses the membrane-associated agent and/or the cargo molecule.
   16. The donor cell of embodiment 15, wherein the donor cell comprises in its genome a gene encoding the membrane associated agent.
   17. The donor cell of embodiment 15, wherein the donor cell comprises in its genome a gene encoding the cargo molecule.
   18. The donor cell of any of the preceding embodiments, wherein the donor cell is or is derived from a Treg.
   19. The method or composition of embodiment 18, wherein the membrane associated agent comprises a CAR.
   20. The method or composition of embodiment 18, wherein the Treg is a human Treg (e.g., an HLA-02 positive Treg).
   21. The method or composition of any of embodiments 1-17, wherein the donor cell is:
(i) an adipogenic cell, e.g., an adipocyte stem cell (ASC),
(ii) an CD34+ adipocyte, or
(iii) a mature adipocyte.
   22. The method or composition of any of embodiments 1-17, wherein the donor cell is a mesenchymal stem cell (MSC).
   23. The donor cell of any of the preceding embodiments, which is genetically engineered to reduce TCR or MHC expression.
   24. The donor cell of any of the preceding embodiments, which comprises an antisense nucleic acid that reduces TCR or MHC expression.
   25. A donor cell comprising:
(a) a membrane-associated agent comprising: (i) a membrane-associated moiety, (ii) a first docking moiety, and optionally (iii) an intracellular moiety; and
(b) a cargo molecule (e.g., an exogenous cargo molecule);
wherein the membrane-associated agent comprises a CAR or an exogenous TCR; and
wherein the donor cell substantially lacks cytotoxic activity, e.g., wherein the donor cell is a regulatory T cell (Treg).
   26. A donor cell comprising:
(a) a membrane-associated agent comprising: (i) a membrane-associated moiety, (ii) a first docking moiety, and optionally (iii) an intracellular moiety; and
(b) a cargo molecule (e.g., an exogenous cargo molecule);
wherein the donor cell has one or more (e.g., 1, 2, 3, 4, 5, 6, 7, 8, 9, or 10) of the following characteristics:
   (i) the cargo molecule is attached to (e.g., covalently or noncovalently bound to) a cell-penetrating peptide;
   (ii) the membrane-associated agent is attached to (e.g., covalently or noncovalently bound to) a cell-penetrating peptide;
   (iii) the donor cell comprises a cell-penetrating peptide;
   (iv) the donor cell further comprises a fusogen (e.g., a vsv-g protein, e.g., as described in Example 25);
   (v) the donor cell expresses a secreted exogenous effector;
   (vi) the donor cell comprises a virus-like particle (VLP), optionally wherein the VLP infects the acceptor cell;
   (vii) the donor cell inserts a tunneling nanotube to an acceptor cell upon binding of the first docking moiety of the membrane-associated agent to a second docking moiety of the acceptor cell;
   (vii) the donor cell comprises: (1) a nucleic acid molecule comprising a promoter (e.g., a tissue-specific promoter) and a gene encoding the membrane-associated agent and/or a gene encoding the cargo molecule; or (2) a nucleic acid molecule (e.g., an RNA, e.g., an mRNA) encoding the membrane-associated agent and/or a nucleic acid molecule (e.g., an RNA, e.g., an mRNA) encoding the cargo molecule;
   (viii) the donor cell comprises an inducible cell death agent (e.g., an inducible cell death protein, e.g., an inducible caspase, e.g., inducible Caspase 9); and/or
   (ix) the donor cell is or is derived from (e.g., differentiated from) an induced pluripotent stem cell (iPSC) or hematopoietic stem cell (HSC).
      27. A preparation comprising:
a) a first plurality of donor cells according to any of the preceding embodiments, and
b) a second plurality of cells that are not donor cells according to any of the preceding embodiments.
   28. The preparation of embodiment 27, wherein the cells of the second plurality:
a) do not comprise a membrane-associated agent;
b) do not comprise a cargo molecule;
c) are not TCR deficient; and/or
d) are not MHC deficient.
   29. The preparation of embodiment 27 or 28, wherein at least 50%, 60%, 70%, 80%, 85%, 90%, 95%, 96%, 97%, 98%, or 99% of the cells in the preparation are cells of the first plurality.
   30. The preparation of embodiment 27 or 28, wherein at least 1%, 2%, 3%, 4%, 5%, 10%, 15%, 20%, 25%, 30%, 40%, or 50% of the cells in the preparation are cells of the first plurality.
   31. The preparation of any of embodiments 27-30, which is substantially free of CD4+ T cells (e.g., CD4+ helper T cells), CD8+ T cells, NK cells, or any combination thereof.
   32. The preparation of any of embodiments 27-31, which is substantially free of cells having effector T cell activity.
   33. The preparation of any of embodiments 27-32, which is substantially free of cells having cytotoxic T cell activity.
   34. The preparation of any of embodiments 27-33, which comprises fewer than 1, 2, 3, 4, 5, 6, 7, 8, 9, 10, 20, 30, 40, 50, 60, 70, 80, 90, 100, 200, 300, 400, or 500 CD4+ T cells (e.g., CD4+ helper T cells), CD8+ T cells, NK cells, or any combination thereof per mL of the preparation.
   35. The preparation of any of embodiments 27-34, which comprises fewer than 1, 2, 3, 4, 5, 6, 7, 8, 9, 10, 20, 30, 40, 50, 60, 70, 80, 90, 100, 200, 300, 400, or 500 cells having effector T cell activity per mL of the preparation.
   36. The preparation of any of embodiments 27-35, which comprises fewer than 1, 2, 3, 4, 5, 6, 7, 8, 9, 10, 20, 30, 40, 50, 60, 70, 80, 90, 100, 200, 300, 400, or 500 cells having cytotoxic T cell activity per mL of the preparation.
   37. The preparation of any of embodiments 27-36, wherein less than 0.001%, 0.01%, 1%, 2%, 3%, 4%, 5%, 6%, 7%, 8%, 9%, 10%, 15%, 20%, 25%, 30%, 40%, or 50% of the cells in the preparation are CD4+ T cells (e.g., CD4+ helper T cells), CD8+ T cells, NK cells, or any combination thereof.
   38. The preparation of any of embodiments 27-37, wherein less than 0.001%, 0.01%, 1%, 2%, 3%, 4%, 5%, 6%, 7%, 8%, 9%, 10%, 15%, 20%, 25%, 30%, 40%, or 50% of the cells in the preparation are cells having effector T cell activity.
   39. The preparation of any of embodiments 27-38, wherein less than 0.001%, 0.01%, 1%, 2%, 3%, 4%, 5%, 6%, 7%, 8%, 9%, 10%, 15%, 20%, 25%, 30%, 40%, or 50% of the cells in the preparation are cells having cytotoxic T cell activity.
   40. A method of treating a disease or disorder in a subject or patient, the method comprising:
administering to the subject a donor cell or preparation of any of the preceding embodiments,
wherein the donor cells or preparation are administered in an amount and/or time such that the disease or disorder is treated.
   41. The method of embodiment 40, wherein the disease or disorder is not a disease or disorder associated with the second docking moiety or a molecule comprising the second docking moiety (e.g., a disease or disorder associated with CD19).
   42. A method of modifying an acceptor cell, the method comprising:
contacting the acceptor cell with a donor cell of any of the preceding embodiments under conditions suitable for transfer of the membrane-associated agent and/or the cargo molecule to the acceptor cell.
   43. A method of modifying a plurality of acceptor cells, the method comprising:
contacting the plurality of acceptor cells with a preparation of any of the preceding embodiments under conditions suitable for transfer of the membrane-associated agent and/or the cargo molecule to the plurality of acceptor cells.
   44. A method of modifying an acceptor cell, the method comprising:
contacting the acceptor cell with a donor cell under conditions suitable for transfer of a membrane-associated agent and/or a cargo molecule to the acceptor cell;
wherein the donor cell comprises:
   (a) a membrane-associated agent comprising: (i) a membrane-associated moiety, (ii) a first docking moiety, and optionally (iii) an intracellular moiety; and
   (b) a cargo molecule (e.g., an exogenous cargo molecule);
wherein the donor cell is deficient in at least one endogenous function, or is T cell receptor (TCR)-deficient, or is MHC-deficient;
wherein the acceptor cell comprises a second docking moiety that binds specifically to the first docking moiety; and
wherein after the transfer the acceptor cell comprises an increased amount of the membrane-associated agent and/or cargo molecule;
thereby modifying the acceptor cell.
   45. The method of embodiment 44, wherein the endogenous function comprises cytotoxic activity (e.g., wherein the donor cell does not have substantial cytotoxic activity).
   46. A method of modifying an acceptor cell, the method comprising:
contacting the acceptor cell with a donor cell under conditions suitable for transfer of a membrane-associated agent and/or a cargo molecule to the acceptor cell;
wherein the donor cell comprises:
   (a) a membrane-associated agent comprising: (i) a membrane-associated moiety, (ii) a first docking moiety, and optionally (iii) an intracellular moiety; and
   (b) a cargo molecule (e.g., an exogenous cargo molecule);
wherein the donor cell substantially lacks cytotoxic activity, e.g., wherein the donor cell is a regulatory T cell (Treg);
wherein the acceptor cell comprises a second docking moiety that binds specifically to the first docking moiety; and
wherein after the transfer the acceptor cell comprises an increased amount of the membrane-associated agent and/or cargo molecule;
thereby modifying the acceptor cell.
   47. A method of modifying an acceptor cell, the method comprising:
contacting the acceptor cell with a donor cell under conditions suitable for transfer of a membrane-associated agent and/or a cargo molecule to the acceptor cell;
wherein the donor cell comprises:
   (a) a membrane-associated agent comprising: (i) a membrane-associated moiety, (ii) a first docking moiety, and optionally (iii) an intracellular moiety, and
   (b) a cargo molecule (e.g., an exogenous cargo molecule);
wherein the donor cell has one or more (e.g., 1, 2, 3, 4, 5, 6, 7, 8, 9, or 10) of the following characteristics:
   (i) the cargo molecule is attached to (e.g., covalently or noncovalently bound to) a cell-penetrating peptide;
   (ii) the membrane-associated agent is attached to (e.g., covalently or noncovalently bound to) a cell-penetrating peptide;
   (iii) the donor cell comprises a cell-penetrating peptide;
   (iv) the donor cell further comprises a fusogen (e.g., a vsv-g protein, e.g., as described in Example 25);
   (v) the donor cell expresses a secreted exogenous effector;
   (vi) the donor cell comprises a virus-like particle (VLP), optionally wherein the VLP infects the acceptor cell;
   (vii) the donor cell inserts a tunneling nanotube to an acceptor cell upon binding of the first docking moiety of the membrane-associated agent to a second docking moiety of the acceptor cell;
   (vii) the donor cell comprises: (1) a nucleic acid molecule comprising a promoter (e.g., a tissue-specific promoter) and a gene encoding the membrane-associated agent and/or a gene encoding the cargo molecule; or (2) a nucleic acid molecule (e.g., an RNA, e.g., an mRNA) encoding the membrane-associated agent and/or a nucleic acid molecule (e.g., an RNA, e.g., an mRNA) encoding the cargo molecule;
   (viii) the donor cell comprises an inducible cell death agent (e.g., an inducible cell death protein, e.g., an inducible caspase, e.g., inducible Caspase 9); and/or
   (ix) the donor cell is or is derived from (e.g., differentiated from) an induced pluripotent stem cell (iPSC) or hematopoietic stem cell (HSC);
wherein the acceptor cell comprises a second docking moiety that binds specifically to the first docking moiety; and
wherein after the transfer the acceptor cell comprises an increased amount of the membrane-associated agent and/or cargo molecule;
thereby modifying the acceptor cell.
   48. The method of any of the preceding embodiments, wherein the donor cell is TCR deficient.
   49. The method of any of the preceding embodiments, wherein the donor cell is MHC deficient.
   50. The method of any of the preceding embodiments, wherein the donor cell comprises a knockout of at least one TCR gene.
   51. The method of embodiment 50, wherein the donor cell comprises a knockout of a TCRα gene.
   52. The method of embodiment 50 or 51, wherein the donor cell comprises a knockout of a TCRβ gene.
   53. The method of any of the preceding embodiments, wherein the contacting occurs in vitro.
   54. The method of any of the preceding embodiments, wherein the contacting occurs in vivo.
   55. The method of any of the preceding embodiments, wherein the contacting occurs in a subject (e.g., a mammalian subject, e.g., a human subject).
   56. The method of any of the preceding embodiments, wherein the contacting results in binding between the first docking moiety of the membrane-associated agent and the second docking moiety.
   57. The method of embodiment 56, wherein the binding between the first docking moiety and the second docking moiety modulates (e.g., increases or decreases) a biological activity in the acceptor cell.
   58. The method of embodiment 57, wherein the biological activity comprises:
(i) expression of a target gene by the acceptor cell,
(ii) an increase or decrease in expression level of a target gene by the acceptor cell, and/or
(iii) activation or inhibition of a biological pathway in the acceptor cell.
   59. A method of producing a donor cell (e.g., a donor cell according to any of the preceding embodiments), the method comprising:
(i) inactivating at least one endogenous function of a cell; and
(ii) introducing into or expressing in the cell:
   (a) a membrane-associated agent comprising: (i) a membrane-associated moiety, (ii) a first docking moiety, and optionally (iii) an intracellular moiety; and
   (b) a cargo molecule (e.g., an exogenous cargo molecule);
thereby producing a donor cell.
   60. The method embodiment 59, wherein (i) comprises inactivating the at least one endogenous function in a parent cell and allowing the parent cell to divide one or more times, thereby producing the cell.
   61. A method of producing a donor cell, the method comprising:
(i) providing a cell that substantially lacks cytotoxic activity, e.g., a regulatory T cell (Treg); and
(ii) introducing into or expressing in the cell:
   (a) a membrane-associated agent comprising: (i) a membrane-associated moiety, (ii) a first docking moiety, and optionally (iii) an intracellular moiety; and
   (b) a cargo molecule (e.g., an exogenous cargo molecule);
   wherein the membrane-associated agent comprises a CAR or an exogenous TCR; thereby producing a donor cell.
      62. The method of any of embodiments 59-61, wherein (i) is performed prior to, concurrently with, or after (ii).
      63. The method of any of embodiments 59-62, wherein the introducing of (ii) is performed in a parent cell and the parent cell is allowed to divide one or more times, thereby producing the cell.
      64. The method of any of embodiments 59-63, wherein (i) comprises inactivating expression of at least one endogenous T cell receptor (TCR) gene or MHC gene in a chromosome of a cell.
      65. The method of embodiment 64, wherein the inactivating of (i) comprises introducing into the cell:
(a) a Cas protein (e.g., a Cas9 protein), or a nucleic acid molecule encoding a Cas protein; and
(b) a guide RNA capable of hybridizing to an endogenous TCR locus (e.g., a TCRα locus or a TCRβ locus), or a portion thereof, in the chromosome of the cell.
   66. The method of embodiment 65, wherein the Cas protein and the guide RNA are comprised in a ribonucleoprotein complex (RNP).
   67. The method of any of embodiments 65-66, wherein the Cas protein, the nucleic acid molecule encoding the Cas protein, and/or the guide RNA are introduced into the cell by electroporation, e.g., as described in Example 28.
   68. The method of any of embodiments 65-67, wherein the Cas protein or the nucleic acid molecule encoding the Cas protein are introduced into the cell prior to, concurrently with, or after the guide RNA.
   69. The method of any of embodiments 59-68, wherein the inactivating comprises:
deleting the endogenous TCR or MHC gene, or a portion thereof (e.g., a portion comprising a start codon);
inserting a stop codon into the coding sequence of the endogenous TCR or MHC gene;
inserting a frameshift into the coding sequence of the endogenous TCR or MHC gene;
introducing a mutation (e.g., a deletion, substitution, or addition) into the endogenous TCR or MHC gene, e.g., a loss of function mutation; and/or
introducing a mutation (e.g., a deletion, substitution, or addition) into a cis-acting element of the endogenous TCR or MHC gene, e.g., the promoter of the endogenous TCR or MHC gene.
   70. The method of any of embodiments 59-69, wherein the inactivating comprises inserting a sequence encoding an exogenous membrane-associated agent, or a subunit thereof, into an endogenous TCR locus (e.g., a TCRα locus or a TCRβ locus) or MHC locus (e.g., a β2-microglobulin locus).
   71. The method of embodiment 70, wherein the exogenous membrane-associated agent comprises an antibody molecule (e.g., an antibody or an antigen-binding fragment thereof, e.g., an scFv).
   72. The method of embodiment 70, wherein the exogenous membrane-associated agent comprises an exogenous TCR.
   73. The method of embodiment 70, wherein the exogenous membrane-associated agent comprises a chimeric antigen receptor (CAR).
   74. The method of any of embodiments 70-73, wherein the exogenous membrane-associated agent is inserted into the endogenous TCR locus by a transposase (e.g., a Sleeping Beauty transposase).
   75. The method of any of embodiments 59-74, further comprising immortalizing the cell.
   76. The method of embodiment 75, wherein the immortalizing comprises introducing into the genome of the cell one or more transgenes encoding immortalization factors (e.g., one or more of hTERT, SV40 Large T antigen, CDK4, and/or HPV16-E6/E7).
   77. The method of embodiment 76, wherein the one or more transgenes encoding the immortalization factors is introduced into the cell by a viral particle (e.g., a lentiviral particle), e.g., as described in Example 31.
   78. The method of any of embodiments 75-77, wherein the immortalizing occurs prior to, concurrently with, or after the inactivating expression of the at least one endogenous TCR gene.
   79. The method of any of embodiments 75-78, wherein the immortalizing occurs prior to, concurrently with, or after the introducing into or expressing in the cell of the membrane associated agent and the cargo molecule.
   80. The method of any of embodiments 59-79, wherein the inactivating comprises: expressing a transposase in the cell or introducing a transposase into the cell; and inserting a sequence encoding an exogenous membrane-associated agent, or a subunit thereof, into the genome of the cell using the transposase.
   81. The method of embodiment 80, wherein the transposase is a Sleeping Beauty transposase.
   82. The method of any of embodiments 80-81, wherein the sequence encoding the exogenous membrane-associated agent, or the subunit thereof, is inserted into an endogenous TCR locus (e.g., a TCRα locus or a TCRβ locus) or an MHC locus (e.g., a β2-microglobulin locus), of the cell.
   83. The method of any of embodiments 59-82, further comprising inserting a gene encoding the membrane-associated agent into the genome of the donor cell.
   84. The method of any of embodiments 59-83, further comprising inserting a gene encoding the cargo molecule into the genome of the donor cell.
   85. The method of embodiment 83 or 84, wherein the inserting comprises integration of the gene into the genome of the donor cell using a transposase (e.g., a Sleeping Beauty transposase).
   86. The method of any of embodiments 59-85, wherein the donor cell stably expresses the membrane-associated agent and/or the cargo molecule.
   87. The method of embodiment 86, wherein the donor cell comprises in its genome a gene encoding the membrane associated agent.
   88. The method of embodiment 86, wherein the donor cell comprises in its genome a gene encoding the cargo molecule.
   89. A method of modifying an acceptor cell, the method comprising:
contacting the acceptor cell with the donor cell of any of the preceding embodiments or a donor cell made by a method of any of the preceding embodiments;
wherein the acceptor cell comprises a second docking moiety that binds specifically to the first docking moiety of the membrane-associated agent of the donor cell; and
wherein after the transfer the acceptor cell comprises an increased amount of the membrane-associated agent and/or cargo molecule of the donor cell;
thereby modifying the acceptor cell.
   90. The method or donor cell of any of the preceding embodiments, wherein the membrane-associated agent comprises a chimeric antigen receptor (CAR), or a functional fragment or variant thereof, and wherein the second docking moiety is specifically bound by the CAR, or the functional fragment or variant thereof.
   91. The method or donor cell of embodiment 90, wherein the second docking moiety is CD19, or a functional fragment or variant thereof, and the CAR specifically binds to CD19, or the functional fragment or variant thereof.
   92. The method or donor cell of any of the preceding embodiments, wherein the membrane-associated agent comprises an antibody molecule, or a functional fragment or variant thereof, and wherein the second docking moiety is specifically bound by the antibody molecule, or the functional fragment or variant thereof.
   93. The method or donor cell of embodiment 92, wherein the antibody molecule is an antibody fragment.
   94. The method or donor cell of embodiment 93, wherein the antibody molecule is an scFv.
   95. The method or donor cell of any of embodiments 92-94, wherein the second docking moiety is CD19, or a functional fragment or variant thereof.
   96. The method or donor cell of any of embodiments 92-95, wherein the membrane-associated agent further comprises a label.
   97. The method or donor cell of embodiment 96, wherein the label is a fluorescent protein, e.g., mRuby or GFP.
   98. The method or donor cell of any of the preceding embodiments, wherein the cargo molecule comprises or is bound to a moiety that specifically binds to the membrane-associated agent, or a portion thereof.
   99. The method or donor cell of any of the preceding embodiments, wherein the membrane-associated agent comprises a CD3zeta domain and the cargo molecule comprises a ZAP70 domain.
   100. The method or donor cell of any of the preceding embodiments, wherein the cargo molecule further comprises a label (e.g., a fluorescent protein, e.g., GFP or mRuby).
   101. The method or donor cell of any of the preceding embodiments, wherein the cargo molecule is attached to (e.g., covalently or noncovalently bound to) a cell-penetrating peptide.
   102. The method or donor cell of any of the preceding embodiments, wherein the donor cell further comprises a fusogen (e.g., a vsv-g protein, e.g., as described in Example 25).
   103. The method or donor cell of embodiment 102, wherein the fusogen is comprised in the cell membrane of the donor cell.
   104. The method or donor cell of any of the preceding embodiments, wherein the donor cell expresses a secreted molecule (e.g., a secreted effector, e.g., a secreted exogenous effector).
   105. The method or donor cell of embodiment 104, wherein the cargo molecule comprises the secreted molecule.
   106. The method or donor cell of any of embodiments 104-105, wherein the secreted molecule comprises a cell-penetrating peptide.
   107. The method or donor cell of any of embodiments 104-106, wherein the cargo molecule comprises a secretion signal.
   108. The method or donor cell of any of the preceding embodiments, wherein the donor cell comprises a virus-like particle (VLP).
   109. The method or donor cell of embodiment 108, wherein the VLP is released upon binding of the first docking moiety of the membrane-associated agent to a second docking moiety of an acceptor cell.
   110. The method or donor cell of embodiment 109, wherein the released VLP infects the acceptor cell.
   111. The method or donor cell of any of the preceding embodiments, wherein the donor cell inserts a tunneling nanotube to an acceptor cell upon binding of the first docking moiety of the membrane-associated agent to a second docking moiety of the acceptor cell.
   112. The method or donor cell of any of the preceding embodiments, wherein the donor cell comprises a nucleic acid molecule (e.g., a chromosome) comprising a gene encoding the membrane-associated agent.
   113. The method or donor cell of embodiment 112, wherein the nucleic acid molecule comprising the gene encoding the membrane-associated agent further comprises a promoter (e.g., a tissue-specific promoter, e.g., a FoxP3 promoter).
   114. The method or donor cell of any of the preceding embodiments, wherein the donor cell comprises an inducible cell death protein (e.g., an inducible caspase, e.g., inducible Caspase 9).
   115. The method or donor cell of embodiment 114, wherein the donor cell comprises a nucleic acid molecule (e.g., a chromosome) encoding the inducible cell death protein.
   116. The method or donor cell of embodiment 114 or 115, wherein the inducible cell death protein activates a cell death pathway in the donor cell when contacted with an activating agent (e.g., a dimerizer, e.g., a small molecule dimerizer).
   117. The method or donor cell of any of the preceding embodiments, wherein the donor cell comprises a selectable marker (e.g., a fluorescent protein or an epitope tag, e.g., a FLAG tag).
   118. The method or donor cell of embodiment 117, wherein the membrane-associated agent of the donor cell is associated with the selectable marker (e.g., wherein the selectable marker is attached to, e.g., covalently or noncovalently, to the membrane-associated agent).
   119. The method or donor cell of any of the preceding embodiments, wherein the donor cell is an induced pluripotent stem cell (iPSC).
   120. The method or donor cell of any of the preceding embodiments, wherein the donor cell is derived from (e.g., is differentiated from) an induced pluripotent stem cell (iPSC).
   121. The method or donor cell of any of the preceding embodiments, wherein the donor is allogeneic to the acceptor cell.
   122. The method or donor cell of any of the preceding embodiments, wherein the donor cell is from a different individual from the acceptor cell.
   123. The method or donor cell of any of the preceding embodiments, wherein the donor cell is autologous to the acceptor cell.
   124. The method or donor cell of any of the preceding embodiments, wherein the donor cell is from the same individual as the acceptor cell.
   125. The method or donor cell of any of the preceding embodiments, wherein the donor cell is not from a subject having cancer (e.g., wherein the donor cell is from a healthy subject).
   126. The method or composition of any of the preceding embodiments, wherein the acceptor cell is comprised in the blood, spleen, bone marrow, lungs, or liver of the subject.
   127. The method or composition of embodiment 126, wherein the acceptor cell is a B cell.
   128. The method or donor cell of any of the preceding embodiments, wherein the acceptor cell is not a cancer cell.
   129. The method or donor cell of any of the preceding embodiments, wherein the acceptor cell is from a subject that does not have a disease or disorder associated with the second docking moiety or a molecule comprising the second docking moiety (e.g., a disease or disorder associated with CD19).
   130. The method or donor cell of any of the preceding embodiments, wherein the donor cell is deficient in (e.g., does not express) an MHC class I protein.
   131. The method or donor cell of any of the preceding embodiments, wherein the donor cell is deficient in (e.g., does not express) an MHC class II protein.
   132. The method or donor cell of any of the preceding embodiments, wherein the donor cell is derived from a primary cell (e.g., a primary T cell, e.g., a regulatory T cell, a CD8+ T cell, or a CD4+ T cell, e.g., a CD4+ helper T cell).
   133. The method or donor cell of any of the preceding embodiments, wherein the donor cell is a lymphocyte (e.g., a T cell).
   134. The method or donor cell of any of the preceding embodiments, wherein the donor cell is a regulatory T cell, a CD8+ T cell, or a CD4+ T cell, e.g., a CD4+ helper T cell.
   135. The method or donor cell of any of the preceding embodiments, wherein the cargo molecule is a polypeptide (e.g., a protein) or a nucleic acid molecule (e.g., a DNA molecule and/or an RNA molecule, e.g., an mRNA molecule).
   136. The method or donor cell of embodiment 135, wherein the cargo molecule is an mRNA comprising an MS2 stem loop sequence (e.g., a 3′ MS2 stem loop sequence).
   137. The method or donor cell of any of the preceding embodiments, wherein the donor cell is immortalized.
   138. The method or donor cell of embodiment 137, wherein the donor cell comprises one or more of: hTERT, SV40 Large T antigen, CDK4, and/or HPV16-E6/E7. 139. The method or donor cell of embodiment 137 or 138, wherein the genome of the donor cell comprises one or more transgenes encoding an immortalization factor (e.g., hTERT, SV40 Large T antigen, CDK4, and/or HPV16-E6/E7).
   140. The method or donor cell of any of the preceding embodiments, wherein the donor cell does not substantially stimulate (e.g., promote, activate, or increase) T cell effector activity.
   141. The method or donor cell of any of the preceding embodiments, wherein the intracellular moiety of the membrane-associated agent does not substantially stimulate (e.g., promote, activate, or increase) T cell effector activity (e.g., of the donor cell).
   142. The method or donor cell of any of the preceding embodiments, wherein the membrane-associated agent does not comprise a CD3-zeta domain, or a functional fragment or variant thereof.
   143. The method or donor cell of any of the preceding embodiments, wherein the first docking moiety is noncovalently attached (e.g., noncovalently bound) to the remainder of the membrane-associated agent.
   144. The method or donor cell of any of the preceding embodiments, wherein the first docking moiety is noncovalently attached (e.g., noncovalently bound) to the membrane-associated moiety.
   145. The method or donor cell of embodiment 143 or 144, wherein the first docking moiety comprises an avidin moiety, e.g., a strepavidin moiety (e.g., a mono-streptavidin moiety), that binds to a biotin moiety on the membrane-associated agent.
   146. The method or donor cell of embodiment 143 or 144, wherein the first docking moiety comprises a biotin moiety that binds to an avidin moiety, e.g., a streptavidin moiety (e.g., a mono-streptavidin moiety), on the membrane-associated agent.
   147. The method or donor cell of any of the preceding embodiments, wherein the first docking moiety is covalently attached (e.g., covalently bound) to the remainder of the membrane-associated agent.
   148. The method or donor cell of any of the preceding embodiments, wherein the first docking moiety is covalently attached (e.g., covalently bound) to the membrane-associated moiety.
   149. The method or donor cell of any of the preceding embodiments, wherein the first docking moiety of the membrane-associated agent specifically binds to an antigen presented on an MHC molecule (e.g., an MHC molecule on the surface of an acceptor cell).
   150. The method or donor cell of any of the preceding embodiments, wherein the second docking moiety comprises an antigen presented by an MHC molecule on the acceptor cell.
   151. The method or donor cell of embodiment 149 or 150, wherein the antigen presented by the MHC comprises a peptide (e.g., a peptide comprising the amino acid sequence SIINFEKL).
   152. The method or donor cell of any of the preceding embodiments, wherein the donor cell comprises a fusogen (e.g., a vsv-g protein).
   153. The method or donor cell of any of the preceding embodiments, wherein the donor cell and the acceptor cell are derived from the same cell type (e.g., HEK 293T cells).
   154. The method or donor cell of any of the preceding embodiments, wherein the donor cell is derived from a primary cell (e.g., a primary T cell, e.g., a primary CD3+ T cell, CD4+ T cell, or CD8+ T cell, or Treg).
   155. The method or composition of any of the preceding embodiments, wherein the donor cell is or is derived from a regulatory T cell (Treg).
   156. The method or composition of embodiment 155, wherein the membrane associated agent comprises a CAR.
   157. The method or composition of embodiment 155, wherein the Treg is a human Treg (e.g., an HLA-02 positive Treg).
   158. The method or composition of any of embodiments 1-154, wherein the donor cell is:
(i) an adipogenic cell, e.g., an adipocyte stem cell (ASC),
(ii) an CD34+ adipocyte, or
(iii) a mature adipocyte.
   159. The method or composition of any of embodiments 1-154, wherein the donor cell is a mesenchymal stem cell (MSC).
   160. The method or donor cell of any of the preceding embodiments, wherein the intracellular moiety comprises a CD28 domain (e.g., wherein the intracellular moiety comprises a CD3-zeta domain and a CD28 domain).
   161. The method or donor cell of any of the preceding embodiments, wherein the donor cell stably expresses the membrane-associated agent (e.g., comprises a nucleic acid sequence encoding the membrane-associated agent in its genome).
   162. The method or donor cell of any of the preceding embodiments, wherein the membrane-associated agent comprises a polypeptide encoded by the nucleic acid sequence of SEQ ID NO: 42, or has an amino acid sequence having at least 75%, 80%, 85%, 90%, 95%, 96%, 97%, 98%, or 99% sequence identity to the encoded polypeptide;
optionally wherein:
   (i) the polypeptide does not comprise an mRuby polypeptide, or
   (ii) the polypeptide does not comprise an amino acid sequence encoded by the portion of SEQ ID NO: 42 encoding an mRuby polypeptide.
      163. An isolated cell comprising an insertion at an endogenous TCR locus of the genome, wherein the insertion comprises a nucleic acid sequence encoding a membrane-associated agent (e.g., a CAR),
      164. The isolated cell of embodiment 163, wherein the cell is a regulatory T cell (Treg).
      165. The isolated cell of embodiment 163 or 164, wherein the nucleic acid sequence encoding a membrane-associated agent is inserted into the endogenous TCR locus using CRISPR (e.g., as described herein).
      166. An isolated regulatory T cell (Treg) comprising a nucleic acid sequence encoding a membrane-associated agent (e.g., a CAR).
      167. The isolated Treg of embodiment 166, wherein the nucleic acid sequence encoding the membrane-associated agent is comprised in the genome of the Treg (e.g., inserted into an endogenous locus of the Treg, e.g., an endogenous TCR locus of the Treg).
      168. A preparation of cells, which is enriched for Tregs of embodiment 166 or 167.
      169. A method of making a donor cell of any of the preceding embodiments, the method comprising:
(i) providing a source cell;
(ii) introducing a nucleic acid molecule (e.g., an mRNA) encoding a membrane-associated agent (e.g., a CAR) into the source cell; and
(iii) optionally, introducing a nucleic acid molecule encoding a cargo molecule into the source cell;
thereby making a donor cell.
   170. The method of embodiment 169, further comprising maintaining the source cell under conditions suitable for expression of the membrane-associated agent.
   171. The method of embodiment 169 or 170, wherein the source cell is a regulatory T cell (Treg), e.g., a human Treg.

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Example 1: TCRs Transfer Specifically to Cells that Express Cognate Peptide-MHC

In this example, T cell receptors (TCRs) were transferred from J76-DMF5 T cells (donors) to T2 cells (acceptors) if the T2 cells were first pulsed with MART-1 antigen. MART-1 presented by HLA-A02 on T2 cells is the specific target of the DMF5 TCR. This shows that specific binding of TCR to cognate peptide-MHC on target cells mediated intercellular transfer of the TCR to the target cell.

### Donor Cell Groups:

1) Jurkat: Human T cell line that expresses a TCR that does not recognize MART-1
2) J76-DMF5: Donor cells derived from Jurkat cells. The native TCR has been knocked out and replaced with the DMF5 TCR, which recognizes MART-1 antigen presented by HLA-A02 receptors.
3) No donor

### Acceptor Cell Groups:

1) T2 cells pulsed with MART-1 peptide
2) T2 cells pulsed with no peptide

The T2 cell line was used as an antigen presenting cell. T2 cells include a defect in antigen processing that allows for direct loading of surface MHC receptors with peptides by incubation. In this experiment, we used T2 cells that were either pulsed with MART-1 peptide or no peptide.

### Cell Identity Labeling:

To distinguish donor cells from acceptor cells, both were labeled with unique dyes that covalently crosslinked to cytoplasmic structures (Thermo CellTracker dyes).

### Donor Labeling:

The surface proteins of donor cells were chemically labeled with biotin using Sulfo-NHS-LC-Biotin, which covalently attached biotin to primary amines (lysine residues and N-termini of proteins). The sulfo group prevented membrane permeability, resulting in surface-only biotinylation of proteins.

### Cell-Cell Contact:

Donor and acceptor cells from each of the above groups were subjected to pairwise co-culture (6 combinations). The cells were first monocultured separately in suspension media after labeling. To co-culture, acceptor cells were added to donor cell cultures at 1:1, 5:1, and 10:1 ratios (donor to acceptor). The co-cultured cells were centrifuged at 100 g to force the cells into contact, then incubated at 37° C. for 2 hours, then placed on ice before processing for flow cytometry.

### Measurement:

Transfer of membrane proteins (biotin) and TCR from donors to acceptors was measured by flow cytometry. All co-cultured cells were stained with viability dye, AlexaFluor750-conjugated streptavidin (to identify biotinylated surface proteins), and MART-1 tetramer (to identify DMF5 TCR). Live T2 cells from each co-culture were gated on their unique CellTracker dye then assessed for surface protein and DMF5 TCR acquisition.

### Results

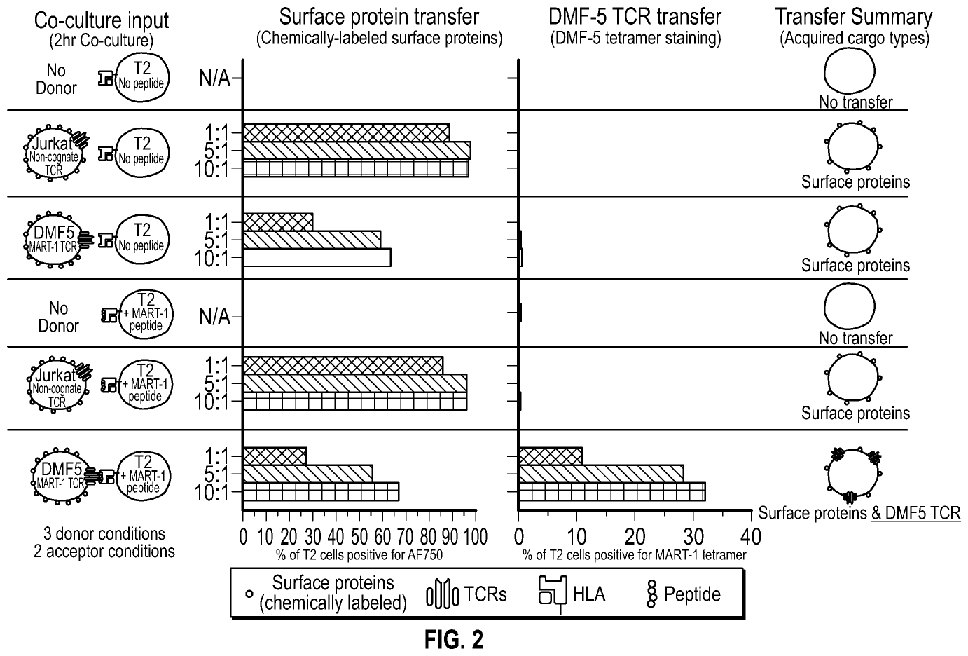

All co-culture pairings resulted in surface protein transfer T2 acceptors (FIGS. 1A and 1B, and FIG. 2, second column). However, DMF5 TCR only transferred to T2 cells that were loaded with cognate MART-1 peptide (FIG. 2, 3^{rd} column). The incidence of both types of transfer increased with increasing donor-to-acceptor ratio. FIG. 2 summarizes the conditions that were tested and resulting transfer of cargo.

### Example 2: T Cell Activation Increases Transfer Incidence and Rate

This example demonstrated that increasing activation of J76-DMF5 donor cells during co-culture resulted in higher transfer rates to T2 acceptors. This may have been due to upregulation of TCR on donors (a non-physiologic response to activation), some other aspect of activation, or both.

### Donor Cells:

1) J76-DMF5: Donor cells derived from Jurkat cells. The native TCR were knocked out and replaced with the DMF5 TCR, which recognized MART-1 antigen presented by HLA-A02 receptors.

### Acceptor Cell Groups:

1) T2 cells pulsed with cognate MART-1 peptide
2) T2 cells pulsed with non-cognate CMV peptide
3) T2 cells pulsed with no peptide

The T2 cell line was used as an antigen presenting cell. It included a defect in antigen processing that allows for direct loading of surface MHC receptors with peptides by incubation. In this experiment, we used T2 cells that were either pulsed with MART-1 peptide or no peptide.

### Cell Identity Labeling:

To distinguish donor cells from acceptor cells, both were labeled with unique dyes that covalently crosslink to cytoplasmic structures (Thermo CellTracker dyes). In this case, donors were labeled with CellTracker Deep Red and acceptors were labeled with CellTracker Violet.

### Cell-Cell Contact Time Course:

Donor and acceptor cells from each of the above groups were subjected to pairwise co-culture (3 combinations). The cells were first monocultured separately in suspension media after labeling. To co-culture, acceptor cells were added to donor cell cultures at a 5:1 ratio (donor to acceptor). The co-cultured cells were centrifuged at 100 g to force the cells into contact, then incubated at 37° C. Each group was sampled at a series of different time points from 15 minutes to 16 hours to assess TCR transfer over time.

### Measurement:

Transfer of TCR from donors to acceptors was measured by flow cytometry. All co-cultured cells were stained with viability dye and MART-1 tetramer (to identify DMF5 TCR). Live T2 cells from each co-culture were gated on CellTracker Violet then assessed DMF5 TCR acquisition.

### Results:

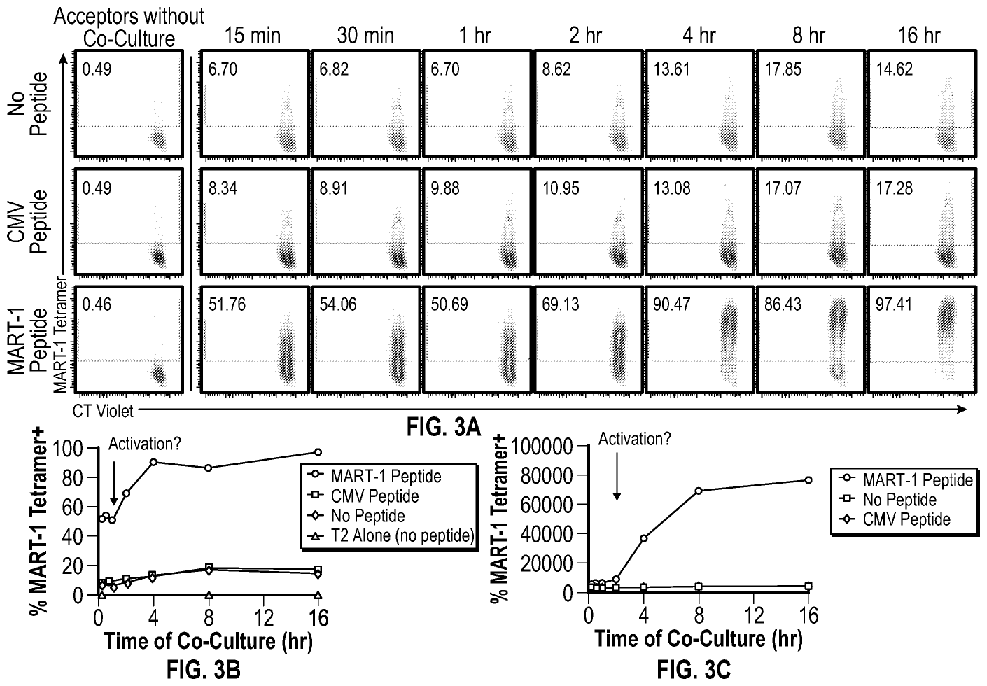

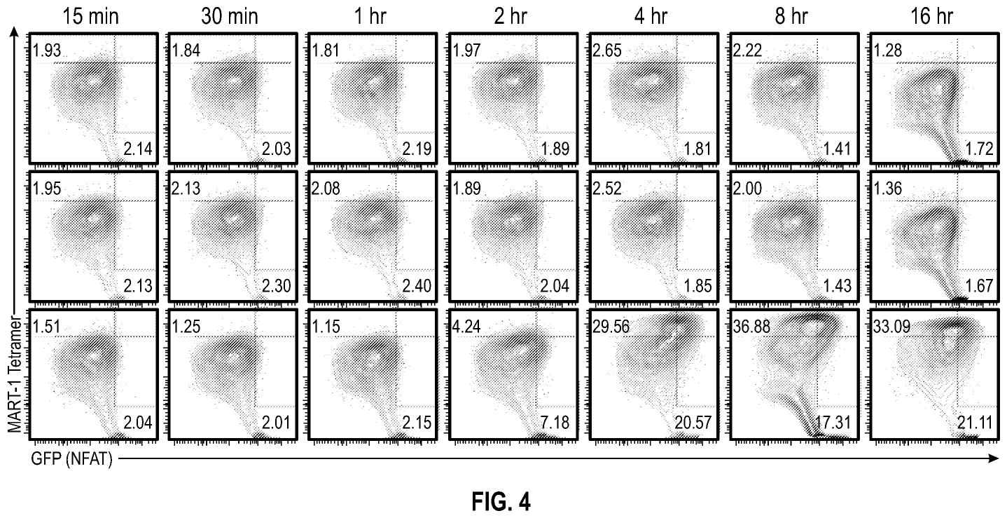

As observed previously, TCR transfer was specific to acceptors that were pulsed with MART-1 peptide (FIG. 3A). Transfer incidence (FIG. 3B) and amount (FIG. 3C) both increased rapidly after 2 hours of co-culture. The increase in transfer correlated with an increase in NFAT signaling in the donors as measured by an NFAT signaling GFP reporter that was built into the J76-DMF5 cell line (FIG. 4). The increased NFAT activation also correlated with upregulation of TCR on the surface of the donors as indicated by MART-1 tetramer staining at later time points. Together, these results indicate that either T cell activation, TCR upregulation, or both, can drive improved transfer of TCR to acceptors.

### Example 3: RNA Cargo Transfer Via Direct and Indirect TCR Fusions to an RNA-Binding Motif

This example demonstrates transfer of mRNA to target cells via binding to a TCR-fused RNA binding protein. It also demonstrates transfer of secondary cargos (a cargo bound to the transferred receptor through another cargo).

### Donor Cells:

1) J76-DMF5: These donor cells were derived from Jurkat cells. The native TCR has been knocked out and replaced with the DMF5 TCR, which recognizes MART-1 antigen presented by HLA-A02 receptors.

### Acceptor Cell Groups:

2) T2 cells pulsed with cognate MART-1 peptide
3) T2 cells pulsed with no peptide

The T2 cell line is commonly used as an antigen presenting cell. It has a defect in antigen processing that allows for direct loading of surface MHC receptors with peptides by incubation. In this experiment, we used T2 cells that were either pulsed with MART-1 peptide or no peptide.

### Cell Identity Labeling:

To distinguish donor cells from acceptor cells, both are labeled with unique dyes that covalently crosslink to cytoplasmic structures (Thermo CellTracker dyes).

### Donor Cargo Expression:

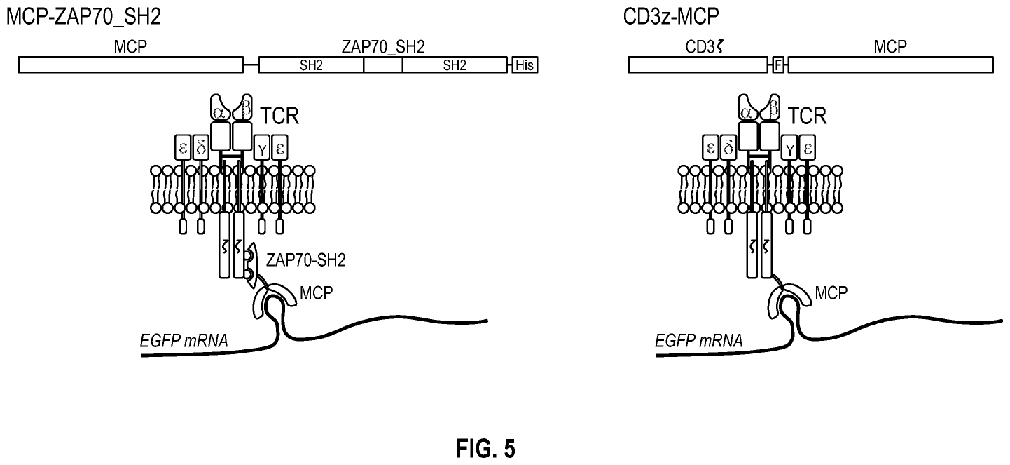

Donor J76-DMF5 cells were transfected with constructs expressing fusion proteins containing a modified RNA binding domain from MS2 coat protein (MCP). As in previous examples, fusion partners included CD3zeta and ZAP70_SH2 domains. (MCP-hZAP70_SH2 and hCD3z-MCP sequences below) (FIG. 5). In the same transfection, mRNA expression constructs were also included. The mRNA expression constructs contained EGFP coding sequence with a 3′ MS2 motif, which binds to the MCP domain. When co-expressed in donor cells, the MS2-containing mRNA binds to the MCP domain of the CD3 and ZAP70 fusion proteins.

### Cell-Cell Contact:

Donor and acceptor cells from each of the above groups were subjected to pairwise co-culture (2 combinations for each construct). The cells were first monocultured separately in suspension media. To co-culture, acceptor cells were added to donor cell cultures at a 5:1 ratio (donor to acceptor). The co-cultured cells were centrifuged at 100 g to force the cells into contact, then incubated at 37° C. for 2 hours.

### Measurement:

EGFP expression was measured in acceptors by flow cytometry. All co-cultured cells were stained with viability dye and MART-1 tetramer (to identify DMF5 TCR). Live T2 cells from each co-culture were gated on CellTracker then assessed for acquisition of DMF5 TCR and EGFP expression.

### Sequences (as Listed in Table 3):

>MCP-ZAP70_SH2
>hCD3z-MCP
>EGFP-MS2 mRNA

### Results:

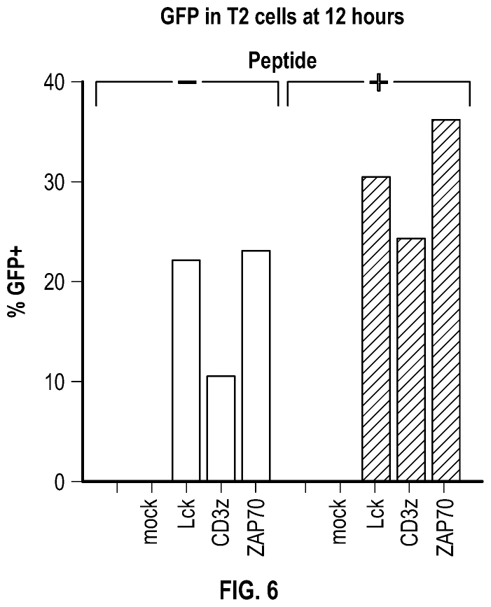

As shown in FIG. 6, GFP signal increased substantially in the peptide-pulsed conditions compared to the no-peptide conditions for each of the Lck, CD3z, and ZAP70 versions of the MCP/EGFP mRNA construct.

### Example 4: Co-Transfer of Cargo During TCR Transfer

This example demonstrated four different strategies for co-transferring a cargo with a TCR.

### Donor Cells:

1) J76-DMF5: Donor cells derived from Jurkat cells. The native TCR has been knocked out and replaced with the DMF5 TCR, which recognized MART-1 antigen presented by HLA-A02 receptors.

### Acceptor Cell Groups:

2) T2 cells pulsed with cognate MART-1 peptide
3) T2 cells pulsed with no peptide

The T2 cell line was used as an antigen presenting cell. It has a defect in antigen processing that allows for direct loading of surface MHC receptors with peptides by incubation. In this experiment, we used T2 cells that were either pulsed with MART-1 peptide or no peptide.

### Cell Identity Labeling:

To distinguish donor cells from acceptor cells, both were labeled with unique dyes that covalently crosslinked to cytoplasmic structures (Thermo CellTracker dyes).

### Donor Cargo Expression:

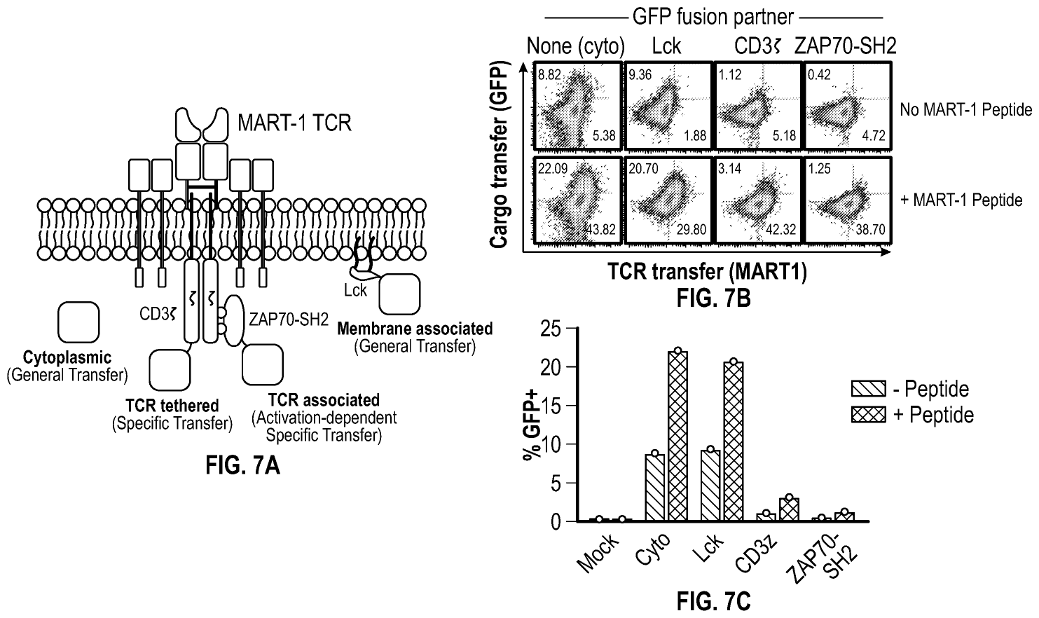

Donor J76-DMF5 cells were transfected with one of four different constructs that expressed EGFP (cargo) associated in different ways with the TCR:
1) Membrane-associated: EGFP fused to the palmitoylation/myristoylation domain of Lek (also referred to as PM-EGFP). This generated an EGFP protein tethered to the inner leaflet of the plasma membrane (FIG. 7A; “Lck”).
2) TCR-tethered: EGFP fused to the c-terminus of human CD3-zeta. This generated an EGFP protein that was covalently linked to the TCR (FIG. 7A).
3) TCR-associated: EGFP fused to the n-terminus of a truncated version of human ZAP70 that contains only its SH2 domains. This generated an EGFP protein that associated with the TCR only when the TCR has been triggered (FIG. 7A).
4) General Transfer: Cytoplasmic EGFP. This generated an EGFP protein with no specific association with TCR beyond co-expression in the same cell (FIG. 7A; “Cytoplasmic” or “Cyto”).

### Sequences (as Listed in Table 3):

>PM-EGFP
>CD3z-EGFP
>EGFP-ZAP70_SH2

### Cell-Cell Contact:

Donor and acceptor cells from each of the above groups were subjected to pairwise co-culture (2 combinations). The cells were first monocultured separately in suspension media. To co-culture, acceptor cells were added to donor cell cultures at a 5:1 ratio (donor to acceptor). The co-cultured cells were centrifuged at 100 g to force the cells into contact, then incubated at 37° C. for 2 hours.

### Measurement:

Transfer of EGFP from donors to acceptors was measured by flow cytometry. All co-cultured cells were stained with viability dye and MART-1 tetramer (to identify DMF5 TCR). Live T2 cells from each co-culture were gated on CellTracker then assessed for acquisition of DMF5 TCR (MART-1 tetramer staining) and EGFP fluorescence.

### Results

Acquisition of DMF5 TCR and EGFP by acceptor T2 cells (with or without pre-pulsing the T2 cells with MART-1 peptide) was measured by flow cytometry. Pre-pulsing with MART-1 peptide resulted in an increase in GFP transfer in all four co-transfer conditions tested (FIGS. 7B and 7C).

### Example 5: Delivery of Receptor-Associated Cargos In Vivo

This example tested whether the cargo transfer receptors outlined in previous examples could deliver cargo to target cells in vivo.

### Donor Cells:

1) Mouse primary CD8 T cells: These donor cells were harvested from perforin knockout mice (Jax stock #002407). Perforin deficiency rendered the cells unable to kill target cells through perforin/granzyme-dependent mechanisms.

### Recipient Animals:

2) RIP-mOVA mice (Jax stock #05431). These mice expressed a surface-tethered version of ovalbumin (via transferrin receptor fusion) under the control of the rat insulin promotor, which drove expression primarily in pancreatic beta cells. There was also some expression in proximal tubule cells of the kidney, and weak expression in the testes. This expression allowed the cells to be targeted by Ova-specific T cells from OT-1 transgenic mice as a model of type 1 diabetes.

### Cell Identity Labeling:

To distinguish donor cells in vivo, they were labeled with a dye that covalently crosslinks to cytoplasmic structures (Thermo CellTracker dye). These dyes have been shown to stably label cells for long-term in vivo tracking.

### Donor Cell Modifications:

Donor T cells were co-transfected with constructs overexpressing OT1 TCR (specific for ovalbumin peptide presented on MHC class I) and cargo transfer receptor systems including the following:
1) mCD3z-EGFP+Lck-Scarlet-I+OT-1 TCR
2) mCD3z-EGFP+Lck-Scarlet-I

### Sequences (as Listed in Table 3):

>OT1 TCR
>mCD3z-EGFP
>Lck-Scarlet-I

### Recipient Animal Conditioning:

RIP-mOVA recipient mice were pre-treated with streptozotocin, which is specifically toxic to pancreatic beta cells. A low dose of streptozotocin generated inflammation without killing beta cells. This was intended to model a pre-diabetic state and to promote migration of OT-1 T cells to pancreatic islets to target beta cells.

### Cell Injections:

The transfected donor cells were injected into recipient animals via tail vein injection.

### Measurement:

On days 1, 2, and 3 post-injection, animals were euthanized and pancreas, kidney, and testes were collected for analysis. From the pancreata, islets were purified and dissociated into single cell suspensions. The suspensions were stained according to the panel in Table 4 and cargo transfer was measured by flow cytometry. Cells were gated on live/dead, then assessed for Cytotracker dye, cell type markers, and EGFP. The kidney and testes were sent for histology to identify t cell infiltration. The tissues were frozen in OCT for cryosectioning and analysis by immunofluorescent or IHC staining.

TABLE 4
Staining for single cell suspensions from pancreatic islets.

| | | | Pancreatic islets + | | | |
|---|---|---|---|---|---|---|
| Channel | Center | width | OTI T flow panel | OT I T | beta cell | Comment |
| VL1 | 440 | 50 | Glucagon-BV421 | − | − | alpha cell ID (intracellular) |
| VL2 | 525 | 50 | | | | |
| VL3 | 610 | 20 | | | | |
| VL4 | 660 | 20 | | | | |
| VL5 | 710 | 50 | CD8-BV711 | | +? | CD8 T cell ID, may transfer |
| VL6 | 780 | 60 | | | | |
| BL1 | 530 | 30 | GFP | ++ | +? | Cargo, to transfer |
| BL2 | 695 | 40 | | | | |
| YL1 | 585 | 16 | | | | |
| YL2 | 620 | 15 | PM-Scarlet | ++ | +? | OT I PM, to transfer to beta cell |
| YL3 | 780 | 60 | tetramer-PECy7 | ++ | +? | OT I TCR, to transfer to beta cell |
| RL1 | 610 | 14 | CT Deep Red | ++ | − | Adoptively-transferred cell primary ID |
| RL2 | 720 | 30 | Insulin-A700 | − | ++ | beta cell ID (intracellular) |
| RL3 | 780 | 60 | L/D NIR | | | viability |

### Results:

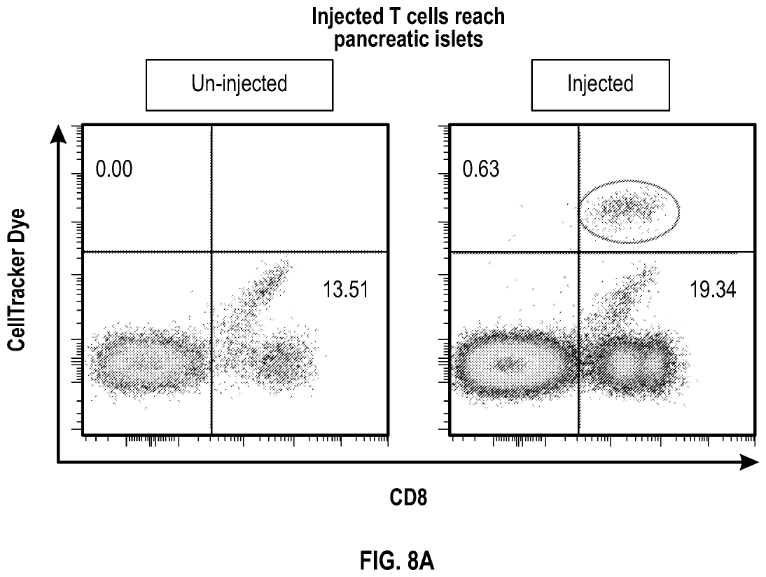

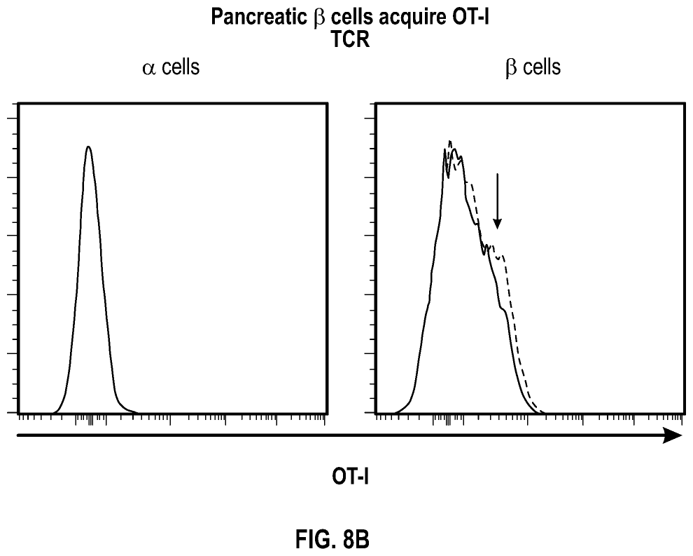

Islets were isolated 2 days post-injection and dissociated into single-cell suspensions. CytoTracker dye-positive cells were detected in islets from an injected mouse, but not a control mouse that was not injected (FIG. 8A). OT-1 TCR (measured by MHC tetramer staining) was detected on pancreatic β cells, identified by intracellular insulin staining (FIG. 8B, right panel). No OT-1 TCR was detected on a cells, identified by intracellular glucagon staining (FIG. 8B, left panel).

### Example 6: Transfer of Chimeric Antigen Receptors to Target B Cells

In this example, whether chimeric antigen receptors (CARs) are transferred to target cells was assessed.

### Donor Cell Groups:

1) J76: Donor cells derived from Jurkat cells. The native TCR has been knocked out.
2) No donor

### Acceptor Cell Groups:

1) Ramos cells: This is a B cell line that expresses CD19.
2) Raji cells.
3) T2 cells.

### Cell Identity Labeling:

To distinguish donor cells from acceptor cells, both were labeled with unique dyes that covalently crosslink to cytoplasmic structures (Thermo CellTracker dyes).

### Donor CAR Expression:

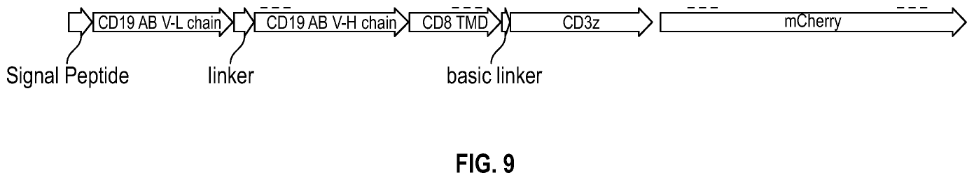

Donor J76 cells were electroporated with a construct expressing a CAR that recognizes CD19 (FIG. 9). The intracellular domain of the CAR contains a signaling domain derived from CD3-zeta and a c-terminal mCherry fluorescent protein.

### Sequence (as Listed in Table 3):

>CD19CAR-CD3z-mCherry

### Cell-Cell Contact:

Donor and acceptor cells from each of the above groups were subjected to pairwise co-culture (2 combinations). The cells were first monocultured separately in suspension media after labeling. To co-culture, acceptor cells were added to donor cell cultures at 1:1, 5:1, and 10:1 ratios (donor to acceptor). The co-cultured cells were centrifuged at 100 g to force the cells into contact, then incubated at 37° C. for 2 hours, then placed on ice before processing for flow cytometry.

### Measurement:

Transfer of the CAR from donors to acceptors was measured by flow cytometry. All co-cultured cells were stained with viability dye. Live T2 cells from each co-culture are gated on CellTracker Violet then assessed for mCherry fluorescence, indicating transfer of the CAR.

### Results:

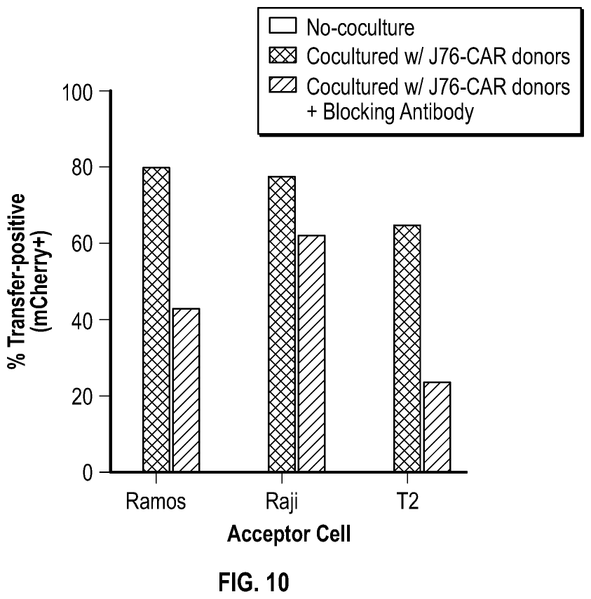

We measured transfer of CD19CAR-CD3z-mCherry to acceptor cells. Transfer incidence, measured as the percent of acceptor cells that acquired the CAR is summarized in FIG. 10 and in the table below:

| | | | Co-culture w/ |
|---|---|---|---|
| | | Co-culure | J76-CAR + CD19 |
| | No co-culture | w/J76-CAR | Blocking Antibody |
| Ramos | 0.26% | 80.27% | 43.11% |
| Raji | 0.23% | 77.93% | 62.13% |
| T2 | 0.45% | 64.99% | 23.85% |

### Example 7: Transfer of Chimeric Antigen Receptors to Other Target Cells

The previous example showed target-specific transfer of CARs to B cells via recognition of CD19. Here, we demonstrate that this CAR transfer is generalizable, and that changing the extracellular targeting domain is sufficient to designate a new target cell type.

### Donor Cells:

1) J76: Donor cells derived from Jurkat cells. The native TCR has been knocked out.
2) No donor

### Acceptor Cell Groups:

1) HEK293 cells
2) HEK293 cells expressing CD19
3) HEK293 cells expressing EGFRt

### Cell Identity Labeling

To distinguish donor cells from acceptor cells, both are labeled with unique dyes that covalently crosslink to cytoplasmic structures (Thermo CellTracker dyes).

### Donor CAR Expression:

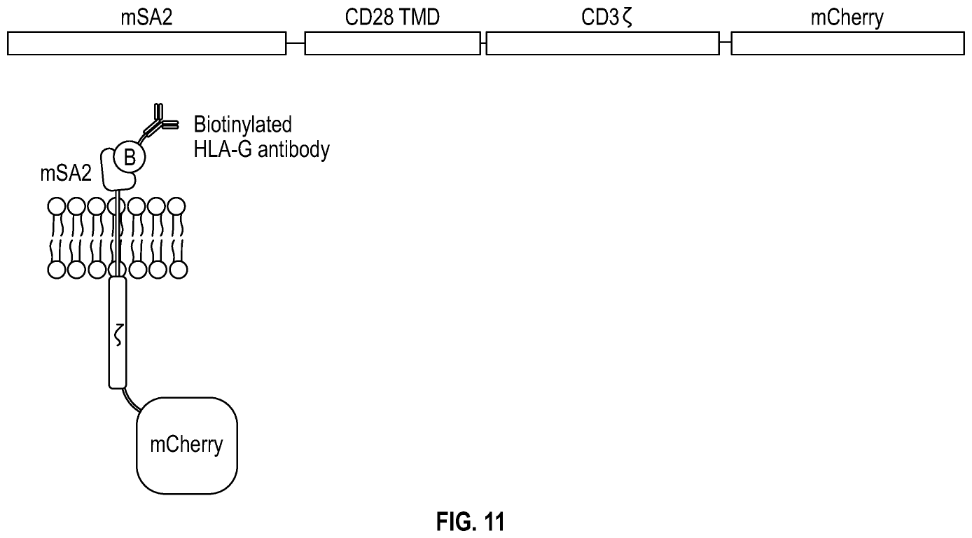

Donor J76 cells are transfected with a construct that expresses a retargetable CAR composed of 3 main domains: an intracellular cargo domain (or cargo-binding domain), a transmembrane domain, and an extracellular target cell binding domain (FIG. 11). In this example, the extracellular domain contains a re-targetable monovalent streptavidin mutant called mSA2 which allows target cell specificity to be re-directed by a biotinylated antibody that recognizes the target (in this example, anti-HLA-G antibody is used). The intracellular portion contains a signaling domain derived from CD3-zeta and mCherry. (mSA2-CAR).

mSA2-CAR Sequence (as Listed in Table 3):
>mSA2 CAR-CD3z-mCherry

### Donor CAR Targeting:

Transfected donor cells are incubated with biotinylated antibodies that recognize either CD19 or EGFRt, depending on the target cell expression. The cells are washed extensively to remove unbound antibody. This step provides a target specificity for the CAR receptors by binding of the mSA2 domain to the biotin of the biotin-HLA-G.

### Cell-Cell Contact:

Donor and acceptor cells from each of the above groups are subjected to pairwise co-culture (6 combinations). The cells are first monocultured separately in suspension media. To co-culture, acceptor cells are added to donor cell cultures at a 5:1 ratio (donor to acceptor). The co-cultured cells are centrifuged at 100 g to force the cells into contact, then incubated at 37° C. for 2 hours.

### Measurement:

Transfer of CAR receptors from donors to acceptors is measured by flow cytometry. All co-cultured cells are stained with viability dye. Live HEK293 cells from each co-culture are gated on CellTracker then assessed for mCherry expression, which indicates CAR transfer.

### Example 8: RNA Cargo Transfer Via Direct and Indirect CAR Fusions to an RNA-Binding Motif

This example demonstrates transfer of mRNA to target cells via binding to a CAR-fused RNA binding protein. It also demonstrates transfer of secondary cargos (a cargo bound to the transferred receptor through another cargo).

### Donor Cell Groups:

1) J76: Donor cells derived from Jurkat cells. The native TCR has been knocked out.
2) No donor

### Acceptor Cell Groups:

1) Ramos cells: This is a B cell line that expresses CD19.

### Cell Identity Labeling:

To distinguish donor cells from acceptor cells, both are labeled with unique dyes that covalently crosslink to cytoplasmic structures (Thermo CellTracker dyes).

### Donor CAR and Cargo Expression:

Donor J76 cells are electroporated with constructs expressing a CD19 CAR (described in Example 6), an RNA binding protein cargo, and an mRNA that encodes EGFP (including 2 MS2 stem loop motifs). The RNA binding protein is the MS2 coat protein (MCP), which binds specifically to the RNA stem-loop motif referred to as an “MS2 site.” Four different cargo transfer strategies are tested:
1) Membrane-associated: MCP fused to the palmitoylation/myristoylation domain of Lek (also referred to as PM-MCP). This generates an MCP protein tethered to the inner leaflet of the plasma membrane.
2) CAR-tethered: MCP fused directly to the c-terminus of the CAR
3) CAR-associated: MCP fused to the n-terminus of a truncated version of human ZAP70 that contains only its SH2 domains. This generates an MCP protein that associates with the CAR only when the CAR has been triggered. The association occurs through phosphorylated residues of the CD3-zeta domain.
4) General Transfer: No MCP protein is provided. This tests whether an un-tethered, cytoplasmic RNA can be transferred.

### Amino Acid Sequence (as Listed in Table 2):

>MCP protein sequence

### Nucleic Acid Sequences (as Listed in Table 3):

>MCP-ZAP70_SH2
>MCP DNA sequence
>hCD3z-MCP
>EGFP-MS2 mRNA

### Cell-Cell Contact:

Donor and acceptor cells from each of the above groups are subjected to pairwise co-culture (2 combinations per cargo, 8 total). The cells are first monocultured separately in suspension media. To co-culture, acceptor cells are added to donor cell cultures at a 5:1 ratio (donor to acceptor). The co-cultured cells are centrifuged at 100 g to force the cells into contact, then incubated at 37° C. for 2 hours.

### Measurement:

Transfer of mRNA cargo from donors to acceptors is measured by flow cytometry. All co-cultured cells are stained with viability dye. Live Ramos cells from each co-culture are gated on CellTracker then assessed for acquisition the CAR by mCherry fluorescence and acquisition of the cargo by EGFP fluorescence (expressed from the transferred mRNA).

### Example 9: Co-Transfer of RNA Cargo with Chimeric Antigen Receptors

This example demonstrates transfer of mRNA to target cells via binding to a CAR-fused RNA binding protein.

### Donor Cells:

2) J76: Donor cells derived from Jurkat cells. The native TCR has been knocked out.

### Acceptor Cell Groups:

1) Ramos cells: This is a B cell line that expresses CD19.

### Cell Identity Labeling:

To distinguish donor cells from acceptor cells, both were labeled with unique dyes that covalently crosslink to cytoplasmic structures (Thermo CellTracker dyes).

### Donor Cargo Expression:

Donor J76 cells were electroporated with either:
1) Mock: electroporated without plasmid
2) EGFP-MS2: GFP mRNA with a 3′ MS2 stem loop sequence
3) CAR-MCP: A CD19 CAR with an intracellular RNA binding protein domain (MCP). An MS2-tagged mRNA encoding GFP is co-transfected.
4) CAR-MCP & GFP-MS2: A CD19 CAR (detectable via mCherry intracellular domain) and a ZAP70 protein fused to MCP. An MS2-tagged mRNA encoding GFP is co-transfected.

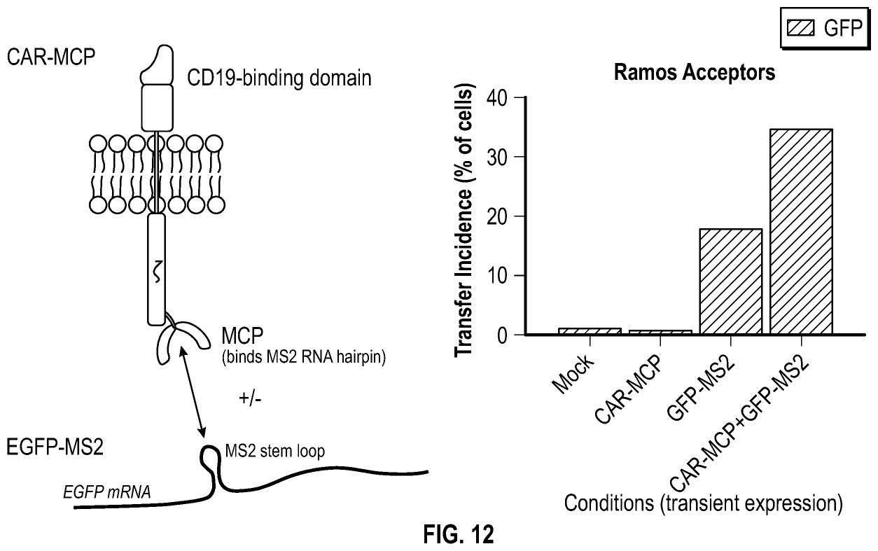

The left panel of FIG. 12 shows a diagram of these parts.

### Amino Acid Sequence (as Listed in Table 2):

>CD19CAR-MCP

### Nucleic Acid Sequences (as Listed in Table 3):

>EGFP-MS2

### Cell-Cell Contact:

Donor and acceptor cells from each of the above groups were subjected to pairwise co-culture (2 combinations for each construct). The cells were first monocultured separately in suspension media. To co-culture, acceptor cells were added to donor cell cultures at a 5:1 ratio (donor to acceptor). The co-cultured cells were centrifuged at 100 g to force the cells into contact, then incubated at 37° C. for 2 hours.

### Measurement:

Transfer of EGFP-encoding RNA from donors to acceptors is measured by flow cytometry at 12 hours after co-culture. All co-cultured cells were stained with viability dye. Live Ramos cells from each co-culture are gated on CellTracker then assessed for acquisition of the EGFP mRNA by measuring EGFP fluorescence.

### Results:

When GFP mRNA alone was expressed in donor cells, we observed transfer to 17.95% of Ramos cells. When GFP mRNA was co-expressed with CAR-MCP, this transfer approximately doubled to 34.83% of Ramos cells (FIG. 12, right panel).

### Example 10: Co-Transfer of Cargo with Chimeric Antigen Receptors

In this example, whether cargo expressed in a CAR-bearing cell can be co-transferred to target cells is assessed.

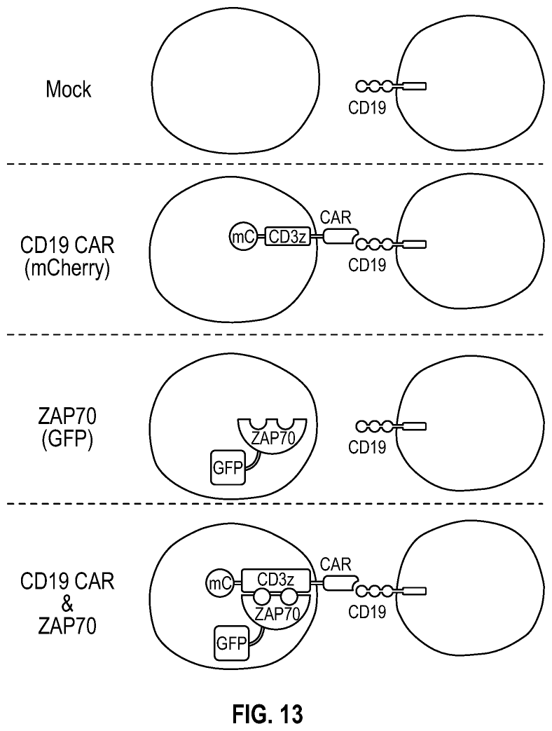

### Donor Cell Groups (as Shown in FIG. 13):

1) J76 (mock electroporated): Donor cells derived from Jurkat cells. The native TCR has been knocked out.
2) J76+CD19 CAR-mCherrry: mCherry fused to the C-terminus of the CAR
3) J76+ZAP70-EGFP: GFP fused to the N-terminus of a truncated version of human ZAP70 that contains only its SH2 domains. This generates an EGFP protein that associates with the CAR only when the CAR has been triggered. This condition does not include the CAR.
4) J76+CD19 CAR-mCherrry+ZAP70-EGFP: Co-transfection of both the CAR and the ZAP70-GFP cargo.

### Acceptor Cell Groups:

2) Ramos cells: This is a B cell line that expresses CD19.

### Cell Identity Labeling:

To distinguish donor cells from acceptor cells, both were labeled with unique dyes that covalently crosslink to cytoplasmic structures (Thermo CellTracker dyes).

### Donor CAR and Cargo Expression:

Donor J76 cells were electroporated with constructs expressing a CD19 CAR (as described in Example 6) and a ZAP70-EGFP fusion cargo (sequence below) according to the donor groups above.

### Sequences (as Listed in Table 3):

>CD19CAR-CD3z-mCherry
>EGFP-ZAP70_SH2

### Cell-Cell Contact:

Donor and acceptor cells from each of the above groups were subjected to pairwise co-culture (2 combinations). The cells were first monocultured separately in suspension media. To co-culture, acceptor cells were added to donor cell cultures at a 5:1 ratio (donor to acceptor). The co-cultured cells were centrifuged at 100 g to force the cells into contact, then incubated at 37° C. for 2 hours.

### Measurement:

Transfer of EGFP from donors to acceptors was measured by flow cytometry. All co-cultured cells were stained with viability dye. Live Ramos cells from each co-culture were gated on CellTracker then assessed for acquisition the CAR by mCherry fluorescence and acquisition of the cargo by EGFP fluorescence.

### Results:

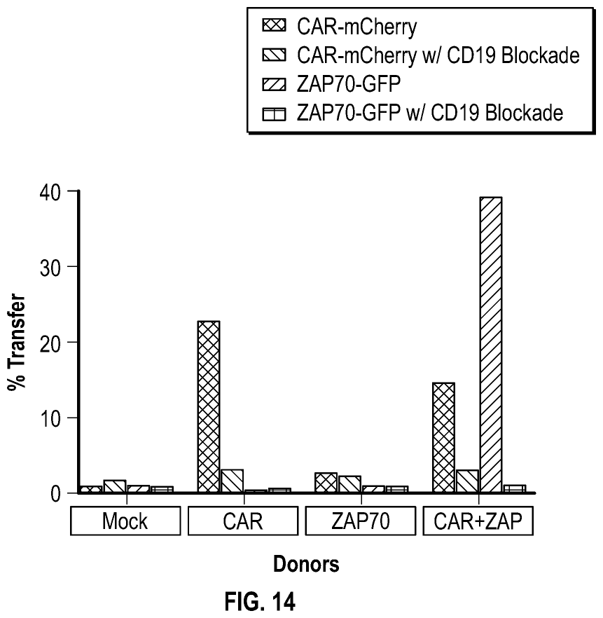

As shown in FIG. 14, we observed transfer of the CAR to CD19-expressing acceptor cells (Ramos), measured by mCherry fluorescence (labeled “CAR” in FIG. 14). ZAP70 was not transferred when expressed alone in J76 cells. (labeled “ZAP70” in FIG. 14). However, when expressed together with the CAR, ZAP70 was efficiently transferred to Ramos acceptors (“CAR+ZAP70” in FIG. 14).

### Example 11: Co-Transfer of Cargo Using a Different Donor Cell

In this example, we tested whether non-T cells can be used as donors for CAR-based transfer.

### Donor Cells:

K562: Human cell line derived from a myeloid leukemia

### Acceptor Cells:

Ramos cells: B cell line that expresses CD19.

### Cell Identity Labeling:

To distinguish donor cells from acceptor cells, both were labeled with unique dyes that covalently crosslink to cytoplasmic structures (Thermo CellTracker dyes).

### Donor CAR and Cargo Expression:

Donor K562 cells were electroporated with constructs expressing a CD19 CAR (e.g., as described in Example 6) and a cargo (FIG. 9). Four different CAR/cargo combinations were tested:
1) Mock: mock electroporation with no CAR or ZAP70-GFP cargo
2) CAR: mCherry fused directly to the c-terminus of the CAR
3) ZAP70-GFP: GFP fused to the n-terminus of a truncated version of human ZAP70 that contains only its SH2 domains. This generates an EGFP protein that associates with the CAR only when the CAR has been triggered. This condition does not include the CAR.
4) CAR+ZAP70-GFP: Co-transfection of both the CAR and the ZAP70-GFP cargo.

### Sequences (as Listed in Table 3):

>CD19CAR-CD3z-mCherry
>EGFP-ZAP70_SH2

### Cell-Cell Contact:

Donor and acceptor cells from each of the above groups were subjected to pairwise co-culture (4 conditions). The cells were first monocultured separately in suspension media. To co-culture, acceptor cells were added to donor cell cultures at a 5:1 ratio (donor to acceptor). The co-cultured cells were centrifuged at 100 g to force the cells into contact, then incubated at 37° C. for 2 hours.

### Measurement:

Transfer of CAR(mCherry) and ZAP-70(EGFP) from donors to acceptors was measured by flow cytometry. All co-cultured cells were stained with viability dye. Live Ramos cells from each co-culture were gated on CellTracker then assessed for acquisition the CAR and/or ZAP70 cargo by mCherry and EGFP fluorescence, respectively.

### Results:

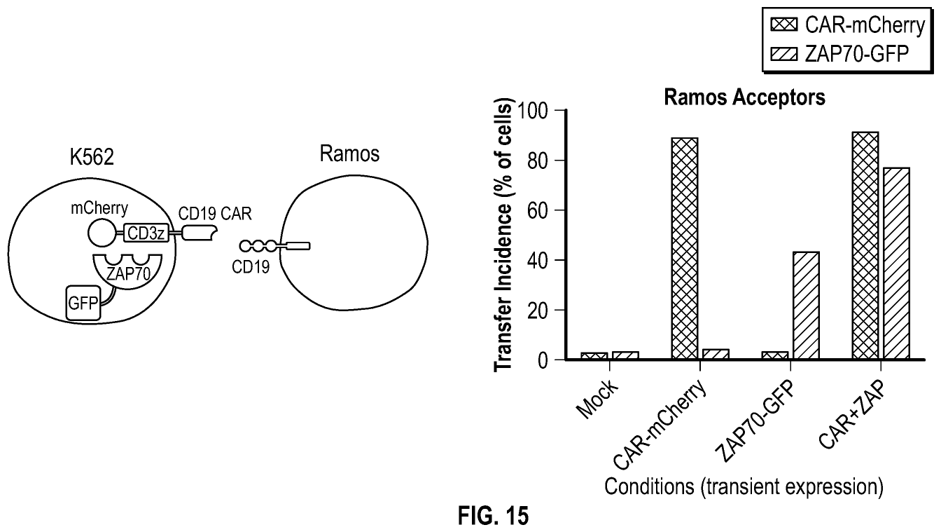

As we observed with T cell donors, K562s were able to co-transfer CD19CAR-CD3z-mCherry and ZAP70-GFP to Ramos acceptors. Unlike the T cells, K562s showed some ZAP70 transfer in the absence of the CAR, but the CAR was able to increase the transfer when co-expressed (FIG. 15).

### Example 12: Delivery of Receptor-Associated Cargos In Vivo

This example tests whether the cargo transfer receptors outlined in previous examples can deliver cargo to target cells in vivo.

### Donor Cells:

1) Mouse primary CD8 T cells: These donor cells are harvested from perforin knockout mice (Jax stock #002407). Perforin deficiency renders the cells unable to kill target cells through perforin/granzyme-dependent mechanisms.

### Recipient Animals:

1) C57BL/6J (Jax stock #000664) or Ai6 (Jax stock #007906) mice fed Ovalbumin or SIINFEKL peptide
2) C57BL/6J or Ai6 mice treated topically with Ovalbumin or SIINFEKL peptide
3) C57BL/6J or Ai6 mice (untreated)

### Cell Identity Labeling:

To distinguish donor cells in vivo, they are labeled with a dye that covalently crosslinks to cytoplasmic structures (Thermo CellTracker dye). These dyes have been shown to stably label cells for long-term in vivo tracking.

### Donor Cell Modifications:

Donor T cells are co-transfected with constructs overexpressing OT1 TCR (specific for ovalbumin peptide presented on MHC class I) and cargo transfer receptor systems including:
1) mCD3z-EGFP
2) EGFP-mZAP70_SH2
3) Cre-mZAP70_SH2
4) MCP-mZAP70_SH2+MS2 Cre mRNA.
   OT1 TCR and cargo transfer receptor sequences are listed below. EGFP-based constructs (1,2) are for use in C57BL/6J recipients, whereas Cre-based constructs (3,4) are for use in Ai6 mice, which are a Cre reporter strain.

### Sequences (as Listed in Table 3):

>OT1 TCR
>mCD3z-EGFP
>EGFP-mZAP70_SH2
>Cre-mZAP70_SH2
>MCP-mZAP70_SH2

### Antigen Pulse:

Mice are pre-treated with antigen or vehicle for 1 week (dietary) or 2 days (topical, 1 application per day) prior to cell injections.

### Cell Injections:

Modified donor cells are injected into recipient animals via tail vein injection.

### Measurement:

Two to four days post-injection, animals are euthanized and skin and gut are collected. Skin samples are taken from topically treated and untreated areas. Cargo transfer is measured by flow cytometry, histology, or whole-mount microscopy. For flow cytometry the tissues are dissociated and stained for cell type specific markers, then assessed for Cytotracker dye, cell type markers, and EGFP or the Cre reporter (ZsGreen). For histology, the tissues are frozen in OCT for cryosectioning and analysis by fluorescent microscopy. For whole, mount, the tissues are left intact and imaged directly for EGFP/ZsGreen and cytotrackers. Cytotracker-negative, EGFP/ZsGreen-positive cells indicate successful in vivo transfer.

### Example 13: Specific Transfer to Target Cells Among a Mixed Population

In this example, transfer specificity was tested by exposing donors to a mixed population containing a minority of target cells. The results (shown below) demonstrate that CAR transfer occurred specifically to B cells rather than to other cells present in the target/acceptor cell mixture of PBMCs.

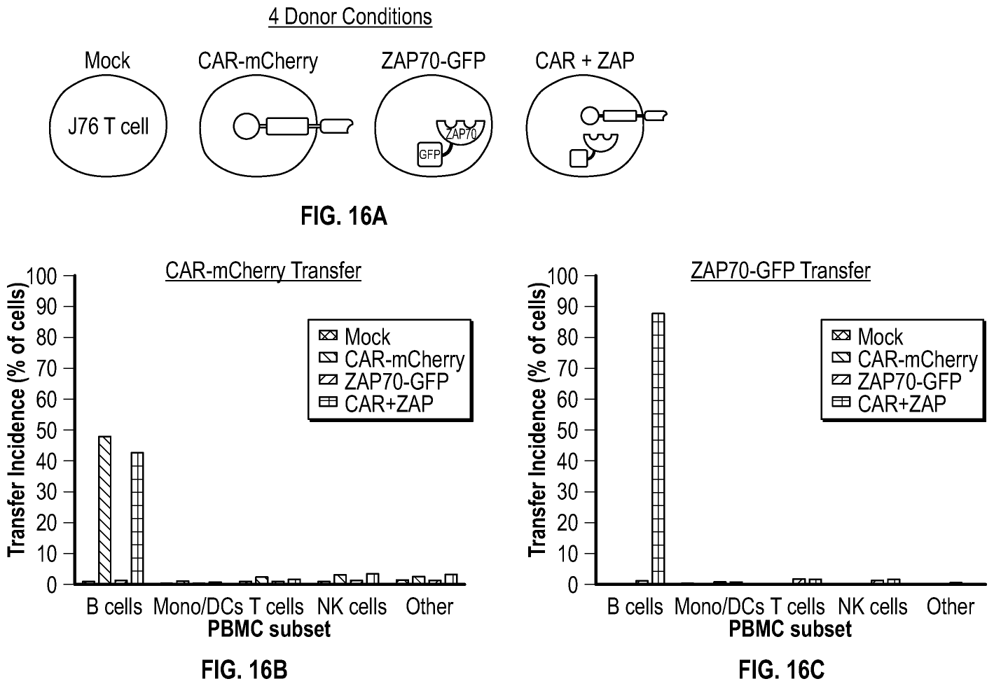

### Donor Cell Groups (as Shown in FIG. 16A):

1) J76 (mock electroporated): Donor cells derived from Jurkat cells. The native TCR has been knocked out.
2) J76+CD19 CAR-mCherrry: mCherry fused to the C-terminus of the CAR
3) J76+ZAP70-GFP: GFP fused to the N-terminus of a truncated version of human ZAP70 that contains only its SH2 domains. This generates an GFP protein that associates with the CAR only when the CAR has been triggered. This condition does not include the CAR.
4) J76+CD19 CAR-mCherrry+ZAP70-GFP: Co-transfection of both the CAR and the ZAP70-GFP cargo.

### Acceptor Cells:

Human peripheral blood mononuclear cells (PBMCs) consisting of approximately 9.5% B cells, 73.5% T cells, 3.5% NK cells, 12.5% Monocytes, and 1% unclassified cells (Other). B cells are the intended target, being the only cells that express the target antigen of the CAR, CD19.

### Cell Identity Labeling:

To distinguish donor cells from acceptor PBMCs, both were labeled with unique dyes that covalently crosslink to cytoplasmic structures (Thermo CellTracker dyes).

### Donor CAR and Cargo Expression:

Donor J76 cells were electroporated with constructs expressing a CD19 CAR (as described in Examples 6 and 10) and a ZAP70-GFP fusion cargo (sequence below) according to the donor groups above.

### Sequences (as Listed in Table 3):

>CD19CAR-CD3z-mCherry
>EGFP-ZAP70_SH2

### Cell-Cell Contact:

Donor and acceptor cells from each of the above groups were subjected to co-culture (4 combinations). The cells were first monocultured separately in suspension media. To co-culture, acceptor cells were added to donor cell cultures at a 5:1 ratio (donor to acceptor). The co-cultured cells were centrifuged at 100 g to force the cells into contact, then incubated at 37° C. for 1 hours.

### Measurement:

Transfer of EGFP from donors to acceptors was measured by flow cytometry. PBMC cell types were identified using specific antibody staining. All co-cultured cells were stained with viability dye. Live PBMCs from each co-culture were gated on CellTracker then assessed for acquisition of the CAR by mCherry fluorescence and acquisition of the cargo by EGFP fluorescence.

### Results:

As shown in FIG. 16B, we observed transfer of the CAR to CD19-expressing B cells whenever the CAR was present in the donors (conditions labeled as “CAR-mCherry” & “CAR+ZAP” in FIG. 16). CAR acquisition was measured by mCherry fluorescence. There was no significant transfer of the CAR to any other cell type. ZAP70 was not transferred when expressed alone in J76 cells (labeled “ZAP70-GFP” in FIG. 16). However, when expressed together with CAR-mCherry, ZAP70-GFP (“CAR+ZAP” in FIG. 16C) was specifically transferred only to the B cells, but no other PBMC subset.

### Example 14. Engineer Cytografts to Transfer Effectors to Target Cells In Vivo

### Donor Cells:

Primary mouse regulatory T cells (Tregs) are isolated from C57BL/6J mice and stimulated with CD3/CD28 Dynabeads, then transduced with lentivirus in 2 groups:
1) Test group receives:
   a. Lentivirus that encodes a shuttle: mCD19scFv-CD28-CD3z-mRuby (SEQ ID NO: 101)
   b. Lentivirus that encodes an effector: mZAP70-EGFP (SEQ ID NO: 102)
2) Control group receives:
   a. Empty lentivirus

### Cell Identity Labeling:

To track the donor cells, they are labeled with dyes that covalently crosslink to cytoplasmic structures (Thermo CellTracker dyes).

### Mouse Injection:

Following transduction, cells are further expanded for a total of 10-20 days before being restimulated with CD3/CD28 Dynabeads. Restimulated cells are suspended in sterile saline solution and injected intravenously into B6.SJL mice at a dose of 1 million cells per mouse.

### Transfer Measurement:

At 1, 3, and 7 days post-injection, mice are euthanized and spleens are harvested. The spleens are dissociated and B cells from the dissociated spleens are analyzed for mRuby (a fluorescent protein included as a component of the shuttle) or EGFP fluorescence (a fluorescent protein included as a component of the effector), which indicates transfer of effectors from donor cells to mouse B cells in vivo.

| Name | Sequence | SEQ ID NO: |
|---|---|---|
| mCD19scFv- | gccaccATGGGAGTGCCTACACAGCTGCTGGGACTGCTGCTGCTGT | 101 |
| CD28-CD3z- | GGATCACAGACGCCATCTGCGACATCCAGATGACACAGAGCCCT | |
| mRuby | GCCAGCCTGAGCACATCTCTGGGAGAGACAGTGACCATCCAGTG | |
| | CCAGGCCAGCGAGGACATCTACTCTGGACTGGCCTGGTATCAGC | |
| | AGAAGCCCGGCAAATCTCCCCAGCTGCTGATCTACGGTGCCAGC | |
| | GATCTGCAGGATGGCGTGCCATCTAGATTCAGCGGCTCTGGCTCT | |
| | GGCACCCAGTACAGCCTGAAGATCACCAGCATGCAGACCGAGG | |
| | ACGAGGGCGTGTACTTCTGTCAGCAAGGCCTGACATACCCCAGA | |
| | ACCTTCGGCGGAGGCACCAAGCTGGAACTTAAAGGCGGCGGAG | |
| | GATCTGGCGGAGGTGGAAGTGGCGGAGGCGGATCTGAAGTTCA | |
| | GCTGCAGCAATCTGGCGCCGAACTCGTCAGACCTGGCACAAGCG | |
| | TGAAGCTGAGCTGCAAGGTGTCCGGCGACACCATCACCTTCTAC | |
| | TACATGCACTTCGTGAAGCAGAGGCCCGGACAGGGACTCGAGTG | |
| | GATCGGAAGAATCGACCCTGAGGACGAGAGCACCAAGTACTCC | |
| | GAGAAGTTCAAGAACAAGGCCACACTGACCGCCGACACCAGCA | |
| | GCAACACAGCCTACCTGAAGCTGTCCAGCCTGACCTCCGAGGAC | |
| | ACCGCCACCTATTTCTGTATCTACGGCGGCTACTACTTCGACTAC | |
| | TGGGGCCAGGGCGTGATGGTCACCGTGTCTAGCACTAGTACCAC | |
| | CACCAAGCCTGTGCTGAGAACCCCTTCTCCTGTGCACCCTACCGG | |
| | CACAAGCCAGCCTCAAAGACCTGAGGACTGCAGACCTAGAGGC | |
| | AGCGTGAAAGGCACAGGCCTGGACTTCGCCTGTGATTTCTGGGC | |
| | TCTTGTGGTGGTGGCTGGCGTGCTGTTTTGTTACGGCCTGCTGGT | |
| | CACAGTGGCCCTGTGTGTGATCTGGACCAACAGCAGAAGAAACA | |
| | GACTGCTGCAGAGCGACTACATGAACATGACCCCTAGAAGGCCT | |
| | GGCCTGACCAGAAAGCCTTACCAGCCTTACGCTCCCGCCAGAGA | |
| | TTTCGCCGCCTACAGACCAAGAGCCAAGTTCTCCAGAAGCGCCG | |
| | AGACAGCCGCTAACCTGCAGGATCCTAACCAGCTGTACAACGAG | |
| | CTGAACCTGGGGAGAAGAGAAGAGTACGACGTGCTGGAAAAGA | |
| | AGAGAGCCAGGGATCCTGAGATGGGCGGCAAGCAGCAGAGAAG | |
| | AAGAAACCCTCAAGAAGGCGTGTACAACGCCCTGCAGAAAGAC | |
| | AAGATGGCCGAGGCCTACAGCGAGATCGGAACTAAGGGCGAAC | |
| | GCAGAAGAGGCAAGGGCCACGATGGACTGTACCAGGGCCTGTCT | |
| | ACAGCCACCAAGGACACATACGATGCCCTGCACATGCAGACACT | |
| | GGCCCCTAGACCTCCAGTGATGCATATGGTGTCCAAGGGCGAAG | |
| | AACTGATCAAAGAAAACATGCGGATGAAGGTGGTCATGGAAGG | |
| | CTCCGTGAACGGCCACCAGTTCAAGTGTACTGGCGAAGGCGAGG | |
| | GCAGACCCTATGAAGGCGTGCAGACCATGAGAATCAAAGTGATC | |
| | GAAGGCGGCCCTCTGCCTTTCGCCTTTGACATCCTGGCCACCAGC | |
| | TTTATGTACGGCAGCAGGACCTTCATCAAGTACCCCGCTGACATC | |
| | CCCGATTTCTTCAAGCAGAGCTTCCCCGAGGGCTTCACATGGGA | |
| | GAGAGTGACAAGATACGAGGACGGCGGCGTCGTGACAGTGACA | |
| | CAGGACACAAGTCTGGAAGATGGCGAGCTGGTGTACAATGTGAA | |
| | AGTGCGGGGCGTGAACTTCCCCAGCAACGGACCTGTGATGCAGA | |
| | AAAAGACCAAAGGCTGGGAGCCCAACACCGAGATGATGTACCC | |
| | TGCTGATGGCGGCCTGAGAGGCTACACAGATATCGCCCTGAAAG | |
| | TGGATGGCGGCGGACATCTGCACTGCAACTTCGTGACCACCTAC | |
| | AGGTCCAAGAAAACCGTGGGCAACATCAAGATGCCCGGGGTGC | |
| | ACGCCGTGGACCACAGACTGGAAAGAATCGAGGAAAGCGACAA | |
| | CGAGACATACGTGGTGCAGCGCGAAGTGGCCGTGGCCAAGTATT | |
| | CTAACCTTGGAGGCGGCATGGACGAACTGTACAAATAA | |
| mZAP70- | GCCACCATGGTGAGCAAGGGCGAGGAGCTGTTCACCGGGGTGGT | 102 |
| EGFP | GCCCATCCTGGTCGAGCTGGACGGCGACGTAAACGGCCACAAGT | |
| | TCAGCGTGTCCGGCGAGGGCGAGGGCGATGCCACCTACGGCAAG | |
| | CTGACCCTGAAGTTCATCTGCACCACCGGCAAGCTGCCCGTGCC | |
| | CTGGCCCACCCTCGTGACCACCCTGACCTACGGCGTGCAGTGCTT | |
| | CAGCCGCTACCCCGACCACATGAAGCAGCACGACTTCTTCAAGT | |
| | CCGCCATGCCCGAAGGCTACGTCCAGGAGCGCACCATCTTCTTC | |
| | AAGGACGACGGCAACTACAAGACCCGCGCCGAGGTGAAGTTCG | |
| | AGGGCGACACCCTGGTGAACCGCATCGAGCTGAAGGGCATCGAC | |
| | TTCAAGGAGGACGGCAACATCCTGGGGCACAAGCTGGAGTACA | |
| | ACTACAACAGCCACAACGTCTATATCATGGCCGACAAGCAGAAG | |
| | AACGGCATCAAGGTGAACTTCAAGATCCGCCACAACATCGAGGA | |
| | CGGCAGCGTGCAGCTCGCCGACCACTACCAGCAGAACACCCCCA | |
| | TCGGCGACGGCCCCGTGCTGCTGCCCGACAACCACTACCTGAGC | |
| | ACCCAGTCCGCCCTGAGCAAAGACCCCAACGAGAAGCGCGATCA | |
| | CATGGTCCTGCTGGAGTTCGTGACCGCCGCCGGGATCACTCTCG | |
| | GCATGGACGAGCTGTATAAGGGACCAGGATCGGCGTCTGGGGA | |
| | AGGACTGCCAGGATCAGCCGGACCAGGAATGCCCGATCCCGCGG | |
| | CGCATCTGCCATTCTTCTATGGCAGCATCTCGCGGGCTGAGGCCG | |
| | AGGAGCACCTGAAGCTGGCAGGCATGGCCGACGGGCTGTTCCTC | |
| | CTGCGCCAGTGTTTGCGCTCCCTGGGCGGCTACGTGCTGTCGTTG | |
| | GTGCACGACGTGCGCTTCCACCATTTCCCCATCGAGCGCCAACTC | |
| | AACGGCACGTACGCCATCGCGGGCGGGAAGGCGCACTGCGGCC | |
| | CGGCCGAGCTCTGCCAGTTCTACTCTCAGGACCCCGATGGGCTG | |
| | CCCTGCAACCTGCGTAAGCCGTGTAACCGGCCGCCCGGACTGGA | |
| | GCCACAGCCCGGGGTCTTCGACTGCCTGCGTGATGCTATGGTGT | |
| | GCGACTACGTGCGCCAGACCTGGAAGCTGGAGGGCGATGCACTA | |
| | GAGCAGGCCATCATCAGCCAGGCCCCACAGGTGGAGAAGCTCAT | |
| | TGCTACCACAGCCCACGAGCGAATGCCCTGGTATCACAGCAGCC | |
| | TGACTCGTGAGGAGGCCGAGCGCAAACTCTATTCCGGCCAGCAG | |
| | ACCGACGGCAAGTTCCTGCTGAGGCCCCGGAAGGAGCAGGGCA | |
| | CATATGCACTGTCCCTGGTCTATGGGAAAACTGTATACCACTATC | |
| | TCATCAGCCAGGACAAGGCTGGCAAGTACTGCATCCCCGAAGGC | |
| | ACCAAGTTTGACACGCTCTGGCAGCTGGTGGAGTACCTGAAGCT | |
| | GAAGGCGGATGGACTAATTTACCGCTTGAAGGAGGTCTGTCCCA | |
| | ACAGCAGCGCCCCAGGGCACCATCATCATCATCATTAA | |

### Example 15. Engineer Cytografts to Express Effectors Upon Binding Antigen and Transfer the Effectors to Target Cells Expressing that Antigen

### Donor Cells:

J76 cells that stably express CD19scFv-CD3z-mRuby3-MCP are electroporated with a gene cassette that expresses EGFP under the control of an NFAT promoter.

### Acceptor Cells:

1) HEK293T cells that have been modified to express CD19
2) Unmodified HEK293T cells

### Cell Identity Labeling:

To distinguish donor and acceptor cells, they are labeled with unique dyes that covalently crosslink to cytoplasmic structures (Thermo CellTracker dyes).

### Cell-Cell Contact:

Donor and acceptor cells from each of the above groups are subjected to pairwise co-culture (2 combinations). The cells are first monocultured separately in suspension media. To coculture, acceptor cells are added to donor cell cultures at a 1:1 ratio. The co-cultured cells are incubated at 37° C. for 24 hours.

### Transfer Measurement:

At 4, 8, and 24 hours of co-culture, cells are analyzed by flow cytometry. EGFP expression in the donor cells indicates activation of the NFAT-GFP gene cassette.

### Example 16. Engineer Cytografts to Transfer Membrane-Penetrating Effectors to Target Cells

### Donor Cells:

J76 cells that stably express CD19scFv-CD3z-mRuby3-MCP are electroporated different constructs as follows:
1) Cre recombinase fused to a cell-penetrating peptide, e.g., as described in Table CP1
2) Zap70-Cre fused to a cell-penetrating peptide

### Acceptor Cells:

1) HEK293T cells that have been modified to express CD19 and a Flex-GFP reporter
2) HEK293T cells that express only the Flex-GFP reporter

### Cell Identity Labeling:

To distinguish donor and acceptor cells, they are labeled with unique dyes that covalently crosslink to cytoplasmic structures (Thermo CellTracker dyes).

### Cell-Cell Contact:

Donor and acceptor cells from each of the above groups are subjected to pairwise co-culture (4 combinations). The cells are first monocultured separately in suspension media. To coculture, acceptor cells are added to donor cell cultures at a 1:1 ratio. The co-cultured cells are incubated at 37° C. for 24 hours.

### Transfer Measurement:

At 4, 8, and 24 hours of co-culture, cells are analyzed by flow cytometry. EGFP expression in the acceptor cells indicates functional transfer of the Cre to acceptor cells.

### Example 17. Engineer Cytografts to Express a Secreted Effector Molecule Upon Binding a Target Antigen

### Donor Cells:

J76 cells are electroporated with expression constructs in the following groups:
1) CD19scFv-miniNotch and insulin under the control of a notch-responsive promoter
2) Insulin under the control of a notch-responsive promoter

### Acceptor Cells:

1) HEK293T cells that have been modified to express CD19
2) Unmodified HEK293T cells

### Cell Identity Labeling:

To distinguish donor and acceptor cells, they are labeled with unique dyes that covalently crosslink to cytoplasmic structures (Thermo CellTracker dyes).

### Cell-Cell Contact:

Donor and acceptor cells from each of the above groups are subjected to pairwise co-culture (4 combinations). The cells are first monocultured separately in suspension media. To coculture, acceptor cells are added to donor cell cultures at a 1:1 ratio. The co-cultured cells are incubated at 37° C. for 24 hours.

### Transfer Measurement:

At 24 hours of co-culture, supernatant is removed and filtered to eliminate any cell contamination. The supernatant is then subjected to ELISA analysis to measure secreted insulin levels.

### Example 18. Engineer Cytografts to Express and Secrete a Cell-Penetrating Protein Upon Binding a Target Antigen

### Donor Cells:

J76 cells that stably express CD19scFv-CD3z-mRuby3-MCP are electroporated a construct that expresses Cre recombinase fused to a cell-penetrating peptide (e.g., as listed in Table CP1) and modified with a secretion signal.

### Acceptor Cells:

1) HEK293T cells that have been modified to express CD19 and a Flex-GFP reporter
2) HEK293T cells that express only the Flex-GFP reporter

### Transwell Cell-Cell Transfer:

Donor and acceptor cells from each of the above groups are subjected to pairwise co-culture (2 combinations) in transwell format. The cells are first monocultured separately in suspension media. To coculture, acceptor cells are added to the bottom chamber and donor cells are added to the top chamber at a 1:1 ratio. The co-cultured cells are incubated at 37° C. for 24 hours.

### Transfer Measurement:

At 4, 8, and 24 hours of co-culture, acceptor cells from the bottom chamber are analyzed by flow cytometry. EGFP expression in the acceptor cells indicates functional transfer of the Cre to acceptor cells.

### Example 19. Engineer Cytografts to Assemble and Release Virus-Like Particles (VLPs) Upon Binding a Target Antigen

### Donor Cells:

HEK293 T cells are calcium-transformed with combinations of constructs comprising:
1) CD19scFv-CD3z-mRuby3-MCP and Cre RNA modified with PEG10 binding sequence
2) CD19scFv-CD3z-mRuby3-MCP, Cre RNA modified with PEG10 binding sequence and an a PEG10 protein driven by an NFAT promoter

### Acceptor Cells:

1) HEK293T cells that have been modified to express CD19 and a Flex-GFP reporter
2) HEK293T cells that express only the Flex-GFP reporter

### Transwell Cell-Cell Transfer:

Donor and acceptor cells from each of the above groups are subjected to pairwise co-culture (4 combinations) in transwell format. The cells are first monocultured separately in suspension media. To coculture, acceptor cells are added to the bottom chamber and donor cells are added to the top chamber at a 1:1 ratio. The co-cultured cells are incubated at 37° C. for 24 hours.

### Transfer Measurement:

At 4, 8, and 24 hours of co-culture, acceptor cells from the bottom chamber are analyzed by flow cytometry. EGFP expression in the acceptor cells indicates functional transfer of the Cre to acceptor cells.

### Example 20. Engineer Tregs to Generate Tunneling Nanotubes Upon Binding Target Antigen

### Donor Cells:

J76 cells that stably express CD19scFv-CD3z-mRuby3-MCP are electroporated with a gene cassette that expresses US3 under the control of an NFAT promoter and constitutive Cre recombinase mRNA

### Acceptor Cells:

1) HEK293T cells that have been modified to express CD19 and a Flex-GFP reporter
2) HEK293T cells that express only the Flex-GFP reporter

### Cell Identity Labeling:

To distinguish donor and acceptor cells, they are labeled with unique dyes that covalently crosslink to cytoplasmic structures (Thermo CellTracker dyes).

### Cell-Cell Contact:

Donor and acceptor cells from each of the above groups are subjected to pairwise co-culture (2 combinations). The cells are first monocultured separately in suspension media. To coculture, acceptor cells are added to donor cell cultures at a 1:1 ratio. The co-cultured cells are incubated at 37° C. for 24 hours.

### Transfer Measurement:

At 4, 8, and 24 hours of co-culture, cells are analyzed by flow cytometry. EGFP expression in the donor cells indicates activation of the NFAT-GFP gene cassette.

### Example 21. Cytografts Generated from a Human T Cell Line by Adding a Shuttle Receptor

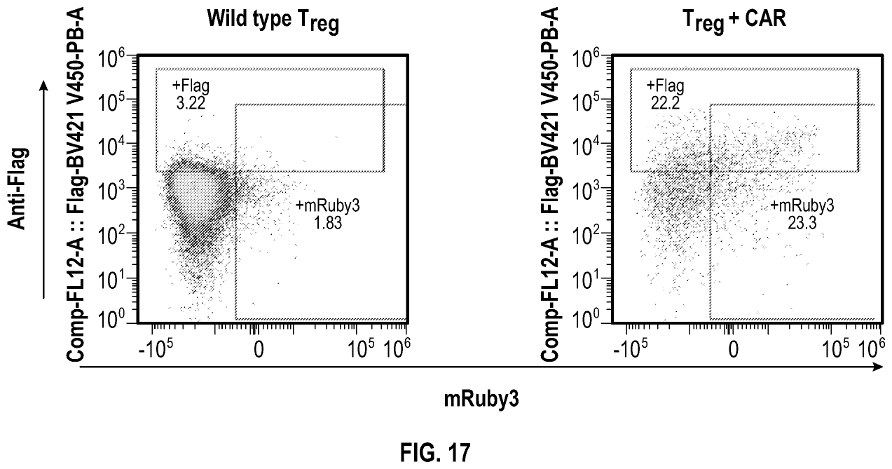

This Example utilizes a simple chassis cell containing a chimeric antigen receptor-like protein, referred to herein as a shuttle. The J76 cell, being derived from the common Jurkat cell line, is non-cytotoxic. In this Example, the shuttle was used to drive transfer of cell membrane and cytoplasmic material from the chassis cell to any cell that expresses the target antigen (in this case CD19, which is expressed on B cells). Electroporation provided temporary expression of the shuttle. Without a shuttle, transfer can occur in co-culture, but in a nonspecific manner. Expression of the CAR was confirmed by flow cytometry (FIG. 17).

### Cell Lines

Cytograft: J76 cell line (a TCR-knockout Jurkat T cell line)
Shuttle: J76 cells were electroporated with an expression construct encoding CD19CAR-mRuby.
   J76 chassis cells have also been generated using multiple other shuttle receptors:
1) SIINFEKL-CAR: targets SIINFEKL presented on MHC class I
2) MOG-CAR: targets MOG (myelin oligodendrocyte glycoprotein)
3) StrepCAR: can be retargeted against any antigen using a biotinylated antibody

TABLE B1
List of exemplary cytografts that have
been generated from cell lines.

| Cytograft/chassis cell | Cargo or Shuttle |
|---|---|
| K562 | Dye-labelled membrane |
| K562 | Biotinylated surface proteins |
| K562 | HLA-G |
| PBMC | Biotinylated surface proteins |
| K562 | HLA-G |
| K562 | Biotinylated surface proteins |
| JEG-3 | Biotinylated surface proteins |
| PBMCs | Biotinylated surface proteins, CD3, CD4, CD8 |
| K562 | Biotinylated surface proteins |
| K562 | Biotinylated surface proteins |
| THP1 | Biotinylated surface proteins |
| Jurkat | Biotinylated surface proteins |
| Jurkat | Biotinylated surface proteins |
| J76 | Biotinylated surface proteins |
| Primary Hepatocytes | Biotinylated surface proteins |
| K562 | HLA-G |
| Jurkat | MART-1 TCR |
| K562 | LCK-GFP (membrane-associated) |
| K562 | CCR7, CXCR3, CCR10 |
| Jurkat (DMF5) | TCR, Biotinylated surface proteins |
| J76 | CD19CAR, ZAP70-GFP |
| J76 | CD19CAR |
| J76 | CD19CAR |
| J76 | CD19CAR, ZAP70-GFP |
| J76 | CD19 CAR, small RNA |
| J76 | CD19CAR, ZAP70-GFP |
| J76 | SIINFEKL CAR |
| J76 | SIINFEKL CAR |

### Example 22. Stable Cytografts Generated from a Human T Cell Line

### Cell Lines

Cytograft: 176 cell line (a TCR-knockout Jurkat T cell line)
Shuttle: 176 cells were electroporated with a sleeping beauty donor construct encoding CD19CAR-mRuby and sleeping beauty transposase.

In this Example, stable cytografts were generated by integrating a shuttle construct into the genomes of J76 cells using the sleeping beauty transposon system. This produces a stable version of the chassis cell described in Example 1. In brief, cells were electroporated with a sleeping beauty donor constructed encoding CD19CAR-mRuby and a sleeping beauty transposase. After electroporation, the cells were assessed for stable expression of CD19CAR-mRuby to confirm that the DNA had been stably integrated into the genome. The resulting cell line expressed CD19CAR-mRuby permanently.

### Example 23. Cytografts Engineered to Deliver Cargos to Target Cells

### Cell Line:

Cytograft: J76 cell line (a TCR-knockout Jurkat T cell line)
Shuttle: J76 cells were electroporated with an expression construct encoding CD19CAR-mRuby.
Effector: The cells were also electroporated with an FL68-engineered fusion protein comprised of the SH2 domains of ZAP70 fused to GFP.

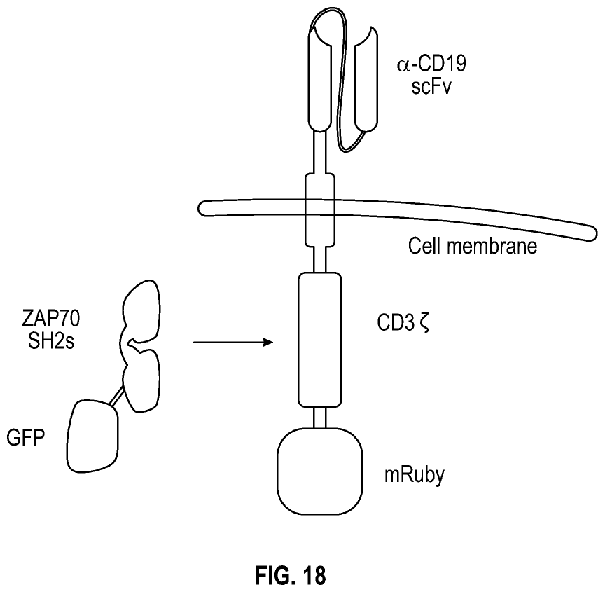

Addition of an effector in this chassis enables the cell to transfer cargos to a target cell in a modular fashion. Zap70 SH2 domains bind to the CD3zeta domain of CD19CAR-mRuby upon binding to target antigen (CD19 in this case). This allows the cytograft to perform antigen-specific delivery of ZAP70-fused cargos to only the target cells that are recognized by the shuttle (FIG. 18). Without wishing to be bound by theory, it is contemplated that any cargo of interest can be fused to ZAP70. In this example, GFP was used as a prototype cargo to be fused to ZAP70.

Several other ZAP70SH2 fusions have been generated:
1) ZAP70SH2-Cre: to deliver Cre recombinase to target cells
2) ZAP70SH2-GFP-Cre: to deliver GFP with Cre recombinase
3) ZAP70SH2-MCP: to deliver MS2-tagged RNAs to target cells

### Example 24. Cytografts Transfer Effectors to Target Cells

### Cell Lines

Cytograft: J76-CD19CAR (the stable cell line generated in example 2)
Shuttle: The cell line stably expresses CD19CAR-mRuby
Effector: The cells were electroporated with constructs expressing ZAP70-EGFP

In this Example, cytografts were co-cultured with a target cell line (Ramos B cells that express CD19) for 1 hour at a 1:1 ratio. Both cell types were labeled separately with dyes before they were mixed to allow tracking of cell identity. After co-culture the cells were subjected to flow cytometry to determine whether transfer of the shuttle and/or effector to target cells occurred.

### Results:

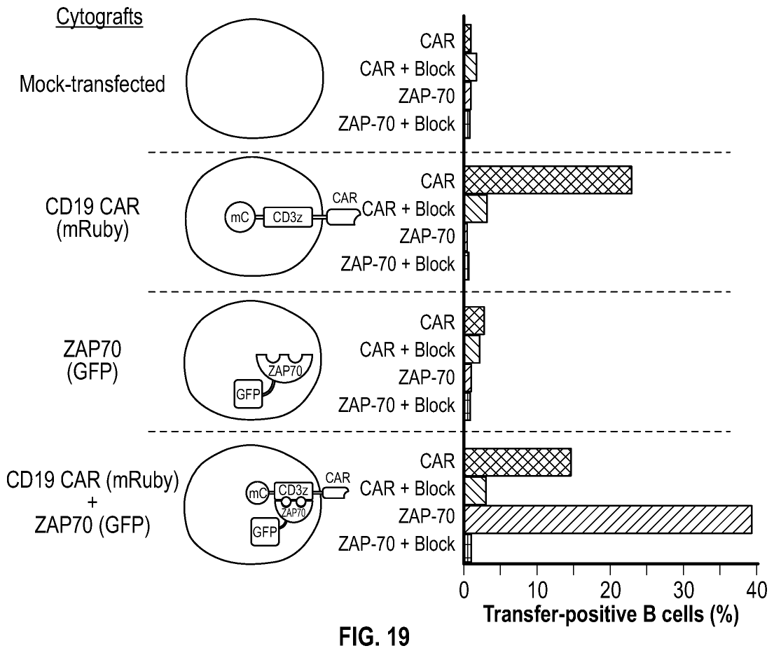

Both the shuttle (tracked by mRuby fluorescence) and the effector (tracked by EGFP fluorescence) were transferred from the cytograft to the target cell when present together in the cytograft. ZAP70-GFP was only capable of transferring to target cells when the shuttle (CD19 CAR) was also present (FIG. 19).

### Example 25. Cytografts, Engineered to Express Fusogens, Transfer Functional Effectors to Target Cells

### Cell Lines

Cytograft: J76-CD19CAR (the stable cell line generated in example 2) or HEK 293t cells
Shuttle: The cell line stably expresses CD19CAR-mRuby. This is shuttle contains an RNA binding domain, MCP, that is capable of binding RNAs that have been tagged with MS2 stem loops.
Effector: The Cytografts cells were transfected with constructs that express Cre mRNA. Half of the cytografts were also transfected with constructs expressing vsv-g, which is a fusogen that is active at low pH. The rationale for vsv-g is that it may allow transferred material to escape endosomes.
Target cells: Target cells were HEK 293t cells that had been calcium-transfected with constructs expressing a Cre reporter called Flex-GFP, which will generate GFP in target cells only if they receive Cre from the Cytografts. The target cells were also transfected with a construct expressing CD19, since HEK 293t cells do not naturally produce CD19. Un-transfected HEK 293t cells served as controls.

In this Example, cytografts were co-cultured with target cells for 24 hours at a 1:1 ratio, then imaged for GFP fluorescence, which indicated whether Cre was transferred functionally to target cells. GFP fluorescence would indicate that Cre was transferred in a way that allowed it to access the nucleus and recombine the DNA.

### Results:

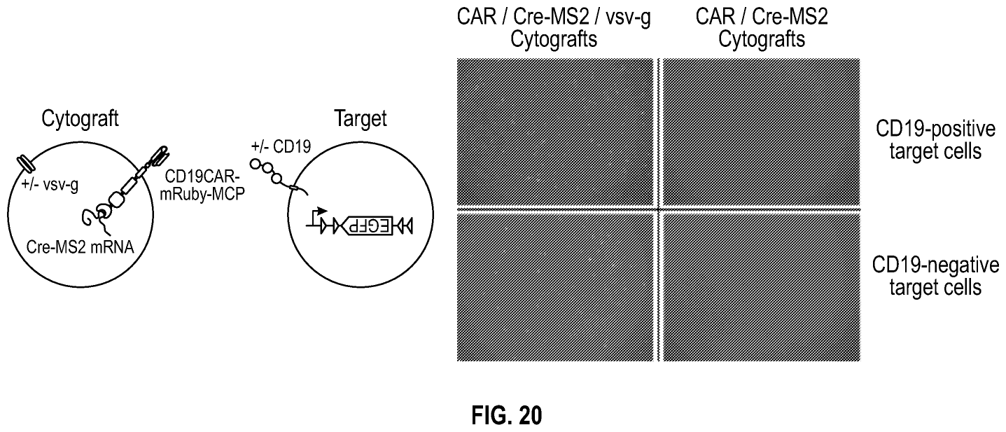

GFP was observed in a fraction of the target cells after 24 hours, indicating functional transfer of Cre. GFP expression was restricted to conditions where the cytograft expressed vsv-g, supporting that fusogen activity may be required for functional transfer (FIG. 20).

### Example 26. Cytografts Generated from Primary Human Cells

### Cell Lines:

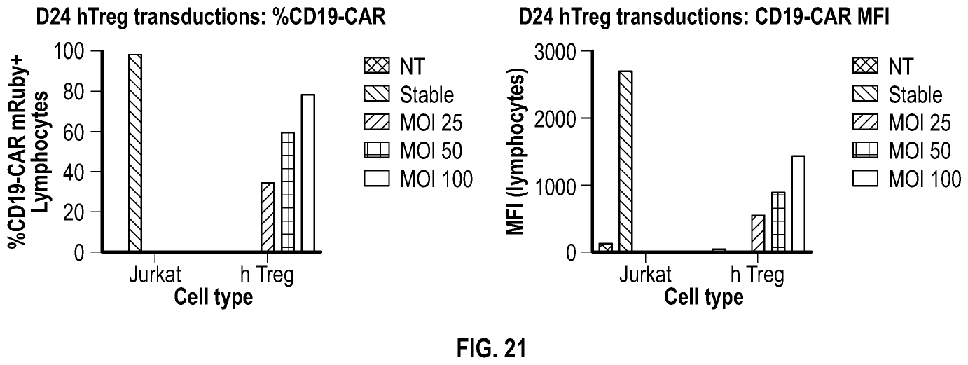

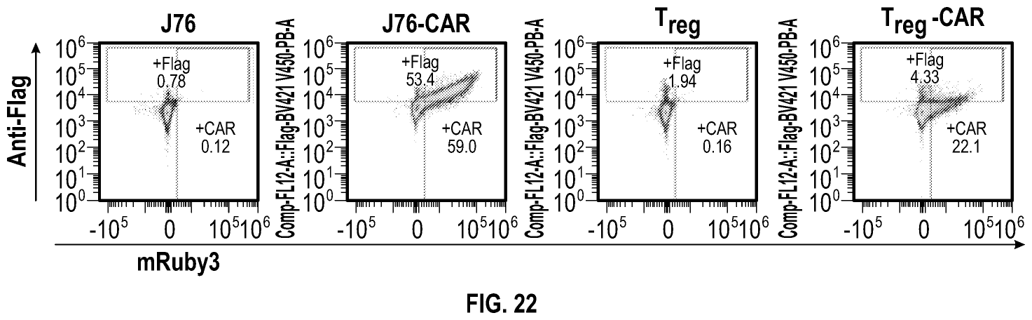

Chassis cell: Human primary CD4+/CD25+ T cells from healthy donors
Shuttle: Cells were transduced with lentiviral constructs expressing CD19CAR-mRuby.
Target cells: Ramos B cells
Co-culture: Cytografts were co-cultured with target cells for 1 hour, then subjected to flow cytometry to measure acquisition of the CAR by Ramos cells. CAR was measured via mRuby fluorescence and flag tag staining, since CD19CAR-mRuby contains a short flag tag in the extracellular domain.
Results: We confirmed expression of the CAR after transduction and ability to transfer the CAR to target cells, as shown in FIGS. 21 and 22, respectively.

### Example 27. Murine Cytografts for Pre-Clinical In Vivo Studies

### Cell Lines

Chassis Cell: primary mouse CD4 T cells
Shuttle: Cytografts are transduced with a murine-pseudotyped lentiviral construct expressing a mouse-specific version of CD19CAR-mRuby.

In this Example, cytografts are produced from primary murine CD4 T cells, transfected with a construct expressing a murine version of CD19CAR-mRuby. The cytografts are injected intravenously into mice to track migration to target tissue sites, including spleen, bone marrow, and lymph nodes. We also measure persistence and expansion of the cells at these sites, which we hypothesize will occur when the cytografts encounter the target of their shuttle receptor, CD19, expressed on B cells at those sites.

### Example 28. Knock Out of Endogenous Genes from Primary Human T Cells Using CRISPR

Cytograft: Human primary CD3+ T cells from healthy donors were thawed, then activated with CD3/CD28 on day 1 post-thaw.

RNP preparation: Ribonucleoprotein complexes (RNPs) were formed by mixing pure Cas9 protein with RNA guide oligonucleotides targeting human TCRα and TCRβ, at a 1:5 ratio.

Electroporation: For each condition, 2 million activated T cells were electroporated with RNPs on day 4 post-thaw. Conditions were varied across 2 different electroporator systems, testing differences in buffers, volumes, and cell densities (table).

### Electroporation Conditions

| System | Buffer | Volume | Cell density | Condition |
|---|---|---|---|---|
| Neon | T | 100 uL | 2e7/mL | 2100-20-1 |
| | | | | 2000-15-2 |
| Lonza | P3 | 20 uL | 1e8/mL | EH-115 |
| | | | | EO-115 |
| | | | | FI-115 |
| | | 100 uL | 2e7/mL | EH-115 |
| | | | | EO-115 |
| | | | | FI-115 |

### Results

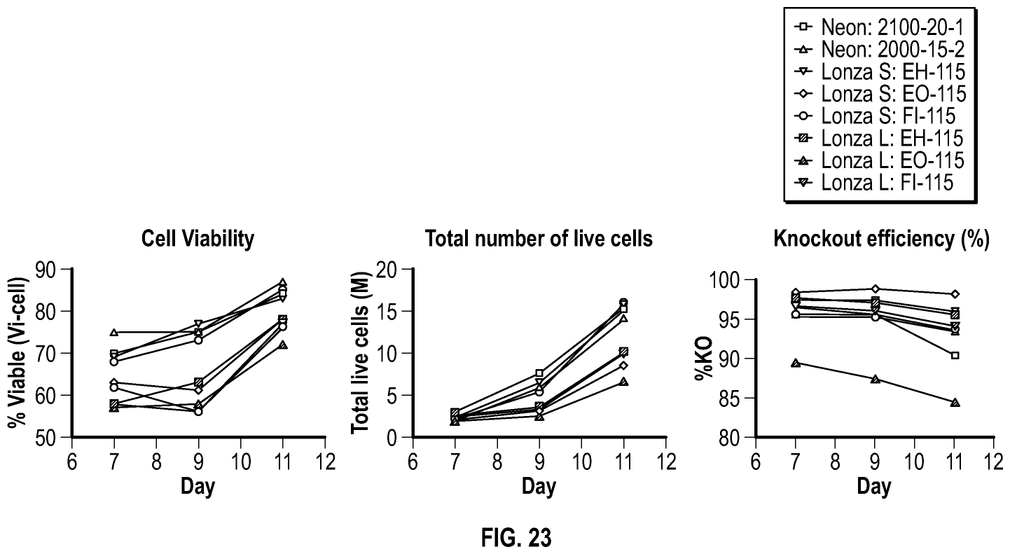

The electroporated T cells were assessed for TCRαβ expression at days 7, 9, and 11 post-thaw using flow cytometry. Cell viability and cell number were also measured at each time point. As shown in FIG. 23, for the various electroporation conditions tested, cell viability was maintained at levels between approximately 55% to 85% over the following two weeks (left panel) and the total number of live cells increased over time (middle panel). Knockout efficiency was greater than 90% for all but one condition tested (FIG. 23, right panel).

### Example 29. CRISPR Knock-In to the TCR Locus

Cytograft: Human primary CD3+ T cells from healthy donors are thawed, then activated with CD3/CD28 on day 1 post-thaw.

RNP preparation: Ribonucleoprotein complexes (RNPs) are formed by mixing pure Cas9 protein with RNA guide oligonucleotides targeting human TCRα and TCRβ, at a 1:5 ratio.

DNA donor template: A DNA cassette encoding CD19CAR-mRuby is flanked by upstream and downstream homology arms from DNA regions upstream and downstream of endogenous TCRα.

Electroporation: For each condition, 2 million activated T cells are electroporated with RNPs and donor template on day 4 post-thaw.

Measurement: Loss of TCR is assessed as described in Example 28. Expression of the CAR is also measured via mRuby fluorescence.

### Example 30. Transposon-Based Editing to Modify Cells

Cytograft: Human primary CD4+/CD25+ T cells from healthy donors are thawed, then activated with CD3/CD28 on day 1 post-thaw.

Transposon mix: Sleeping beauty transposase-encoding mRNA is mixed with a plasmid encoding CD19CAR-mRuby flanked by upstream and downstream LTRs that are recognized by sleeping beauty transposase.

Electroporation: Activated T cells are electroporated with transposon mix on day 4 post-thaw.

Measurement: Expression of the CAR is measured via flow cytometry for mRuby fluorescence.

### Example 31. Immortalization of Primary Human Cells to Generate Universal Cell Lines

Cytograft: Human primary CD4+/CD25+ T cells from healthy donors are thawed, then activated with CD3/CD28 on day 1 post-thaw.

Lentivirus: Lentiviral particles are generated to insert multiple immortalization genes, including different combinations of hTERT, SV40 Large T antigen, CDK4, and HPV16-E6/E7.

Transduction: Activated T cells are spin-transduced with virus.

Measurement: Immortalization is assessed by long-term culture of transduced cells relative to non-transduced control cells.

### Example 32. Lineage-Restricted Shuttles and Effectors

Cytograft: Human primary CD4+/CD25+ T cells from healthy donors are thawed, then activated with CD3/CD28 on day 1 post-thaw.

CRISPR editing: Guide RNA sequences are designed to cut at the human AAV1 locus. A donor DNA template containing-flanking regions homologous to the AAV1 locus is constructed. A FoxP3 promoter is placed in between the flanking regions to control expression of the shuttle and effector proteins detailed below. The proteins are separated by a 2A sequence to allow generation of two separate proteins during translation.
Shuttle: CD19CAR-mRuby
Effector: ZAP70-Cre
Analysis: Edited cells are phenotyped to confirm that only FoxP3-positive cells express the shuttle and effector. Cells are both passaged in vitro and injected into NSG mice. The cells are assessed for maintenance of cell phenotype over time to confirm continued expression of FoxP3. Without wishing to be bound by theory, it is contemplated that cells that lose FoxP3 expression will stop expressing the shuttle and effector.

### Example 33. Installation of Kill-Switches to Allow Termination of Therapeutic Cells after Injection

Cytograft: Human primary CD4+/CD25+ T cells from healthy donors are thawed, then activated with CD3/CD28 on day 1 post-thaw.

Editing: The kill-switch is installed by genetic knock-in using CRISPR as described herein.

Kill switch composition: The kill switch is an inducible caspase 9 (iCasp9). iCasp9 must dimerize in order to initiate apoptosis of the cytograft. To activate the kill switch, a small molecule dimerizer is given to the patient, activating the iCasp9 and driving apoptosis of the cytografts.

### Example 34. Use of Selectable Markers to Isolate Successfully Edited Cytografts

Cytograft: Human primary CD4+/CD25+ T cells from healthy donors are thawed, then activated with CD3/CD28 on day 1 post-thaw.

Selectable marker: A CD19CAR-mRuby shuttle was generated with a Flag tag epitope placed within the hinge domain, between the anti-CD19 scFv and the transmembrane domain. This CD19CAR-flag-mRuby protein was inserted into a lentiviral construct and transduced into the cytograft cells.

Selection: After transduction with the lentivirus, cells were expanded in culture. After expansion, the cells were incubated with anti-Flag magnetic antibodies and placed in a strong magnetic field. Only shuttle-expressing cells bind to the magnetic antibodies, which allows them to be collected in the magnet. Shuttle-negative cells wash away.

Purity analysis: Following magnetic selection, purity is assessed by flow cytometry to measure the percent of cells that express mRuby (the shuttle-positive fraction).

### Example 35. Derivation of Cytografts from Induced Pluripotent Stem Cells

iPSC generation: Somatic cells are collected from health donors and exposed to reprogramming factors to de-differentiate the cells into iPSCs.

iPSC editing: iPSC lines are edited to provide them with the necessary components to conduct cell-targeted transfer. Editing of the iPSCs is performed using CRISPR, transposons, or lentiviral methodologies (for example, as described herein). Edits can include, for example, addition of shuttles and effectors as well as modifications intended to limit immunogenicity or to provide safety mechanisms.

Differentiation of Cytografts into Tregs: iPSCs are differentiated by exposure to growth factors and transcription factors. The combination and timing of these treatments mimics the natural development of Tregs. Alternative protocols may be used to differentiate the iPSCs into other types of cells, resulting in novel cytografts.

Injection: Differentiated cytografts are injected into mice to test for migration, transfer, and therapeutic efficacy.

### Example 36. Transfer Using a Signaling-Deficient CAR

### Donor Cells:

1) J76 (mock electroporated): Donor cells derived from Jurkat cells. The native TCR has been knocked out.
2) J76+CD19 CAR-mRuby: mRuby fused to the C-terminus of the CAR 3) J76+CD19 CAR-mRuby+ZAP70-GFP: same as above with additional ZAP70-GFP cargo.
4) J76+CD19 deadCAR-mRuby: Modification of CD19 CAR-mRuby that lacks a CD3z domain, eliminating downstream signaling and ZAP70 binding
5) J76+CD19 deadCAR-mRuby+ZAP70-GFP

### Acceptor Cells:

Ramos cells: B cell line that expresses CD19.

### Cell Identity Labeling:

To distinguish donor cells from acceptor cells, both were labeled with unique dyes that covalently crosslink to cytoplasmic structures (Thermo CellTracker dyes).

### Cell-Cell Contact:

Donor and acceptor cells from each of the above groups were subjected to pairwise co-culture (5 conditions as listed above). The cells were first monocultured separately in suspension media. To co-culture, acceptor cells were added to donor cell cultures at a 1:1 ratio (donor to acceptor). The co-cultured cells were centrifuged at 100 g to force the cells into contact, then incubated at 37° C. for 1 hour.

### Measurement:

Transfer of CAR(mRuby) and ZAP-70(EGFP) from donors to acceptors was measured by flow cytometry. All co-cultured cells were stained with viability dye. Live Ramos cells from each co-culture were gated on CellTracker then assessed for acquisition the CAR and/or ZAP70 cargo by mRuby and EGFP fluorescence, respectively.

### Results:

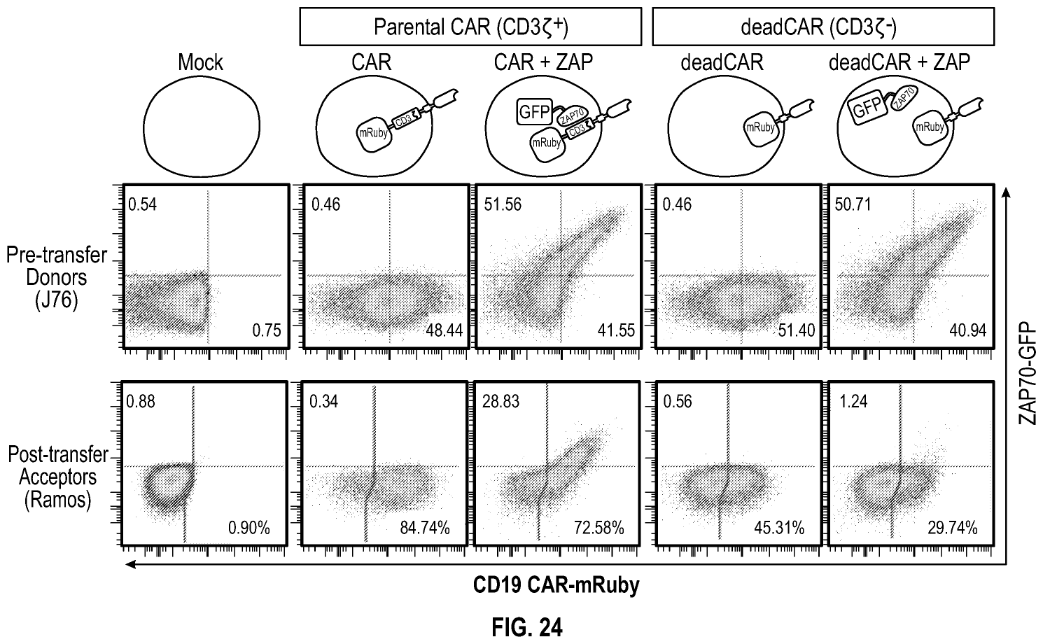

As shown in FIG. 24, CAR transfer occurred in all conditions where the CAR was present, including the deadCAR that lacked CD3z. ZAP70-EGFP transfer depended on presence of the CD3z and did not occur when combined with deadCAR (FIG. 24).

### Example 37. Modular Retargeting Using StrepCAR

### Donor Cells:

1) J76 (mock electroporated): Donor cells derived from Jurkat cells. The native TCR has been knocked out.
2) J76+CD19 CAR-mRuby: mRuby fused to the C-terminus of the CAR
3) J76+StrepCAR-mRuby: StrepCAR-mRuby is identical to CD19 CAR-mRuby except that the extracellular scFv has been replaced with a mono-streptavidin domain
4) J76+StrepCAR-mRuby+nonspecific antibody: Same as #3, but the resulting cells were incubated with a biotinylated antibody that recognizes HLA-G, not present on the acceptor cells
5) J76+StrepCAR-mRuby+specific antibody: Same as #3, but the resulting cells were incubated with a biotinylated antibody that recognizes CD19, which is present on the acceptor cells

### Acceptor Cells:

Ramos cells: B cell line that expresses CD19.

### Cell Identity Labeling:

To distinguish donor cells from acceptor cells, both were labeled with unique dyes that covalently crosslink to cytoplasmic structures (Thermo CellTracker dyes).

### Cell-Cell Contact:

Donor and acceptor cells from each of the above groups were subjected to pairwise co-culture (5 conditions). The cells were first monocultured separately in suspension media. To co-culture, acceptor cells were added to donor cell cultures at a 1:1 ratio (donor to acceptor). The co-cultured cells were centrifuged at 100 g to force the cells into contact, then incubated at 37° C. for 5 minutes

### Measurement:

Transfer of CAR(mRuby) and ZAP-70(EGFP) from donors to acceptors was measured by flow cytometry. All co-cultured cells were stained with viability dye. Live Ramos cells from each co-culture were gated on CellTracker then assessed for acquisition either CAR by mRuby fluorescence.

### Results:

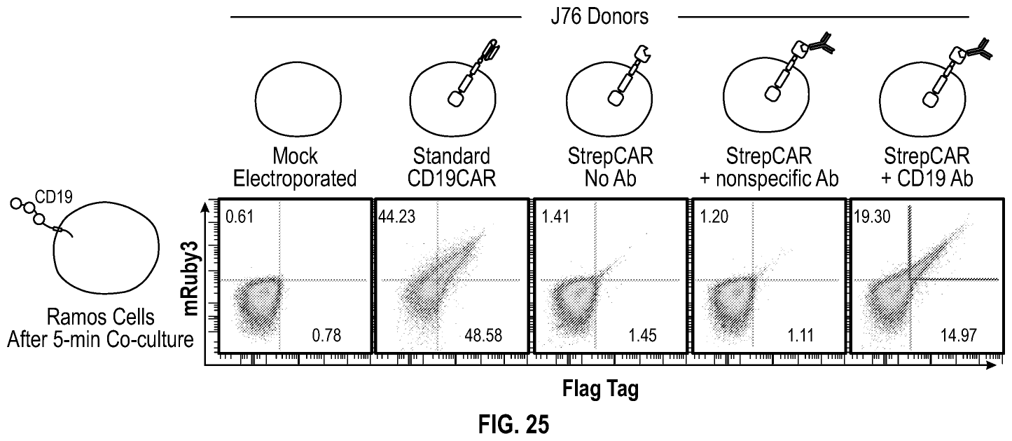

As shown in FIG. 25, transfer of StrepCAR-mRuby occurred only when it was combined with specific antibody targeting CD19, which was present on the acceptor cells. Total transfer for StrepCAR reached approximately half the level observed for CD19 CAR-mRuby. Both CARs transferred significantly to acceptor cells in only 5 minutes of co-culture.

### Example 38. CARs for Targeting MHC-Presented Antigens

### Donor Cells:

1) J76 (mock electroporated): Donor cells derived from Jurkat cells. The native TCR has been knocked out.
2) J76+SIINFEKL CAR-mRuby: This CAR is identical to CD19 CAR-mRuby from previous examples, but with an scFv that recognizes the model antigen SIINFEKL presented on HLA-A2 instead of CD19.
3) J76+StrepCAR-mRuby: StrepCAR-mRuby is identical to CD19 CAR-mRuby except that the extracellular scFv has been replaced with a mono-streptavidin domain
4) J76+StrepCAR-mRuby+nonspecific antibody: Same as #3, but the resulting cells were incubated with a biotinylated antibody that recognizes CD19, which is not expressed by the acceptor cells.
5) J76+StrepCAR-mRuby+specific antibody: Same as #3, but the resulting cells were incubated with a biotinylated antibody that recognizes HLA-presented SIINFEKL, which is present on the antigen-pulsed acceptor cells.

### Acceptor Cells:

1) EL4 cells (no OVA): an HLA-A2 positive cell line, with no antigen
2) EG7 cells (low OVA): an HLA-A2 positive cell line that expresses ovalbumin, resulting in low-level presentation of SIINFEKL
3) EL4 cells (high OVA): same as #1 above, but pulsed with high amounts of SIINFEKL peptide

### Cell Identity Labeling:

To distinguish donor cells from acceptor cells, both were labeled with unique dyes that covalently crosslink to cytoplasmic structures (Thermo CellTracker dyes).

### Cell-Cell Contact:

Donor and acceptor cells from each of the above groups were subjected to pairwise co-culture (15 conditions). The cells were first monocultured separately in suspension media. To co-culture, acceptor cells were added to donor cell cultures at a 1:1 ratio (donor to acceptor). The co-cultured cells were centrifuged at 100 g to force the cells into contact, then incubated at 37° C. for 1 hour or 8 hours.

### Measurement:

Transfer of CAR(mRuby) from donors to acceptors was measured by flow cytometry. All co-cultured cells were stained with viability dye. Live Ramos cells from each co-culture were gated on CellTracker then assessed for acquisition the CAR by mRuby fluorescence.

### Results:

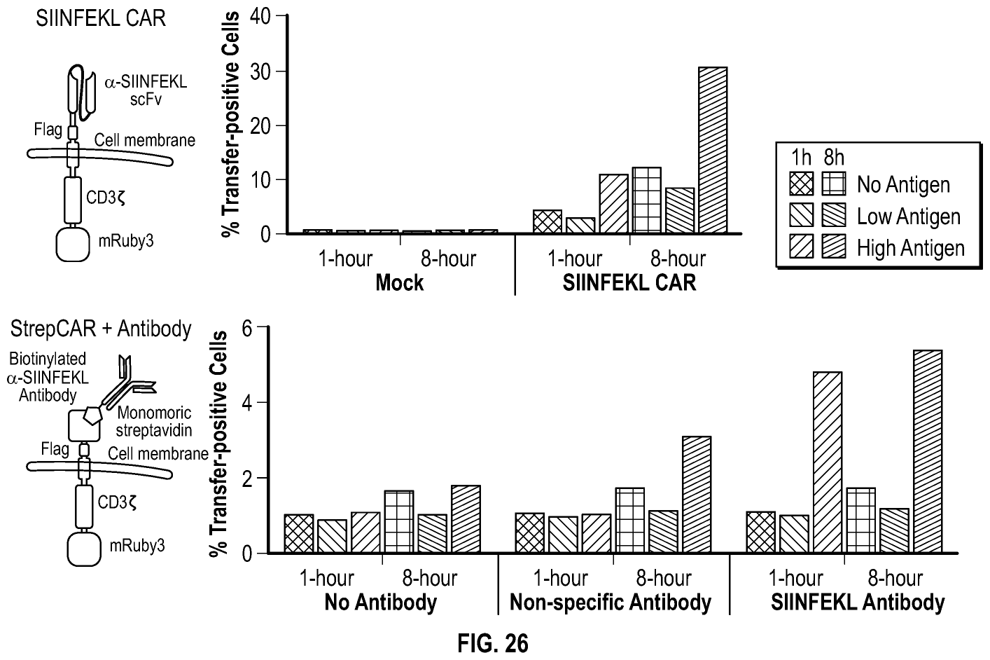

As shown in FIG. 26, CAR transfer occurred in all conditions, but was significantly increased with high antigen and longer incubation.

### Example 39. Transfer of Functional Cre Recombinase to Acceptors

### Donor Cells:

1) HEK 293t cells+CD19 CAR-mRuby (CAR)+Cre-MS2+vsv-g: contains all elements, the CAR, the Cre effector, and the fusogen
2) HEK 293t cells+Cre-MS2+vsv-g: no CAR
3) HEK 293t cells+CAR+Cre-MS2: no fusogen
4) HEK 293t cells+Cre-MS2: no fusogen or CAR

### Acceptor Cells:

1) HEK 293t cells+Flex: Flex is a Cre reporter that expresses EGFP when exposed to Cre
2) HEK 293t cells+Flex+CD19: same as above, but with the antigen recognized by the CAR

### Cell-Cell Contact:

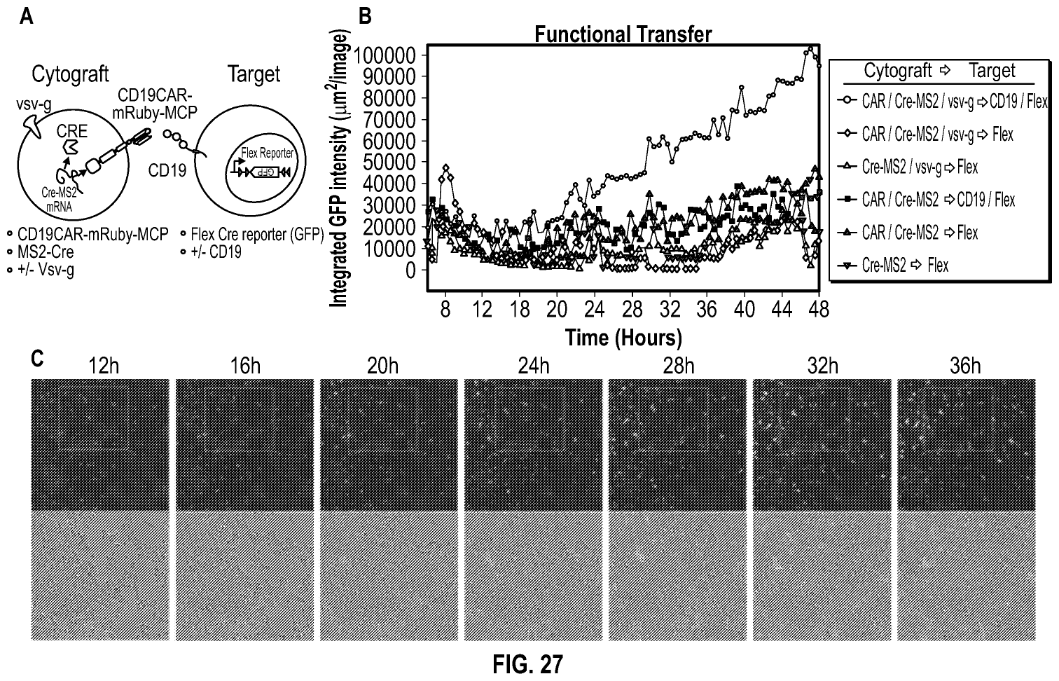

Donor and acceptor cells from each of the above groups were subjected to pairwise co-culture (6 total conditions, shown in the legend of FIG. 27). The cells were first monocultured separately in suspension media. To co-culture, acceptor cells were added to donor cell cultures at a 1:1 ratio (donor to acceptor). The co-cultured cells were incubated at 37° C. for 48 hours.

### Measurement:

EGFP of the acceptors was measured over time in co-culture using an Incucyte live cell imaging system (Sartorius). Cells were imaged every 30 minutes for 48 hours. CAR-positive cells were also measured via mRuby fluorescence. EGFP indicates functional transfer of Cre.

### Results:

As shown in FIG. 27, donor J76 cells containing the CAR, MS2-Cre, and vsv-g induced GFP expression in CD19+ target cells starting around 20 hours of co-culture (Red trace in B and images in C). We saw minimal background fluorescence in all control conditions over the 48 hours. As hoped, GFP+ cell counts continued to increase through the end of the experiment.

### Example 40. CAR-Mediated Transfer by Human Primary Regulatory T Cells (Tregs)

### Donor Cells:

1) J76: Donor cells derived from Jurkat cells. The native TCR has been knocked out.
2) J76-CAR: stable T cell line expressing CD19 CAR-mRuby
3) Isolated human Tregs (CD4+, CD25+)
4) Isolated human Tregs (CD4+, CD25+) transduced with CD19 CAR-mRuby lentivirus

### Acceptor Cells:

Ramos cells: B cell line that expresses CD19.

### Cell Identity Labeling:

To distinguish donor cells from acceptor cells, both were labeled with unique dyes that covalently crosslink to cytoplasmic structures (Thermo CellTracker dyes).

### Cell-Cell Contact:

Donor and acceptor cells from each of the above groups were subjected to pairwise co-culture (4 conditions). The cells were first monocultured separately in suspension media. To co-culture, acceptor cells were added to donor cell cultures at a 1:1 ratio (donor to acceptor). The co-cultured cells were incubated at 37° C. for 1 hour.

### Measurement:

Transfer of CAR(mRuby) from donors to acceptors was measured by flow cytometry. All co-cultured cells were stained with viability dye. Live Ramos cells from each co-culture were gated on CellTracker then assessed for acquisition the CAR by mRuby fluorescence.

### Results:

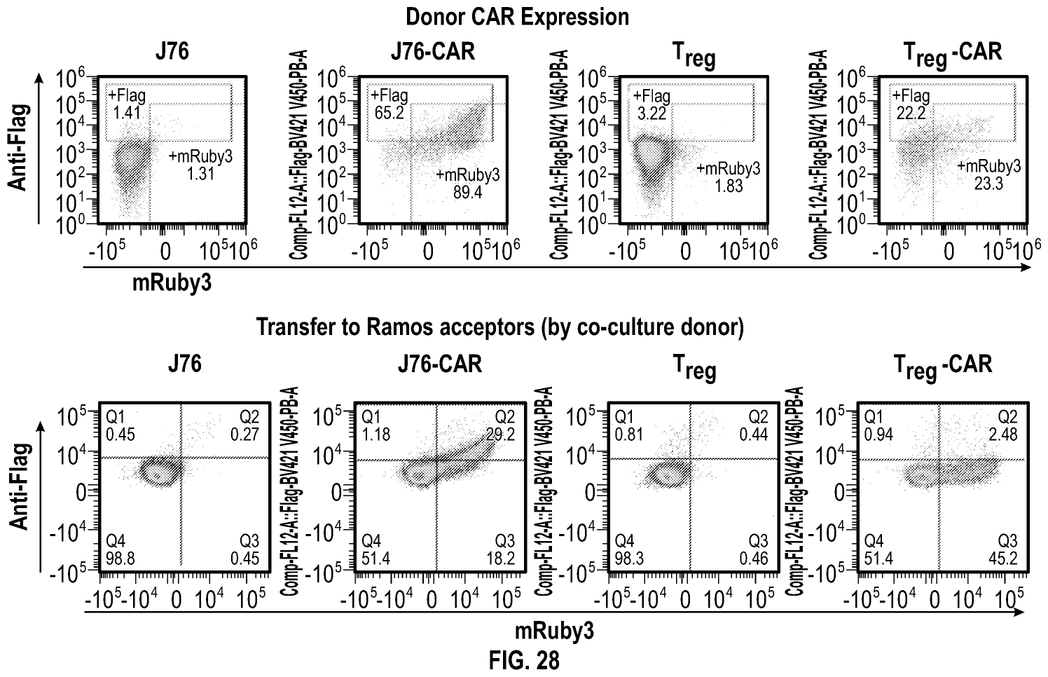

As shown in FIG. 28 (upper panels), transduced Tregs transferred expressed the CAR in a similar pattern to stable J76-CAR cells. The transduced Tregs were also able to transfer the CAR to acceptor Ramos cells (FIG. 28, lower panels).

### Example 41. Second Generation CARs Containing Both CD3z and CD28 Domains

CAR design: The CD19 CAR-mRuby CAR is modified to include a CD28 intracellular domain in between the transmembrane domain and the CD3z domain. This second generation CAR is inserted into a lentiviral vector to allow transduction of primary cells.

In vivo testing: The second generation CAR is compared to the original CAR for in vivo migration and persistence. Primary human Tregs are transduced with virus, expanded and introduced into humanized mice.

Analysis: Tissues are collected at regular time points to test relative survival and expansion of the CAR Tregs.

### Example 42. Generation of a Stable CAR-Expressing Jurkat Cell Line Using Transposons

Cells: J76 cell line (a TCR-knockout Jurkat T cell line)

Modifications: J76 cells were electroporated with a sleeping beauty donor construct encoding CD19CAR-mRuby and sleeping beauty transposase.

After electroporation, the cells were assessed for stable expression of CD19CAR-mRuby to confirm that the DNA had been stably integrated into the genome. The resulting cell line expressed CD19CAR-mRuby permanently.

### Example 43. Targeted Delivery to B Cells In Vivo

In this example, donor cells were engineered to transfer a CAR to human B cells in humanized mice with the goal of demonstrating that cells can be programmed to transfer to B cells in vivo.

### Donor Cells:

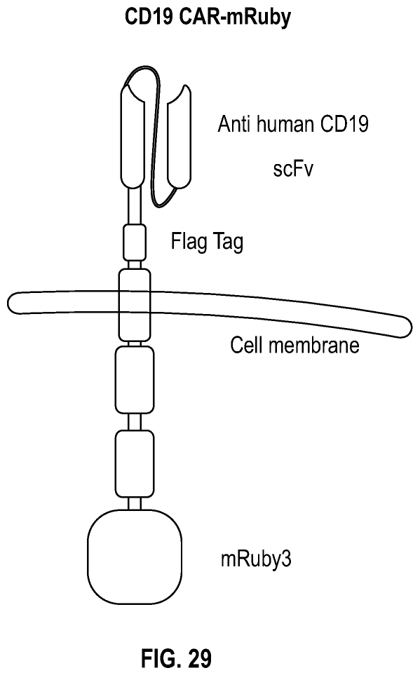

1) J76: Donor cells derived from Jurkat cells. The native TCR has been knocked out.
2) J76-CAR: stable T cell line expressing CD19 CAR-mRuby.
3) hTreg-CAR: primary human Tregs transduced with lentivirus to express CD19 CAR-mRuby. The hTregs were HLA-02 positive.
   (CD19 CAR-mRuby sequence is attached and FIG. 29 shows a schematic of the structure.)

Sequence:

>CD19 CAR-mRuby
ATGGCCCTGCCCGTGACCGCCCTCCTCCTGCCACTCGCCCTGCTGCTGCATGCAGCTCGGCCTGACATC
CAGATGACCCAGACTACATCTTCCCTGAGCGCAAGCCTGGGGGATAGGGTGACAATATCCTGCCGGGC
CAGCCAGGATATCAGCAAATACCTGAACTGGTATCAGCAGAAGCCCGATGGAACCGTGAAGCTGCTC
ATCTATCACACGAGCCGGCTGCACTCAGGCGTGCCCAGCAGGTTCTCTGGCTCTGGCAGCGGTACTGA
TTATTCACTGACAATCAGCAATCTGGAACAGGAGGACATCGCCACCTACTTCTGCCAGCAGGGTAACA
CCCTGCCCTACACTTTTGGCGGGGGAACCAAGCTGGAGATCACCGGCGGCGGCGGCAGCGGCGGGGG
AGGCTCCGGTGGGGGCGGGTCCGAGGTGAAGCTGCAAGAGAGCGGCCCCGGCCTCGTGGCCCCTTCTC
AGAGCCTGTCCGTGACCTGCACTGTGTCAGGAGTGAGCCTGCCAGATTATGGGGTGAGCTGGATCAGA
CAGCCCCCCCGGAAGGGCCTGGAGTGGCTGGGCGTGATCTGGGGTAGCGAGACCACATACTATAACTC
TGCCCTGAAAAGCAGACTGACCATCATTAAGGATAACAGCAAGTCTCAGGTGTTCCTGAAAATGAACT
CCCTTCAGACAGACGACACAGCCATCTATTACTGCGCAAAACACTACTACTATGGGGGCTCCTACGCT
ATGGATTATTGGGGCCAAGGCACCAGCGTGACTGTGAGCAGTGACTATAAGGATGACGACGATAAGA
CCGGTACCCCTGCTCCTAGACCTCCTACACCAGCTCCTACAATCGCCAGCCAGCCTCTGTCTCTGAGGC
CAGAAGCTTGTAGACCTGCTGCTGGCGGAGCCGTGCATACAAGAGGACTGGATTTCGCCTGCGACTTC
TGGGTGCTCGTGGTTGTTGGCGGAGTGCTGGCCTGTTACAGCCTGCTGGTTACCGTGGCCTTCATCATC
TTTTGGGTCCGAAGCAAGCGGAGCCGGCTGCTGCACAGCGACTACATGAACATGACCCCTAGACGGCC
CGGACCTACCAGAAAGCACTACCAGCCTTACGCTCCTCCTAGAGACTTCGCCGCCTACAGATCTAGAG
TGAAGTTTAGCCGGAGCGCCGATGCCCCTGCCTATCAGCAGGGACAGAACCAGCTGTATAACGAGCTG
AACCTGGGCAGGAGAGAGGAGTACGATGTGCTGGATAAACGCCGCGGAAGAGACCCCGAAATGGGC
GGCAAGCCACGCCGCAAAAACCCACAGGAGGGCCTTTACAACGAGCTGCAGAAGGACAAGATGGCCG
AGGCCTACTCCGAAATTGGCATGAAAGGGGAACGGCGCAGGGGAAAGGGCCACGACGGCCTGTACCA
GGGCCTGTCAACCGCCACCAAGGATACCTACGATGCTCTGCATATGCAGGCCCTGCCACCTCGCCCCC
CTGTGGCTACAATGGTGAGTAAGGGCGAGGAGCTGATCAAGGAGAATATGAGAATGAAGGTCGTGAT
GGAGGGCTCCGTGAACGGACACCAGTTTAAGTGTACAGGAGAGGGGGAGGGCAGACCGTACGAGGG
GGTGCAGACCATGCGCATTAAGGTGATCGAGGGCGGGCCTCTGCCCTTTGCTTTCGACATCCTGGCAA
CCAGCTTCATGTATGGCTCCAGGACATTTATCAAGTACCCTGCCGACATCCCCGATTTTTTCAAGCAGA
GCTTCCCCGAGGGCTTCACCTGGGAGAGAGTCACCCGCTACGAGGACGGAGGGGTGGTGACAGTGAC
ACAGGACACCAGCCTGGAGGATGGAGAGCTGGTGTACAACGTCAAGGTGCGGGGAGTGAATTTCCCA
TCAAATGGACCCGTCATGCAGAAGAAGACCAAGGGATGGGAGCCTAATACTGAGATGATGTACCCTG
CCGACGGAGGCCTGAGGGGATACACAGATATCGCCCTGAAGGTGGACGGGGGAGGCCATCTGCATTG
CAATTTCGTGACCACATACCGGAGCAAGAAGACCGTCGGCAACATCAAGATGCCCGGCGTGCATGCA
GTGGACCACAGGCTGGAGCGGATCGAAGAGTCTGATAACGAGACCTACGTGGTGCAGAGGGAGGTGG
CCGTCGCCAAATACAGCAATCTCGGCGGGGGCATGGACGAGCTGTACAAGTAA (SEQ ID NO: 42)

### Recipient Animals:

Hu-CD34+NSG (Jax stock #000664): Hu-CD34+NSG are NSG (NOD.Cg-Prkdcscid Il2rgtm1Wjl, Jackson Labs Stock No. 005557) mice engrafted with cord blood-dcrivcd CD34+ hematopoietic stem cells (HSCs) from healthy humans that were HLA-02 negative. CD34+ humanized-NSG mice exhibit stable, multi-lineage engraftment of functional human hematopoietic cells without GvHD. This includes B cells, which were the target cell population in this example.

### Experimental Groups:

Hu-CD34⁺ mice were injected with each of the donor cells groups as outlined in the table below:

| Donor Cell Type | Group 1 | Group 2 | Group 3 |
|---|---|---|---|
| (# of cells) | (1 hr) | (24 hr) | (48 hr) |
| J76 | 2 mice | 2 mice | 2 mice |
| (10 million) | | | |
| J76-CAR | 3 mice | 3 mice | 3 mice |
| (10 million) | | | |
| h Treg-CAR | 2 mice | 2 mice | 2 mice |
| (6.5 million) | | | |

### Cell Identity Labeling:

To distinguish donor cells in vivo, they were labeled with a dye that covalently crosslinked to cytoplasmic structures (Thermo CellTracker dye). These dyes have been shown to stably label cells for in vivo tracking.

### Cell Injections:

Donor cells (in 100 uL of sterile saline) were injected into recipient animals via tail vein injection.

### Measurement:

At 1 hour, 1 days and 2 days post-injection, animals were euthanized and tissues were harvested, including blood, spleen, bone marrow, lungs, and liver. The tissues were each dissociated into single-cell suspensions, stained with antibodies to distinguish cell types, and subjected to flow cytometry. The presence of donor cells in the tissues was measured as well as the incidence of transfer of the CD19 CAR-mRuby to human B cells. The CAR was identified by measuring mRuby (fused to the C-terminus of the CAR) fluorescence (FIG. 29).

### Results:

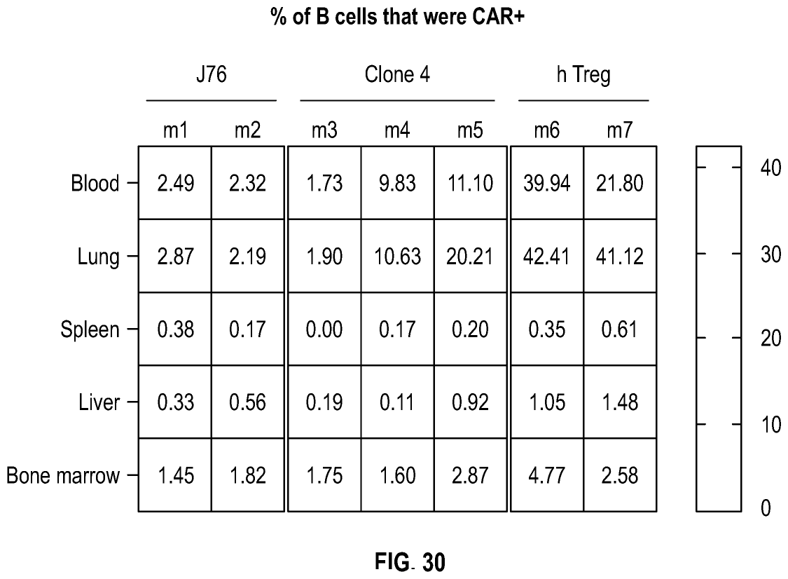

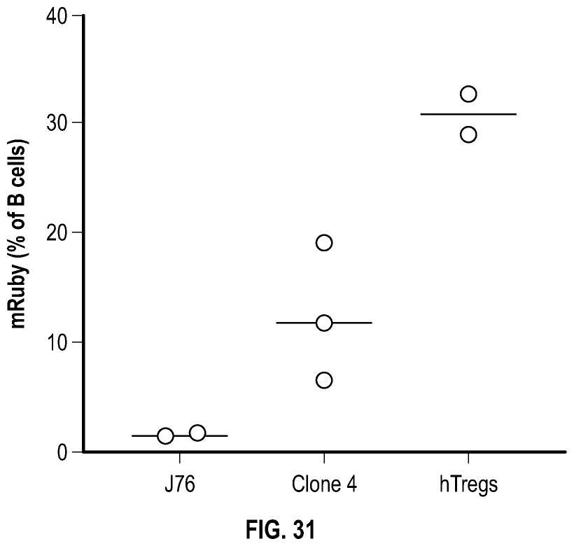

Significant transfer to B cells of the blood and lung was observed in the 1 hour group (FIG. 30). Little transfer was evident at the two later time points, likely due to loss of the donor cells, which may have been killed by allogeneic T cells of the humanized mice. FIG. 30 shows data for the 1 hour time point across all tissues. At 1 hour post-adoptive transfer, a substantially high frequency of human B cells that had received the CAR (mRuby) were detected in both the blood and the lungs (FIG. 31), with the highest transfer occurring in the hTreg group. The hTreg group also showed the greatest mean fluorescence intensity (MFI) among transfer-positive B cells, which is a measure of the amount of CAR transferred to each target cell.

### Example 44. Transfer Using Adipocyte Stem Cells (ASCs) as Donor Cells

In this example, adiocyte stem cells (ASCs), also referred to as adipose-derived mesenchymal stem cells, were engineered to express and transfer a CD19-targeted CAR to a human, CD19+ cell line in vitro. ASCs were derived from healthy, non-obese donor adipose tissue. After isolation from adipose tissue, ASCs were able to be passaged and expanded in culture. This example demonstrates that ASCs can be used as donor cells.

### Donor Cells:

1) Primary human ASCs
2) Primary human ASCs engineered with CD19CAR-mRuby
   (CD19 CAR-mRuby sequence is as shown in Example 21. A schematic of the structure is shown in FIG. 29.)

### Target Cells:

T2 cells (a human cell line that expresses CD19).

### Cell Identity Labeling:

To distinguish donor cells from targets, both were labeled with a dye that covalently crosslinks to cytoplasmic structures (Thermo CellTracker dye).

### Cell-Cell Contact:

Donor and acceptor cells from each of the above groups were subjected to pairwise co-culture (2 combinations). The cells were first monocultured separately in suspension media after labeling. To co-culture, acceptor cells were added to donor cell cultures at a 1:1 ratio (donor to acceptor). The co-cultured cells were centrifuged at 100 g to force the cells into contact, then incubated at 37° C. for 1 hour, then placed on ice before processing for flow cytometry.

### Measurement:

Transfer of the CAR from donors to acceptors was measured by flow cytometry. All co-cultured cells were stained with viability dye. Live target cells from each co-culture are gated on the appropriate CellTracker then assessed for mRuby fluorescence, indicating transfer of the CAR.

### Results:

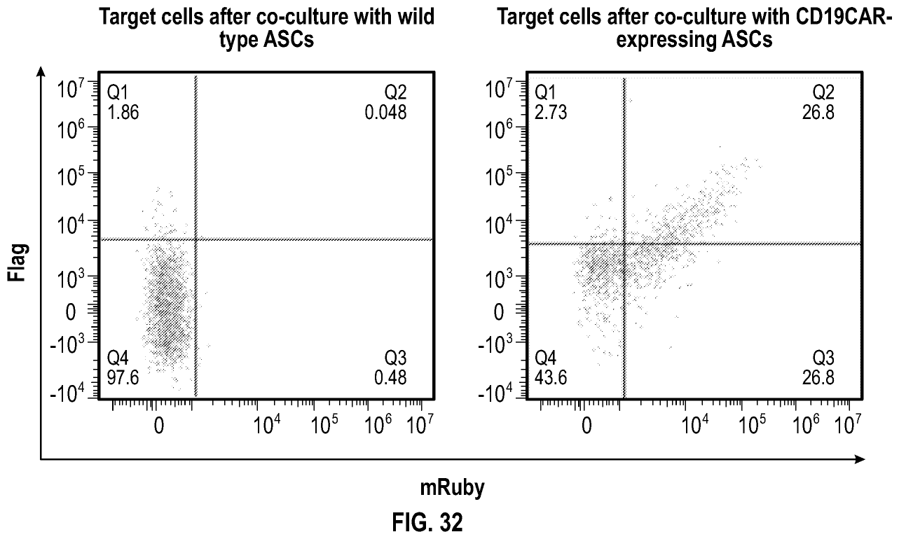

Transfer of CD19CAR-mRuby to acceptor cells was measured. Transfer incidence, measured as the percent of acceptor cells that acquired the CAR is summarized in FIG. 32. As shown in FIG. 32, approximately 54% of target cells were positive for the CAR (i.e., mRuby+).

### Example 45: CRISPR Knock-In to the TCR Locus

Donor cell: Human primary regulatory T cells from healthy donors

RNP preparation: Ribonucleoprotein complexes (RNPs) were formed by mixing pure CAS-9 protein with RNA guide oligonucleotides targeting human TCRα and TCRβ, at a 1:5 ratio.

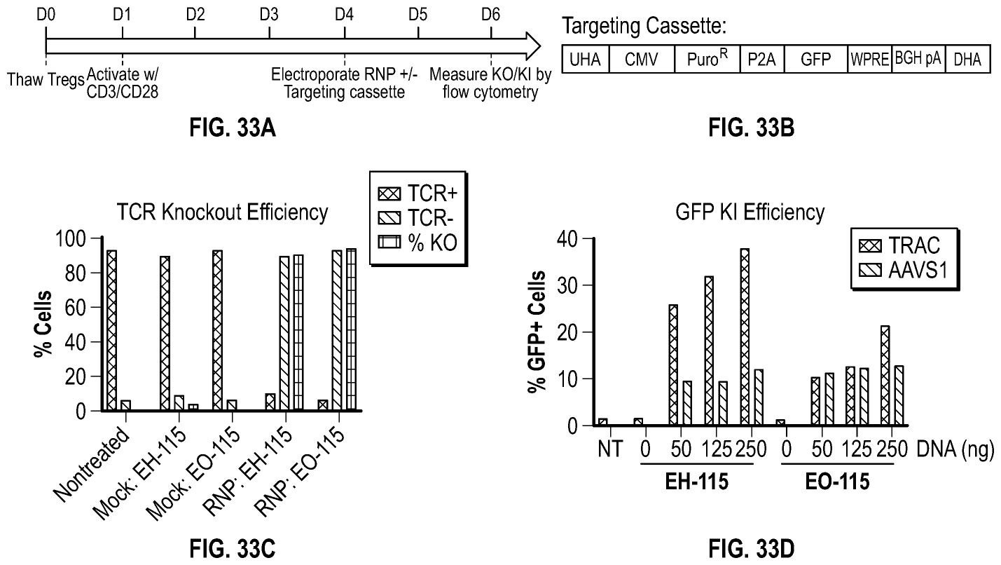

DNA donor template: A DNA cassette encoding Puromycin and GFP was flanked by upstream and downstream homology arms from DNA regions upstream and downstream of endogenous TCRα (FIG. 33B).

Procedure: Purchased CD4/CD25 Tregs were thawed, then one day later, were activated with CD3/CD28 Dynabeads. For each condition, 2 million activated T cells were electroporated with RNPs and donor template, 2 days after activation. Knockout and knock-in were evaluated 2 days after electroporation (FIG. 33A).

Measurement: Loss of TCR was assessed using flow cytometry for CD3, which requires the TCR for proper surface expression. Expression of GFP was also measured to assess knock-in efficiency.

Results: TCR knockout efficiency was very high for all the RNP-electroporated conditions. Maximum knockout efficiency was around 94%, with minimal differences observed between the two electroporation conditions tested (FIG. 33C). GFP expression was detected in up to 38% of Tregs when the targeting cassette was co-delivered with RNP. Efficiency increased with increasing dose of the donor DNA (FIG. 33D).

### Example 46. mRNA Delivery of CARs to Primary Tregs

Donor cell: Human primary regulatory T cells from healthy donors

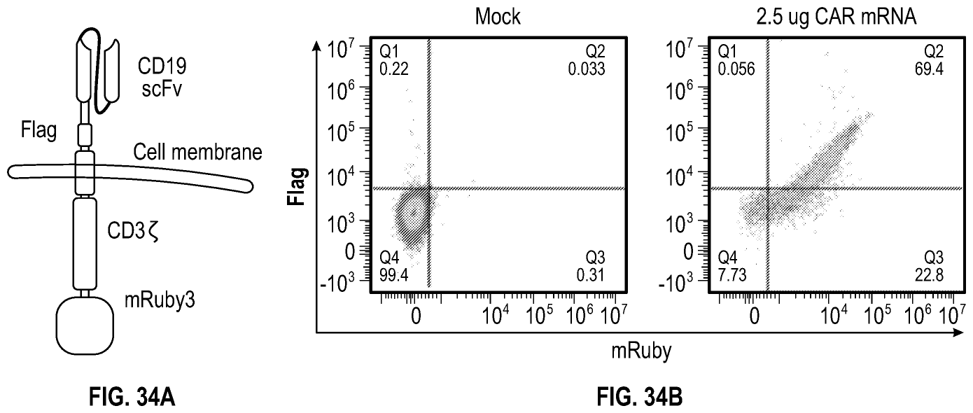

mRNA: mRNA encoding a CD19 CAR-mRuby was synthesized by TriLink. The CAR includes an extracellular Flag tag and an intracellular fusion to mRuby3 (FIG. 34A).

Procedure: Purchased CD4/CD25 Tregs were thawed, then one day later, were activated with CD3/CD28 Dynabeads. For each condition, 2 million activated T cells were electroporated with mRNA, 2 days after activation.

Measurement: CAR expression was measured using flow cytometry for mRuby3 and antibody-labeled Flag tag.

Results: Strong expression of the CAR was measured by both mRuby and Flag staining. Notably, the mRuby indicated total levels of the CAR, while Flag-staining only detected CAR that was present on the surface of the cell, available for antibody binding. This gave a good indication of the ratio of functional surface CAR relative to CAR that was transiting through endosomal and secretory pathways. In total, CAR was detected on greater than 90% of the cells, with the majority being surface-exposed (FIG. 34B).

1. A donor cell comprising:
(a) a membrane-associated agent comprising: (i) a membrane-associated moiety, (ii) a first docking moiety, and (iii) an intracellular moiety; and
(b) a cargo molecule;
wherein the donor cell is deficient for cytotoxic activity, is T cell receptor (TCR)-deficient, or is MHC-deficient.

2. The donor cell of claim 1, wherein the donor cell is T cell receptor deficient (TCR deficient).

3. The donor cell of claim 1, wherein the donor cell is MHC deficient.

4. The donor cell of claim 1, wherein the cargo molecule comprises a therapeutic agent.

5. The donor cell of claim 1, wherein the cargo molecule is an exogenous cargo molecule that binds to the intracellular moiety.

6. The donor cell of claim 1, wherein the cargo molecule comprises a gene modifying polypeptide.

7. The donor cell of claim 1, wherein the cargo molecule comprises a recombinase.

8. The donor cell of claim 1, wherein the cargo molecule comprises a retrotransposase.

9. The donor cell of claim 1, wherein the cargo molecule comprises a protein comprising a CRISPR/Cas9 domain.

10. The donor cell of claim 1, wherein the cargo molecule comprises a membrane protein.

11. The donor cell of claim 1, wherein the cargo molecule comprises a protein comprising a nuclear localization signal.

12. The donor cell of claim 1, wherein the cargo molecule comprises one or more of:
(i) a cytosolic protein;
(ii) a membrane protein;
(iii) a secreted protein;
(iv) a nuclear protein; and/or
(v) an organellar protein.

13-16. (canceled)

17. A donor cell comprising:
(a) a membrane-associated agent comprising: (i) a membrane-associated moiety, (ii) a first docking moiety, and (iii) an intracellular moiety; and
(b) a cargo molecule;
wherein the membrane-associated agent comprises a CAR or an exogenous TCR; and
wherein the donor cell substantially lacks cytotoxic activity, optionally wherein the donor cell is a regulatory T cell (Treg).

18. A donor cell comprising:
(a) a membrane-associated agent comprising: (i) a membrane-associated moiety, (ii) a first docking moiety, and (iii) an intracellular moiety; and
(b) a cargo molecule;
wherein the donor cell has one or more of the following characteristics:
(i) the cargo molecule is attached to a cell-penetrating peptide;
(ii) the membrane-associated agent is attached to a cell-penetrating peptide;
(iii) the donor cell comprises a cell-penetrating peptide;
(iv) the donor cell comprises a fusogen;
(vii) the donor cell comprises: (1) a nucleic acid molecule comprising a promoter and a gene encoding the membrane-associated agent and/or a gene encoding the cargo molecule; or (2) a nucleic acid molecule encoding the membrane-associated agent and/or a nucleic acid molecule encoding the cargo molecule;
(viii) the donor cell comprises an inducible cell death agent; and/or
(ix) the donor cell is or is derived from an induced pluripotent stem cell (iPSC).

19. A preparation comprising:
a) a first plurality of donor cells according to claim 1, and
b) a second plurality of cells that are not donor cells according to claim 1.

20. The preparation of claim 19, wherein at least 50%, 60%, 70%, 80%, 85%, 90%, 95%, 96%, 97%, 98%, or 99% of the cells in the preparation are cells of the first plurality.

21. The preparation of claim 19, wherein at least 1%, 2%, 3%, 4%, 5%, 10%, 15%, 20%, 25%, 30%, 40%, or 50% of the cells in the preparation are cells of the first plurality.

22. A method of modifying an acceptor cell, the method comprising:
contacting the acceptor cell with a donor cell of claim 1 under conditions suitable for transfer of the membrane-associated agent and/or the cargo molecule to the acceptor cell.

23. A method of producing the donor cell of claim 1, the method comprising:
(i) inactivating at least one endogenous function of a cell; and
(ii) introducing into or expressing in the cell:
(a) a membrane-associated agent comprising: (i) a membrane-associated moiety, (ii) a first docking moiety, and (iii) an intracellular moiety; and
(b) a cargo molecule;
thereby producing the donor cell of claim 1.
